(19) United States
(12) Patent Application Publication
(10) Pub. No.: US 2024/0067738 A1
(43) Pub. Date: Feb. 29, 2024

(54) ANTI-IL4 RECEPTOR ANTIBODIES FOR VETERINARY USE

(71) Applicant: Elanco US Inc., Greenfield, IN (US)

(72) Inventors: Shyr Jiann LI, Burlingame, CA (US); Lam NGUYEN, Burlingame, CA (US); Richard CHIN, Burlingame, CA (US); Hangjun ZHAN, Burlingame, CA (US); Qingyi CHU, Burlingame, CA (US)

(21) Appl. No.: 17/906,297

(22) PCT Filed: Mar. 17, 2021
(86) PCT No.: PCT/US2021/022706

§ 371 Date: Sep. 14, 2022

Related U.S. Application Data
(60) Provisional application No. 62/991,568, filed on Mar. 18, 2020.

(51) Int. Cl. C07K 16/28; A61P 37/02; G01N 33/68

(52) U.S. Cl. CPC C07K 16/2866; A61P 37/02; G01N 33/68; C07K 2317/14; C07K 2317/31; C07K 2317/33; C07K 2317/52; C07K 2317/565

(57) ABSTRACT

Provided are various embodiments relating to anti-IL4R antibodies that bind to canine IL4R. In various embodiments, such anti-IL4R antibodies can be used in methods to treat IL4/IL13-induced conditions, such as atopic dermatitis, allergic dermatitis, pruritus, asthma, psoriasis, scleroderma and eczema, in companion animals, such as canines and felines. Also provided are various embodiments relating to variant IgG Fc polypeptides and variant light chain constant regions of companion animal species for the preparation of antibodies or bispecific antibodies.

This application claims the benefit of U.S. Provisional Application No. 62/991,568, filed Mar. 18, 2020, which is incorporated by reference herein in its entirety for any purpose.

### SEQUENCE LISTING

This application is filed with a Sequence Listing in electronic format. The Sequence Listing is provided as a file entitled “01157-0032-00PCT_ST25.txt” created on Mar. 17, 2021, which is 774 KB in size. The information in the electronic format of the sequence listing is incorporated herein by reference in its entirety.

### FIELD

This disclosure relates to isolated anti-IL4 receptor (IL4R) antibodies, for example, binding to canine or feline IL4R and reducing binding with IL4 or IL13, and methods of using the same, for example, treating IL4-induced and/or IL13-induced conditions or reducing IL4 or IL13 signaling function in cells, for instance in companion animals, such as canines and felines.

### BACKGROUND

Interleukin 4 (IL4) is a cytokine that induces naïve T helper cells to differentiate to Th2 cells. IL4 can also stimulate activated B cell and T cell proliferation and induce B cell class switching to IgE. IL13 has similar effect on immune cells as IL4. Both cytokines are associated with allergies.

IL4 receptor is known as IL4Ralpha or IL4R. IL4R can pair with a common gamma chain receptor and specifically bind IL4. IL4R can also pair with IL13Ra1 and together they can bind either IL4 or IL13. Thus, blocking binding sites on IL4R can potentially reduce binding of IL4 and/or IL13 and reduce signaling effect of these two cytokines.

Companion animals, such as cats, dogs, and horses, suffer from many skin diseases similar to human skin diseases, including atopic dermatitis and allergic conditions. There remains a need, therefore, for methods and compounds that can be used specifically to block companion animal IL4 or/and IL13 for treating IL4-induced and/or IL13-induced conditions and for reducing IL4/IL13 signaling.

### SUMMARY

Embodiment 1. An isolated antibody that binds to canine IL4R or feline IL4R, wherein the antibody binds to an epitope comprising the amino acid sequence of LX₁₀ FMGSENX₁₁T (SEQ ID NO: 85), wherein X₁₀ is D or N and X₁₁ is H or R, and wherein the antibody comprises a variant canine or feline IgG Fc polypeptide capable of binding to neonatal Fc receptor (FcRn) with an increased affinity relative to the wild-type Fc polypeptide.

Embodiment 2. An isolated antibody that binds to canine IL4R or feline IL4R, wherein the antibody binds to an epitope comprising the amino acid sequence of RLSYQLX₁₀FMGSENX₁₁TCYPEN (SEQ ID NO: 86), wherein X₁₀ is D or N and X₁₁ is H or R, wherein the antibody comprises a variant canine or feline IgG Fc polypeptide capable of binding to neonatal Fc receptor (FcRn) with an increased affinity relative to the wild-type Fc polypeptide.

Embodiment 3. The isolated antibody of embodiment 2, wherein the antibody binds to an epitope comprising the amino acid sequence of LX₁₀FMGSENX₁₁T (SEQ ID NO: 85), wherein X₁₀ is D or N and X₁₁ is H or R.

Embodiment 4. The isolated antibody of any one of the preceding embodiments, wherein the antibody binds to an epitope comprising the amino acid sequence of SEQ ID NO: 88 or SEQ ID NO: 91.

Embodiment 5. The isolated antibody of any one of the preceding embodiments, wherein the antibody binds to an epitope comprising the amino acid sequence of SEQ ID NO: 89 or SEQ ID NO: 92.

Embodiment 6. The isolated antibody of any one of the preceding embodiments, wherein the antibody binds to an epitope comprising the amino acid sequence of SMX₁₂X₁₃DDX₁₄VEADVYQLX₁₅LWAGX₁₆Q (SEQ ID NO: 87), wherein X₁₂ is P or L, X₁₃ is I or M, X₁₄ is A or F, X₁₅ is D or H, and X₁₆ is Q or T.

Embodiment 7. An isolated antibody that binds to canine IL4R or feline IL4R, wherein the antibody binds to an epitope comprising the amino acid sequence of SMX₁₂X₁₃DDX₁₄VEADVYQLX₁₅LWAGX₁₆Q (SEQ ID NO: 87), wherein X₁₂ is P or L, X₁₃ is I or M, X₁₄ is A or F, X₁₅ is D or H, and X₁₆ is Q or T, and wherein the antibody comprises a variant canine or feline IgG Fc polypeptide capable of binding to neonatal Fc receptor (FcRn) with an increased affinity relative to the wild-type Fc polypeptide.

Embodiment 8. The isolated antibody of any one of the preceding embodiments, wherein the antibody binds to an epitope comprising the amino acid sequence of SEQ ID NO: 90 or SEQ ID NO: 93.

Embodiment 9. The isolated antibody of any one of the preceding embodiments, wherein the antibody binds to canine IL4R or feline IL4R with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁹ M, less than 1×10⁻¹⁹ M, less than 5×10⁻¹¹ M, less than 1×10⁻¹¹ M, less than 5×10⁻¹² M, or less than 1×10⁻¹² M, as measured by biolayer interferometry.

Embodiment 10. The antibody of any one of the preceding embodiments, wherein the antibody binds to canine IL4R or feline IL4R as determined by immunoblot analysis or biolayer interferometry.

Embodiment 11. The isolated antibody of any one of the preceding embodiments, wherein the antibody reduces binding of a canine and/or feline IL4 polypeptide and/or a canine and/or feline IL13 polypeptide to canine IL4R and/or feline IL4R, as measured by biolayer interferometry.

Embodiment 12. The isolated antibody of any one of the preceding embodiments, wherein the antibody competes with monoclonal Clone B or Clone I antibody in binding to canine IL4R or feline IL4R.

Embodiment 13. The isolated antibody of any one of the preceding embodiments, wherein the antibody is a monoclonal antibody.

Embodiment 14. The isolated antibody of any one of the preceding embodiments, wherein the antibody is a canine, a caninized, a feline, a felinized, or a chimeric antibody.

Embodiment 15. The isolated antibody of any one of the preceding embodiments, wherein the antibody is a chimeric antibody comprising one or more murine variable heavy chain framework regions or one or more murine variable light chain framework regions.

Embodiment 16. An isolated antibody that binds to canine IL4R or feline IL4R, wherein the antibody comprises a heavy chain comprising:
a) a CDR-H1 sequence having at least 85%, at least 90%, at least 95%, at least 98%, or 100% sequence identity to the amino acid sequence of GYTFTSYVMX₁ (SEQ ID NO: 1), wherein X1 is H or N;
b) a CDR-H2 sequence having at least 85%, at least 90%, at least 95%, at least 98%, or 100% sequence identity to the amino acid sequence of YINPX₂NDGTFYX₃GX₄X₅X₆G (SEQ ID NO: 2), wherein X₂ is K, A, or N, X₃ is N or A, X₄ is K or A, X₅ is F or V, and X₆ is K or Q, or YINPX₂NDGT (SEQ ID NO: 268), wherein X₂ is K, A, or N; and
c) a CDR-H3 sequence having at least 85%, at least 90%, at least 95%, at least 98%, or 100% sequence identity to the amino acid sequence of FX₇YGX₈AY (SEQ ID NO: 3), wherein X₇ is N or Y, and X₈ s I or F; and
wherein the antibody comprises a variant canine or feline IgG Fc polypeptide capable of binding to neonatal Fc receptor (FcRn) with an increased affinity relative to the wild-type Fc polypeptide.

Embodiment 17. The isolated antibody of any one of the preceding embodiments, comprising a heavy chain comprising:
a) a CDR-H1 sequence having at least 85%, at least 90%, at least 95%, at least 98%, or 100% sequence identity to the amino acid sequence of GYTFTSYVMX₁ (SEQ ID NO: 1), wherein X₁ is H or N;
b) a CDR-H2 sequence having at least 85%, at least 90%, at least 95%, at least 98%, or 100% sequence identity to the amino acid sequence of YINPX₂NDGTFYX₃GX₄X₅X₆G (SEQ ID NO: 2), wherein X ₂ is K, A, or N, X ₃ is N or A, X ₄ is K or A, X₅ is F or V, and X₆ is K or Q, or YINPX₂NDGT (SEQ ID NO: 268), wherein X₂ is K, A, or N; and
c) a CDR-H3 sequence having at least 85%, at least 90%, at least 95%, at least 98%, or 100% sequence identity to the amino acid sequence of FX₇YGX₈AY (SEQ ID NO: 3), wherein X₇ is N or Y, and X₈ s I or F.

Embodiment 18. The isolated antibody of any one of the preceding embodiments, comprising a heavy chain comprising:
a) a CDR-H1 sequence having at least 85%, at least 90%, at least 95%, at least 98%, or 100% sequence identity to the amino acid sequence of SEQ ID NO: 7, SEQ ID NO: 29, or SEQ ID NO: 358;
b) a CDR-H2 sequence having at least 85%, at least 90%, at least 95%, at least 98%, or 100% sequence identity to the amino acid sequence of SEQ ID NO: 8, SEQ ID NO: 269, SEQ ID NO: 30, SEQ ID NO: 271, SEQ ID NO: 359, or SEQ ID NO: 272; and
c) a CDR-H3 sequence having at least 85%, at least 90%, at least 95%, at least 98%, or 100% sequence identity to the amino acid sequence of SEQ ID NO: 9 or SEQ ID NO: 31.

Embodiment 19. The isolated antibody of any one of the preceding embodiments, comprising a heavy chain comprising:
a) a CDR-H1 comprising the amino acid sequence of SEQ ID NO:7, SEQ ID NO: 29, or SEQ ID NO: 358;
b) a CDR-H2 comprising the amino acid sequence of SEQ ID NO: 8, SEQ ID NO: 269, SEQ ID NO: 30, SEQ ID NO: 271, SEQ ID NO: 359, or SEQ ID NO: 272; and
c) a CDR-H3 comprising the amino acid sequence of SEQ ID NO: 9 or SEQ ID NO: 31.

Embodiment 20. An isolated antibody that binds to canine IL4R or feline IL4R, wherein the antibody comprises a light chain comprising:
a) a CDR-L1 sequence having at least 85%, at least 90%, at least 95%, at least 98%, or 100% sequence identity to the amino acid sequence of RASQEISGYLX₉ (SEQ ID NO: 4) wherein X₉ is S or A;
b) a CDR-L2 sequence having at least 85%, at least 90%, at least 95%, at least 98%, or 100% sequence identity to the amino acid sequence of AASX₁₀X₁₁DX₁₂ (SEQ ID NO: 5), wherein X₁₀ is T or N, X₁₁ is R or L, and X₁₂ is S or T; and
c) a CDR-L3 sequence having at least 85%, at least 90%, at least 95%, at least 98%, or 100% sequence identity to the amino acid sequence of X₁₃QYASYPWT (SEQ ID NO: 6), wherein X₁₃ is V or L; and
wherein the antibody comprises a variant canine or feline IgG Fc polypeptide capable of binding to neonatal Fc receptor (FcRn) with an increased affinity relative to the wild-type Fc polypeptide.

Embodiment 21. The isolated antibody of any one of the preceding embodiments, comprising a light chain comprising:
a) a CDR-L1 sequence having at least 85%, at least 90%, at least 95%, at least 98%, or 100% sequence identity to the amino acid sequence of RASQEISGYLX₉ (SEQ ID NO: 4) wherein X₉ is S or A;
b) a CDR-L2 sequence having at least 85%, at least 90%, at least 95%, at least 98%, or 100% sequence identity to the amino acid sequence of AASX₁₀X₁₁DX₁₂ (SEQ ID NO: 5), wherein X₁₀ is T or N, X₁₁ is R or L, and X₁₂ is S or T; and
c) a CDR-L3 sequence having at least 85%, at least 90%, at least 95%, at least 98%, or 100% sequence identity to the amino acid sequence of X₁₃QYASYPWT (SEQ ID NO: 6), wherein X ₁₃ is V or L.

Embodiment 22. An isolated antibody that binds to canine IL4R or feline IL4R, wherein the antibody comprises:
a) a CDR-H1 sequence having at least 85%, at least 90%, at least 95%, at least 98%, or 100% sequence identity to the amino acid sequence of GYTFTSYVMX₁ (SEQ ID NO: 1), wherein X₁ is H or N;
b) a CDR-H2 sequence having at least 85%, at least 90%, at least 95%, at least 98%, or 100% sequence identity to the amino acid sequence of YINPX₂NDGTFYX₃GX₄X₅X₆G (SEQ ID NO: 2), wherein X₂ is K, A, or N, X₃ is N or A, X₄ is K or A, X₅ is F or V, and X₆ is K or Q, or YINPX₂NDGT (SEQ ID NO: 268), wherein X₂ is K, A, or N; and
c) a CDR-H3 sequence having at least 85%, at least 90%, at least 95%, at least 98%, or 100% sequence identity to the amino acid sequence of FX₇YGX₈AY (SEQ ID NO: 3), wherein X ₇ is N or Y, and X₈ s I or F;
d) a CDR-L1 sequence having at least 85%, at least 90%, at least 95%, at least 98%, or 100% sequence identity to the amino acid sequence of RASQEISGYLX₉ (SEQ ID NO: 4) wherein X₉ is S or A;
e) a CDR-L2 sequence having at least 85%, at least 90%, at least 95%, at least 98%, or 100% sequence identity to the amino acid sequence of AASX₁₀X₁₁DX₁₂ (SEQ ID NO: 5), wherein X₁₀ is T or N, X₁₁ is R or L, and X₁₂ is S or T; and
f) a CDR-L3 sequence having at least 85%, at least 90%, at least 95%, at least 98%, or 100% sequence identity to the amino acid sequence of X₁₃QYASYPWT (SEQ ID NO: 6), wherein X₁₃ is V or L; and
wherein the antibody comprises a variant canine or feline IgG Fc polypeptide capable of binding to neonatal Fc receptor (FcRn) with an increased affinity relative to the wild-type Fc polypeptide.

Embodiment 23. The isolated antibody of any one of the preceding embodiments, comprising a light chain comprising:
a) a CDR-L1 sequence having at least 85%, at least 90%, at least 95%, at least 98%, or 100% sequence identity to the amino acid sequence of SEQ ID NO: 14, SEQ ID NO: 36, or SEQ ID NO: 360;
b) a CDR-L2 sequence having at least 85%, at least 90%, at least 95%, at least 98%, or 100% sequence identity to the amino acid sequence of SEQ ID NO: 15, SEQ ID NO: 37, SEQ ID NO: 361, or SEQ ID NO: 362; and
c) a CDR-L3 sequence having at least 85%, at least 90%, at least 95%, at least 98%, or 100% sequence identity to the amino acid sequence of SEQ ID NO: 16 or SEQ ID NO: 38.

Embodiment 24. The isolated antibody of any one of the preceding embodiments, comprising a light chain comprising:
a) a CDR-L1 comprising the amino acid sequence of SEQ ID NO: 14, SEQ ID NO: 36, or SEQ ID NO: 360;
b) a CDR-L2 comprising the amino acid sequence of SEQ ID NO: 15, SEQ ID NO: 37, SEQ ID NO: 361, or SEQ ID NO: 362; and
c) a CDR-L3 comprising the amino acid sequence of SEQ ID NO:16 or SEQ ID NO: 38.

Embodiment 25. The antibody of any one of embodiments 16 to 24, further comprising one or more of (a) a variable region heavy chain framework 1 (HC-FR1) sequence of SEQ ID NO: 10 or SEQ ID NO: 32; (b) a HC-1-R2 sequence of SEQ ID NO: 11 or SEQ ID NO: 33; (c) a HC-FR3 sequence of SEQ ID NO: 12, SEQ ID NO: 270, SEQ ID NO: 34, SEQ ID NO: 273; (d) a HC-FR4 sequence of SEQ ID NO: 13 or SEQ ID NO: 35; (e) a variable region light chain framework 1 (LC-FR1) sequence of SEQ ID NO: 17 or SEQ ID NO: 39; (f) an LC-FR2 sequence of SEQ ID NO: 18 or SEQ ID NO: 40; (g) an LC-FR3 sequence of SEQ ID NO: 19 or SEQ ID NO: 41; or (h) an LC-FR4 sequence of SEQ ID NO: 20 or SEQ ID NO: 42.

Embodiment 26. The antibody of any one of the preceding embodiments, wherein the antibody comprises:
a. (i) a variable heavy chain sequence having at least 85%, at least 90%, at least 95%, at least 98%, or 100% sequence identity to the amino acid sequence of SEQ ID NO: 21 or SEQ ID NO: 43; (ii) a variable light chain sequence having at least 85%, at least 90%, at least 95%, at least 98%, or 100% sequence identity to the amino acid sequence of SEQ ID NO: 22 or SEQ ID NO: 44; or (iii) a variable heavy chain sequence as in (i) and a variable light chain sequence as in (ii); or
b. (i) a variable heavy chain sequence having at least 85%, at least 90%, at least 95%, at least 98%, or 100% sequence identity to the amino acid sequence of SEQ ID NO: 59, SEQ ID NO: 60, SEQ ID NO: 63, SEQ ID NO: 64, SEQ ID NO: 274, or SEQ ID NO: 363; (ii) a variable light chain sequence having at least 85%, at least 90%, at least 95%, at least 98%, or 100% sequence identity to the amino acid sequence of SEQ ID NO: 61, SEQ ID NO: 62, SEQ ID NO: 65, SEQ ID NO: 66, SEQ ID NO: 275, or SEQ ID NO: 364; or (iii) a variable heavy chain sequence as in (i) and a variable light chain sequence as in (ii); or
c. (i) a variable heavy chain sequence having at least 85%, at least 90%, at least 95%, at least 98%, or 100% sequence identity to the amino acid sequence of SEQ ID NO: 67 or SEQ ID NO: 69; (ii) a variable light chain sequence having at least 85%, at least 90%, at least 95%, at least 98%, or 100% sequence identity to the amino acid sequence of SEQ ID NO: 68 or SEQ ID NO: 70; or (iii) a variable heavy chain sequence as in (i) and a variable light chain sequence as in (ii).

Embodiment 27. The antibody of any one of the preceding embodiments, wherein the antibody comprises a variable heavy chain sequence comprising the amino acid sequence of SEQ ID NO: 21, SEQ ID NO: 43, SEQ ID NO: 59, SEQ ID NO: 60, SEQ ID NO: 63, SEQ ID NO: 64, SEQ ID NO: 274, SEQ ID NO: 363, SEQ ID NO: 67, or SEQ ID NO: 69.

Embodiment 28. The antibody of any one of the preceding embodiments, wherein the antibody comprises a variable light chain sequence comprising the amino acid sequence of SEQ ID NO: 22, SEQ ID NO: 44, SEQ ID NO: 61, SEQ ID NO: 62, SEQ ID NO: 65, SEQ ID NO: 66, SEQ ID NO: 275, SEQ ID NO: 364, SEQ ID NO: 68, or SEQ ID NO: 70.

Embodiment 29. The antibody of any one of the preceding embodiments, wherein the antibody comprises:
a) a variable heavy chain sequence comprising the amino acid sequence of SEQ ID NO: 21 or SEQ ID NO: 43, and a variable light chain sequence comprising the amino acid sequence of SEQ ID NO: 22 or SEQ ID NO: 44;
b) a variable heavy chain sequence comprising the amino acid sequence of SEQ ID NO: 59, SEQ ID NO: 60, SEQ ID NO: 63, SEQ ID NO: 64, SEQ ID NO: 274, SEQ ID NO: 363, and a variable light chain sequence comprising the amino acid sequence of SEQ ID NO: 61, SEQ ID NO: 62, SEQ ID NO: 65, SEQ ID NO: 66, SEQ ID NO: 275, SEQ ID NO: 364; or
c) a variable heavy chain sequence comprising the amino acid sequence of SEQ ID NO: 67, SEQ ID NO: 69, SEQ ID NO: 365, or SEQ ID NO: 366, and a variable light chain sequence comprising the amino acid sequence of SEQ ID NO: 68, SEQ ID NO: 70, SEQ ID NO: 367, SEQ ID NO: 368, or SEQ ID NO: 369.

Embodiment 30. An isolated antibody, wherein the antibody comprises:
a) a variable heavy chain sequence comprising the amino acid sequence of SEQ ID NO: 21 or SEQ ID NO: 43, and a variable light chain sequence comprising the amino acid sequence of SEQ ID NO: 22 or SEQ ID NO: 44;
b) a variable heavy chain sequence comprising the amino acid sequence of SEQ ID NO: 59, SEQ ID NO: 60, SEQ ID NO: 63, SEQ ID NO: 64, SEQ ID NO: 274, or SEQ ID NO: 363, and a variable light chain sequence comprising the amino acid sequence of SEQ ID NO: 61, SEQ ID NO: 62, SEQ ID NO: 65, SEQ ID NO: 66, SEQ ID NO: 275, or SEQ ID NO: 364; or
c) a variable heavy chain sequence comprising the amino acid sequence of SEQ ID NO: 67, SEQ ID NO: 69, SEQ ID NO: 365, or SEQ ID NO: 366, and a variable light chain sequence comprising the amino acid sequence of SEQ ID NO: 68, SEQ ID NO: 70, SEQ ID NO: 367, SEQ ID NO: 368, or SEQ ID NO: 369, and
wherein the antibody comprises a variant canine or feline IgG Fc polypeptide capable of binding to neonatal Fc receptor (FcRn) with an increased affinity relative to the wild-type Fc polypeptide.

Embodiment 31. An isolated antibody that binds to canine IL4R or feline IL4R, wherein the antibody binds to an epitope comprising the amino acid sequence of SEQ ID NO: 354, and wherein the antibody comprises a variant canine or feline IgG Fc polypeptide capable of binding to neonatal Fc receptor (FcRn) with an increased affinity relative to the wild-type Fc polypeptide.

Embodiment 32. The isolated antibody of embodiment 31, wherein the antibody binds to canine IL4R or feline IL4R with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁹ M, less than 1×10⁻¹⁹ M, less than 5×10⁻¹¹ M, less than 1×10⁻¹¹ M, less than 5×10⁻¹² M, or less than 1×10⁻¹² M, as measured by biolayer interferometry.

Embodiment 33. The antibody of embodiment 31 or embodiment 32, wherein the antibody binds to canine IL4R or feline IL4R as determined by immunoblot analysis or biolayer interferometry.

Embodiment 34. The isolated antibody of any one of embodiments 31 to 33, wherein the antibody reduces binding of a canine and/or feline IL4 polypeptide and/or a canine and/or feline IL13 polypeptide to canine IL4R and/or feline IL4R, as measured by biolayer interferometry.

Embodiment 35. The isolated antibody of any one of embodiments 31 to 34, wherein the antibody competes with monoclonal M3 antibody in binding to canine IL4R or feline IL4R.

Embodiment 36. The isolated antibody of any one of embodiments 31 to 35, wherein the antibody is a monoclonal antibody.

Embodiment 37. The isolated antibody of any one of embodiments 31 to 36, wherein the antibody is a canine, a caninized, a feline, a felinized, or a chimeric antibody.

Embodiment 38. The isolated antibody of any one of embodiments 31 to 37, wherein the antibody is a chimeric antibody comprising one or more murine variable heavy chain framework regions or one or more murine variable light chain framework regions.

Embodiment 39. The isolated antibody of any one of embodiments 31 to 38, comprising a heavy chain comprising:
a) a CDR-H1 sequence having at least 85%, at least 90%, at least 95%, at least 98%, or 100% sequence identity to the amino acid sequence of SEQ ID NO: 278;
b) a CDR-H2 sequence having at least 85%, at least 90%, at least 95%, at least 98%, or 100% sequence identity to the amino acid sequence of SEQ ID NO: 279; and
c) a CDR-H3 sequence having at least 85%, at least 90%, at least 95%, at least 98%, or 100% sequence identity to the amino acid sequence of SEQ ID NO: 280.

Embodiment 40. The isolated antibody of any one of embodiments 31 to 39, comprising a light chain comprising:
a) a CDR-L1 sequence having at least 85%, at least 90%, at least 95%, at least 98%, or 100% sequence identity to the amino acid sequence of SEQ ID NO: 285;
b) a CDR-L2 sequence having at least 85%, at least 90%, at least 95%, at least 98%, or 100% sequence identity to the amino acid sequence of SEQ ID NO: 286; and
c) a CDR-L3 sequence having at least 85%, at least 90%, at least 95%, at least 98%, or 100% sequence identity to the amino acid sequence of SEQ ID NO: 287.

Embodiment 41. The isolated antibody of any one of embodiments 31 to 40, further comprising one or more of (a) a variable region heavy chain framework 1 (HC-FR1) sequence of SEQ ID NO: 281; (b) a HC-FR2 sequence of SEQ ID NO: 282; (c) a HC-FR3 sequence of SEQ ID NO: 283; (d) a HC-FR4 sequence of SEQ ID NO: 284; (e) a variable region light chain framework 1 (LC-FR1) sequence of SEQ ID NO: 288; (f) an LC-FR2 sequence of SEQ ID NO: 289; (g) an LC-FR3 sequence of SEQ ID NO: 290; or (h) an LC-FR4 sequence of SEQ ID NO: 291.

Embodiment 42. The isolated antibody of any one of embodiments 31 to 41, wherein the antibody comprises:
a. (i) a variable heavy chain sequence having at least 85%, at least 90%, at least 95%, at least 98%, or 100% sequence identity to the amino acid sequence of SEQ ID NO: 292; (ii) a variable light chain sequence having at least 85%, at least 90%, at least 95%, at least 98%, or 100% sequence identity to the amino acid sequence of SEQ ID NO: 293; or (iii) a variable heavy chain sequence as in (i) and a variable light chain sequence as in (ii); or
b. (i) a variable heavy chain sequence having at least 85%, at least 90%, at least 95%, at least 98%, or 100% sequence identity to the amino acid sequence of SEQ ID NO: 342 or SEQ ID NO: 343; (ii) a variable light chain sequence having at least 85%, at least 90%, at least 95%, at least 98%, or 100% sequence identity to the amino acid sequence of SEQ ID NO: 344; or (iii) a variable heavy chain sequence as in (i) and a variable light chain sequence as in (ii).

Embodiment 43. The isolated antibody of any one of embodiments 31 to 42, wherein the antibody comprises a variable heavy chain sequence comprising the amino acid sequence of SEQ ID NO: 292, SEQ ID NO: 342, or SEQ ID NO: 343.

Embodiment 44. The isolated antibody of any one of embodiments 31 to 43, wherein the antibody comprises a variable light chain sequence comprising the amino acid sequence of SEQ ID NO: 293 or SEQ ID NO: 344.

Embodiment 45. The isolated antibody of any one of embodiments 31 to 44, wherein the antibody comprises:
a) a variable heavy chain sequence comprising the amino acid sequence of SEQ ID NO: 292, and a variable light chain sequence comprising the amino acid sequence of SEQ ID NO: 293; and/or
b) a variable heavy chain sequence comprising the amino acid sequence of SEQ ID NO: 342 or SEQ ID NO: 343, and a variable light chain sequence comprising the amino acid sequence of SEQ ID NO: 344.

Embodiment 46. An isolated antibody comprising:
a) a variable heavy chain sequence comprising the amino acid sequence of SEQ ID NO: 292, and a variable light chain sequence comprising the amino acid sequence of SEQ ID NO: 293; or
b) a variable heavy chain sequence comprising the amino acid sequence of SEQ ID NO: 342 or SEQ ID NO: 343, and a variable light chain sequence comprising the amino acid sequence of SEQ ID NO: 344;
and wherein the antibody comprises a variant canine or feline IgG Fc polypeptide capable of binding to neonatal Fc receptor (FcRn) with an increased affinity relative to the wild-type Fc polypeptide.

Embodiment 47. An isolated antibody that binds to canine IL4R or feline IL4R, wherein the antibody binds to an epitope comprising the amino acid sequence of SEQ ID NO: 355 and/or an epitope comprising the amino acid sequence of SEQ ID NO: 356, and wherein the antibody comprises a variant canine or feline IgG Fc polypeptide capable of binding to neonatal Fc receptor (FcRn) with an increased affinity relative to the wild-type Fc polypeptide.

Embodiment 48. The isolated antibody of embodiment 47, wherein the antibody binds to canine IL4R or feline IL4R with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁹ M, less than 1×10⁻¹⁹ M, less than 5×10⁻¹¹ M, less than 1×10⁻¹¹ M, less than 5×10⁻¹² M, or less than 1×10⁻¹² M, as measured by biolayer interferometry.

Embodiment 49. The antibody of embodiment 47 or embodiment 48, wherein the antibody binds to canine IL4R or feline IL4R as determined by immunoblot analysis or biolayer interferometry.

Embodiment 50. The isolated antibody of any one of embodiments 47 to 49, wherein the antibody reduces binding of a canine and/or feline IL4 polypeptide and/or a canine and/or feline IL13 polypeptide to canine IL4R and/or feline IL4R, as measured by biolayer interferometry.

Embodiment 51. The isolated antibody of any one of embodiments 47 to 50, wherein the antibody competes with monoclonal M8 antibody in binding to canine IL4R or feline IL4R.

Embodiment 52. The isolated antibody of any one of embodiments 47 to 51, wherein the antibody is a monoclonal antibody.

Embodiment 53. The isolated antibody of any one of embodiments 47 to 52, wherein the antibody is a canine, a caninized, a feline, a felinized, or a chimeric antibody.

Embodiment 54. The isolated antibody of any one of embodiments 47 to 53, wherein the antibody is a chimeric antibody comprising one or more murine variable heavy chain framework regions or one or more murine variable light chain framework regions.

Embodiment 55. The isolated antibody of any one of embodiments 47 to 54, comprising a heavy chain comprising:
a) a CDR-H1 sequence having at least 85%, at least 90%, at least 95%, at least 98%, or 100% sequence identity to the amino acid sequence of SEQ ID NO: 310;
b) a CDR-H2 sequence having at least 85%, at least 90%, at least 95%, at least 98%, or 100% sequence identity to the amino acid sequence of SEQ ID NO: 311; and
c) a CDR-H3 sequence having at least 85%, at least 90%, at least 95%, at least 98%, or 100% sequence identity to the amino acid sequence of SEQ ID NO: 312.

Embodiment 56. The isolated antibody of any one of embodiments 47 to 55, comprising a light chain comprising:
a) a CDR-L1 sequence having at least 85%, at least 90%, at least 95%, at least 98%, or 100% sequence identity to the amino acid sequence of SEQ ID NO: 317;
b) a CDR-L2 sequence having at least 85%, at least 90%, at least 95%, at least 98%, or 100% sequence identity to the amino acid sequence of SEQ ID NO: 318; and
c) a CDR-L3 sequence having at least 85%, at least 90%, at least 95%, at least 98%, or 100% sequence identity to the amino acid sequence of SEQ ID NO: 319.

Embodiment 57. The isolated antibody of any one of embodiments 47 to 56, further comprising one or more of (a) a variable region heavy chain framework 1 (HC-FR1) sequence of SEQ ID NO: 313; (b) a HC-FR2 sequence of SEQ ID NO: 314; (c) a HC-FR3 sequence of SEQ ID NO: 315; (d) a HC-FR4 sequence of SEQ ID NO: 316; (e) a variable region light chain framework 1 (LC-FR1) sequence of SEQ ID NO: 320; (f) an LC-FR2 sequence of SEQ ID NO: 321; (g) an LC-1-R3 sequence of SEQ ID NO: 322; or (h) an LC-FR4 sequence of SEQ ID NO: 323.

Embodiment 58. The isolated antibody of any one of embodiments 47 to 57, wherein the antibody comprises:
(a) a variable heavy chain sequence having at least 85%, at least 90%, at least 95%, at least 98%, or 100% sequence identity to the amino acid sequence of SEQ ID NO: 324;
(b) a variable light chain sequence having at least 85%, at least 90%, at least 95%, at least 98%, or 100% sequence identity to the amino acid sequence of SEQ ID NO: 325; or
(c) a variable heavy chain sequence as in (a) and a variable light chain sequence as in (b).

Embodiment 59. The isolated antibody of any one of embodiments 47 to 58, wherein the antibody comprises a variable heavy chain sequence comprising the amino acid sequence of SEQ ID NO: 324.

Embodiment 60. The isolated antibody of any one of embodiments 47 to 59, wherein the antibody comprises a variable light chain sequence comprising the amino acid sequence of SEQ ID NO: 325.

Embodiment 61. The isolated antibody of any one of embodiments 47 to 60, wherein the antibody comprises: a variable heavy chain sequence comprising the amino acid sequence of SEQ ID NO: 324, and a variable light chain sequence comprising the amino acid sequence of SEQ ID NO: 325.

Embodiment 62. An isolated antibody comprising: a variable heavy chain sequence comprising the amino acid sequence of SEQ ID NO: 324, and a variable light chain sequence comprising the amino acid sequence of SEQ ID NO: 325, and wherein the antibody comprises a variant canine or feline IgG Fc polypeptide capable of binding to neonatal Fc receptor (FcRn) with an increased affinity relative to the wild-type Fc polypeptide.

Embodiment 63. An isolated antibody that binds to canine IL4R, wherein the antibody binds to an epitope comprising the amino acid sequence of SEQ ID NO: 357, and wherein the antibody comprises a variant canine or feline IgG Fc polypeptide capable of binding to neonatal Fc receptor (FcRn) with an increased affinity relative to the wild-type Fc polypeptide.

Embodiment 64. The isolated antibody of embodiment 63, wherein the antibody binds to canine IL4R with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁹ M, less than 1×10⁻¹⁹ M, less than 5×10⁻¹¹ M, less than 1×10⁻¹¹ M, less than 5×10⁻¹² M, or less than 1×10⁻¹² M, as measured by biolayer interferometry.

Embodiment 65. The antibody of embodiment 63 or embodiment 64, wherein the antibody binds to canine IL4R as determined by immunoblot analysis or biolayer interferometry.

Embodiment 66. The isolated antibody of any one of embodiments 63 to 65, wherein the antibody reduces binding of a canine IL4 polypeptide and/or a canine IL13 polypeptide to canine IL4R, as measured by biolayer interferometry.

Embodiment 67. The isolated antibody of any one of embodiments 63 to 66, wherein the antibody competes with monoclonal M9 antibody in binding to canine IL4R.

Embodiment 68. The isolated antibody of any one of embodiments 63 to 67, wherein the antibody is a monoclonal antibody.

Embodiment 69. The isolated antibody of any one of embodiments 63 to 68, wherein the antibody is a canine, a caninized, a feline, a felinized, or a chimeric antibody.

Embodiment 70. The isolated antibody of any one of embodiments 63 to 69, wherein the antibody is a chimeric antibody comprising one or more murine variable heavy chain framework regions or one or more murine variable light chain framework regions.

Embodiment 71. The isolated antibody of any one of embodiments 63 to 70, comprising a heavy chain comprising:
a) a CDR-H1 sequence having at least 85%, at least 90%, at least 95%, at least 98%, or 100% sequence identity to the amino acid sequence of SEQ ID NO: 326 or SEQ ID NO: 407;
b) a CDR-H2 sequence having at least 85%, at least 90%, at least 95%, at least 98%, or 100% sequence identity to the amino acid sequence of SEQ ID NO: 327; and
c) a CDR-H3 sequence having at least 85%, at least 90%, at least 95%, at least 98%, or 100% sequence identity to the amino acid sequence of SEQ ID NO: 328.

Embodiment 72. The isolated antibody of any one of embodiments 63 to 71, comprising a light chain comprising:
a) a CDR-L1 sequence having at least 85%, at least 90%, at least 95%, at least 98%, or 100% sequence identity to the amino acid sequence of SEQ ID NO: 333;
b) a CDR-L2 sequence having at least 85%, at least 90%, at least 95%, at least 98%, or 100% sequence identity to the amino acid sequence of SEQ ID NO: 334; and
c) a CDR-L3 sequence having at least 85%, at least 90%, at least 95%, at least 98%, or 100% sequence identity to the amino acid sequence of SEQ ID NO: 335.

Embodiment 73. The isolated antibody of any one of embodiments 63 to 72, further comprising one or more of (a) a variable region heavy chain framework 1 (HC-FR1) sequence of SEQ ID NO: 329; (b) a HC-FR2 sequence of SEQ ID NO: 330; (c) a HC-FR3 sequence of SEQ ID NO: 331; (d) a HC-FR4 sequence of SEQ ID NO: 332; (e) a variable region light chain framework 1 (LC-FR1) sequence of SEQ ID NO: 336; (f) an LC-FR2 sequence of SEQ ID NO: 337; (g) an LC-1-R3 sequence of SEQ ID NO: 338; or (h) an LC-FR4 sequence of SEQ ID NO: 339.

Embodiment 74. The isolated antibody of any one of embodiments 63 to 73, wherein the antibody comprises:
a. (i) a variable heavy chain sequence having at least 85%, at least 90%, at least 95%, at least 98%, or 100% sequence identity to the amino acid sequence of SEQ ID NO: 340; (ii) a variable light chain sequence having at least 85%, at least 90%, at least 95%, at least 98%, or 100% sequence identity to the amino acid sequence of SEQ ID NO: 341; or (iii) a variable heavy chain sequence as in (i) and a variable light chain sequence as in (ii); or
b. (i) a variable heavy chain sequence having at least 85%, at least 90%, at least 95%, at least 98%, or 100% sequence identity to the amino acid sequence of SEQ ID NO: 345 or SEQ ID NO: 346; (ii) a variable light chain sequence having at least 85%, at least 90%, at least 95%, at least 98%, or 100% sequence identity to the amino acid sequence of SEQ ID NO: 347 or SEQ ID NO: 408; or (iii) a variable heavy chain sequence as in (i) and a variable light chain sequence as in (ii).

Embodiment 75. The isolated antibody of any one of embodiments 63 to 74, wherein the antibody comprises a variable heavy chain sequence comprising the amino acid sequence of SEQ ID NO: 340, SEQ ID NO: 345, or SEQ ID NO: 346.

Embodiment 76. The isolated antibody of any one of embodiments 63 to 75, wherein the antibody comprises a variable light chain sequence comprising the amino acid sequence of SEQ ID NO: 341, SEQ ID NO: 347, or SEQ ID NO: 408.

Embodiment 77. The isolated antibody of any one of embodiments 63 to 76, wherein the antibody comprises:
a) a variable heavy chain sequence comprising the amino acid sequence of SEQ ID NO: 340, and a variable light chain sequence comprising the amino acid sequence of SEQ ID NO: 341; and/or
b) a variable heavy chain sequence comprising the amino acid sequence of SEQ ID NO: 345 or SEQ ID NO: 346, and a variable light chain sequence comprising the amino acid sequence of SEQ ID NO: 347 or SEQ ID NO: 408.

Embodiment 78. An isolated antibody comprising:
a) a variable heavy chain sequence comprising the amino acid sequence of SEQ ID NO: 340, and a variable light chain sequence comprising the amino acid sequence of SEQ ID NO: 341; or
b) a variable heavy chain sequence comprising the amino acid sequence of SEQ ID NO: 345, or SEQ ID NO: 346 and a variable light chain sequence comprising the amino acid sequence of SEQ ID NO: 347 or SEQ ID NO: 408, and wherein the antibody comprises a variant canine or feline IgG Fc polypeptide capable of binding to neonatal Fc receptor (FcRn) with an increased affinity relative to the wild-type Fc polypeptide.

Embodiment 79. An isolated antibody that binds to canine IL4R, comprising a heavy chain comprising:
a) a CDR-H1 sequence having at least 85%, at least 90%, at least 95%, at least 98%, or 100% sequence identity to the amino acid sequence of SEQ ID NO: 294;
b) a CDR-H2 sequence having at least 85%, at least 90%, at least 95%, at least 98%, or 100% sequence identity to the amino acid sequence of SEQ ID NO: 295; and
c) a CDR-H3 sequence having at least 85%, at least 90%, at least 95%, at least 98%, or 100% sequence identity to the amino acid sequence of SEQ ID NO: 296; and
wherein the antibody comprises a variant canine or feline IgG Fc polypeptide capable of binding to neonatal Fc receptor (FcRn) with an increased affinity relative to the wild-type Fc polypeptide.

Embodiment 80. An isolated antibody that binds to canine IL4R, comprising a light chain comprising:
a) a CDR-L1 sequence having at least 85%, at least 90%, at least 95%, at least 98%, or 100% sequence identity to the amino acid sequence of SEQ ID NO: 301;
b) a CDR-L2 sequence having at least 85%, at least 90%, at least 95%, at least 98%, or 100% sequence identity to the amino acid sequence of SEQ ID NO: 302; and
c) a CDR-L3 sequence having at least 85%, at least 90%, at least 95%, at least 98%, or 100% sequence identity to the amino acid sequence of SEQ ID NO: 303; and
wherein the antibody comprises a variant canine or feline IgG Fc polypeptide capable of binding to neonatal Fc receptor (FcRn) with an increased affinity relative to the wild-type Fc polypeptide.

Embodiment 81. The isolated antibody of embodiment 79, comprising a light chain comprising:
a) a CDR-L1 sequence having at least 85%, at least 90%, at least 95%, at least 98%, or 100% sequence identity to the amino acid sequence of SEQ ID NO: 301;
b) a CDR-L2 sequence having at least 85%, at least 90%, at least 95%, at least 98%, or 100% sequence identity to the amino acid sequence of SEQ ID NO: 302; and
c) a CDR-L3 sequence having at least 85%, at least 90%, at least 95%, at least 98%, or 100% sequence identity to the amino acid sequence of SEQ ID NO: 303.

Embodiment 82. The isolated antibody of any one of embodiments 79 to 81, wherein the antibody binds to canine IL4R with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁹ M, less than 1×10⁻¹⁹ M, less than 5×10⁻¹¹ M, less than 1×10⁻¹¹ M, less than 5×10⁻¹² M, or less than 1×10⁻¹² M, as measured by biolayer interferometry.

Embodiment 83. The antibody of any one of embodiments 79 to 82, wherein the antibody binds to canine IL4R as determined by immunoblot analysis or biolayer interferometry.

Embodiment 84. The isolated antibody of any one of embodiments 79 to 83, wherein the antibody reduces binding of a canine IL4 polypeptide and/or a canine IL13 polypeptide to canine IL4R, as measured by biolayer interferometry.

Embodiment 85. The isolated antibody of any one of embodiments 79 to 84, wherein the antibody competes with monoclonal M5 antibody in binding to canine IL4R.

Embodiment 86. The isolated antibody of any one of embodiments 79 to 85, wherein the antibody is a monoclonal antibody.

Embodiment 87. The isolated antibody of any one of embodiments 79 to 86, wherein the antibody is a canine, a caninized, a feline, a felinized, or a chimeric antibody.

Embodiment 88. The isolated antibody of any one of embodiments 79 to 87, wherein the antibody is a chimeric antibody comprising one or more murine variable heavy chain framework regions or one or more murine variable light chain framework regions.

Embodiment 89. The isolated antibody of any one of embodiments 79 to 88, further comprising one or more of (a) a variable region heavy chain framework 1 (HC-FR1) sequence of SEQ ID NO: 297; (b) a HC-FR2 sequence of SEQ ID NO: 298; (c) a HC-FR3 sequence of SEQ ID NO: 299; (d) a HC-FR4 sequence of SEQ ID NO: 300; (e) a variable region light chain framework 1 (LC-FR1) sequence of SEQ ID NO: 304; (f) an LC-FR2 sequence of SEQ ID NO: 305; (g) an LC-1-R3 sequence of SEQ ID NO: 306; or (h) an LC-FR4 sequence of SEQ ID NO: 307.

Embodiment 90. The isolated antibody of any one of embodiments 79 to 89, wherein the antibody comprises:
(a) a variable heavy chain sequence having at least 85%, at least 90%, at least 95%, at least 98%, or 100% sequence identity to the amino acid sequence of SEQ ID NO: 308;
(b) a variable light chain sequence having at least 85%, at least 90%, at least 95%, at least 98%, or 100% sequence identity to the amino acid sequence of SEQ ID NO: 309; or
(c) a variable heavy chain sequence as in (a) and a variable light chain sequence as in (b).

Embodiment 91. The isolated antibody of any one of embodiments 79 to 90, wherein the antibody comprises a variable heavy chain sequence comprising the amino acid sequence of SEQ ID NO: 308.

Embodiment 92. The isolated antibody of any one of embodiments 79 to 91, wherein the antibody comprises a variable light chain sequence comprising the amino acid sequence of SEQ ID NO: 309.

Embodiment 93. The isolated antibody of any one of embodiments 79 to 92, wherein the antibody comprises: a variable heavy chain sequence comprising the amino acid sequence of SEQ ID NO: 308, and a variable light chain sequence comprising the amino acid sequence of SEQ ID NO: 309.

Embodiment 94. An isolated antibody comprising: a variable heavy chain sequence comprising the amino acid sequence of SEQ ID NO: 308, and a variable light chain sequence comprising the amino acid sequence of SEQ ID NO: 309, and wherein the antibody comprises a variant canine or feline IgG Fc polypeptide capable of binding to neonatal Fc receptor (FcRn) with an increased affinity relative to the wild-type Fc polypeptide.

Embodiment 95. The antibody of any one of the preceding embodiments, wherein the variant IgG Fc polypeptide is a variant canine IgG-A Fc polypeptide; a variant canine IgG-B Fc polypeptide; a variant IgG-C Fc polypeptide; a variant IgG-D Fc polypeptide; a variant feline IgG1a Fc polypeptide; a variant feline IgG1b Fc polypeptide; or a variant feline IgG2 Fc polypeptide.

Embodiment 96. The antibody of any one of the preceding embodiments, wherein the variant IgG Fc polypeptide comprises at least one amino acid modification relative to a wild-type IgG Fc polypeptide, wherein the variant IgG Fc polypeptide has increased binding affinity to Protein A relative to the wild-type IgG Fc polypeptide; reduced binding affinity to C1q relative to the wild-type IgG Fc polypeptide; and/or reduced binding affinity to CD16 relative to the wild-type IgG Fc polypeptide.

Embodiment 97. The antibody of any one of the preceding embodiments, wherein the variant IgG Fc polypeptide comprises at least one amino acid modification to a hinge region relative to a wild-type IgG Fc polypeptide, wherein the variant IgG Fc polypeptide has increased recombinant production and/or increased hinge disulfide formation relative to the wild-type IgG Fc polypeptide, as determined by SDS-PAGE analysis under reducing and/or nonreducing conditions.

Embodiment 98. The antibody of any one of the preceding embodiments, wherein the variant IgG Fc polypeptide comprises:
a) at least one amino acid substitution at a position corresponding to position 21, 23, 25, 80, 205, and/or 207 of SEQ ID NO: 162;
b) at least one amino acid substitution at a position corresponding to position 5, 38, 39, 94, 97, and/or 98 of SEQ ID NO: 163;
c) at least one amino acid substitution at a position corresponding to position 5, 21, 23, 24, 38, 39, 93, 97, and/or 98 of SEQ ID NO: 165;
d) at least one amino acid substitution at a position corresponding to position 21, 23, 25, 80, and/or 207 of SEQ ID NO: 167;
e) at least one amino acid substitution at a position corresponding to position 16 and/or 198 of SEQ ID NO: 203, SEQ ID NO: 204, SEQ ID NO: 205, or SEQ ID NO: 206; and/or
f) at least one amino acid substitution at a position corresponding to position 14 and/or 16 of SEQ ID NO: 207.

Embodiment 99. The antibody of any one of the preceding embodiments, wherein the variant IgG Fc polypeptide comprises:
a) at least one amino acid substitution at position 21, 23, 25, 80, 205, and/or 207 of SEQ ID NO: 162;
b) at least one amino acid substitution at position 5, 38, 39, 94, 97, and/or 98 of SEQ ID NO: 163;
c) at least one amino acid substitution at position 5, 21, 23, 24, 38, 39, 93, 97, and/or 98 of SEQ ID NO: 164;
d) at least one amino acid substation at position 21, 23, 25, 80, and/or 207 of SEQ ID NO: 165;
e) at least one amino acid substitution at position 16 and/or 198 of SEQ ID NO: 203, SEQ ID NO: 204, SEQ ID NO: 205, or SEQ ID NO: 206; and/or
f) at least one amino acid substitution at position 14 and/or 16 of SEQ ID NO: 207.

Embodiment 100. The antibody of any one of the preceding embodiments, wherein the variant IgG Fc polypeptide comprises:
a) a threonine at a position corresponding to position 21 of SEQ ID NO: 162, a leucine at a position corresponding to position 23 of SEQ ID NO: 162, an alanine at a position corresponding to position 25 of SEQ ID NO: 162, a glycine at a position corresponding to position 80 of SEQ ID NO: 162, an alanine at a position corresponding to position 205 of SEQ ID NO: 162, and/or a histidine at a position corresponding to position 207 of SEQ ID NO: 162;
b) a proline at a position corresponding to position 5 of SEQ ID NO: 163, a glycine at a position corresponding to position 38 of SEQ ID NO: 163, an arginine at a position corresponding to position 39 of SEQ ID NO: 163, an arginine at a position corresponding to position 93 of SEQ ID NO: 163, an isoleucine at a position corresponding to position 97 of SEQ ID NO: 163, and/or a glycine at a position corresponding to position 98 of SEQ ID NO: 163;
c) a proline at a position corresponding to position 5 of SEQ ID NO: 164, a threonine at a position corresponding to position 21 of SEQ ID NO: 164, a leucine at a position corresponding to position 23 of SEQ ID NO: 164, an isoleucine at a position corresponding to position 24 of SEQ ID NO: 164, a glycine at a position corresponding to position 38 of SEQ ID NO: 164, an arginine at a position corresponding to position 39 of SEQ ID NO: 164, an arginine at a position corresponding to position 93 of SEQ ID NO: 164, an isoleucine at a position corresponding to position 97 of SEQ ID NO: 164, and/or a glycine at a position corresponding to position 98 of SEQ ID NO: 164;
d) a threonine at a position corresponding to position 21 of SEQ ID NO: 165, a leucine at a position corresponding to position 23 of SEQ ID NO: 165, an alanine at a position corresponding to position 25 of SEQ ID NO: 165, a glycine at a position corresponding to position 80 of SEQ ID NO: 165, and/or a histidine at a position corresponding to position 207 of SEQ ID NO: 165;
e) a proline at a position corresponding to position 16 of SEQ ID NO: 203, SEQ ID NO: 204, SEQ ID NO: 205, or SEQ ID NO: 206 and/or an alanine at a position corresponding to position 198 of S SEQ ID NO: 203, SEQ ID NO: 204, SEQ ID NO: 205, or SEQ ID NO: 206; and/or
f) a cysteine at a position corresponding to position 14 of SEQ ID NO: 207 and/or a proline at a position corresponding to position 16 of SEQ ID NO: 207.

Embodiment 101. The antibody of any one of the preceding embodiments, wherein the variant IgG Fc polypeptide comprises:
a) a threonine at position 21 of SEQ ID NO: 162, a leucine at position 23 of SEQ ID NO: 162, an alanine at position 25 of SEQ ID NO: 162, a glycine at position 80 of SEQ ID NO: 162, an alanine at position 205 of SEQ ID NO: 162, and/or a histidine at position 207 of SEQ ID NO: 162;
b) a proline at position 5 of SEQ ID NO: 163, a glycine at position 38 of SEQ ID NO: 163, an arginine at position 39 of SEQ ID NO: 163, an arginine at position 93 of SEQ ID NO: 163, an isoleucine at position 97 of SEQ ID NO: 163, and/or a glycine at position 98 of SEQ ID NO: 163;
c) a proline at position 5 of SEQ ID NO: 164, a threonine at position 21 of SEQ ID NO: 164, a leucine at position 23 of SEQ ID NO: 164, an isoleucine at position 24 of SEQ ID NO: 164, a glycine at position 38 of SEQ ID NO: 164, an arginine at position 39 of SEQ ID NO: 164, an arginine at position 93 of SEQ ID NO: 164, an isoleucine at position 97 of SEQ ID NO: 164, and/or a glycine at position 98 of SEQ ID NO: 164;
d) a threonine at position 21 of SEQ ID NO: 165, a leucine at position 23 of SEQ ID NO: 165, an alanine at position 25 of SEQ ID NO: 165, a glycine at position 80 of SEQ ID NO: 165, and/or a histidine at position 207 of SEQ ID NO: 165;
e) a proline at position 16 of SEQ ID NO: 203, SEQ ID NO: 204, SEQ ID NO: 205, or SEQ ID NO: 206 and/or an alanine at position 198 of SEQ ID NO: 203, SEQ ID NO: 204, SEQ ID NO: 205, or SEQ ID NO: 206; and/or
f) a cysteine at position 14 of SEQ ID NO: 207 and/or a proline at position 16 of SEQ ID NO: 207.

Embodiment 102. The antibody of any one of the preceding embodiments, wherein the variant IgG Fc polypeptide comprises a CH1 region comprising at least one amino acid modification relative to a wild-type canine or feline IgG CH1 region, wherein the variant IgG Fc polypeptide comprises:
a) at least one amino acid substitution at a position corresponding to position 24 and/or position 30 of SEQ ID NO: 227, SEQ ID NO: 228, SEQ ID NO: 229, SEQ ID NO: 230, or SEQ ID NO: 237; or
b) at least one amino acid substitution at a position corresponding to position 24 and/or position 29 of SEQ ID NO: 238.

Embodiment 103. An antibody comprising a variant IgG Fc polypeptide comprising a CH1 region comprising at least one amino acid modification relative to a wild-type canine or feline IgG CH1 region, wherein the variant IgG Fc polypeptide comprises:
a) at least one amino acid substitution at a position corresponding to position 24 and/or position 30 of SEQ ID NO: 227, SEQ ID NO: 228, SEQ ID NO: 229, SEQ ID NO: 230, or SEQ ID NO: 237; or
b) at least one amino acid substitution at a position corresponding to position 24 and/or position 29 of SEQ ID NO: 238; and
wherein the antibody comprises a variant canine or feline IgG Fc polypeptide capable of binding to neonatal Fc receptor (FcRn) with an increased affinity relative to the wild-type Fc polypeptide.

Embodiment 104. The antibody of any one of the preceding embodiments, wherein the variant IgG Fc polypeptide comprises a CH1 region comprising at least one amino acid modification relative to a wild-type canine or feline IgG CH1 region, wherein the variant IgG Fc polypeptide comprises:
a) at least one amino acid substitution at position 24 and/or position 30 of SEQ ID NO: 227, SEQ ID NO: 228, SEQ ID NO: 229, SEQ ID NO: 230, or SEQ ID NO: 237; or
b) at least one amino acid substitution at position 24 and/or position 29 of SEQ ID NO: 238.

Embodiment 105. The antibody of any one of the preceding embodiments, wherein the variant IgG Fc polypeptide comprises a CH1 region comprising at least one amino acid modification relative to a wild-type canine or feline IgG CH1 region, wherein the variant IgG Fc polypeptide comprises:
a) a leucine at a position corresponding to position 24 and/or an asparagine at a position corresponding to position 30 of SEQ ID NO: 227, SEQ ID NO: 228, SEQ ID NO: 229, SEQ ID NO: 230, or SEQ ID NO: 237; or
b) a leucine at a position corresponding to position 24 and/or an asparagine at a position corresponding to position 29 of SEQ ID NO: 238.

Embodiment 106. The antibody of any one of the preceding embodiments, wherein the variant IgG Fc polypeptide comprises a CH1 region comprising at least one amino acid modification relative to a wild-type canine or feline IgG CH1 region, wherein the variant IgG Fc polypeptide comprises:
a) a leucine at position 24 and/or an asparagine at position 30 of SEQ ID NO: 227, SEQ ID NO: 228, SEQ ID NO: 229, SEQ ID NO: 230, or SEQ ID NO: 237; or
b) a leucine at position 24 and/or an asparagine at position 29 of SEQ ID NO: 238.

Embodiment 107. The antibody of any one of the preceding embodiments, wherein the antibody comprises a wild-type or a variant canine or feline light chain constant region.

Embodiment 108. The antibody of any one of the preceding embodiments, wherein the antibody comprises a wild-type or a variant canine or feline light chain κ constant region.

Embodiment 109. The antibody of embodiment 107 or embodiment 108, wherein the variant light chain constant region comprises at least one amino acid modification relative to a wild-type canine or feline light chain κ constant region comprising:
a) at least one amino acid substitution at a position corresponding to position 11 and/or position 22 of SEQ ID NO: 235; or
b) at least one amino acid substitution at a position corresponding to position 11 and/or position 22 of SEQ ID NO: 241.

Embodiment 110. An antibody comprising a variant light chain constant region comprises at least one amino acid modification relative to a wild-type canine or feline light chain κ constant region comprising:
a) at least one amino acid substitution at a position corresponding to position 11 and/or position 22 of SEQ ID NO: 235; or
b) at least one amino acid substitution at a position corresponding to position 11 and/or position 22 of SEQ ID NO: 241; and
wherein the antibody comprises a variant canine or feline IgG Fc polypeptide capable of binding to neonatal Fc receptor (FcRn) with an increased affinity relative to the wild-type Fc polypeptide.

Embodiment 111. The antibody of any one of embodiments 107 to 110, wherein the variant light chain constant region comprises at least one amino acid modification relative to a wild-type feline or canine light chain κ constant region comprising:
a) an alanine at a position corresponding to position 11 and/or an arginine at a position corresponding to position 22 of SEQ ID NO: 235; or
b) an alanine at a position corresponding to position 11 and/or an arginine at a position corresponding to position 22 of SEQ ID NO: 241.

Embodiment 112. The antibody of any one of embodiments 107 to 111, wherein the variant light chain constant region comprises at least one amino acid modification relative to a wild-type feline or canine light chain κ constant region comprising:
a) an alanine at position 11 and/or an arginine at position 22 of SEQ ID NO: 235; or
b) an alanine at position 11 and/or an arginine at position 22 of SEQ ID NO: 241.

Embodiment 113. The antibody of any one of embodiments 107 to 112, wherein the light chain constant region comprises an amino acid sequence of SEQ ID NO: 235, 236, 241, and/or 242.

Embodiment 114. The antibody of any one of the preceding embodiments, wherein the antibody is a bispecific antibody.

Embodiment 115. The antibody of any one of the preceding embodiments, wherein the antibody is a bispecific antibody comprising:
i) a first variant canine IgG Fc polypeptide comprising at least one amino acid modification relative to a first wild-type canine IgG Fc polypeptide and a second variant canine IgG Fc polypeptide comprising at least one amino acid modification relative to a second wild-type canine IgG Fc polypeptide, wherein:
   a) the first variant canine IgG Fc polypeptide comprises an amino acid substitution at a position corresponding to position 138 of SEQ ID NO: 162, position 137 of SEQ ID NO: 163, position 137 of SEQ ID NO: 165, or position 138 of SEQ ID NO: 167, and/or
   b) the second variant canine IgG Fc polypeptide comprises an amino acid substitution at a position corresponding to position 138 and/or position 140 of SEQ ID NO: 162, position 137 and/or position 139 of SEQ ID NO: 163, position 137 and/or position 139 of SEQ ID NO: 165, or position 138 and/or position 140 of SEQ ID NO: 167; or
ii) a first variant feline IgG Fc polypeptide comprising at least one amino acid modification relative to a first wild-type feline IgG Fc polypeptide and a second variant feline IgG Fc polypeptide comprising at least one amino acid modification relative to a second wild-type feline IgG Fc polypeptide, wherein:
   a) the first variant feline IgG Fc polypeptide comprises an amino acid substitution at a position corresponding to position 154 of SEQ ID NO: 203, SEQ ID NO: 204, SEQ ID NO: 205, SEQ ID NO: 206, or SEQ ID NO: 207, and/or
   b) the second variant feline IgG Fc polypeptide comprises an amino acid substitution at a position corresponding to position 154 and/or position 156 of SEQ ID NO: 203, SEQ ID NO: 204, SEQ ID NO: 205, SEQ ID NO: 206, or SEQ ID NO: 207.

Embodiment 116. An antibody or a bispecific antibody comprising:
i) a first variant canine IgG Fc polypeptide comprising at least one amino acid modification relative to a first wild-type canine IgG Fc polypeptide and a second variant canine IgG Fc polypeptide comprising at least one amino acid modification relative to a second wild-type canine IgG Fc polypeptide, wherein:
   a) the first variant canine IgG Fc polypeptide comprises an amino acid substitution at a position corresponding to position 138 of SEQ ID NO: 162, position 137 of SEQ ID NO: 163, position 137 of SEQ ID NO: 165, or position 138 of SEQ ID NO: 167, and/or
   b) the second variant canine IgG Fc polypeptide comprises an amino acid substitution at a position corresponding to position 138 and/or position 140 of SEQ ID NO: 162, position 137 and/or position 139 of SEQ ID NO: 163, position 137 and/or position 139 of SEQ ID NO: 165, or position 138 and/or position 140 of SEQ ID NO: 167;
ii) a first variant feline IgG Fc polypeptide comprising at least one amino acid modification relative to a first wild-type feline IgG Fc polypeptide and a second variant feline IgG Fc polypeptide comprising at least one amino acid modification relative to a second wild-type feline IgG Fc polypeptide, wherein:
   a) the first variant feline IgG Fc polypeptide comprises an amino acid substitution at a position corresponding to position 154 of SEQ ID NO: 203, SEQ ID NO: 204, SEQ ID NO: 205, SEQ ID NO: 206, or SEQ ID NO: 207, and/or
   b) the second variant feline IgG Fc polypeptide comprises an amino acid substitution at a position corresponding to position 154 and/or position 156 of SEQ ID NO: 203, SEQ ID NO: 204, SEQ ID NO: 205, SEQ ID NO: 206, or SEQ ID NO: 207; or
iii) a first variant equine IgG Fc polypeptide comprising at least one amino acid modification relative to a first wild-type equine IgG Fc polypeptide and a second variant equine IgG Fc polypeptide comprising at least one amino acid modification relative to a second wild-type equine IgG Fc polypeptide, wherein:
   a) the first variant equine IgG Fc polypeptide comprises an amino acid substitution at a position corresponding to position 130 of SEQ ID NO: 254, SEQ ID NO: 255, SEQ ID NO: 256, SEQ ID NO: 257, SEQ ID NO: 258, SEQ ID NO: 259, or SEQ ID NO: 260, and/or
   b) the second variant equine IgG Fc polypeptide comprises an amino acid substitution at a position corresponding to position 130 and/or position 132 of SEQ ID NO: 254, SEQ ID NO: 255, SEQ ID NO: 256, SEQ ID NO: 257, SEQ ID NO: 258, SEQ ID NO: 259, or SEQ ID NO: 260; and
wherein the antibody comprises a variant canine or feline IgG Fc polypeptide capable of binding to neonatal Fc receptor (FcRn) with an increased affinity relative to the wild-type Fc polypeptide.

Embodiment 117. The antibody of embodiment 115 or embodiment 116, wherein:
a) the first variant canine IgG Fc polypeptide comprises a tryptophan at a position corresponding to position 138 of SEQ ID NO: 162, position 137 of SEQ ID NO: 163, position 137 of SEQ ID NO: 165, or position 138 of SEQ ID NO: 167;
b) the second variant canine IgG Fc polypeptide comprises a serine at a position corresponding to position 138 and/or an alanine at a position corresponding to position 140 of SEQ ID NO: 162, a serine at a position corresponding to position 137 and/or an alanine at a position corresponding to position 139 of SEQ ID NO: 163, a serine at a position corresponding to position 137 and/or an alanine at a position corresponding to position 139 of SEQ ID NO: 165, or a serine at a position corresponding to position 138 and/or an alanine at a position corresponding to position 140 of SEQ ID NO: 167;
c) the first variant feline IgG Fc polypeptide comprises a tryptophan at a position corresponding to position 154 of SEQ ID NO: 203, SEQ ID NO: 204, SEQ ID NO: 205, SEQ ID NO: 206, or SEQ ID NO: 207;
d) the second variant feline IgG Fc polypeptide comprises a serine at a position corresponding to position 154 and/or an alanine at a position corresponding to position 156 of SEQ ID NO: 203, SEQ ID NO: 204, SEQ ID NO: 205, SEQ ID NO: 206, or SEQ ID NO: 207;
e) the first variant equine IgG Fc polypeptide comprises a tryptophan at a position corresponding to position 130 of SEQ ID NO: 254, SEQ ID NO: 255, SEQ ID NO: 256, SEQ ID NO: 257, SEQ ID NO: 258, SEQ ID NO: 259, or SEQ ID NO: 260; and/or
f) the second variant equine IgG Fc polypeptide comprises a serine at a position corresponding to position 130 and/or an alanine at a position corresponding to position 132 of SEQ ID NO: 254, SEQ ID NO: 255, SEQ ID NO: 256, SEQ ID NO: 257, SEQ ID NO: 258, SEQ ID NO: 259, or SEQ ID NO: 260.

Embodiment 118. The antibody of any one of embodiments 115 to 117, wherein:
a) the first variant canine IgG Fc polypeptide comprises an amino acid substitution at position 138 of SEQ ID NO: 162, position 137 of SEQ ID NO: 163, position 137 of SEQ ID NO: 165, or position 138 of SEQ ID NO: 167;
b) the second variant canine IgG Fc polypeptide comprises an amino acid substitution at position 138 and/or position 140 of SEQ ID NO: 162, an amino acid substitution at position 137 and/or position 139 of SEQ ID NO: 163, an amino acid substitution at position 137 and/or position 139 of SEQ ID NO: 165, or an amino acid substitution at position 138 and/or position 140 of SEQ ID NO: 167;
c) the first variant feline IgG Fc polypeptide comprises an amino acid substitution at position 154 of SEQ ID NO: 203, SEQ ID NO: 204, SEQ ID NO: 205, SEQ ID NO: 206, or SEQ ID NO: 207;
d) the second variant feline IgG Fc polypeptide comprises an amino acid substitution at position 154 and/or position 156 of SEQ ID NO: 203, SEQ ID NO: 204, SEQ ID NO: 205, SEQ ID NO: 206, or SEQ ID NO: 207;
e) the first variant equine IgG Fc polypeptide comprises an amino acid substitution at position 130 of SEQ ID NO: 254, SEQ ID NO: 255, SEQ ID NO: 256, SEQ ID NO: 257, SEQ ID NO: 258, SEQ ID NO: 259, or SEQ ID NO: 260; and/or
f) the second variant equine IgG Fc polypeptide comprises an amino acid substitution at position 130 and/or position 132 of SEQ ID NO: 254, SEQ ID NO: 255, SEQ ID NO: 256, SEQ ID NO: 257, SEQ ID NO: 258, SEQ ID NO: 259, or SEQ ID NO: 260.

Embodiment 119. The antibody of any one of embodiments 115 to 118, wherein:
a) the first variant canine IgG Fc polypeptide comprises a tryptophan at position 138 of SEQ ID NO: 162, position 137 of SEQ ID NO: 163, position 137 of SEQ ID NO: 165, or position 138 of SEQ ID NO: 167;
b) the second variant canine IgG Fc polypeptide comprises a serine at position 138 and/or an alanine at position 140 of SEQ ID NO: 162, a serine at position 137 and/or an alanine at position 139 of SEQ ID NO: 163, a serine at position 137 and/or an alanine at position 139 of SEQ ID NO: 165, or a serine at position 138 and/or an alanine at position 140 of SEQ ID NO: 167;
c) the first variant feline IgG Fc polypeptide comprises a tryptophan at position 154 of SEQ ID NO: 203, SEQ ID NO: 204, SEQ ID NO: 205, SEQ ID NO: 206, or SEQ ID NO: 207;
d) the second variant feline IgG Fc polypeptide comprises a serine at position 154 and/or an alanine at position 156 of SEQ ID NO: 203, SEQ ID NO: 204, SEQ ID NO: 205, SEQ ID NO: 206, or SEQ ID NO: 207;
e) the first variant equine IgG Fc polypeptide comprises a tryptophan at position 130 of SEQ ID NO: 254, SEQ ID NO: 255, SEQ ID NO: 256, SEQ ID NO: 257, SEQ ID NO: 258, SEQ ID NO: 259, or SEQ ID NO: 260; and/or
f) the second variant equine IgG Fc polypeptide comprises a serine at position 130 and/or an alanine at position 132 of SEQ ID NO: 254, SEQ ID NO: 255, SEQ ID NO: 256, SEQ ID NO: 257, SEQ ID NO: 258, SEQ ID NO: 259, or SEQ ID NO: 260.

Embodiment 120. The antibody of any one of embodiments 115 to 119, wherein the first wild-type IgG Fc polypeptide and the second wild-type IgG Fc polypeptide are from the same IgG subtype.

Embodiment 121. The antibody of any one of embodiments 115 to 117, wherein the first wild-type IgG Fc polypeptide and the second wild-type IgG Fc polypeptide are from a different IgG subtype.

Embodiment 122. The antibody of any one of the preceding embodiments, wherein the variant Fc polypeptide binds to FcRn with an affinity greater than the wild-type IgG Fc polypeptide, as measured by biolayer interferometry, surface plasmon resonance, or any protein-protein interaction tool at a pH in the range of from about 5.0 to about 6.5, such as at a pH of about 5.0, a pH of about 5.2, a pH of about 5.5, a pH of about 6.0, a pH of about 6.2, or a pH of about 6.5.

Embodiment 123. The antibody of any one of the preceding embodiments, wherein the variant IgG Fc polypeptide binds to FcRn with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁹ M, less than 1×10⁻¹⁹ M, less than 5×10⁻¹¹ M, less than 1×10⁻¹¹ M, less than 5×10⁻¹² M, or less than 1×10⁻¹² M, as measured by biolayer interferometry, surface plasmon resonance, or any protein-protein interaction tool at a pH in the range of from about 5.0 to about 6.5, such as at a pH of about 5.0, a pH of about 5.5, a pH of about 6.0, or a pH of about 6.5.

Embodiment 124. The antibody of any one of the preceding embodiments, wherein the antibody has increased serum half-life relative to the antibody with a wild-type IgG Fc polypeptide.

Embodiment 125. The antibody of any one of the preceding embodiments, wherein the variant IgG Fc polypeptide comprises:
a) a tyrosine or a phenylalanine at a position corresponding to position 23 of SEQ ID NO: 163;
b) a tyrosine at a position corresponding to position 82 of SEQ ID NO: 163;
c) a tyrosine at a position corresponding to position 82 and a histidine at a position corresponding to position 207 of SEQ ID NO: 163;
d) a tyrosine at a position corresponding to position 82 and a tyrosine at a position corresponding to position 207 of SEQ ID NO: 163; or
e) a tyrosine at a position corresponding to position 207 of SEQ ID NO: 163.

Embodiment 126. The antibody of any one of the preceding embodiments, wherein the variant IgG Fc polypeptide comprises:
a) a tyrosine or a phenylalanine at position 23 of SEQ ID NO: 163;
b) a tyrosine at position 82 of SEQ ID NO: 163;
c) a tyrosine at position 82 and a histidine at position 207 of SEQ ID NO: 163;
d) a tyrosine at position 82 and a tyrosine at position 207 of SEQ ID NO: 163, or
e) a tyrosine at position 207 of SEQ ID NO: 163.

Embodiment 127. The antibody of any one of the preceding embodiments, wherein the antibody comprises an IgG Fc polypeptide comprising an amino acid sequence of SEQ ID NO: 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 237, 238, 239, 240, 254, 255, 256, 257, 258, 259, 260, 261, 262, 263, 264, 265, 266, 267, 381, 382, 383, 384, 385, 386, 387, 388, 389, 390, 391, 392, 393, and/or 394.

Embodiment 128. The antibody of any one of the preceding embodiments, wherein the antibody comprises:
a) (i) a heavy chain amino acid sequence of SEQ ID NO: 25 or SEQ ID NO: 47; (ii) a light chain amino acid sequence of SEQ ID NO: 26 or SEQ ID NO: 48; or (iii) a heavy chain amino acid sequence as in (i) and a light chain sequence as in (ii);
b) (i) a heavy chain amino acid sequence of SEQ ID NO: 51 or SEQ ID NO: 55; (ii) a light chain amino acid sequence of SEQ ID NO: 52 or SEQ ID NO: 56; or (iii) a heavy chain amino acid sequence as in (i) and a light chain sequence as in (ii);
c) (i) a heavy chain amino acid sequence of SEQ ID NO: 71, SEQ ID NO: 72, SEQ ID NO: 75, SEQ ID NO: 76, SEQ ID NO: 276, SEQ ID NO: 370, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, or SEQ ID NO: 421; (ii) a light chain amino acid sequence of SEQ ID NO: 73, SEQ ID NO: 74, SEQ ID NO: 77, SEQ ID NO: 78, SEQ ID NO: 277, SEQ ID NO: 371; or (iii) a heavy chain amino acid sequence as in (i) and a light chain sequence as in (ii);
d) (i) a heavy chain amino acid sequence of SEQ ID NO: 79, SEQ ID NO: 80, SEQ ID NO: 82, SEQ ID NO: 372, SEQ ID NO: 373, SEQ ID NO: 83, SEQ ID NO: 374, or SEQ ID NO: 375; (ii) a light chain amino acid sequence of SEQ ID NO: 81, SEQ ID NO: 84, SEQ ID NO: 376, SEQ ID NO: 377, SEQ ID NO: 378; or (iii) a heavy chain amino acid sequence as in (i) and a light chain sequence as in (ii);
e) (i) a heavy chain amino acid sequence of SEQ ID NO: 243; (ii) a light chain amino acid sequence of SEQ ID NO: 244; or (iii) a heavy chain amino acid sequence as in (i) and a light chain sequence as in (ii);
f) (i) a heavy chain amino acid sequence of SEQ ID NO: 348 or SEQ ID NO: 349; (ii) a light chain amino acid sequence of SEQ ID NO: 350; or (iii) a heavy chain amino acid sequence as in (i) and a light chain sequence as in (ii); or
g) (i) a heavy chain amino acid sequence of SEQ ID NO: 351 or SEQ ID NO: 352; (ii) a light chain amino acid sequence of SEQ ID NO: 253; or (iii) a heavy chain amino acid sequence as in (i) and a light chain sequence as in (ii).

Embodiment 129. An isolated antibody that binds to canine IL4R or feline IL4R, wherein the antibody comprises a variable light chain amino acid sequence of SEQ ID NO: 408 and/or a light chain amino sequence of SEQ ID NO: 409.

Embodiment 130. The antibody of any one of the preceding embodiments, wherein the antibody is a bispecific antibody that binds to IL4R and one or more antigens selected from IL17, IL31, TNFα, CD20, CD19, CD25, IL4, IL13, IL23, IgE, CD11α, IL6R, α4-Intergrin, IL12, IL1β, or BlyS.

Embodiment 131. The antibody of any one of the preceding embodiments, wherein the antibody comprises (i) a heavy chain amino acid sequence of SEQ ID NO: 245; (ii) a light chain amino acid sequence of SEQ ID NO: 246; or (iii) a heavy chain amino acid sequence as in (i) and a light chain sequence as in (ii).

Embodiment 132. The antibody of any one of the preceding embodiments, wherein the antibody is an antibody fragment, such as an Fv, scFv, Fab, Fab′, F(ab′)2, or Fab′-SH fragment.

Embodiment 133. An isolated nucleic acid encoding the antibody of any one of the preceding embodiments.

Embodiment 134. A host cell comprising the nucleic acid of embodiments 133.

Embodiment 135. A host cell that expresses the antibody of any one of embodiments 1 to 134.

Embodiment 136. A method of producing an antibody comprising culturing the host cell of embodiment 134 or embodiment 135 and isolating the antibody.

Embodiment 137. A pharmaceutical composition comprising the antibody of any one of embodiments 1 to 132 and a pharmaceutically acceptable carrier.

Embodiment 138. A method of treating a companion animal species having an IL4/IL13-induced condition, the method comprising administering to the companion animal species a therapeutically effective amount of the antibody of any one of embodiments 1 to 132 or the pharmaceutical composition of embodiment 137.

Embodiment 139. The method of embodiment 138, wherein the companion animal species is canine, feline, or equine.

Embodiment 140. The method of embodiment 138 or embodiment 138, wherein the IL4/IL13-induced condition is a pruritic or allergic condition.

Embodiment 141. The method of any one of embodiment 138 to 140, wherein the IL4/IL13-induced condition is selected from atopic dermatitis, allergic dermatitis, pruritus, asthma, psoriasis, scleroderma, and eczema.

Embodiment 142. The method of any one of embodiments 138 to 141, wherein the antibody or the pharmaceutical composition is administered parenterally.

Embodiment 143. The method of any one of embodiments 138 to 142, wherein the antibody or the pharmaceutical composition is administered by an intramuscular route, an intraperitoneal route, an intracerebrospinal route, a subcutaneous route, an intra-arterial route, an intrasynovial route, an intrathecal route, or an inhalation route.

Embodiment 144. The method of any one of embodiments 138 to 143, wherein the method comprises administering in combination with the antibody or the pharmaceutical composition a Jak inhibitor, a PI3K inhibitor, an ERK inhibitor.

Embodiment 145. The method of any one of embodiments 138 to 144, wherein the method comprises administering in combination with the antibody or the pharmaceutical composition one or more antibodies selected from an anti-IL17 antibody, an anti-IL31 antibody, an anti-TNFα antibody, an anti-CD20 antibody, an anti-CD19 antibody, an anti-CD25 antibody, an anti-IL4 antibody, an anti-IL13 antibody, an anti-IL23 antibody, an anti-IgE antibody, an anti-CD11a antibody, anti-IL6R antibody, anti-α4-Intergrin antibody, an anti-IL12 antibody, an anti-IL 1β antibody, and an anti-BlyS antibody.

Embodiment 146. A method of reducing IL4 and/or IL13 signaling function in a cell, the method comprising exposing to the cell the antibody of any one of embodiments 1 to 132 or the pharmaceutical composition of embodiment 137 under conditions permissive for binding of the antibody to extracellular IL4 and/or IL13, thereby reducing binding of IL4 and/or IL13 to IL4R and/or reducing IL4 and/or IL13 signaling function by the cell.

Embodiment 147. The method of embodiment 146, wherein the cell is exposed to the antibody or the pharmaceutical composition ex vivo.

Embodiment 148. The method of embodiment 146, wherein the cell is exposed to the antibody or the pharmaceutical composition in vivo.

Embodiment 149. The method of any one of embodiment 146 to 148, wherein the cell is a canine cell or a feline cell.

Embodiment 150. The method of any one of embodiment 146 to 149, wherein the antibody reduces IL4 and/or IL13 signaling function in the cell, as determined by a reduction in STAT6 phosphorylation.

Embodiment 151. The method of any one of embodiments 146 to 150, wherein the cell is a canine DH82 cell.

Embodiment 152. A method for detecting IL4R in a sample from a companion animal species comprising contacting the sample with the antibody of any one of embodiments 1 to 132 or the pharmaceutical composition of embodiment 137 under conditions permissive for binding of the antibody to IL4R.

Embodiment 153. The method of embodiment 152, wherein the sample is a biological sample obtained from a canine or a feline.

### BRIEF DESCRIPTION OF THE DRAWINGS

FIG. 1 is an alignment of heavy and light chain amino acid sequences of Clone B and Clone I mouse monoclonal antibody clones.

FIG. 2A and FIG. 2B are graphs of canine IL4R competitive epitope binding analyses with Clone B followed by Clone I (FIG. 2A) and with Clone I followed by Clone B.

FIGS. 3A, 3B, 3C, and 3D are graphs of canine IL4R competitive binding analyses with Clone B or Clone I followed by canine IL4 (FIG. 3A); with Clone B or Clone I followed by canine IL13 (FIG. 3B); with canine IL4 followed by Clone B or Clone I (FIG. 3C); and with canine IL13 followed by Clone B or Clone I (FIG. 3D).

FIGS. 4A and 4B are immunoblots of feline, equine, murine, human, and canine IL4R ECD polypeptides probed with Clone I (FIG. 4A) and anti-human Fc antibody as a control (FIG. 4B) under non-reducing (−DTT, left panel) and reducing (+DTT, right panel) conditions.

FIG. 5A is an illustration of canine/human IL4R ECD hybrid polypeptides used for canine IL4R epitope mapping analyses. FIGS. 5B and 5C are immunoblots of canine IL4R ECD, human IL4R ECD, the various canine/human IL4R ECD hybrid polypeptides illustrated in 5A probed with Clone I (FIG. 5B) and anti-human Fc antibody as a control (FIG. 5C).

FIG. 6A is an illustration of canine/human IL4R ECD hybrid polypeptides used for additional canine IL4R epitope mapping analyses. FIGS. 6B and 6C are immunoblots of canine IL4R ECD, human IL4R ECD, and the various canine/human IL4R ECD hybrid polypeptides illustrated in 6A probed with Clone I (FIG. 6B) and anti-human Fc antibody as a control (FIG. 6C).

FIG. 7A identifies canine IL4R ECD alanine mutant polypeptides further described in Table 1, which were used for additional canine IL4R epitope mapping analyses.

FIGS. 7B and 7C are immunoblots of human IL4R ECD, canine IL4R ECD, and the various canine IL4R ECD alanine mutant polypeptides probed with Clone I (FIG. 7B) and anti-human Fc antibody as a control (FIG. 7C).

FIG. 8 is a three-dimensional model of a complex of canine IL4, canine IL4R ECD, and canine IL13R ECD. A first epitope is identified by the arrow.

FIG. 9A is an illustration of canine/human IL4R ECD hybrid polypeptides used for canine IL4R epitope mapping analyses. FIG. 9B summarizes western blotting analysis of canine IL4R ECD, human IL4R ECD, and various canine/human IL4R ECD hybrid polypeptides probed with M3, M8, and M9 antibodies.

FIG. 10 shows a Biacore sensorgram of various concentrations of canine FcRn (12.5, 25, 50, 100, and 200 nM) binding to wild-type canine IgG-B Fc polypeptide.

FIG. 11 shows a Biacore sensorgram of various concentrations of canine FcRn (12.5, 25, 50, 100, and 200 nM) binding to variant canine IgG-B Fc polypeptide L(23)Y.

FIG. 12 shows a Biacore sensorgram of various concentrations of canine FcRn (12.5, 25, 50, 100, and 200 nM) binding to variant canine IgG-B Fc polypeptide L(23)F.

FIG. 13 shows a Biacore sensorgram of various concentrations of canine FcRn (12.5, 25, 50, 100, and 200 nM) binding to variant canine IgG-B Fc polypeptide L(23)M.

FIG. 14 shows a Biacore sensorgram of various concentrations of canine FcRn (12.5, 25, 50, 100, and 200 nM) binding to variant canine IgG-B Fc polypeptide YTE.

FIG. 15 is a OctetRed sensorgram of chimeric variant canine IgG-A Fc F00 antibody (A) and IgG-D Fc F00 antibody (B) binding to canine FcRn compared to that of chimeric variant canine IgG-A Fc without the Phe mutation (C) and IgG-D Fc without the Phe mutation (D).

FIG. 16 shows the serum pharmacokinetics profiles for chimeric variant canine IgG-A F00 antibody (“IgG-A F00”; n=2) and chimeric variant canine IgG-A without the Phe mutation (“IgG-A”; n=2) after subcutaneous administration to rats at 2 mg/kg.

FIG. 17 is a OctetRed sensorgram of chimeric antibodies with variant canine IgG-B Fcs (0Y0, 0YH, 0YY, or 00Y) binding to canine FcRn compared to that of chimeric antibody with a wild-type canine IgG-B.

FIG. 18 is a chart showing percent antibody normalized over time resulting from the in vivo pharmacokinetic study in dog as described in Example 27.

### DESCRIPTION OF CERTAIN SEQUENCES

Table 1 provides a listing of certain sequences referenced herein.

TABLE 1
Description of the Sequences

| SEQ ID | | DESCRIPTION |
|---|---|---|
| NO: | SEQUENCE | |
| 1 | GYTFTSYVMX1 | CDR-H1 |
| | wherein X₁ is H or N | |
| 2 | YINPX₂NDGTFYX₃GX₄X₅X₆G | CDR-H2 |
| | wherein X₂ is K, A, or N; X₃ is N or A; | |
| | X₄ K or A; X₅ is F or V; and X₆ is K or Q | |
| 268 | YINPX₂NDGT | Alternative CDR-H2 |
| | wherein X₂ is K, A, or N | |
| 3 | FX₇YGX₈AY | CDR-H3 |
| | wherein X₇ is N or Y; and X₈ is I or F | |
| 4 | RASQEISGYLX₉ | CDR-L1 |
| | wherein X₉ is S or A | |
| 5 | AASX₁₀X₁₁DX₁₂ | CDR-L2 |
| | wherein X₁₀ is T or N; X₁₁ is R or L; and | |
| | X₁₂ is S or T | |
| 6 | X₁₃QYASYPWT | CDR-L3 |
| | Wherein X₁₃ is V or L | |
| 7 | GYTFTSYVMH | Clone B CDR-H1 |
| 8 | YINPKNDGTFYNGKFKG | Clone B CDR-H2 |
| 269 | YINPKNDGT | Alternative Clone B |
| | | CDR-H2 |
| 9 | FNYGIAY | Clone B CDR-H3 |
| 10 | EVKLEESGPELVKPGASVKMSCKAS | Clone B HC-FR1 |
| 11 | WVKQKPGQGLEWIG | Clone B HC-FR2 |
| 12 | KATLTSDKSSSTAYMELSSLTSEDSAVYYCAA | Clone B HC-FR3 |
| 270 | FYNGKFKGKATLTSDKSSSTAYMELSSLTSEDSAVYYCA | Alternative Clone B |
| | A | HC-FR3 |
| 13 | WGQGTLVTVSS | Clone B HC-FR4 |
| 14 | RASQEISGYLS | Clone B CDR-L1 |
| 15 | AASTLDS | Clone B CDR-L2 |
| 16 | VQYASYPWT | Clone B CDR-L3 |
| 17 | DIVLTQTPSSLSASLGERVSLTCRAS | Clone B LC-FR1 |
| 18 | WLQQKPDGTIKRLIY | Clone B LC-FR2 |
| 19 | GVPKRFSGSRSGSDFSLTISSLESEDFADYYC | Clone B LC-FR3 |
| 20 | FGGGARLEIK | Clone B LC-FR4 |
| 21 | EVKLEESGPELVKPGASVKMSCKASGYTFTSYVMHWVKQ | Clone B variable HC |
| | KPGQGLEWIGYINPKNDGTFYNGKFKGKATLTSDKSSST | |
| | AYMELSSLTSEDSAVYYCAAFNYGIAYWGQGTLVTVSS | |
| 22 | IVLTQTPSSLSASLGERVSLTCRASQEISGYLSWLQQKP | Clone B variable LC |
| | DGTIKRLIYAASTLDSGVPKRFSGSRSGSDFSLTISSLE | |
| | SEDFADYYCVQYASYPWTFGGGARLEIK | |
| 23 | MAVLGLLLCLVTFPSCVLSEVKLEESGPELVKPGASVKM | Clone B variable HC |
| | SCKASGYTFTSYVMHWVKQKPGQGLEWIGYINPKNDGTF | with leader sequence |
| | YNGKFKGKATLTSDKSSSTAYMELSSLTSEDSAVYYCAA | |
| | FNYGIAYWGQGTLVTVSS | |
| 24 | METDTLLLWVLLLWVPGSTGIVLTQTPSSLSASLGERVS | Clone B variable LC |
| | LTCRASQEISGYLSWLQQKPDGTIKRLIYAASTLDSGVP | with leader sequence |
| | KRFSGSRSGSDFSLTISSLESEDFADYYCVQYASYPWTF | |
| | GGGARLEIK | |
| 25 | EVKLEESGPELVKPGASVKMSCKASGYTFTSYVMHWVKQ | Clone B HC |
| | KPGQGLEWIGYINPKNDGTFYNGKFKGKATLTSDKSSST | |
| | AYMELSSLTSEDSAVYYCAAFNYGIAYWGQGTLVTVSSA | |
| | KTTPPSVYPLAPGSAAQTNSMVTLGCLVKGYFPEPVTVT | |
| | WNSGSLSSGVHTFPAVLQSDLYTLSSSVTVPSSPRPSET | |
| | VTCNVAHPASSTKVDKKIVPRDCGCKPCICTVPEVSSVF | |
| | IFPPKPKDVLTITLTPKVTCVVVDISKDDPEVQFSWFVD | |
| | DVEVHTAQTQPREEQFNSTFRSVSELPIMHQDWLNGKEF | |
| | KCRVNSAAFPAPIEKTISKTKGRPKAPQVYTIPPPKEQM | |
| | AKDKVSLTCMITDFFPEDITVEWQWNGQPAENYKNTQPI | |
| | MNTNGSYFVYSKLNVQKSNWEAGNTFTCSVLHEGLHNHH | |
| | TEKSLSHSPGK | |
| 26 | IVLTQTPSSLSASLGERVSLTCRASQEISGYLSWLQQKP | Clone B LC |
| | DGTIKRLIYAASTLDSGVPKRFSGSRSGSDFSLTISSLE | |
| | SEDFADYYCVQYASYPWTFGGGARLEIKRADAAPTVSIF | |
| | PPSSEQLTSGGASVVCFLNNFYPKDINVKWKIDGSERQN | |
| | GVLNSWTDQDSKDSTYSMSSTLTLTKDEYERHNSYTCEA | |
| | THKTSTSPIVKSFNRNEC | |
| 27 | MAVLGLLLCLVTFPSCVLSEVKLEESGPELVKPGASVKM | Clone B HC with leader |
| | SCKASGYTFTSYVMHWVKQKPGQGLEWIGYINPKNDGTF | sequence |
| | YNGKFKGKATLTSDKSSSTAYMELSSLTSEDSAVYYCAA | |
| | FNYGIAYWGQGTLVTVSSAKTTPPSVYPLAPGSAAQTNS | |
| | MVTLGCLVKGYFPEPVTVTWNSGSLSSGVHTFPAVLQSD | |
| | LYTLSSSVTVPSSPRPSETVTCNVAHPASSTKVDKKIVP | |
| | RDCGCKPCICTVPEVSSVFIFPPKPKDVLTITLTPKVTC | |
| | VVVDISKDDPEVQFSWFVDDVEVHTAQTQPREEQFNSTF | |
| | RSVSELPIMHQDWLNGKEFKCRVNSAAFPAPIEKTISKT | |
| | KGRPKAPQVYTIPPPKEQMAKDKVSLTCMITDFFPEDIT | |
| | VEWQWNGQPAENYKNTQPIMNINGSYFVYSKLNVQKSNW | |
| | EAGNTFTCSVLHEGLHNHHTEKSLSHSPGK | |
| 28 | METDTLLLWVLLLWVPGSTGIVLTQTPSSLSASLGERVS | Clone B LC with leader |
| | LTCRASQEISGYLSWLQQKPDGTIKRLIYAASTLDSGVP | sequence |
| | KRFSGSRSGSDFSLTISSLESEDFADYYCVQYASYPWTF | |
| | GGGARLEIKRADAAPTVSIFPPSSEQLTSGGASVVCFLN | |
| | NFYPKDINVKWKIDGSERQNGVLNSWTDQDSKDSTYSMS | |
| | STLTLTKDEYERHNSYTCEATHKTSTSPIVKSFNRNEC | |
| 29 | GYTFTSYVMH | Clone I CDR-H1 |
| 358 | GYTFTSYVMN | Alternative Clone I |
| | | CDR-H1 |
| 30 | YINPNNDGTFYNGKFKG | Clone I CDR-H2 |
| 271 | YINPNNDGTFYNGKFQG | Alternative Clone I |
| | | CDR-H2 |
| 359 | YINPNNDGTFYADSVKG | Alternative Clone I |
| | | CDR-H2 |
| 272 | YINPNNDGT | Alternative Clone I |
| | | CDR-H2 |
| 31 | FYYGFAY | Clone I CDR-H3 |
| 32 | EVQLQQSGPELVKPGASVKMSCKAS | Clone I HC-FR1 |
| 33 | WVKQKPGQGLEWIG | Clone I HC-FR2 |
| 34 | KATLTSDKSSSTAYMELSSLTSEDSAVYYCAA | Clone I HC-FR3 |
| 273 | FYNGKFKGKATLISDKSSSTAYMELSSLTSEDSAVYYCA | Alternative Clone I HC- |
| | A | FR3 |
| 35 | WGQGTLVTVSA | Clone I HC-FR4 |
| 36 | RASQEISGYLS | Clone I CDR-L1 |
| 360 | RASQEISGYLA | Alternative Clone I |
| | | CDR-L1 |
| 37 | AASTLDS | Clone I CDR-L2 |
| 361 | AASTLQT | Alternative Clone I |
| | | CDR-L2 |
| 362 | AASTLDT | Alternative Clone I |
| | | CDR-L2 |
| 38 | LQYASYPWT | Clone I CDR-L3 |
| 39 | DIVLTQSPSSLSASLGERVSLTCRAS | Clone I LC-FR1 |
| 40 | WLQQKPDGTIKRLIY | Clone I LC-FR2 |
| 41 | GVPKRFSGSRSGSDFSLTISSLESEDFADYYC | Clone I LC-FR3 |
| 42 | FGGGAKLEIK | Clone I LC-FR4 |
| 43 | EVQLQQSGPELVKPGASVKMSCKASGYTFTSYVMHWVKQ | Clone I variable HC |
| | KPGQGLEWIGYINPNNDGTFYNGKFKGKATLTSDKSSST | |
| | AYMELSSLTSEDSAVYYCAAFYYGFAYWGQGTLVTVSA | |
| 44 | DIVLTQSPSSLSASLGERVSLTCRASQEISGYLSWLQQK | Clone I variable LC |
| | PDGTIKRLIYAASTLDSGVPKRFSGSRSGSDFSLTISSL | |
| | ESEDFADYYCLQYASYPWTFGGGAKLEIK | |
| 45 | MAVLGLLLCLVTFPSCVLSEVQLQQSGPELVKPGASVKM | Clone I variable HC |
| | SCKASGYTFTSYVMHWVKQKPGQGLEWIGYINPNNDGTF | with leader sequence |
| | YNGKFKGKATLTSDKSSSTAYMELSSLTSEDSAVYYCAA | |
| | FYYGFAYWGQGTLVTVSA | |
| 46 | METDTLLLWVLLLWVPGSTGDIVLTQSPSSLSASLGERV | Clone I variable LC with |
| | SLTCRASQEISGYLSWLQQKPDGTIKRLIYAASTLDSGV | leader sequence |
| | PKRFSGSRSGSDFSLTISSLESEDFADYYCLQYASYPWT | |
| | FGGGAKLEIK | |
| 47 | EVQLQQSGPELVKPGASVKMSCKASGYTFTSYVMHWVKQ | Clone I HC |
| | KPGQGLEWIGYINPNNDGTFYNGKFKGKATLTSDKSSST | |
| | AYMELSSLTSEDSAVYYCAAFYYGFAYWGQGTLVTVSAA | |
| | KTTPPSVYPLAPGSAAQTNSMVTLGCLVKGYFPEPVTVT | |
| | WNSGSLSSGVHTFPAVLQSDLYTLSSSVTVPSSPRPSET | |
| | VTCNVAHPASSTKVDKKIVPRDCGCKPCICTVPEVSSVF | |
| | IFPPKPKDVLTITLTPKVTCVVVDISKDDPEVQFSWFVD | |
| | DVEVHTAQTQPREEQFNSTFRSVSELPIMHQDWLNGKEF | |
| | KCRVNSAAFPAPIEKTISKTKGRPKAPQVYTIPPPKEQM | |
| | AKDKVSLTCMITDFFPEDITVEWQWNGQPAENYKNTQPI | |
| | MNTNGSYFVYSKLNVQKSNWEAGNTFTCSVLHEGLHNHH | |
| | TEKSLSHSPGK | |
| 48 | DIVLTQSPSSLSASLGERVSLTCRASQEISGYLSWLQQK | Clone ILC |
| | PDGTIKRLIYAASTLDSGVPKRFSGSRSGSDFSLTISSL | |
| | ESEDFADYYCLQYASYPWTFGGGAKLEIKRADAAPTVSI | |
| | FPPSSEQLTSGGASVVCFLNNFYPKDINVKWKIDGSERQ | |
| | NGVLNSWTDQDSKDSTYSMSSTLTLTKDEYERHNSYTCE | |
| | ATHKTSTSPIVKSFNRNEC | |
| 49 | MAVLGLLLCLVTFPSCVLSEVQLQQSGPELVKPGASVKM | Clone I HC with leader |
| | SCKASGYTFTSYVMHWVKQKPGQGLEWIGYINPNNDGTF | sequence |
| | YNGKFKGKATLTSDKSSSTAYMELSSLTSEDSAVYYCAA | |
| | FYYGFAYWGQGTLVTVSAAKTTPPSVYPLAPGSAAQTNS | |
| | MVTLGCLVKGYFPEPVTVTWNSGSLSSGVHTFPAVLQSD | |
| | LYTLSSSVTVPSSPRPSETVTCNVAHPASSTKVDKKIVP | |
| | RDCGCKPCICTVPEVSSVFIFPPKPKDVLTITLTPKVTC | |
| | VVVDISKDDPEVQFSWFVDDVEVHTAQTQPREEQFNSTF | |
| | RSVSELPIMHQDWLNGKEFKCRVNSAAFPAPIEKTISKT | |
| | KGRPKAPQVYTIPPPKEQMAKDKVSLTCMITDFFPEDIT | |
| | VEWQWNGQPAENYKNTQPIMNINGSYFVYSKLNVQKSNW | |
| | EAGNTFTCSVLHEGLHNHHTEKSLSHSPGK | |
| 50 | METDTLLLWVLLLWVPGSTGDIVLTQSPSSLSASLGERV | Clone I LC with leader |
| | SLTCRASQEISGYLSWLQQKPDGTIKRLIYAASTLDSGV | sequence |
| | PKRFSGSRSGSDFSLTISSLESEDFADYYCLQYASYPWT | |
| | FGGGAKLEIKRADAAPTVSIFPPSSEQLTSGGASVVCFL | |
| | NNFYPKDINVKWKIDGSERQNGVLNSWTDQDSKDSTYSM | |
| | SSTLTLTKDEYERHNSYTCEATHKTSTSPIVKSENRNEC | |
| 51 | EVKLEESGPELVKPGASVKMSCKASGYTFTSYVMHWVKQ | Chimeric Clone B |
| | KPGQGLEWIGYINPKNDGTFYNGKFKGKATLTSDKSSST | variable HC and canine |
| | AYMELSSLTSEDSAVYYCAAFNYGIAYWGQGTLVTVSSA | IgG-B |
| | STTAPSVFPLAPSCGSTSGSTVALACLVSGYFPEPVTVS | (Chimeric B HC) |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EMLGGPSVFIFPPKPKDTLLIARTPEVTCVVVDLDPEDP | |
| | EVQISWFVDGKQMQTAKTQPREEQFNGTYRVVSVLPIGH | |
| | QDWLKGKQFTCKVNNKALPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDIFIC | |
| | AVMHEALHNHYTQESLSHSPGK | |
| 52 | IVLTQTPSSLSASLGERVSLTCRASQEISGYLSWLQQKP | Chimeric Clone B |
| | DGTIKRLIYAASTLDSGVPKRFSGSRSGSDFSLTISSLE | variable LC and canine κ |
| | SEDFADYYCVQYASYPWTFGGGARLEIKRNDAQPAVYLF | light constant region |
| | QPSPDQLHTGSASVVCLLNSFYPKDINVKWKVDGVIQDT | (Chimeric B LC) |
| | GIQESVTEQDKDSTYSLSSTLTMSSTEYLSHELYSCEIT | |
| | HKSLPSTLIKSFQRSECQRVD | |
| 53 | MAVLGLLLCLVTFPSCVLSEVKLEESGPELVKPGASVKM | Chimeric Clone B |
| | SCKASGYTFTSYVMHWVKQKPGQGLEWIGYINPKNDGTF | variable HC and canine |
| | YNGKFKGKATLTSDKSSSTAYMELSSLTSEDSAVYYCAA | IgG-B with leader |
| | FNYGIAYWGQGTLVTVSSASTTAPSVFPLAPSCGSTSGS | sequence |
| | TVALACLVSGYFPEPVTVSWNSGSLTSGVHTFPSVLQSS | |
| | GLYSLSSMVTVPSSRWPSETFTCNVAHPASKTKVDKPVP | |
| | KRENGRVPRPPDCPKCPAPEMLGGPSVFIFPPKPKDILL | |
| | IARTPEVTCVVVDLDPEDPEVQISWFVDGKQMQTAKTQP | |
| | REEQFNGTYRVVSVLPIGHQDWLKGKQFTCKVNNKALPS | |
| | PIERTISKARGQAHQPSVYVLPPSREELSKNTVSLTCLI | |
| | KDFFPPDIDVEWQSNGQQEPESKYRTTPPQLDEDGSYFL | |
| | YSKLSVDKSRWQRGDTFICAVMHEALHNHYTQESLSHSP | |
| | GK | |
| 54 | METDTLLLWVLLLWVPGSTGIVLTQTPSSLSASLGERVS | Chimeric Clone B |
| | LTCRASQEISGYLSWLQQKPDGTIKRLIYAASTLDSGVP | variable LC and canine κ |
| | KRFSGSRSGSDFSLTISSLESEDFADYYCVQYASYPWTF | light constant region |
| | GGGARLEIKRNDAQPAVYLFQPSPDQLHTGSASVVCLLN | with leader sequence |
| | SFYPKDINVKWKVDGVIQDTGIQESVTEQDKDSTYSLSS | |
| | TLTMSSTEYLSHELYSCEITHKSLPSTLIKSFQRSECQR | |
| | VD | |
| 55 | EVQLQQSGPELVKPGASVKMSCKASGYTFTSYVMHWVKQ | Chimeric Clone I |
| | KPGQGLEWIGYINPNNDGTFYNGKFKGKATLTSDKSSST | variable HC and canine |
| | AYMELSSLTSEDSAVYYCAAFYYGFAYWGQGTLVTVSAA | IgG-B |
| | STTAPSVFPLAPSCGSTSGSTVALACLVSGYFPEPVTVS | (Chimeric I HC) |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EMLGGPSVFIFPPKPKDTLLIARTPEVTCVVVDLDPEDP | |
| | EVQISWFVDGKQMQTAKTQPREEQFNGTYRVVSVLPIGH | |
| | QDWLKGKQFTCKVNNKALPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 56 | DIVLTQSPSSLSASLGERVSLTCRASQEISGYLSWLQQK | Chimeric Clone I |
| | PDGTIKRLIYAASTLDSGVPKRFSGSRSGSDFSLTISSL | variable LC and canine κ |
| | ESEDFADYYCLQYASYPWTFGGGAKLEIKRNDAQPAVYL | light constant region |
| | FQPSPDQLHTGSASVVCLLNSFYPKDINVKWKVDGVIQD | (Chimeric I LC) |
| | TGIQESVTEQDKDSTYSLSSTLTMSSTEYLSHELYSCEI | |
| | THKSLPSTLIKSFQRSECQRVD | |
| 57 | MAVLGLLLCLVTFPSCVLSEVQLQQSGPELVKPGASVKM | Chimeric Clone I |
| | SCKASGYTFTSYVMHWVKQKPGQGLEWIGYINPNNDGTF | variable HC and canine |
| | YNGKFKGKATLTSDKSSSTAYMELSSLTSEDSAVYYCAA | IgG-B with leader |
| | FYYGFAYWGQGTLVTVSAASTTAPSVFPLAPSCGSTSGS | sequence |
| | TVALACLVSGYFPEPVTVSWNSGSLTSGVHTFPSVLQSS | |
| | GLYSLSSMVTVPSSRWPSETFTCNVAHPASKTKVDKPVP | |
| | KRENGRVPRPPDCPKCPAPEMLGGPSVFIFPPKPKDTLL | |
| | IARTPEVTCVVVDLDPEDPEVQISWFVDGKQMQTAKTQP | |
| | REEQFNGTYRVVSVLPIGHQDWLKGKQFTCKVNNKALPS | |
| | PIERTISKARGQAHQPSVYVLPPSREELSKNTVSLTCLI | |
| | KDFFPPDIDVEWQSNGQQEPESKYRTTPPQLDEDGSYFL | |
| | YSKLSVDKSRWQRGDTFICAVMHEALHNHYTQESLSHSP | |
| | GK | |
| 58 | METDTLLLWVLLLWVPGSTGDIVLTQSPSSLSASLGERV | Chimeric Clone I |
| | SLTCRASQEISGYLSWLQQKPDGTIKRLIYAASTLDSGV | variable LC and canine κ |
| | PKRFSGSRSGSDFSLTISSLESEDFADYYCLQYASYPWT | light constant region |
| | FGGGAKLEIKRNDAQPAVYLFQPSPDQLHTGSASVVCLL | with leader sequence |
| | NSFYPKDINVKWKVDGVIQDTGIQESVTEQDKDSTYSLS | |
| | STLTMSSTEYLSHELYSCEITHKSLPSTLIKSFQRSECQ | |
| | RVD | |
| 59 | EVQLVESGGDLVKPGGSLRLSCKASGYTFTSYVMHWVRQ | Caninized Clone B |
| | APGQGLEWVAYINPKNDGTFYNGAVKGRFTISRDNARNT | variable HC v1 |
| | LYLQMNSLRSEDTAVYYCAAFNYGIAYWGQGTLVTVSS | |
| 60 | EVQLVQSGAEVKKPGASVKVSCKASGYTFTSYVMHWVRQ | Caninized Clone B |
| | APGQGLEWMGYINPKNDGTFYNGKFQGRVTLTADTSTST | variable HC v2 |
| | AYMELSSLRAGDIAVYYCAAFNYGIAYWGQGTLVTVSS | |
| 61 | EIVMTQSPASLSLSQEEKVTITCRASQEISGYLSWLQQK | Caninized Clone B |
| | PGGTIKRLIYAASNRDTGVPSRFSGSGSGTDFSFTISSL | variable LC v1 |
| | EPEDVAVYYCVQYASYPWTFGGGAKLEIK | |
| 62 | DIVMTQTPLSLSVSPGETASISCRASQEISGYLSWLQQK | Caninized Clone B |
| | PGGTIKRLIYAASNRDTGVPDRFSGSGSGTDFTLRISRV | variable LC v2 |
| | EADDTGVYYCVQYASYPWTFGGGTKVELK | |
| 63 | EVQLVESGGDLVKPGGSLRLSCKASGYTFTSYVMHWVRQ | Caninized Clone I |
| | APGQGLEWVAYINPNNDGTFYNGAVKGRFTISRDNARNT | variable HC v1 |
| | LYLQMNSLRSEDTAVYYCAAFYYGFAYWGQGTLVTVSS | |
| 64 | EVQLVQSGAEVKKPGASVKVSCKASGYTFTSYVMHWVRQ | Caninized Clone I |
| | APGQGLEWMGYINPNNDGTFYNGKFQGRVTLTADTSTST | variable HC v2 |
| | AYMELSSLRAGDIAVYYCAAFYYGFAYWGQGTLVTVSS | |
| 274 | EVQLVQSAAEVKKPGASVKVSCKASGYTFTSYVMHWVRQ | Caninized Clone I |
| | APGQGLEWIGYINPNNDGTFYNGKFQGRVTLTADTSTGT | variable HC v3 |
| | TYTELSSLRAEDTAVYYCAAFYYGFAYWGQGTLVTVSS | |
| 363 | EVQLVQSGAEVKKPGASVKVSCKASGYTFTSYVMHWVRQ | Caninized Clone I |
| | APGQGLEWMGYINPNNDGTFYNGKFQGRVTLTADTSTST | variable HC v4 |
| | AYMELSSLRAGDIAVYYCAAFYYGFAYWGQGTLVTVSS | |
| 65 | EIVMTQSPASLSLSQEEKVTITCRASQEISGYLSWLQQK | Caninized Clone I |
| | PGGTIKRLIYAASNRDTGVPSRFSGSGSGTDFSFTISSL | variable LC v1 |
| | EPEDVAVYYCLQYASYPWTFGGGAKLEIK | |
| 66 | DIVMTQTPLSLSVSPGETASISCRASQEISGYLSWLQQK | Caninized Clone I |
| | PGGTIKRLIYAASNRDTGVPDRFSGSGSGTDFTLRISRV | variable LC v2 |
| | EADDTGVYYCLQYASYPWTFGGGTKVELK | |
| 275 | DIVMTQTPLSLSVSPGETASISCRASQEISGYLSWLQQK | Caninized Clone I |
| | PDGTIKRLIYAASTLDSGVPDRFSGSGSGTDFTLRISRV | variable LC v3 |
| | EADDTGVYYCLQYASYPWTFGAGTKVELK | |
| 364 | DIVMTQTPLSLSVSPGETASISCRASQEISGYLSWLQQK | Caninized Clone I |
| | PGGTIKRLIYAASTLDSGVPDRFSGSRSGTDFTLRISRV | variable LC v4 |
| | EADDTGVYYCLQYASYPWTFGGGTKVELK | |
| 67 | QVLLVQSGAEVRKPGASVKIFCKASGYTFTSYVMHWLRQ | Felinized Clone B |
| | APAQGLEWMGYINPKNDGTFYNGKFQGRLTLTADTSTNT | variable HC |
| | AYMELSSLRSADTAVYYCAAFNYGIAYWGQGTLVTVSS | |
| 68 | DITMTQSPGSLAGSPGQQVTMNCRASQEISGYLSWLQQK | Felinized Clone B |
| | PGGTIKRLIYAASTLDSGVPDRFSGSGSGTDFTLTISNL | variable LC |
| | QAEDVASYYCVQYASYPWTFGGGTKLEIK | |
| 69 | QVLLVQSGAEVRKPGASVKIFCKASGYTFTSYVMHWLRQ | Felinized Clone I |
| | APAQGLEWMGYINPNNDGTFYNGKFQGRLTLTADTSTNT | variable HC v1 |
| | AYMELSSLRSADTAVYYCAAFYYGFAYWGQGTLVTVSS | |
| 365 | DVQLVESGGDLVKPGGSLRLTCKASGYTFTSYVMNWVRQ | Felinized Clone I |
| | APKQGLQWVAYINPNNDGTFYADSVKGRFTISRDNAKNT | variable HC v2 |
| | LYLQMNSLKTEDTATYYCAAFYYGFAYWGQGTLVTVSS | |
| 366 | DVQLVESGGDLVKPGGSLRLTCKASGYTFTSYVMHWVKQ | Felinized Clone I |
| | KPGQGLEWIGYINPNNDGTFYNGKFKGRFTISRDNAKNT | variable HC v3 |
| | LYLQMNSLKTEDTATYYCAAFYYGFAYWGQGTLVTVSS | |
| 70 | DITMTQSPGSLAGSPGQQVTMNCRASQEISGYLSWLQQK | Felinized Clone I |
| | PGGTIKRLIYAASTLDSGVPDRFSGSGSGTDFTLTISNL | variable LC v1 |
| | QAEDVASYYCLQYASYPWTFGGGTKLEIK | |
| 367 | EIQMTQSPSSLSASPGDRVTITCRASQEISGYLAWYQQK | Felinized Clone I |
| | PGKVPKLLIYAASTLQTGVPSRFSGSGSGTDFTLTISSL | variable LC v2 |
| | EPEDAATYYCLQYASYPWTFGQGTKLEIK | |
| 368 | EIQMTQSPSSLSASPGDRVTITCRASQEISGYLSWLQQK | Felinized Clone I |
| | PDGTIKRLIYAASTLDTGVPSRFSGSGSGTDFTLTISSL | variable LC v3 |
| | EPEDAATYYCLQYASYPWTFGQGTKLEIK | |
| 369 | EITMTQSPGSLAGSPGQQVTMNCRASQEISGYLSWLQQK | Felinized Clone I |
| | PDGTIKRLIYAASTLDSGVPDRFSGSGSGTDFTLTISNL | variable LC v4 |
| | QAEDVASYYCLQYASYPWTFGQGTKLEIK | |
| 71 | EVQLVESGGDLVKPGGSLRLSCKASGYTFTSYVMHWVRQ | Caninized Clone B |
| | APGQGLEWVAYINPKNDGTFYNGAVKGRFTISRDNARNT | variable HC v1 and |
| | LYLQMNSLRSEDTAVYYCAAFNYGIAYWGQGTLVTVSSA | variant canine IgG-B |
| | STTAPSVFPLAPSCGSTSGSTVALACLVSGYFPEPVTVS | Clq−, CD16− |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EPLGGPSVFIFPPKPKDTLLIARTPEVTCVVVDLDREDP | |
| | EVQISWFVDGKQMQTAKTQPREEQFNGTYRVVSVLPIGH | |
| | QDWLKGKQFTCRVNNKALPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 72 | EVQLVQSGAEVKKPGASVKVSCKASGYTFTSYVMHWVRQ | Caninized Clone B |
| | APGQGLEWMGYINPKNDGTFYNGKFQGRVTLTADTSTST | variable HC v2 and |
| | AYMELSSLRAGDIAVYYCAAFNYGIAYWGQGTLVTVSSA | variant canine IgG-B |
| | STTAPSVFPLAPSCGSTSGSTVALACLVSGYFPEPVTVS | Clq−, CD16− |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EPLGGPSVFIFPPKPKDTLLIARTPEVTCVVVDLDREDP | |
| | EVQISWFVDGKQMQTAKTQPREEQFNGTYRVVSVLPIGH | |
| | QDWLKGKQFTCRVNNKALPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 73 | EIVMTQSPASLSLSQEEKVTITCRASQEISGYLSWLQQK | Caninized Clone B |
| | PGGTIKRLIYAASNRDTGVPSRFSGSGSGTDFSFTISSL | variable LC v1 and |
| | EPEDVAVYYCVQYASYPWTFGGGAKLEIKRNDAQPAVYL | canine κ light constant |
| | FQPSPDQLHTGSASVVCLLNSFYPKDINVKWKVDGVIQD | region |
| | TGIQESVTEQDKDSTYSLSSTLTMSSTEYLSHELYSCEI | |
| | THKSLPSTLIKSFQRSECQRVD | |
| 74 | DIVMTQTPLSLSVSPGETASISCRASQEISGYLSWLQQK | Caninized Clone B |
| | PGGTIKRLIYAASNRDTGVPDRFSGSGSGTDFTLRISRV | variable LC v2 and |
| | EADDTGVYYCVQYASYPWTFGGGTKVELKRNDAQPAVYL | canine κ light constant |
| | FQPSPDQLHTGSASVVCLLNSFYPKDINVKWKVDGVIQD | region |
| | TGIQESVTEQDKDSTYSLSSTLTMSSTEYLSHELYSCEI | |
| | THKSLPSTLIKSFQRSECQRVD | |
| 75 | EVQLVESGGDLVKPGGSLRLSCKASGYTFTSYVMHWVRQ | Caninized Clone I |
| | APGQGLEWVAYINPNNDGTFYNGAVKGRFTISRDNARNT | variable HC v1 and |
| | LYLQMNSLRSEDTAVYYCAAFYYGFAYWGQGTLVTVSSA | variant canine IgG-B |
| | STTAPSVEPLAPSCGSTSGSTVALACLVSGYFPEPVTVS | C1q−, CD16− |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EPLGGPSVFIFPPKPKDTLLIARTPEVTCVVVDLDREDP | |
| | EVQISWFVDGKQMQTAKTQPREEQFNGTYRVVSVLPIGH | |
| | QDWLKGKQFTCRVNNKALPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 76 | EVQLVQSGAEVKKPGASVKVSCKASGYTFTSYVMHWVRQ | Caninized Clone I |
| | APGQGLEWMGYINPNNDGTFYNGKFQGRVTLTADTSTST | variable HC v2 and |
| | AYMELSSLRAGDIAVYYCAAFYYGFAYWGQGTLVTVSSA | variant canine IgG-B |
| | STTAPSVFPLAPSCGSTSGSTVALACLVSGYFPEPVTVS | C1q−, CD16− |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EPLGGPSVFIFPPKPKDTLLIARTPEVTCVVVDLDREDP | |
| | EVQISWFVDGKQMQTAKTQPREEQFNGTYRVVSVLPIGH | |
| | QDWLKGKQFTCRVNNKALPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 276 | EVQLVQSAAEVKKPGASVKVSCKASGYTFTSYVMHWVRQAPGQ | Caninized Clone I |
| | GLEWIGYINPNNDGTFYNGKFQGRVTLTADTSTGTTYTELSSL | variable HC v3 and |
| | RAEDTAVYYCAAFYYGFAYWGQGTLVTVSSASTTAPSVFPLAP | variant canine IgG-B |
| | SCGSTSGSTVALACLVSGYFPEPVTVSWNSGSLTSGVHTFPSV | C1q−, CD16− |
| | LQSSGLYSLSSMVTVPSSRWPSETFTCNVAHPASKTKVDKPVP | |
| | KRENGRVPRPPDCPKCPAPEPLGGPSVFIFPPKPKDTLLIART | |
| | PEVTCVVVDLDREDPEVQISWFVDGKQMQTAKTQPREEQFNGT | |
| | YRVVSVLPIGHQDWLKGKQFTCRVNNKALPSPIERTISKARGQ | |
| | AHQPSVYVLPPSREELSKNTVSLTCLIKDFFPPDIDVEWQSNG | |
| | QQEPESKYRTTPPQLDEDGSYFLYSKLSVDKSRWQRGDTFICA | |
| | VMHEALHNHYTQESLSHSPGK | |
| 370 | EVQLVQSGAEVKKPGASVKVSCKASGYTFTSYVMHWVRQAPGQ | Caninized Clone I |
| | GLEWMGYINPNNDGTFYNGKFQGRVTLTADTSTSTAYMELSSL | variable HC v4 and |
| | RAGDIAVYYCAAFYYGFAYWGQGTLVTVSSASTTAPSVFPLAP | variant canine IgG-B |
| | SCGSTSGSTVALACLVSGYFPEPVTVSWNSGSLTSGVHTFPSV | C1q−, CD16− |
| | LQSSGLYSLSSMVTVPSSRWPSETFTCNVAHPASKTKVDKPVP | |
| | KRENGRVPRPPDCPKCPAPEMLGGPSVFIFPPKPKDTLLIART | |
| | PEVTCVVVDLDPEDPEVQISWFVDGKQMQTAKTQPREEQFNGT | |
| | YRVVSVLPIGHQDWLKGKQFTCRVNNIGLPSPIERTISKARGQ | |
| | AHQPSVYVLPPSREELSKNTVSLTCLIKDFFPPDIDVEWQSNG | |
| | QQEPESKYRTTPPQLDEDGSYFLYSKLSVDKSRWQRGDTFICA | |
| | VMHEALHNHYTQESLSHSPGK | |
| 77 | EIVMTQSPASLSLSQEEKVTITCRASQEISGYLSWLQQK | Caninized Clone I |
| | PGGTIKRLIYAASNRDTGVPSRFSGSGSGTDFSFTISSL | variable LC v1 and |
| | EPEDVAVYYCLQYASYPWTFGGGAKLEIKRNDAQPAVYL | canine κ light constant |
| | FQPSPDQLHTGSASVVCLLNSFYPKDINVKWKVDGVIQD | region |
| | TGIQESVTEQDKDSTYSLSSTLTMSSTEYLSHELYSCEI | |
| | THKSLPSTLIKSFQRSECQRVD | |
| 78 | DIVMTQTPLSLSVSPGETASISCRASQEISGYLSWLQQK | Caninized Clone I |
| | PGGTIKRLIYAASNRDTGVPDRFSGSGSGTDFTLRISRV | variable LC v2 and |
| | EADDTGVYYCLQYASYPWTFGGGTKVELKRNDAQPAVYL | canine κ light constant |
| | FQPSPDQLHTGSASVVCLLNSFYPKDINVKWKVDGVIQD | region |
| | TGIQESVTEQDKDSTYSLSSTLTMSSTEYLSHELYSCEI | |
| | THKSLPSTLIKSFQRSECQRVD | |
| 277 | DIVMTQTPLSLSVSPGETASISCRASQEISGYLSWLQQK | Caninized Clone I |
| | PDGTIKRLIYAASTLDSGVPDRFSGSGSGTDFTLRISRV | variable LC v3 and |
| | EADDTGVYYCLQYASYPWTFGAGTKVELKRNDAQPAVYL | canine κ light constant |
| | FQPSPDQLHTGSASVVCLLNSFYPKDINVKWKVDGVIQD | region |
| | TGIQESVTEQDKDSTYSLSSTLTMSSTEYLSHELYSCEI | |
| | THKSLPSTLIKSFQRSECQRVD | |
| 371 | MSVPTQVLGLLLLWLTDARCDIVMTQTPLSLSVSPGETA | Caninized Clone I |
| | SISCRASQEISGYLSWLQQKPGGTIKRLIYAASTLDSGV | variable LC v4 and |
| | PDRFSGSRSGTDFTLRISRVEADDTGVYYCLQYASYPWT | canine κ light constant |
| | FGGGTKVELKRNDAQPAVYLFQPSPDQLHTGSASVVCLL | region |
| | NSFYPKDINVKWKVDGVIQDIGIQESVTEQDKDSTYSLS | |
| | STLTMSSTEYLSHELYSCEITHKSLPSTLIKSFQRSECQ | |
| | RVD | |
| 79 | QVLLVQSGAEVRKPGASVKIFCKASGYTFTSYVMHWLRQ | Felinized Clone B |
| | APAQGLEWMGYINPKNDGTFYNGKFQGRLTLTADTSTNT | variable HC and variant |
| | AYMELSSLRSADTAVYYCAAFNYGIAYWGQGTLVTVSSA | feline IgG2 with hinge |
| | STTASSVFPLAPSCGTTSGATVALACLVLGYFPEPVTVS | Cys modification |
| | WNSGALTSGVHTFPSVLQASGLYSLSSMVTVPSSRWLSD | |
| | TFTCNVAHRPSSTKVDKTVPKTASTIESKTGECPKCPVP | |
| | EIPGAPSVFIFPPKPKDILSISRTPEVTCLVVDLGPDDS | |
| | NVQITWFVDNTEMHTAKTRPREEQFNSTYRVVSVLPILH | |
| | QDWLKGKEFKCKVNSKSLPSAMERTISKAKGQPHEPQVY | |
| | VLPPTQEELSENKVSVTCLIKGFHPPDIAVEWEITGQPE | |
| | PENNYQTTPPQLDSDGTYFLYSRLSVDRSHWQRGNTYTC | |
| | SVSHEALHSHHTQKSLTQSPGK | |
| 80 | QVLLVQSGAEVRKPGASVKIFCKASGYTFTSYVMHWLRQ | Felinized Clone B |
| | APAQGLEWMGYINPKNDGTFYNGKFQGRLTLTADTSTNT | variable HC and variant |
| | AYMELSSLRSADTAVYYCAAFNYGIAYWGQGTLVTVSSA | feline IgG2 with feline |
| | STTASSVFPLAPSCGTTSGATVALACLVLGYFPEPVTVS | IgG1hinge |
| | WNSGALTSGVHTFPSVLQASGLYSLSSMVTVPSSRWLSD | |
| | TFTCNVAHRPSSTKVDKTVRKTDHPPGPKPCDCPKCPPP | |
| | EMLGGPSVFIFPPKPKDTLSISRTPEVTCLVVDLGPDDS | |
| | NVQITWFVDNTEMHTAKTRPREEQFNSTYRVVSVLPILH | |
| | QDWLKGKEFKCKVNSKSLPSAMERTISKAKGQPHEPQVY | |
| | VLPPTQEELSENKVSVTCLIKGFHPPDIAVEWEITGQPE | |
| | PENNYQTTPPQLDSDGTYFLYSRLSVDRSHWQRGNTYTC | |
| | SVSHEALHSHHTQKSLTQSPGK | |
| 81 | DITMTQSPGSLAGSPGQQVTMNCRASQEISGYLSWLQQK | Felinized Clone B |
| | PGGTIKRLIYAASTLDSGVPDRFSGSGSGTDFTLTISNL | variable LC and feline |
| | QAEDVASYYCVQYASYPWTFGGGTKLEIKRSDAQPSVFL | K light constant region |
| | FQPSLDELHTGSASIVCILNDFYPKEVNVKWKVDGVVQN | |
| | KGIQESTTEQNSKDSTYSLSSTLTMSSTEYQSHEKFSCE | |
| | VTHKSLASTLVKSFNRSECQRE | |
| 82 | QVLLVQSGAEVRKPGASVKIFCKASGYTFTSYVMHWLRQ | Felinized Clone I |
| | APAQGLEWMGYINPNNDGTFYNGKFQGRLTLTADTSTNT | variable HC v1 and |
| | AYMELSSLRSADTAVYYCAAFYYGFAYWGQGTLVTVSSA | variant feline IgG2 with |
| | STTASSVFPLAPSCGTTSGATVALACLVLGYFPEPVTVS | hinge Cys modification |
| | WNSGALTSGVHTFPSVLQASGLYSLSSMVTVPSSRWLSD | |
| | TFTCNVAHRPSSTKVDKTVPKTASTIESKTGECPKCPVP | |
| | EIPGAPSVFIFPPKPKDTLSISRTPEVTCLVVDLGPDDS | |
| | NVQITWFVDNTEMHTAKTRPREEQFNSTYRVVSVLPILH | |
| | QDWLKGKEFKCKVNSKSLPSAMERTISKAKGQPHEPQVY | |
| | VLPPTQEELSENKVSVTCLIKGFHPPDIAVEWEITGQPE | |
| | PENNYQTTPPQLDSDGTYFLYSRLSVDRSHWQRGNTYTC | |
| | SVSHEALHSHHTQKSLTQSPGK | |
| 372 | DVQLVESGGDLVKPGGSLRLTCKASGYTFTSYVMNWVRQ | Felinized Clone I |
| | APKQGLQWVAYINPNNDGTFYADSVKGRFTISRDNAKNT | variable HC v2 and |
| | LYLQMNSLKTEDTATYYCAAFYYGFAYWGQGTLVTVSSA | variant feline IgG2 with |
| | STTASSVFPLAPSCGTTSGATVALACLVLGYFPEPVTVS | hinge Cys modification |
| | WNSGALTSGVHTFPSVLQASGLYSLSSMVTVPSSRWLSD | |
| | TFTCNVAHRPSSTKVDKTVPKTASTIESKTGECPKCPVP | |
| | EIPGAPSVFIFPPKPKDTLSISRTPEVTCLVVDLGPDDS | |
| | NVQITWFVDNTEMHTAKTRPREEQFNSTYRVVSVLPILH | |
| | QDWLKGKEFKCKVNSKSLPSAMERTISKAKGQPHEPQVY | |
| | VLPPTQEELSENKVSVTCLIKGFHPPDIAVEWEITGQPE | |
| | PENNYQTTPPQLDSDGTYFLYSRLSVDRSHWQRGNTYTC | |
| | SVSHEALHSHHTQKSLTQSPGK | |
| 373 | DVQLVESGGDLVKPGGSLRLTCKASGYTFTSYVMHWVKQ | Felinized Clone I |
| | KPGQGLEWIGYINPNNDGTFYNGKFKGRFTISRDNAKNT | variable HC v3 and |
| | LYLQMNSLKTEDTATYYCAAFYYGFAYWGQGTLVTVSSA | variant feline IgG2 with |
| | STTASSVFPLAPSCGTTSGATVALACLVLGYFPEPVTVS | hinge Cys modification |
| | WNSGALTSGVHTFPSVLQASGLYSLSSMVTVPSSRWLSD | |
| | TFTCNVAHRPSSTKVDKTVPKTASTIESKTGECPKCPVP | |
| | EIPGAPSVFIFPPKPKDILSISRTPEVTCLVVDLGPDDS | |
| | NVQITWFVDNTEMHTAKTRPREEQFNSTYRVVSVLPILH | |
| | QDWLKGKEFKCKVNSKSLPSAMERTISKAKGQPHEPQVY | |
| | VLPPTQEELSENKVSVTCLIKGFHPPDIAVEWEITGQPE | |
| | PENNYQTTPPQLDSDGTYFLYSRLSVDRSHWQRGNTYTC | |
| | SVSHEALHSHHTQKSLTQSPGK | |
| 83 | QVLLVQSGAEVRKPGASVKIFCKASGYTFTSYVMHWLRQ | Felinized Clone I |
| | APAQGLEWMGYINPNNDGTFYNGKFQGRLTLTADTSTNT | variable HC v1 and |
| | AYMELSSLRSADTAVYYCAAFYYGFAYWGQGTLVTVSSA | variant feline IgG2 with |
| | STTASSVFPLAPSCGTTSGATVALACLVLGYFPEPVTVS | feline IgG1 hinge |
| | WNSGALTSGVHTFPSVLQASGLYSLSSMVTVPSSRWLSD | |
| | TFTCNVAHRPSSTKVDKTVRKTDHPPGPKPCDCPKCPPP | |
| | EMLGGPSVFIFPPKPKDTLSISRTPEVTCLVVDLGPDDS | |
| | NVQITWFVDNTEMHTAKTRPREEQFNSTYRVVSVLPILH | |
| | QDWLKGKEFKCKVNSKSLPSAMERTISKAKGQPHEPQVY | |
| | VLPPTQEELSENKVSVTCLIKGFHPPDIAVEWEITGQPE | |
| | PENNYQTTPPQLDSDGTYFLYSRLSVDRSHWQRGNTYTC | |
| | SVSHEALHSHHTQKSLTQSPGK | |
| 374 | DVQLVESGGDLVKPGGSLRLTCKASGYTFTSYVMNWVRQ | Felinized Clone I |
| | APKQGLQWVAYINPNNDGTFYADSVKGRFTISRDNAKNT | variable HC v2 and |
| | LYLQMNSLKTEDTATYYCAAFYYGFAYWGQGTLVTVSSA | variant feline IgG2 with |
| | STTASSVFPLAPSCGTTSGATVALACLVLGYFPEPVTVS | feline IgG1 hinge |
| | WNSGALTSGVHTFPSVLQASGLYSLSSMVTVPSSRWLSD | |
| | TFTCNVAHRPSSTKVDKTVRKTDHPPGPKPCDCPKCPPP | |
| | EMLGGPSVFIFPPKPKDTLSISRTPEVTCLVVDLGPDDS | |
| | NVQITWFVDNTEMHTAKTRPREEQFNSTYRVVSVLPILH | |
| | QDWLKGKEFKCKVNSKSLPSAMERTISKAKGQPHEPQVY | |
| | VLPPTQEELSENKVSVTCLIKGFHPPDIAVEWEITGQPE | |
| | PENNYQTTPPQLDSDGTYFLYSRLSVDRSHWQRGNTYTC | |
| | SVSHEALHSHHTQKSLTQSPGK | |
| 375 | DVQLVESGGDLVKPGGSLRLTCKASGYTFTSYVMHWVKQ | Felinized Clone I |
| | KPGQGLEWIGYINPNNDGTFYNGKFKGRFTISRDNAKNT | variable HC v3 and |
| | LYLQMNSLKTEDTATYYCAAFYYGFAYWGQGTLVTVSSA | variant feline IgG2 with |
| | STTASSVFPLAPSCGTTSGATVALACLVLGYFPEPVTVS | feline IgG1 hinge |
| | WNSGALTSGVHTFPSVLQASGLYSLSSMVTVPSSRWLSD | |
| | TFTCNVAHRPSSTKVDKTVRKTDHPPGPKPCDCPKCPPP | |
| | EMLGGPSVFIFPPKPKDTLSISRTPEVTCLVVDLGPDDS | |
| | NVQITWFVDNTEMHTAKTRPREEQFNSTYRVVSVLPILH | |
| | QDWLKGKEFKCKVNSKSLPSAMERTISKAKGQPHEPQVY | |
| | VLPPTQEELSENKVSVTCLIKGFHPPDIAVEWEITGQPE | |
| | PENNYQTTPPQLDSDGTYFLYSRLSVDRSHWQRGNTYTC | |
| | SVSHEALHSHHTQKSLTQSPGK | |
| 84 | DITMTQSPGSLAGSPGQQVTMNCRASQEISGYLSWLQQK | Felinized Clone I |
| | PGGTIKRLIYAASTLDSGVPDRFSGSGSGTDFTLTISNL | variable LC v1 and |
| | QAEDVASYYCLQYASYPWTFGGGTKLEIKRSDAQPSVFL | feline κ light constant |
| | FQPSLDELHTGSASIVCILNDFYPKEVNVKWKVDGVVQN | region |
| | KGIQESTTEQNSKDSTYSLSSTLTMSSTEYQSHEKFSCE | |
| | VTHKSLASTLVKSFNRSECQRE | |
| 376 | EIQMTQSPSSLSASPGDRVTITCRASQEISGYLAWYQQK | Felinized Clone I |
| | PGKVPKLLIYAASTLQTGVPSRFSGSGSGTDFTLTISSL | variable LC v2 and |
| | EPEDAATYYCLQYASYPWTFGQGTKLEIKRSDAQPSVFL | feline κ light constant |
| | FQPSLDELHTGSASIVCILNDFYPKEVNVKWKVDGVVQN | region |
| | KGIQESTTEQNSKDSTYSLSSTLTMSSTEYQSHEKFSCE | |
| | VTHKSLASTLVKSFNRSECQRE | |
| 377 | EIQMTQSPSSLSASPGDRVTITCRASQEISGYLSWLQQK | Felinized Clone I |
| | PDGTIKRLIYAASTLDTGVPSRFSGSGSGTDFTLTISSL | variable LC v3 and |
| | EPEDAATYYCLQYASYPWTFGQGTKLEIKRSDAQPSVFL | feline κ light constant |
| | FQPSLDELHTGSASIVCILNDFYPKEVNVKWKVDGVVQN | region |
| | KGIQESTTEQNSKDSTYSLSSTLTMSSTEYQSHEKFSCE | |
| | VTHKSLASTLVKSFNRSECQRE | |
| 378 | EITMTQSPGSLAGSPGQQVTMNCRASQEISGYLSWLQQK | Felinized Clone I |
| | PDGTIKRLIYAASTLDSGVPDRFSGSGSGTDFTLTISNL | variable LC v4 and |
| | QAEDVASYYCLQYASYPWTFGQGTKLEIKRSDAQPSVFL | feline κ light constant |
| | FQPSLDELHTGSASIVCILNDFYPKEVNVKWKVDGVVQN | region |
| | KGIQESTTEQNSKDSTYSLSSTLTMSSTEYQSHEKFSCE | |
| | VTHKSLASTLVKSFNRSECQRE | |
| 85 | LX₁₀FMGSENX11T | IL4R epitope 1 |
| | wherein X₁₀ is D or N and X₁₁ is H or R | (minimal sequence) |
| 86 | RLSYQLX₁₀FMGSENX₁₁TCVPEN | IL4R epitope 1 |
| | wherein X₁₀ is D or N and X₁₁ is H or R | (expanded sequence) |
| 87 | SMX₁₂X₁₃DDX₁₄VEADVYQLX₁₅LWAGX₁₆Q | IL4R epitope 2 |
| | wherein X₁₂ is P or L, X₁₃ is I or M, X₁₄ | |
| | is A or F, X₁₅ is D or H, and X₁₆ is Q or | |
| | T | |
| 88 | LDFMGSENHT | Canine IL4R epitope 1 |
| | | (minimal sequence) |
| 89 | RLSYQLDFMGSENHTCVPEN | Canine IL4R epitope 1 |
| | | (expanded sequence) |
| 90 | SMPIDDAVEADVYQLDLWAGQQ | Canine IL4R epitope 2 |
| 91 | LNFMGSENRT | Feline IL4R epitope 1 |
| | | (minimal sequence) |
| 92 | RLSYQLNFMGSENRTCVPEN | Feline IL4R epitope 1 |
| | | (expanded sequence) |
| 93 | SMLMDDFVEADVYQLHLWAGTQ | Feline IL4R epitope 2 |
| 94 | MGRLCSGLTFPVSCLVLVWVASSGSVKVLHEPSCFSDYI | Canine IL4R |
| | STSVCQWKMDHPTNCSAELRLSYQLDFMGSENHTCVPEN | NCBI Reference |
| | REDSVCVCSMPIDDAVEADVYQLDLWAGQQLLWSGSFQP | Sequence: |
| | SKHVKPRTPGNLTVHPNISHTWLLMWTNPYPTENHLHSE | XP_0139700003.1 |
| | LTYMVNVSNDNDPEDFKVYNVTYMGPTLRLAASTLKSGA | Interleukin-4 receptor |
| | SYSARVRAWAQTYNSTWSDWSPSTTWLNYYEPWEQHLPL | subunit alpha isoform |
| | GVSISCLVILAICLSCYFSIIKIKKGWWDQIPNPAHSPL | X1 [Canis lupus |
| | VAIVIQDSQVSLWGKRSRGQEPAKCPHWKTCLTKLLPCL | familiaris] |
| | LEHGLGREEESPKTAKNGPLQGPGKPAWCPVEVSKTILW | |
| | PESISVVQCVELSEAPVDNEEEEEVEEDKRSLCPSLEGS | |
| | GGSFQEGREGIVARLTESLFLDLLGGENGGFCPQGLEES | |
| | CLPPPSGSVGAQMPWAQFPRAGPRAAPEGPEQPRRPESA | |
| | LQASPTQSAGSSAFPEPPPVVTDNPAYRSFGSFLGQSSD | |
| | PGDGDSDPELADRPGEADPGIPSAPQPPEPPAALQPEPE | |
| | SWEQILRQSVLQHRAAPAPGPGPGSGYREFTCAVKQGSA | |
| | PDAGGPGFGPSGEAGYKAFCSLLPGGATCPGTSGGEAGS | |
| | GEGGYKPFQSLTPGCPGAPTPVPVPLFTFGLDTEPPGSP | |
| | QDSLGAGSSPEHLGVEPAGKEEDSRKTLLAPEQATDPLR | |
| | DDLASSIVYSALTCHLCGHLKQWHDQEERGKAHIVPSPC | |
| | CGCCCGDRSSLLLSPLRAPNVLPGGVLLEASLSPASLVP | |
| | SGVSKEGKSSPFSQPASSSAQSSSQTPKKLAVLSTEPTC | |
| | MSAS | |
| 95 | MGRLCSGLTFPVSCLILMWAAGSGSVKVLRAPTCFSDYF | Feline IL4R |
| | STSVCQWNMDAPTNCSAELRLSYQLNFMGSENRTCVPEN | NCBI Reference |
| | GEGAACACSMLMDDFVEADVYQLHLWAGTQLLWSGSFKP | Sequence: |
| | SSHVKPRAPGNLTVHPNVSHTWLLRWSNPYPPENHLHAE | XP_023102076.1 |
| | LTYMVNISSEDDPTDSRIYNVTYMGPTLRVAASTLTSGA | Interleukin-4 receptor |
| | SYSARVRAWAQSYNSTWSEWSPSTKWLNHYEPWEQHLPL | subunit alpha [Felis |
| | GVSISCLVILAVCLSCYLSVIKIKKEWWDQIPNPAHSHL | catus] |
| | VAIVIQDPQVSLWGKRSRGQEPAKCSHWKTCLRKLLPCL | |
| | LEHGMERKEDPSKIARNGPSQGSGKSAWCPVEVSKTILW | |
| | PESISVVRCVELLEAPVESEEEEEEEEEDKGSFCPSPVN | |
| | LEDSFQEGREGIAARLTESLFMDLLGVEKGGFGPQGSLE | |
| | SWFPPPSGSAGAQMPWAEFPGPGPQEASPQGKEQPFDPR | |
| | SDPLATLPQSPASPTFPETPPVVTDNPAYRSFGTFQGRS | |
| | SGPGECDSGPELAGRLGEADPGIPAAPQPSEPPSALQPE | |
| | AETWEQILRQRVLQHRGAPAPAPGSGYREFVCAVRQGST | |
| | QDSRVGDFGPSEEAGYKAFSSLLTSGAVCPETSGGEAGS | |
| | GDGGYKPFQSLTPGCPGAPAPVPVPLFTFGLDAEPPHCP | |
| | QDSPLPGSSPEPAGKAQDSHKTPPAPEQAADPLRDDLAS | |
| | GIVYSALTCHLCGHLKQCHGQEEGGEAHPVASPCCGCCC | |
| | GDRSSPLVSPLRAPDPLPGGVPLEASLSPASPAPLAVSE | |
| | EGPPSLCFQPALSHAHSSSQTPKKVAMLSPEPTCTMAS | |
| 96 | MGCLCPGLTLPVSCLILVWAAGSGSVKVLRLTACFSDYI | Equine IL4R |
| | SASTCEWKMDRPTNCSAQLRLSYQLNDEFSDNLTCIPEN | NCBI Reference |
| | REDEVCVCRMLMDNIVSEDVYELDLWAGNQLLWNSSFKP | Sequence: |
| | SRHVKPRAPQNLTVHAISHTWLLTWSNPYPLKNHLWSEL | NP_001075243.1 |
| | TYLVNISKEDDPTDFKIYNVTYMDPTLRVTASTLKSRAT | Interleukin-4 receptor |
| | YSARVKARAQNYNSTWSEWSPSTTWHNYYEQPLEQRLPL | subunit alpha precursor |
| | GVSISCVVILAICLSCYFSIIKIKKEWWDQIPNPAHSPL | [Equus caballus] |
| | VAIVLQDSQVSLWGKQSRGQEPAKCPRWKTCLTKLLPCL | |
| | LEHGLQKEEDSSKTVRNGPFQSPGKSAWHTVEVNHTILR | |
| | PEIISVVPCVELCEAQVESEEEEVEEDRGSFCPSPESSG | |
| | SGFQEGREGVAARLTESLFLGLLGAENGALGESCLLPPL | |
| | GSAHMPWARISSAGPQEAASQGEEQPLNPESNPLATLTQ | |
| | SPGSLAFTEAPAVVADNPAYRSFSNSLSQPRGPGELDSD | |
| | PQLAEHLGQVDPSIPSAPQPSEPPTALQPEPETWEQMLR | |
| | QSVLQQGAAPAPASAPTGGYREFAQVVKQGGGAAGSGPS | |
| | GEAGYKAFSSLLAGSAVCPGQSGVEASSGEGGYRPYESP | |
| | DPGAPAPVPVPLFTFGLDVEPPHSPQNSLLPGGSPELPG | |
| | PEPTVKGEDPRKPLLSAQQATDSLRDDLGSGIVYSALTC | |
| | HLCGHLKQCHGQEEHGEAHTVASPCCGCCCGDRSSPPVS | |
| | PVRALDPPPGGVPLEAGLSLASLGSLGLSEERKPSLFFQ | |
| | PAPGNAQSSSQTPLTVAMLSTGPTCTSAS | |
| 97 | MGRLCTKFLTSVGCLILLLVTGSGSIKVLGEPTCFSDYI | Murine IL4R |
| | RTSTCEWFLDSAVDCSSQLCLHYRLMFFEFSENLTCIPR | NCBI Reference |
| | NSASTVCVCHMEMNRPVQSDRYQMELWAEHRQLWQGSFS | Sequence: |
| | PSGNVKPLAPDNLTLHTNVSDEWLLTWNNLYPSNNLLYK | NP_001008700.1 |
| | DLISMVNISREDNPAEFIVYNVTYKEPRLSFPINILMSG | Interleukin-4 receptor |
| | VYYTARVRVRSQILTGTWSEWSPSITWYNHFQLPLIQRL | subunit alpha isoform 1 |
| | PLGVTISCLCIPLFCLFCYFSITKIKKIWWDQIPTPARS | precursor [Mus |
| | PLVAIIIQDAQVPLWDKQTRSQESTKYPHWKTCLDKLLP | musculus] |
| | CLLKHRVKKKTDFPKAAPTKSLQSPGKAGWCPMEVSRTV | |
| | LWPENVSVSVVRCMELFEAPVQNVEEEEDEIVKEDLSMS | |
| | PENSGGCGFQESQADIMARLTENLFSDLLEAENGGLGQS | |
| | ALAESCSPLPSGSGQASVSWACLPMGPSEEATCQVTEQP | |
| | SHPGPLSGSPAQSAPTLACTQVPLVLADNPAYRSFSDCC | |
| | SPAPNPGELAPEQQQADHLEEEEPPSPADPHSSGPPMQP | |
| | VESWEQILHMSVLQHGAAAGSTPAPAGGYQEFVQAVKQG | |
| | AAQDPGVPGVRPSGDPGYKAFSSLLSSNGIRGDTAAAGT | |
| | DDGHGGYKPFQNPVPNQSPSSVPLFTFGLDTELSPSPLN | |
| | SDPPKSPPECLGLELGLKGGDWVKAPPPADQVPKPFGDD | |
| | LGFGIVYSSLTCHLCGHLKQHHSQEEGGQSPIVASPGCG | |
| | CCYDDRSPSLGSLSGALESCPEGIPPEANLMSAPKTPSN | |
| | LSGEGKGPGHSPVPSQTTEVPVGALGIAVS | |
| 98 | MGWLCSGLLFPVSCLVLLQVASSGNMKVLQEPTCVSDYM | Human IL4R |
| | SISTCEWKMNGPTNCSTELRLLYQLVFLLSEAHTCIPEN | NCBI Reference |
| | NGGAGCVCHLLMDDVVSADNYTLDLWAGQQLLWKGSFKP | Sequence: |
| | SEHVKPRAPGNLTVHINVSDILLLTWSNPYPPDNYLYNH | NP_000409.1 |
| | LTYAVNIWSENDPADFRIYNVTYLEPSLRIAASTLKSGI | Interleukin-4 receptor |
| | SYRARVRAWAQCYNTTWSEWSPSTKWHNSYREPFEQHLL | subunit alpha isoform a |
| | LGVSVSCIVILAVCLLCYVSITKIKKEWWDQIPNPARSR | precursor [Homo |
| | LVAIIIQDAQGSQWEKRSRGQEPAKCPHWKNCLTKLLPC | sapiens] |
| | FLEHNMKRDEDPHKAAKEMPFQGSGKSAWCPVEISKTVL | |
| | WPESISVVRCVELFEAPVECEEEEEVEEEKGSFCASPES | |
| | SRDDFQEGREGIVARLTESLFLDLLGEENGGFCQQDMGE | |
| | SCLLPPSGSTSAHMPWDEFPSAGPKEAPPWGKEQPLHLE | |
| | PSPPASPTQSPDNLTCTETPLVIAGNPAYRSFSNSLSQS | |
| | PCPRELGPDPLLARHLEEVEPEMPCVPQLSEPTTVPQPE | |
| | PETWEQILRRNVLQHGAAAAPVSAPTSGYQEFVHAVEQG | |
| | GTQASAVVGLGPPGEAGYKAFSSLLASSAVSPEKCGFGA | |
| | SSGEEGYKPFQDLIPGCPGDPAPVPVPLFTFGLDREPPR | |
| | SPQSSHLPSSSPEHLGLEPGEKVEDMPKPPLPQEQATDP | |
| | LVDSLGSGIVYSALTCHLCGHLKQCHGQEDGGQTPVMAS | |
| | PCCGCCCGDRSSPPTTPLRAPDPSPGGVPLEASLCPASL | |
| | APSGISEKSKSSSSFHPAPGNAQSSSQTPKIVNFVSVGP | |
| | TYMRVS | |
| 99 | GSVKVLHEPSCFSDYISTSVCQWKMDHPTNCSAELRLSY | Canine IL4R_ECD |
| | QLDFMGSENHTCVPENREDSVCVCSMPIDDAVEADVYQL | |
| | DLWAGQQLLWSGSFQPSKHVKPRTPGNLTVHPNISHTWL | |
| | LMWTNPYPTENHLHSELTYMVNVSNDNDPEDFKVYNVTY | |
| | MGPTLRLAASTLKSGASYSARVRAWAQTYNSTWSDWSPS | |
| | TTWLNYYE | |
| 100 | GSVKVLRAPTCFSDYFSTSVCQWNMDAPTNCSAELRLSY | Feline IL4R_ECD |
| | QLNFMGSENRTCVPENGEGAACACSMLMDDFVEADVYQL | |
| | HLWAGTQLLWSGSFKPSSHVKPRAPGNLTVHPNVSHTWL | |
| | LRWSNPYPPENHLHAELTYMVNISSEDDPTDSRIYNVTY | |
| | MGPTLRVAASTLTSGASYSARVRAWAQSYNSTWSEWSPS | |
| | TKWLNHYE | |
| 101 | VKVLRLTACFSDYISASTCEWKMDRPTNCSAQLRLSYQL | Equine IL4R_ECD |
| | NDEFSDNLTCIPENREDEVCVCRMLMDNIVSEDVYELDL | |
| | WAGNQLLWNSSFKPSRHVKPRAPQNLTVHAISHTWLLTW | |
| | SNPYPLKNHLWSELTYLVNISKEDDPTDFKIYNVTYMDP | |
| | TLRVTASTLKSRATYSARVKARAQNYNSTWSEWSPSTTW | |
| | HNYYEQPLEQR | |
| 102 | IKVLGEPTCFSDYIRTSTCEWFLDSAVDCSSQLCLHYRL | Murine IL4R_ECD |
| | MFFEFSENLTCIPRNSASTVCVCHMEMNRPVQSDRYQME | |
| | LWAEHRQLWQGSFSPSGNVKPLAPDNLTLHTNVSDEWLL | |
| | TWNNLYPSNNLLYKDLISMVNISREDNPAEFIVYNVTYK | |
| | EPRLSFPINILMSGVYYTARVRVRSQILTGTWSEWSPSI | |
| | TWYNHFQLPLIQRLPLGVTISCLCIPLFCLFCYFSITKI | |
| | KKIW | |
| 103 | GNMKVLQEPTCVSDYMSISTCEWKMNGPTNCSTELRLLY | Human IL4R_ECD |
| | QLVFLLSEAHTCIPENNGGAGCVCHLLMDDVVSADNYTL | |
| | DLWAGQQLLWKGSFKPSEHVKPRAPGNLTVHTNVSDILL | |
| | LTWSNPYPPDNYLYNHLTYAVNIWSENDPADFRIYNVTY | |
| | LEPSLRIAASTLKSGISYRARVRAWAQCYNTTWSEWSPS | |
| | TKWHNSYREPFEQH | |
| 104 | MGRLCSGLTFPVSCLVLVWVASSGSVKVLHEPSCFSDYI | Canine IL4R_C-FLAG |
| | STSVCQWKMDHPTNCSAELRLSYQLDFMGSENHTCVPEN | with leader |
| | REDSVCVCSMPIDDAVEADVYQLDLWAGQQLLWSGSFQP | |
| | SKHVKPRTPGNLTVHPNISHTWLLMWTNPYPTENHLHSE | |
| | LTYMVNVSNDNDPEDFKVYNVTYMGPTLRLAASTLKSGA | |
| | SYSARVRAWAQTYNSTWSDWSPSTTWLNYYEPWEQHLPL | |
| | GVSISCLVILAICLSCYFSIIKIKKGWWDQIPNPAHSPL | |
| | VAIVIQDSQVSLWGKRSRGQEPAKCPHWKTCLTKLLPCL | |
| | LEHGLGREEESPKTAKNGPLQGPGKPAWCPVEVSKTILW | |
| | PESISVVQCVELSEAPVDNEEEEEVEEDKRSLCPSLEGS | |
| | GGSFQEGREGIVARLTESLFLDLLGGENGGFCPQGLEES | |
| | CLPPPSGSVGAQMPWAQFPRAGPRAAPEGPEQPRRPESA | |
| | LQASPTQSAGSSAFPEPPPVVTDNPAYRSFGSFLGQSSD | |
| | PGDGDSDPELADRPGEADPGIPSAPQPPEPPAALQPEPE | |
| | SWEQILRQSVLQHRAAPAPGPGPGSGYREFTCAVKQGSA | |
| | PDAGGPGFGPSGEAGYKAFCSLLPGGATCPGTSGGEAGS | |
| | GEGGYKPFQSLTPGCPGAPTPVPVPLFTFGLDTEPPGSP | |
| | QDSLGAGSSPEHLGVEPAGKEEDSRKTLLAPEQATDPLR | |
| | DDLASSIVYSALTCHLCGHLKQWHDQEERGKAHIVPSPC | |
| | CGCCCGDRSSLLLSPLRAPNVLPGGVLLEASLSPASLVP | |
| | SGVSKEGKSSPFSQPASSSAQSSSQTPKKLAVLSTEPTC | |
| | MSASGSGSDYKDDDDK | |
| 105 | GSVKVLHEPSCFSDYISTSVCQWKMDHPINCSAELRLSY | Canine IL4R_C-FLAG |
| | QLDFMGSENHTCVPENREDSVCVCSMPIDDAVEADVYQL | |
| | DLWAGQQLLWSGSFQPSKHVKPRTPGNLTVHPNISHTWL | |
| | LMWTNPYPTENHLHSELTYMVNVSNDNDPEDFKVYNVTY | |
| | MGPTLRLAASTLKSGASYSARVRAWAQTYNSTWSDWSPS | |
| | TTWLNYYEPWEQHLPLGVSISCLVILAICLSCYFSIIKI | |
| | KKGWWDQIPNPAHSPLVAIVIQDSQVSLWGKRSRGQEPA | |
| | KCPHWKTCLTKLLPCLLEHGLGREEESPKTAKNGPLQGP | |
| | GKPAWCPVEVSKTILWPESISVVQCVELSEAPVDNEEEE | |
| | EVEEDKRSLCPSLEGSGGSFQEGREGIVARLTESLFLDL | |
| | LGGENGGFCPQGLEESCLPPPSGSVGAQMPWAQFPRAGP | |
| | RAAPEGPEQPRRPESALQASPTQSAGSSAFPEPPPVVTD | |
| | NPAYRSFGSFLGQSSDPGDGDSDPELADRPGEADPGIPS | |
| | APQPPEPPAALQPEPESWEQILRQSVLQHRAAPAPGPGP | |
| | GSGYREFTCAVKQGSAPDAGGPGFGPSGEAGYKAFCSLL | |
| | PGGATCPGTSGGEAGSGEGGYKPFQSLTPGCPGAPTPVP | |
| | VPLFTFGLDTEPPGSPQDSLGAGSSPEHLGVEPAGKEED | |
| | SRKTLLAPEQATDPLRDDLASSIVYSALTCHLCGHLKQW | |
| | HDQEERGKAHIVPSPCCGCCCGDRSSLLLSPLRAPNVLP | |
| | GGVLLEASLSPASLVPSGVSKEGKSSPFSQPASSSAQSS | |
| | SQTPKKLAVLSTEPTCMSASGSGSDYKDDDDK | |
| 106 | MDMRVPAQLLGLLLLWLRGARCSGSVKVLHEPSCFSDYI | Canine IL4R-ECD_C- |
| | STSVCQWKMDHPTNCSAELRLSYQLDFMGSENHTCVPEN | His6 with leader |
| | REDSVCVCSMPIDDAVEADVYQLDLWAGQQLLWSGSFQP | |
| | SKHVKPRTPGNLTVHPNISHTWLLMWTNPYPTENHLHSE | |
| | LTYMVNVSNDNDPEDFKVYNVTYMGPTLRLAASTLKSGA | |
| | SYSARVRAWAQTYNSTWSDWSPSTTWLNYYEPGSGSHHH | |
| | HHH | |
| 107 | GSVKVLHEPSCFSDYISTSVCQWKMDHPINCSAELRLSY | Canine IL4R-ECD_C- |
| | QLDFMGSENHTCVPENREDSVCVCSMPIDDAVEADVYQL | His6 |
| | DLWAGQQLLWSGSFQPSKHVKPRTPGNLTVHPNISHTWL | |
| | LMWTNPYPTENHLHSELTYMVNVSNDNDPEDFKVYNVTY | |
| | MGPTLRLAASTLKSGASYSARVRAWAQTYNSTWSDWSPS | |
| | TTWLNYYEPGSGSHHHHHH | |
| 108 | MDMRVPAQLLGLLLLWLRGARCSGSVKVLHEPSCFSDYI | Canine IL4R-ECD_ |
| | STSVCQWKMDHPTNCSAELRLSYQLDFMGSENHTCVPEN | C-HuFc_His6 with |
| | REDSVCVCSMPIDDAVEADVYQLDLWAGQQLLWSGSFQP | leader |
| | SKHVKPRTPGNLTVHPNISHTWLLMWTNPYPTENHLHSE | |
| | LTYMVNVSNDNDPEDFKVYNVTYMGPTLRLAASTLKSGA | |
| | SYSARVRAWAQTYNSTWSDWSPSTTWLNYYEPGSENLYF | |
| | QGPKSCDKTHTCPPCPAPELLGGPSVFLFPPKPKDTLMI | |
| | SRTPEVTCVVVDVSHEDPEVKFNWYVDGVEVHNAKTKPR | |
| | EEQYNSTYRVVSVLTVLHQDWLNGKEYKCKVSNKALPAP | |
| | IEKTISKAKGQPREPQVYTLPPSRDELTKNQVSLTCLVK | |
| | GFYPSDIAVEWESNGQPENNYKTTPPVLDSDGSFFLYSK | |
| | LTVDKSRWQQGNVFSCSVMHEALHNHYTQKSLSLSPGKH | |
| | HHHHH | |
| 109 | GSVKVLHEPSCFSDYISTSVCQWKMDHPINCSAELRLSY | Canine IL4R-ECD |
| | QLDFMGSENHTCVPENREDSVCVCSMPIDDAVEADVYQL | C-HuFc_His6 |
| | DLWAGQQLLWSGSFQPSKHVKPRTPGNLTVHPNISHTWL | |
| | LMWTNPYPTENHLHSELTYMVNVSNDNDPEDFKVYNVTY | |
| | MGPTLRLAASTLKSGASYSARVRAWAQTYNSTWSDWSPS | |
| | TTWLNYYEPGSENLYFQGPKSCDKTHTCPPCPAPELLGG | |
| | PSVFLFPPKPKDTLMISRTPEVTCVVVDVSHEDPEVKEN | |
| | WYVDGVEVHNAKTKPREEQYNSTYRVVSVLTVLHQDWLN | |
| | GKEYKCKVSNKALPAPIEKTISKAKGQPREPQVYTLPPS | |
| | RDELTKNQVSLTCLVKGFYPSDIAVEWESNGQPENNYKT | |
| | TPPVLDSDGSFFLYSKLTVDKSRWQQGNVFSCSVMHEAL | |
| | HNHYTQKSLSLSPGKHHHHHH | |
| 110 | MDMRVPAQLLGLLLLWLRGARCGSVKVLRAPTCFSDYFS | Feline IL4R-ECD_ |
| | TSVCQWNMDAPTNCSAELRLSYQLNFMGSENRTCVPENG | C-HuFc_His6 with |
| | EGAACACSMLMDDFVEADVYQLHLWAGTQLLWSGSFKPS | leader |
| | SHVKPRAPGNLTVHPNVSHTWLLRWSNPYPPENHLHAEL | |
| | TYMVNISSEDDPTDSRIYNVTYMGPTLRVAASTLTSGAS | |
| | YSARVRAWAQSYNSTWSDWSPSTTWLNYYEGSENLYFQG | |
| | PKSCDKTHTCPPCPAPELLGGPSVFLFPPKPKDTLMISR | |
| | TPEVTCVVVDVSHEDPEVKFNWYVDGVEVHNAKTKPREE | |
| | QYNSTYRVVSVLIVLHQDWLNGKEYKCKVSNKALPAPIE | |
| | KTISKAKGQPREPQVYTLPPSRDELTKNQVSLTCLVKGF | |
| | YPSDIAVEWESNGQPENNYKTTPPVLDSDGSFFLYSKLT | |
| | VDKSRWQQGNVFSCSVMHEALHNHYTQKSLSLSPGKHHH | |
| | HHH | |
| 111 | GSVKVLRAPTCFSDYFSTSVCQWNMDAPTNCSAELRLSY | Feline IL4R-ECD |
| | QLNFMGSENRTCVPENGEGAACACSMLMDDFVEADVYQL | C-HuFc_His6 |
| | HLWAGTQLLWSGSFKPSSHVKPRAPGNLTVHPNVSHTWL | |
| | LRWSNPYPPENHLHAELTYMVNISSEDDPTDSRIYNVTY | |
| | MGPTLRVAASTLTSGASYSARVRAWAQSYNSTWSDWSPS | |
| | TTWLNYYEGSENLYFQGPKSCDKTHTCPPCPAPELLGGP | |
| | SVFLFPPKPKDTLMISRTPEVTCVVVDVSHEDPEVKENW | |
| | YVDGVEVHNAKTKPREEQYNSTYRVVSVLTVLHQDWLNG | |
| | KEYKCKVSNKALPAPIEKTISKAKGQPREPQVYTLPPSR | |
| | DELTKNQVSLTCLVKGFYPSDIAVEWESNGQPENNYKTT | |
| | PPVLDSDGSFFLYSKLTVDKSRWQQGNVFSCSVMHEALH | |
| | NHYTQKSLSLSPGKHHHHHH | |
| 112 | MPSSVSWGILLLAGLCCLVPVSLAVKVLRLTACFSDYIS | Equine IL4R-ECD |
| | ASTCEWKMDRPTNCSAQLRLSYQLNDEFSDNLTCIPENR | C-HuFc_His6 with |
| | EDEVCVCRMLMDNIVSEDVYELDLWAGNQLLWNSSFKPS | leader |
| | RHVKPRAPQNLTVHAISHTWLLTWSNPYPLKNHLWSELT | |
| | YLVNISKEDDPTDFKIYNVTYMDPTLRVTASTLKSRATY | |
| | SARVKARAQNYNSTWSEWSPSTTWHNYYEQPLEQRGGGS | |
| | GGGSENLYFQGPKSCDKTHTCPPCPAPELLGGPSVFLFP | |
| | PKPKDTLMISRTPEVTCVVVDVSHEDPEVKFNWYVDGVE | |
| | VHNAKTKPREEQYNSTYRVVSVLIVLHQDWLNGKEYKCK | |
| | VSNKALPAPIEKTISKAKGQPREPQVYTLPPSRDELTKN | |
| | QVSLTCLVKGFYPSDIAVEWESNGQPENNYKTTPPVLDS | |
| | DGSFFLYSKLTVDKSRWQQGNVFSCSVMHEALHNHYTQK | |
| | SLSLSPGKHHHHHH | |
| 113 | VKVLRLTACFSDYISASTCEWKMDRPTNCSAQLRLSYQL | Equine IL4R-ECD |
| | NDEFSDNLTCIPENREDEVCVCRMLMDNIVSEDVYELDL | C-HuFc_His6 |
| | WAGNQLLWNSSFKPSRHVKPRAPQNLTVHAISHTWLLTW | |
| | SNPYPLKNHLWSELTYLVNISKEDDPTDFKIYNVTYMDP | |
| | TLRVTASTLKSRATYSARVKARAQNYNSTWSEWSPSTTW | |
| | HNYYEQPLEQRGGGSGGGSENLYFQGPKSCDKTHTCPPC | |
| | PAPELLGGPSVFLFPPKPKDTLMISRTPEVTCVVVDVSH | |
| | EDPEVKFNWYVDGVEVHNAKTKPREEQYNSTYRVVSVLT | |
| | VLHQDWLNGKEYKCKVSNKALPAPIEKTISKAKGQPREP | |
| | QVYTLPPSRDELTKNQVSLTCLVKGFYPSDIAVEWESNG | |
| | QPENNYKTTPPVLDSDGSFFLYSKLTVDKSRWQQGNVFS | |
| | CSVMHEALHNHYTQKSLSLSPGKHHHHHH | |
| 114 | MGWLCSGLLFPVSCLVLLQVASSIKVLGEPTCFSDYIRT | Murine IL4R-ECD_ |
| | STCEWFLDSAVDCSSQLCLHYRLMFFEFSENLTCIPRNS | C-HuFc_His6 with |
| | ASTVCVCHMEMNRPVQSDRYQMELWAEHRQLWQGSFSPS | leader |
| | GNVKPLAPDNLTLHTNVSDEWLLTWNNLYPSNNLLYKDL | |
| | ISMVNISREDNPAEFIVYNVTYKEPRLSFPINILMSGVY | |
| | YTARVRVRSQILTGTWSEWSPSITWYNHFQLPLIQRLPL | |
| | GVTISCLCIPLFCLFCYFSITKIKKIWGSENLYFQGPKS | |
| | CDKTHTCPPCPAPELLGGPSVFLFPPKPKDTLMISRTPE | |
| | VTCVVVDVSHEDPEVKFNWYVDGVEVHNAKTKPREEQYN | |
| | STYRVVSVLTVLHQDWLNGKEYKCKVSNKALPAPIEKTI | |
| | SKAKGQPREPQVYTLPPSRDELTKNQVSLTCLVKGFYPS | |
| | DIAVEWESNGQPENNYKTTPPVLDSDGSFFLYSKLTVDK | |
| | SRWQQGNVFSCSVMHEALHNHYTQKSLSLSPGKHHHHHH | |
| 115 | IKVLGEPTCFSDYIRTSTCEWFLDSAVDCSSQLCLHYRL | Murine IL4R-ECD |
| | MFFEFSENLTCIPRNSASTVCVCHMEMNRPVQSDRYQME | C-HuFc_His6 |
| | LWAEHRQLWQGSFSPSGNVKPLAPDNLTLHTNVSDEWLL | |
| | TWNNLYPSNNLLYKDLISMVNISREDNPAEFIVYNVTYK | |
| | EPRLSFPINILMSGVYYTARVRVRSQILIGTWSEWSPSI | |
| | TWYNHFQLPLIQRLPLGVTISCLCIPLFCLFCYFSITKI | |
| | KKIWGSENLYFQGPKSCDKTHTCPPCPAPELLGGPSVFL | |
| | FPPKPKDTLMISRTPEVTCVVVDVSHEDPEVKENWYVDG | |
| | VEVHNAKTKPREEQYNSTYRVVSVLTVLHQDWLNGKEYK | |
| | CKVSNKALPAPIEKTISKAKGQPREPQVYTLPPSRDELT | |
| | KNQVSLTCLVKGFYPSDIAVEWESNGQPENNYKTTPPVL | |
| | DSDGSFFLYSKLTVDKSRWQQGNVFSCSVMHEALHNHYT | |
| | QKSLSLSPGKHHHHHH | |
| 116 | MGWLCSGLLFPVSCLVLLQVASSGNMKVLQEPTCVSDYM | Human IL4R-ECD |
| | SISTCEWKMNGPTNCSTELRLLYQLVFLLSEAHTCIPEN | C-HuFc_His6 |
| | NGGAGCVCHLLMDDVVSADNYTLDLWAGQQLLWKGSFKP | with leader |
| | SEHVKPRAPGNLTVHTNVSDTLLLTWSNPYPPDNYLYNH | |
| | LTYAVNIWSENDPADFRIYNVTYLEPSLRIAASTLKSGI | |
| | SYRARVRAWAQCYNTTWSEWSPSTKWHNSYREPFEQHGS | |
| | ENLYFQGPKSCDKTHTCPPCPAPELLGGPSVFLFPPKPK | |
| | DTLMISRTPEVTCVVVDVSHEDPEVKFNWYVDGVEVHNA | |
| | KTKPREEQYNSTYRVVSVLTVLHQDWLNGKEYKCKVSNK | |
| | ALPAPIEKTISKAKGQPREPQVYTLPPSRDELTKNQVSL | |
| | TCLVKGFYPSDIAVEWESNGQPENNYKTTPPVLDSDGSF | |
| | FLYSKLTVDKSRWQQGNVFSCSVMHEALHNHYTQKSLSL | |
| | SPGKHHHHHH | |
| 117 | GNMKVLQEPTCVSDYMSISTCEWKMNGPTNCSTELRLLY | Human IL4R-ECD_ |
| | QLVFLLSEAHTCIPENNGGAGCVCHLLMDDVVSADNYTL | C-HuFc_His6 |
| | DLWAGQQLLWKGSFKPSEHVKPRAPGNLTVHTNVSDTLL | |
| | LTWSNPYPPDNYLYNHLTYAVNIWSENDPADFRIYNVTY | |
| | LEPSLRIAASTLKSGISYRARVRAWAQCYNTTWSEWSPS | |
| | TKWHNSYREPFEQHGSENLYFQGPKSCDKTHTCPPCPAP | |
| | ELLGGPSVFLFPPKPKDTLMISRTPEVTCVVVDVSHEDP | |
| | EVKFNWYVDGVEVHNAKTKPREEQYNSTYRVVSVLTVLH | |
| | QDWLNGKEYKCKVSNKALPAPIEKTISKAKGQPREPQVY | |
| | TLPPSRDELTKNQVSLTCLVKGFYPSDIAVEWESNGQPE | |
| | NNYKTTPPVLDSDGSFFLYSKLTVDKSRWQQGNVFSCSV | |
| | MHEALHNHYTQKSLSLSPGKHHHHHH | |
| 118 | MGLTSQLIPTLVCLLALTSTFVHGHNFNITIKEIIKMLN | Canine IL4 precursor |
| | ILTARNDSCMELTVKDVFTAPKNTSDKEIFCRAATVLRQ | NCBI Ref: |
| | IYTHNCSNRYLRGLYRNLSSMANKTCSMNEIKKSTLKDF | NP_001003159.1 |
| | LERLKVIMQKKYYRH | |
| 119 | MGLTYQLIPALVCLLAFTSTFVHGQNENNTLKEIIKTLN | Feline IL4 precursor |
| | ILTARNDSCMELTVMDVLAAPKNTSDKEIFCRATTVLRQ | UniProtKB/Swiss-Prot |
| | IYTHHNCSTKFLKGLDRNLSSMANRTCSVNEVKKCTLKD | Ref: P55030.1 |
| | FLERLKAIMQKKYSKH | |
| 120 | MGLTYQLIPALVCLLACTSNFIQGCKYDITLQEIIKTLN | Equine IL4 precursor |
| | NLTDGKGKNSCMELTVADAFAGPKNTDGKEICRAAKVLQ | NCBI Ref: |
| | QLYKRHDRSLIKECLSGLDRNLKGMANGTCCTVNEAKKS | NP_001075988.1 |
| | TLKDFLERLKTIMKEKYSKC | |
| 121 | MGLTSQLIPTLVCLLALISTFVHGHNFNITIKEIIKMLN | Canine IL4 precursor |
| | ILTARNDSCMELTVKDVFTAPKNTSDKEIFCRAATVLRQ | NCBI Ref: |
| | IYTHNCSNRYLRGLYRNLSSMANKTCSMNEIKKSTLKDF | NP_001003159.1 |
| | LERLKVIMQKKYYRH | |
| 122 | MDLTSQLIPALVCLLAFTSTFVHGQNFNNTLKEIIKTLN | Feline IL4 precursor |
| | ILTARNDSCMELTVMDVLAAPKNTSDKEIFCRATTVLRQ | NCBI Ref: |
| | IYTHHNCSTKFLKGLDRNLSSMANRTCSVNEVKKCTLKD | NP_001036804.1 |
| | FLERLKAIMQKKYSKH | |
| 123 | MGLISQLIPALVCLLACTSNFIQGCKYDITLQEIIKTLN | Predicted Equine IL4 |
| | NLTDGKGKNSCMELTVADAFAGPKNTDGKEICRAAKVLQ | precursor |
| | QLYKRHDRSLIKECLSGLDRNLKGMANGTCCTVNEAKKS | NCBI Ref: |
| | TLKDFLERLKTIMKEKYSKCQS | XP_008536927.1 |
| 124 | MGLTSQLIPTLVCLLALTSTFVHGSSHHHHHHSSGLVPR | Canine IL4_N-His6 with |
| | GSHMHNFNITIKEIIKMLNILTARNDSCMELTVKDVFTA | leader |
| | PKNTSDKEIFCRAATVLRQIYTHNCSNRYLRGLYRNLSS | |
| | MANKTCSMNEIKKSTLKDFLERLKVIMQKKYYRH | |
| 125 | SHHHHHHSSGLVPRGSHMHNFNITIKEIIKMLNILTARN | Canine IL4_N-His6 |
| | DSCMELTVKDVFTAPKNTSDKEIFCRAATVLRQIYTHNC | |
| | SNRYLRGLYRNLSSMANKTCSMNEIKKSTLKDFLERLKV | |
| | IMQKKYYRH | |
| 126 | MGLTSQLIPTLVCLLALTSTFVHGHNFNITIKEIIKMLN | Canine IL4_C-His6 with |
| | ILTARNDSCMELTVKDVFTAPKNTSDKEIFCRAATVLRQ | leader |
| | IYTHNCSNRYLRGLYRNLSSMANKTCSMNEIKKSTLKDF | |
| | LERLKVIMQKKYYRHHHHHH | |
| 127 | HNFNITIKEIIKMLNILTARNDSCMELTVKDVFTAPKNT | Canine IL4_C-His6 |
| | SDKEIFCRAATVLRQIYTHNCSNRYLRGLYRNLSSMANK | |
| | TCSMNEIKKSTLKDFLERLKVIMQKKYYRGSGSHHHHHH | |
| 128 | MDLTSQLIPALVCLLAFTSTFVHGQNFNNTLKEIIKTLN | Feline IL4_C-His6 with |
| | ILTARNDSCMELTVMDVLAAPKNTSDKEIFCRATTVLRQ | leader |
| | IYTHHNCSTKFLKGLDRNLSSMANRTCSVNEVKKCTLKD | |
| | FLERLKAIMQKKYSKHGSGSHHHHHH | |
| 129 | QNFNNTLKEIIKTLNILTARNDSCMELTVMDVLAAPKNT | Feline IL4_C-His6 |
| | SDKEIFCRATTVLRQIYTHHNCSTKFLKGLDRNLSSMAN | |
| | RTCSVNEVKKCTLKDFLERLKAIMQKKYSKHGSGSHHHH | |
| | HH | |
| 130 | MGLISQLIPALVCLLACTSNFIQGCKYDITLQEIIKTLN | Equine IL4_C-His6 with |
| | NLTDGKGKNSCMELTVADAFAGPKNTDGKEICRAAKVLQ | leader |
| | QLYKRHDRSLIKECLSGLDRNLKGMANGTCCTVNEAKKS | |
| | TLKDFLERLKTIMKEKYSKCQGSGSHHHHHH | |
| 131 | CKYDITLQEIIKTLNNLTDGKGKNSCMELTVADAFAGPK | Equine IL4_C-His6 |
| | NTDGKEICRAAKVLQQLYKRHDRSLIKECLSGLDRNLKG | |
| | MANGTCCTVNEAKKSTLKDFLERLKTIMKEKYSKCQGSG | |
| | SHHHHHH | |
| 132 | MDMRVPAQLLGLLLLWLRGARC | exemplary leader |
| | | sequence |
| 133 | GSVKVLHEPSCFSDYISTSVCQWKMDHPTNCSAELRLSY | Canine/Human IL4R- |
| | QLDFMGSENHTCVPENNGGAGCVCHLLMDDVVSADNYTL | ECD_C-HuFc_His6 |
| | DLWAGQQLLWKGSFKPSEHVKPRAPGNLTVHTNVSDTLL | Hybrid1 |
| | LTWSNPYPPDNYLYNHLTYAVNIWSENDPADFRIYNVTY | (Hybrid 1) |
| | LEPSLRIAASTLKSGISYRARVRAWAQCYNTTWSEWSPS | Canine IL4R ECD G1- |
| | TKWHNSYREPFEQHGSENLYFQGPKSCDKTHTCPPCPAP | N55 (bold) |
| | ELLGGPSVFLFPPKPKDTLMISRTPEVTCVVVDVSHEDP | Human IL4R ECD N56- |
| | EVKFNWYVDGVEVHNAKTKPREEQYNSTYRVVSVLTVLH | H209 (italic) |
| | QDWLNGKEYKCKVSNKALPAPIEKTISKAKGQPREPQVY | Human Fc_His6 |
| | TLPPSRDELTKNQVSLTCLVKGFYPSDIAVEWESNGQPE | (underline) |
| | NNYKTTPPVLDSDGSFFLYSKLTVDKSRWQQGNVFSCSV | |
| | MHEALHNHYTQKSLSLSPGKHHHHHH | |
| 134 | GNMKVLQEPTCVSDYMSISTCEWKMNGPTNCSTELRLLY | Canine/Human IL4R- |
| | QLVFLLSEAHTCIPENREDSVCVCSMPIDDAVEADVYQL | ECD_C-HuFc_His6 |
| | DLWAGQQLLWSGSFQPSKHVKPRTPGNLTVHTNVSDTLL | Hybrid2 |
| | LTWSNPYPPDNYLYNHLTYAVNIWSENDPADFRIYNVTY | (Hybrid 2) |
| | LEPSLRIAASTLKSGISYRARVRAWAQCYNTTWSEWSPS | Human IL4R ECD G1- |
| | TKWHNSYREPFEQHGSENLYFQGPKSCDKTHTCPPCPAP | N55 and T110-H209 |
| | ELLGGPSVFLFPPKPKDTLMISRTPEVTCVVVDVSHEDP | (italic) |
| | EVKFNWYVDGVEVHNAKTKPREEQYNSTYRVVSVLTVLH | Canine IL4R ECD R56- |
| | QDWLNGKEYKCKVSNKALPAPIEKTISKAKGQPREPQVY | H109 (bold) |
| | TLPPSRDELTKNQVSLTCLVKGFYPSDIAVEWESNGQPE | Human Fc_His6 |
| | NNYKTTPPVLDSDGSFFLYSKLTVDKSRWQQGNVFSCSV | (underline) |
| | MHEALHNHYTQKSLSLSPGKHHHHHH | |
| 135 | GNMKVLQEPTCVSDYMSISTCEWKMNGPTNCSTELRLLY | Canine/Human IL4R- |
| | QLVFLLSEAHTCIPENNGGAGCVCHLLMDDVVSADNYTL | ECD_C-HuFc_His6 |
| | DLWAGQQLLWKGSFKPSEHVKPRAPGNLTVHPNISHTWL | Hybrid3 |
| | LMWTNPYPTENHLHSELTYMVNVSNDNDPEDFKVYNVTY | (Hybrid 3) |
| | MGPTLRLAASTLKSGASYSARVRAWAQTYNSTWSDWSPS | Human IL4R ECD G1- |
| | TTWLNYYEPGSENLYFQGPKSCDKTHTCPPCPAPELLGG | H109 (italic) |
| | PSVFLFPPKPKDTLMISRTPEVTCVVVDVSHEDPEVKEN | Canine IL4R ECD |
| | WYVDGVEVHNAKTKPREEQYNSTYRVVSVLTVLHQDWLN | P110-P204 (bold) |
| | GKEYKCKVSNKALPAPIEKTISKAKGQPREPQVYTLPPS | Human Fc_His6 |
| | RDELTKNQVSLTCLVKGFYPSDIAVEWESNGQPENNYKT | (underline) |
| | TPPVLDSDGSFFLYSKLTVDKSRWQQGNVFSCSVMHEAL | |
| | HNHYTQKSLSLSPGKHHHHHH | |
| 136 | GSVKVLHEPSCFSDYISTSVCQWKMDHPTNCSAELRLSY | Canine/Human IL4R- |
| | QLDFMGSENHTCVPENREDSVCVCSMPIDDAVEADVYQL | ECD_C-HuFc_His6_ |
| | DLWAGQQLLWSGSFQPSKHVKPRTPGNLTVHTNVSDTLL | Hybrid4 |
| | LTWSNPYPPDNYLYNHLTYAVNIWSENDPADFRIYNVTY | (Hybrid 4) |
| | LEPSLRIAASTLKSGISYRARVRAWAQCYNTTWSEWSPS | Canine IL4R ECD G1- |
| | TKWHNSYREPFEQHGSENLYFQGPKSCDKTHTCPPCPAP | H109 (bold) |
| | ELLGGPSVFLFPPKPKDTLMISRTPEVTCVVVDVSHEDP | Human ILAR ECD |
| | EVKFNWYVDGVEVHNAKTKPREEQYNSTYRVVSVLTVLH | T110-H209 (italic) |
| | QDWLNGKEYKCKVSNKALPAPIEKTISKAKGQPREPQVY | Human Fc_His6 |
| | TLPPSRDELTKNQVSLTCLVKGFYPSDIAVEWESNGQPE | (underline) |
| | NNYKTTPPVLDSDGSFFLYSKLTVDKSRWQQGNVFSCSV | |
| | MHEALHNHYTQKSLSLSPGKHHHHHH | |
| 137 | GNMKVLQEPTCVSDYMSISTCEWKMNGPTNCSTELRLLY | Canine/Human IL4R- |
| | QLVFLLSEAHTCIPENREDSVCVCSMPIDDAVEADVYQL | ECD_C-HuFc_His6 |
| | DLWAGQQLLWSGSFQPSKHVKPRTPGNLTVHPNISHTWL | Hybrid5 |
| | LMWTNPYPTENHLHSELTYMVNVSNDNDPEDFKVYNVTY | (Hybrid 5) |
| | MGPTLRLAASTLKSGASYSARVRAWAQTYNSTWSDWSPS | Human IL4R ECD G1- |
| | TTWLNYYEPGSENLYFQGPKSCDKTHTCPPCPAPELLGG | N55 (italic) |
| | PSVFLFPPKPKDTLMISRTPEVTCVVVDVSHEDPEVKEN | Canine IL4R ECD R56- |
| | WYVDGVEVHNAKTKPREEQYNSTYRVVSVLTVLHQDWLN | P204 |
| | GKEYKCKVSNKALPAPIEKTISKAKGQPREPQVYTLPPS | (bold) |
| | RDELTKNQVSLTCLVKGFYPSDIAVEWESNGQPENNYKT | Human Fc_His6 |
| | TPPVLDSDGSFFLYSKLTVDKSRWQQGNVFSCSVMHEAL | (underline) |
| | HNHYTQKSLSLSPGKHHHHHH | |
| 138 | GSVKVLHEPSCFSDYISTSVCQWKMDHPTNCSAELRLSY | Canine/Human IL4R- |
| | QLDFMGSENHTCVPENNGGAGCVCHLLMDDVVSADNYTL | ECD_C-HuFc_His6_ |
| | DLWAGQQLLWKGSFKPSEHVKPRAPGNLTVHPNISHTWL | Hybrid6 |
| | LMWTNPYPTENHLHSELTYMVNVSNDNDPEDFKVYNVTY | (Hybrid 6) |
| | MGPTLRLAASTLKSGASYSARVRAWAQTYNSTWSDWSPS | Canine IL4R ECD G1- |
| | TTWLNYYEPGSENLYFQGPKSCDKTHTCPPCPAPELLGG | N55, P110-P204 (bold) |
| | PSVFLFPPKPKDTLMISRTPEVTCVVVDVSHEDPEVKEN | Human IL4R ECD N56- |
| | WYVDGVEVHNAKTKPREEQYNSTYRVVSVLTVLHQDWLN | H109 (italic) |
| | GKEYKCKVSNKALPAPIEKTISKAKGQPREPQVYTLPPS | Human Fc_His6 |
| | RDELTKNQVSLTCLVKGFYPSDIAVEWESNGQPENNYKT | (underline) |
| | TPPVLDSDGSFFLYSKLTVDKSRWQQGNVFSCSVMHEAL | |
| | HNHYTQKSLSLSPGKHHHHHH | |
| 139 | GNMKVLQEPTCVSDYMSISTCEWKMNGPTNCSAELRLSY | Canine/Human IL4R- |
| | QLDFMGSENHTCVPENREDSVCVCSMPIDDAVEADVYQL | ECD_C-HuFc_His6 |
| | DLWAGQQLLWSGSFQPSKHVKPRTPGNLTVHPNISHTWL | Hybrid7 |
| | LMWTNPYPTENHLHSELTYMVNVSNDNDPEDEKVYNVTY | (Hybrid 7) |
| | MGPTLRLAASTLKSGASYSARVRAWAQTYNSTWSDWSPS | Human IL4R ECD G1- |
| | TTWLNYYEPGSENLYFQGPKSCDKTHTCPPCPAPELLGG | N30 (italic) |
| | PSVFLFPPKPKDTLMISRTPEVTCVVVDVSHEDPEVKEN | Canine IL4R ECD C31- |
| | WYVDGVEVHNAKTKPREEQYNSTYRVVSVLTVLHQDWLN | P204 (bold) |
| | GKEYKCKVSNKALPAPIEKTISKAKGQPREPQVYTLPPS | Human Fc_His6 |
| | RDELTKNQVSLTCLVKGFYPSDIAVEWESNGQPENNYKT | (underline) |
| | TPPVLDSDGSFFLYSKLTVDKSRWQQGNVFSCSVMHEAL | |
| | HNHYTQKSLSLSPGKHHHHHH | |
| 140 | GSVKVLHEPSCFSDYISTSVCQWKMDHPTNCSTELRLLY | Canine/Human IL4R- |
| | QLVFLLSEAHTCIPENREDSVCVCSMPIDDAVEADVYQL | ECD_C-HuFc_His6 |
| | DLWAGQQLLWSGSFQPSKHVKPRTPGNLTVHPNISHTWL | Fusion8 |
| | LMWTNPYPTENHLHSELTYMVNVSNDNDPEDEKVYNVTY | (Fusion 8) |
| | MGPTLRLAASTLKSGASYSARVRAWAQTYNSTWSDWSPS | Canine IL4R ECD G1- |
| | TTWLNYYEPGSENLYFQGPKSCDKTHTCPPCPAPELLGG | N30 and R56-P204 |
| | PSVFLFPPKPKDTLMISRTPEVTCVVVDVSHEDPEVKEN | (bold) |
| | WYVDGVEVHNAKTKPREEQYNSTYRVVSVLTVLHQDWLN | Human IL4R ECD C31- |
| | GKEYKCKVSNKALPAPIEKTISKAKGQPREPQVYTLPPS | N55 (italic) |
| | RDELTKNQVSLTCLVKGFYPSDIAVEWESNGQPENNYKT | Human Fc_His6 |
| | TPPVLDSDGSFFLYSKLTVDKSRWQQGNVFSCSVMHEAL | (underline) |
| | HNHYTQKSLSLSPGKHHHHHH | |
| 141 | GSVKVLHEPSCFSDYISTSVCQWKMDHPTNCSTELRLLY | Canine/Human IL4R- |
| | QLDFMGSENHTCVPENREDSVCVCSMPIDDAVEADVYQL | ECD_C-HuFc_His6_ |
| | DLWAGQQLLWSGSFQPSKHVKPRTPGNLTVHPNISHTWL | Hybrid9 |
| | LMWTNPYPTENHLHSELTYMVNVSNDNDPEDFKVYNVTY | (Hybrid 9) |
| | MGPTLRLAASTLKSGASYSARVRAWAQTYNSTWSDWSPS | Canine IL4R ECD G1- |
| | TTWLNYYEPGSENLYFQGPKSCDKTHTCPPCPAPELLGG | N30 and D42-P204 |
| | PSVFLFPPKPKDTLMISRTPEVTCVVVDVSHEDPEVKEN | (bold) |
| | WYVDGVEVHNAKTKPREEQYNSTYRVVSVLIVLHQDWLN | Human IL4R ECD C31- |
| | GKEYKCKVSNKALPAPIEKTISKAKGQPREPQVYTLPPS | L41 (italic) |
| | RDELTKNQVSLTCLVKGFYPSDIAVEWESNGQPENNYKT | Human Fc_His6 |
| | TPPVLDSDGSFFLYSKLTVDKSRWQQGNVFSCSVMHEAL | (underline) |
| | HNHYTQKSLSLSPGKHHHHHH | |
| 142 | GSVKVLHEPSCFSDYISTSVCQWKMDHPTNCSAELRLSY | Canine/Human IL4R- |
| | QLVFLLSEAHTCIPENREDSVCVCSMPIDDAVEADVYQL | ECD_C-HuFc_His6_ |
| | DLWAGQQLLWSGSFQPSKHVKPRTPGNLTVHPNISHTWL | Hybrid10 |
| | LMWTNPYPTENHLHSELTYMVNVSNDNDPEDFKVYNVTY | (Hybrid 10) |
| | MGPTLRLAASTLKSGASYSARVRAWAQTYNSTWSDWSPS | Canine IL4R ECD G1- |
| | TTWLNYYEPGSENLYFQGPKSCDKTHTCPPCPAPELLGG | L41 and R56-P204 |
| | PSVFLFPPKPKDTLMISRTPEVTCVVVDVSHEDPEVKEN | (bold) |
| | WYVDGVEVHNAKTKPREEQYNSTYRVVSVLTVLHQDWLN | Human IL4R ECD V42- |
| | GKEYKCKVSNKALPAPIEKTISKAKGQPREPQVYTLPPS | N55 (italic) |
| | RDELTKNQVSLTCLVKGFYPSDIAVEWESNGQPENNYKT | Human Fc_His6 |
| | TPPVLDSDGSFFLYSKLTVDKSRWQQGNVFSCSVMHEAL | (underline) |
| | HNHYTQKSLSLSPGKHHHHHH | |
| 143 | GSVKVLHEPSCFSDYISTSVCQWKMDHPTNCSAELRLSY | Canine/Human IL4R- |
| | QLDFMGSENHTCVPENNGGAGCVCHLLMDDVVEADVYQL | ECD_C-HuFc_His6_ |
| | DLWAGQQLLWSGSFQPSKHVKPRTPGNLTVHPNISHTWL | Hybrid11 |
| | LMWTNPYPTENHLHSELTYMVNVSNDNDPEDFKVYNVTY | (Hybrid 11) |
| | MGPTLRLAASTLKSGASYSARVRAWAQTYNSTWSDWSPS | Canine IL4R ECD G1- |
| | TTWLNYYEPGSENLYFQGPKSCDKTHTCPPCPAPELLGG | N55 and E72-P204 |
| | PSVFLFPPKPKDTLMISRTPEVTCVVVDVSHEDPEVKEN | (bold) |
| | WYVDGVEVHNAKTKPREEQYNSTYRVVSVLTVLHQDWLN | Human IL4R ECD N56- |
| | GKEYKCKVSNKALPAPIEKTISKAKGQPREPQVYTLPPS | V71 (italic) |
| | RDELTKNQVSLTCLVKGFYPSDIAVEWESNGQPENNYKT | Human Fc_His6 |
| | TPPVLDSDGSFFLYSKLTVDKSRWQQGNVFSCSVMHEAL | (underline) |
| | HNHYTQKSLSLSPGKHHHHHH | |
| 144 | GSVKVLHEPSCFSDYISTSVCQWKMDHPTNCSAELRLSY | Canine/Human IL4R- |
| | QLDFMGSENHTCVPENREDSVCVCSMPIDDAVSADNYTL | ECD_C-HuFc_His6 |
| | DLWAGQQLLWKGSFQPSKHVKPRTPGNLTVHPNISHTWL | Hybrid12 |
| | LMWTNPYPTENHLHSELTYMVNVSNDNDPEDFKVYNVTY | (Hybrid 12) |
| | MGPTLRLAASTLKSGASYSARVRAWAQTYNSTWSDWSPS | Canine IL4R ECD G1- |
| | TTWLNYYEPGSENLYFQGPKSCDKTHTCPPCPAPELLGG | V71 and G90-P204 |
| | PSVFLFPPKPKDTLMISRTPEVTCVVVDVSHEDPEVKEN | (bold) |
| | WYVDGVEVHNAKTKPREEQYNSTYRVVSVLTVLHQDWLN | Human IL4R ECD S72- |
| | GKEYKCKVSNKALPAPIEKTISKAKGQPREPQVYTLPPS | K89 (italic) |
| | RDELTKNQVSLTCLVKGFYPSDIAVEWESNGQPENNYKT | Human Fc_His6 |
| | TPPVLDSDGSFFLYSKLTVDKSRWQQGNVFSCSVMHEAL | (underline) |
| | HNHYTQKSLSLSPGKHHHHHH | |
| 145 | GSVKVLHEPSCFSDYISTSVCQWKMDHPTNCSAELRLSY | Canine/Human IL4R- |
| | QLDFMGSENHTCVPENREDSVCVCSMPIDDAVEADVYQL | ECD_C-HuFc_His6 |
| | DLWAGQQLLWSGSFKPSEHVKPRAPGNLTVHPNISHTWL | Hybrid13 |
| | LMWTNPYPTENHLHSELTYMVNVSNDNDPEDFKVYNVTY | (Hybrid 13) |
| | MGPTLRLAASTLKSGASYSARVRAWAQTYNSTWSDWSPS | Canine IL4R ECD G1- |
| | TTWLNYYEPGSENLYFQGPKSCDKTHTCPPCPAPELLGG | S89 and P110-P204 |
| | PSVFLFPPKPKDTLMISRTPEVTCVVVDVSHEDPEVKEN | (bold) |
| | WYVDGVEVHNAKTKPREEQYNSTYRVVSVLTVLHQDWLN | Human IL4R ECD G90- |
| | GKEYKCKVSNKALPAPIEKTISKAKGQPREPQVYTLPPS | H109 (italic) |
| | RDELTKNQVSLTCLVKGFYPSDIAVEWESNGQPENNYKT | Human Fc_His6 |
| | TPPVLDSDGSFFLYSKLTVDKSRWQQGNVFSCSVMHEAL | (underline) |
| | HNHYTQKSLSLSPGKHHHHHH | |
| 146 | GSVKVLHEPSCFSDYISTSVCQWKMDHPTNCSAELRLSY | Canine/Human IL4R- |
| | QLDFMGSENHTCVPENREDSVCVCSMPIDDVVSADNYTL | ECD_C-HuFc_His6 |
| | DLWAGQQLLWKGSFKPSKHVKPRTPGNLTVHPNISHTWL | Hybrid14 |
| | LMWTNPYPTENHLHSELTYMVNVSNDNDPEDFKVYNVTY | (Hybrid 14) |
| | MGPTLRLAASTLKSGASYSARVRAWAQTYNSTWSDWSPS | Canine IL4R ECD G1- |
| | TTWLNYYEPGSENLYFQGPKSCDKTHTCPPCPAPELLGG | I67 and S95-P204 (bold) |
| | PSVFLFPPKPKDTLMISRTPEVTCVVVDVSHEDPEVKEN | Human IL4R ECD D68- |
| | WYVDGVEVHNAKTKPREEQYNSTYRVVSVLTVLHQDWLN | P94 (italic) |
| | GKEYKCKVSNKALPAPIEKTISKAKGQPREPQVYTLPPS | Human Fc_His6 |
| | RDELTKNQVSLTCLVKGFYPSDIAVEWESNGQPENNYKT | (underline) |
| | TPPVLDSDGSFFLYSKLTVDKSRWQQGNVFSCSVMHEAL | |
| | HNHYTQKSLSLSPGKHHHHHH | |
| 147 | GSVKVLHEPSCFSDYISTSVCQWKMDHPINCSTELRLSY | Canine IL4R-ECD |
| | QLDFMGSENHTCVPENREDSVCVCSMPIDDAVEADVYQL | C-HuFc_His6.A33T |
| | DLWAGQQLLWSGSFQPSKHVKPRTPGNLTVHPNISHTWL | (Mutant 1) |
| | LMWTNPYPTENHLHSELTYMVNVSNDNDPEDFKVYNVTY | |
| | MGPTLRLAASTLKSGASYSARVRAWAQTYNSTWSDWSPS | |
| | TTWLNYYEPGSENLYFQGPKSCDKTHTCPPCPAPELLGG | |
| | PSVFLFPPKPKDTLMISRTPEVTCVVVDVSHEDPEVKEN | |
| | WYVDGVEVHNAKTKPREEQYNSTYRVVSVLTVLHQDWLN | |
| | GKEYKCKVSNKALPAPIEKTISKAKGQPREPQVYTLPPS | |
| | RDELTKNQVSLTCLVKGFYPSDIAVEWESNGQPENNYKT | |
| | TPPVLDSDGSFFLYSKLTVDKSRWQQGNVFSCSVMHEAL | |
| | HNHYTQKSLSLSPGKHHHHHH | |
| 148 | GSVKVLHEPSCFSDYISTSVCQWKMDHPINCSAELRLSY | Canine IL4R-ECD_ |
| | QLDFAGSENHTCVPENREDSVCVCSMPIDDAVEADVYQL | C-HuFc_His6.M44A |
| | DLWAGQQLLWSGSFQPSKHVKPRTPGNLTVHPNISHTWL | (Mutant 2) |
| | LMWTNPYPTENHLHSELTYMVNVSNDNDPEDFKVYNVTY | |
| | MGPTLRLAASTLKSGASYSARVRAWAQTYNSTWSDWSPS | |
| | TTWLNYYEPGSENLYFQGPKSCDKTHTCPPCPAPELLGG | |
| | PSVFLFPPKPKDTLMISRTPEVTCVVVDVSHEDPEVKEN | |
| | WYVDGVEVHNAKTKPREEQYNSTYRVVSVLTVLHQDWLN | |
| | GKEYKCKVSNKALPAPIEKTISKAKGQPREPQVYTLPPS | |
| | RDELTKNQVSLTCLVKGFYPSDIAVEWESNGQPENNYKT | |
| | TPPVLDSDGSFFLYSKLTVDKSRWQQGNVFSCSVMHEAL | |
| | HNHYTQKSLSLSPGKHHHHHH | |
| 149 | GSVKVLHEPSCFSDYISTSVCQWKMDHPINCSAELRLSY | Canine IL4R-ECD_ |
| | QLDFMASENHTCVPENREDSVCVCSMPIDDAVEADVYQL | C-HuFc_His6.G45A |
| | DLWAGQQLLWSGSFQPSKHVKPRTPGNLTVHPNISHTWL | (Mutant 3) |
| | LMWTNPYPTENHLHSELTYMVNVSNDNDPEDFKVYNVTY | |
| | MGPTLRLAASTLKSGASYSARVRAWAQTYNSTWSDWSPS | |
| | TTWLNYYEPGSENLYFQGPKSCDKTHTCPPCPAPELLGG | |
| | PSVFLFPPKPKDTLMISRTPEVTCVVVDVSHEDPEVKEN | |
| | WYVDGVEVHNAKTKPREEQYNSTYRVVSVLTVLHQDWLN | |
| | GKEYKCKVSNKALPAPIEKTISKAKGQPREPQVYTLPPS | |
| | RDELTKNQVSLTCLVKGFYPSDIAVEWESNGQPENNYKT | |
| | TPPVLDSDGSFFLYSKLTVDKSRWQQGNVFSCSVMHEAL | |
| | HNHYTQKSLSLSPGKHHHHHH | |
| 150 | GSVKVLHEPSCFSDYISTSVCQWKMDHPINCSAELRLSY | Canine IL4R-ECD_ |
| | QLDFMGSEAHTCVPENREDSVCVCSMPIDDAVEADVYQL | |
| | DLWAGQQLLWSGSFQPSKHVKPRTPGNLTVHPNISHTWL | |
| | LMWTNPYPTENHLHSELTYMVNVSNDNDPEDFKVYNVTY | |
| | MGPTLRLAASTLKSGASYSARVRAWAQTYNSTWSDWSPS | |
| | TTWLNYYEPGSENLYFQGPKSCDKTHTCPPCPAPELLGG | |
| | PSVFLFPPKPKDTLMISRTPEVTCVVVDVSHEDPEVKEN | C-HuFc_His6.N48A |
| | WYVDGVEVHNAKTKPREEQYNSTYRVVSVLTVLHQDWLN | (Mutant 4) |
| | GKEYKCKVSNKALPAPIEKTISKAKGQPREPQVYTLPPS | |
| | RDELTKNQVSLTCLVKGFYPSDIAVEWESNGQPENNYKT | |
| | TPPVLDSDGSFFLYSKLTVDKSRWQQGNVFSCSVMHEAL | |
| | HNHYTQKSLSLSPGKHHHHHH | |
| 151 | GSVKVLHEPSCFSDYISTSVCQWKMDHPINCSAELRLAY | Canine IL4R-ECD |
| | QLDFMGSENHTCVPENREDSVCVCSMPIDDAVEADVYQL | C-HuFc_His6.S38A |
| | DLWAGQQLLWSGSFQPSKHVKPRTPGNLTVHPNISHTWL | (Mutant 5) |
| | LMWTNPYPTENHLHSELTYMVNVSNDNDPEDFKVYNVTY | |
| | MGPTLRLAASTLKSGASYSARVRAWAQTYNSTWSDWSPS | |
| | TTWLNYYEPGSENLYFQGPKSCDKTHTCPPCPAPELLGG | |
| | PSVFLFPPKPKDTLMISRTPEVTCVVVDVSHEDPEVKEN | |
| | WYVDGVEVHNAKTKPREEQYNSTYRVVSVLTVLHQDWLN | |
| | GKEYKCKVSNKALPAPIEKTISKAKGQPREPQVYTLPPS | |
| | RDELTKNQVSLTCLVKGFYPSDIAVEWESNGQPENNYKT | |
| | TPPVLDSDGSFFLYSKLTVDKSRWQQGNVFSCSVMHEAL | |
| | HNHYTQKSLSLSPGKHHHHHH | |
| 152 | GSVKVLHEPSCFSDYISTSVCQWKMDHPINCSAELRLSY | Canine IL4R-ECD_ |
| | QLAFMGSENHTCVPENREDSVCVCSMPIDDAVEADVYQL | |
| | DLWAGQQLLWSGSFQPSKHVKPRTPGNLTVHPNISHTWL | |
| | LMWTNPYPTENHLHSELTYMVNVSNDNDPEDFKVYNVTY | |
| | MGPTLRLAASTLKSGASYSARVRAWAQTYNSTWSDWSPS | |
| | TTWLNYYEPGSENLYFQGPKSCDKTHTCPPCPAPELLGG | |
| | PSVFLFPPKPKDTLMISRTPEVTCVVVDVSHEDPEVKEN | |
| | WYVDGVEVHNAKTKPREEQYNSTYRVVSVLTVLHQDWLN | C-HuFc_His6.D42A |
| | GKEYKCKVSNKALPAPIEKTISKAKGQPREPQVYTLPPS | (Mutant 6) |
| | RDELTKNQVSLTCLVKGFYPSDIAVEWESNGQPENNYKT | |
| | TPPVLDSDGSFFLYSKLTVDKSRWQQGNVFSCSVMHEAL | |
| | HNHYTQKSLSLSPGKHHHHHH | |
| 153 | GSVKVLHEPSCFSDYISTSVCQWKMDHPINCSAELRLSY | Canine IL4R-ECD |
| | QLDFMGSENATCVPENREDSVCVCSMPIDDAVEADVYQL | |
| | DLWAGQQLLWSGSFQPSKHVKPRTPGNLTVHPNISHTWL | |
| | LMWTNPYPTENHLHSELTYMVNVSNDNDPEDFKVYNVTY | |
| | MGPTLRLAASTLKSGASYSARVRAWAQTYNSTWSDWSPS | |
| | TTWLNYYEPGSENLYFQGPKSCDKTHTCPPCPAPELLGG | |
| | PSVFLFPPKPKDTLMISRTPEVTCVVVDVSHEDPEVKEN | C-HuFc_His6.H49A |
| | WYVDGVEVHNAKTKPREEQYNSTYRVVSVLTVLHQDWLN | (Mutant 7) |
| | GKEYKCKVSNKALPAPIEKTISKAKGQPREPQVYTLPPS | |
| | RDELTKNQVSLTCLVKGFYPSDIAVEWESNGQPENNYKT | |
| | TPPVLDSDGSFFLYSKLTVDKSRWQQGNVFSCSVMHEAL | |
| | HNHYTQKSLSLSPGKHHHHHH | |
| 154 | MALWLTVVIALTCLGGLASPSPVTPSPTLKELIEELVNI | Canine IL13 precursor |
| | TQNQASLCNGSMVWSVNLTAGMYCAALESLINVSDCSAI | NCBI Ref: |
| | QRTQRMLKALCSQKPAAGQISSERSRDTKIEVIQLVKNL | NP_001003384 |
| | LTYVRGVYRHGNFR | |
| 155 | MWFLDSTRQSGDQGGRRHTWPIKATARGQGHKPLSLGQP | Feline IL13 precursor |
| | TCPLLAPPVLALGSMALWLTVVIALTCLGGLASPGPHSR | NCBI Ref: |
| | RELKELIEELVNITQNQVSLCNGSMVWSVNLTTGMQYCA | XP_006927648 |
| | ALESLINVSDCTAIQRTQRMLKALCTQKPSAGQTASERS | |
| | RDTKIEVIQLVKNLLNHLRRNFRHGNFK | |
| 156 | MALWLTVVIALTCLGGLASPSPVTPSPTLKELIEELVNI | Canine IL13_C-His6 |
| | TQNQASLCNGSMVWSVNLTAGMYCAALESLINVSDCSAI | with leader |
| | QRTQRMLKALCSQKPAAGQISSERSRDTKIEVIQLVKNL | |
| | LTYVRGVYRHGNFRGSHHHHHH | |
| 157 | SPSPVTPSPTLKELIEELVNITQNQASLCNGSMVWSVNL | Canine IL13_C-His6 |
| | TAGMYCAALESLINVSDCSAIQRTQRMLKALCSQKPAAG | |
| | QISSERSRDTKIEVIQLVKNLLTYVRGVYRHGNFRGSHH | |
| | HHHH | |
| 158 | MALWLTVVIALTCLGGLASPGPHSRRELKELIEELVNIT | Feline IL13_C-His6 |
| | QNQLLQVSLCNGSMVWSVNLTTGMQYCAALESLINVSDC | with leader |
| | TAIQRTQRMLKALCTQKPSAGQTASERSRDTKIEVIQLV | |
| | KNLLNHLRRNFRHGNFKGSGSHHHHHH | |
| 159 | SPGPHSRRELKELIEELVNITQNQLLQVSLCNGSMVWSV | Feline IL13_C-His6 |
| | NLTTGMQYCAALESLINVSDCTAIQRTQRMLKALCTQKP | |
| | SAGQTASERSRDTKIEVIQLVKNLLNHLRRNFRHGNFKG | |
| | SGSHHHHHH | |
| 160 | MERPARLCGLWALLLCAAGGRGGGVAAPTETQPPVTNLSVSVE | Canine IL13R |
| | NLCTVIWTWDPPEGASPNCTLRYFSHFDNKQDKKIAPETHRSK | NCBI Ref: |
| | EVPLNERICLQVGSQCSTNESDNPSILVEKCTPPPEGDPESAV | XP_538150.3 |
| | TELQCVWHNLSYMKCTWLPGRNTSPDTNYTLYYWHSSLGKILQ | |
| | CEDIYREGQHIGCSFALTNLKDSSFEQHSVQIVVKDNAGKIRP | |
| | SFNIVPLTSHVKPDPPHIKRLFFQNGNLYVQWKNPQNFYSRCL | |
| | SYQVEVNNSQTETNDIFYVEEAKCQNSEFEGNLEGTICFMVPG | |
| | VLPDTLNTVRIRVRTNKLCYEDDKLWSNWSQAMSIGENTDPTF | |
| | YITMLLATPVIVAGAIIVLLLYLKRLKIIIFPPIPDPGKIFKE | |
| | MFGDQNDDTLHWRKYDIYEKQTKEETDSVVLIENLKKASQ | |
| 161 | TETQPPVTNLSVSVENLCTVIWTWDPPEGASPNCTLRYFSHFD | Canine IL13R ECD |
| | NKQDKKIAPETHRSKEVPLNERICLQVGSQCSTNESDNPSILV | |
| | EKCTPPPEGDPESAVTELQCVWHNLSYMKCTWLPGRNTSPDTN | |
| | YTLYYWHSSLGKILQCEDIYREGQHIGCSFALTNLKDSSFEQH | |
| | SVQIVVKDNAGKIRPSFNIVPLTSHVKPDPPHIKRLFFQNGNL | |
| | YVQWKNPQNFYSRCLSYQVEVNNSQTETNDIFYVEEAKCQNSE | |
| | FEGNLEGTICFMVPGVLPDTLNTVRIRVRINKLCYEDDKLWSN | |
| | WSQAMSIGENTDPT | |
| 162 | PVPEPLGGPSVLIFPPKPKDILRITRTPEVTCVVLDLGR | Exemplary wild-type |
| | EDPEVQISWFVDGKEVHTAKTQSREQQFNGTYRVVSVLP | canine IgG-A Fc |
| | IEHQDWLTGKEFKCRVNHIDLPSPIERTISKARGRAHKP | Protein A− |
| | SVYVLPPSPKELSSSDTVSITCLIKDFYPPDIDVEWQSN | C1q− |
| | GQQEPERKHRMTPPQLDEDGSYFLYSKLSVDKSRWQQGD | CD16− |
| | PFTCAVMHETLQNHYTDLSLSHSPGK | |
| 163 | PAPEMLGGPSVFIFPPKPKDTLLIARTPEVTCVVVDLDP | Exemplary wild-type |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Protein A+ |
| | SVYVLPPSREELSKNTVSLTCLIKDFFPPDIDVEWQSNG | C1q+ |
| | QQEPESKYRTTPPQLDEDGSYFLYSKLSVDKSRWQRGDT | CD16+ |
| | FICAVMHEALHNHYTQESLSHSPGK | |
| 164 | PKRENGRVPRPPDCPKCPAPEMLGGPSVFIFPPKPKDTLL | Exemplary wild-type |
| | IARTPEVTCVVVDLDPEDPEVQISWFVDGKQMQTAKTQP | canine IgG-B Fc with |
| | REEQFNGTYRVVSVLPIGHQDWLKGKQFTCKVNNKALPS | hinge |
| | PIERTISKARGQAHQPSVYVLPPSREELSKNTVSLTCLI | Protein A+ |
| | KDFFPPDIDVEWQSNGQQEPESKYRTTPPQLDEDGSYFL | C1q+ |
| | YSKLSVDKSRWQRGDTFICAVMHEALHNHYTQESLSHSP | CD16+ |
| | GK | |
| 165 | PGCGLLGGPSVFIFPPKPKDILVTARTPTVTCVVVDLDP | Exemplary wild-type |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | Protein A− |
| | NVYVLPPSRDEMSKNTVTLTCLVKDFFPPEIDVEWQSNG | Clq+ |
| | QQEPESKYRMTPPQLDEDGSYFLYSKLSVDKSRWQRGDT | CD16+ |
| | FICAVMHEALHNHYTQISLSHSPGK | |
| 166 | AKECECKCNCNNCPCPGCGLLGGPSVFIFPPKPKDILVT | Exemplary wild-type |
| | ARTPTVTCVVVDLDPENPEVQISWFVDSKQVQTANTQPR | canine IgG-C Fc with |
| | EEQSNGTYRVVSVLPIGHQDWLSGKQFKCKVNNKALPSP | hinge |
| | IEEIISKTPGQAHQPNVYVLPPSRDEMSKNTVTLTCLVK | Protein A− |
| | DFFPPEIDVEWQSNGQQEPESKYRMTPPQLDEDGSYFLY | C1q+ |
| | SKLSVDKSRWQRGDTFICAVMHEALHNHYTQISLSHSPG | CD16+ |
| | K | |
| 167 | PVPESLGGPSVFIFPPKPKDILRITRTPEITCVVLDLGR | Exemplary wild-type |
| | EDPEVQISWFVDGKEVHTAKTQPREQQFNSTYRVVSVLP | canine IgG-D Fc |
| | IEHQDWLTGKEFKCRVNHIGLPSPIERTISKARGQAHQP | Protein A− |
| | SVYVLPPSPKELSSSDTVTLTCLIKDFFPPEIDVEWQSN | C1q− |
| | GQPEPESKYHTTAPQLDEDGSYFLYSKLSVDKSRWQQGD | CD16− |
| | TFTCAVMHEALQNHYTDLSLSHSPGK | |
| 168 | PVPEPLGGPSVLIFPPKPKDTLLIARTPEVTCVVLDLGR | Exemplary variant |
| | EDPEVQISWFVDGKEVHTAKTQSREQQFNGTYRVVSVLP | canine IgG-A Fc |
| | IGHQDWLTGKEFKCRVNHIDLPSPIERTISKARGRAHKP | C1q− |
| | SVYVLPPSPKELSSSDTVSITCLIKDFYPPDIDVEWQSN | Protein A+ |
| | GQQEPERKHRMTPPQLDEDGSYFLYSKLSVDKSRWQQGD | I(21)T |
| | PFTCAVMHEALHNHYTDLSLSHSPGK | R(23)L |
| | | T(25)A |
| | | E(80)G |
| | | T(205)A |
| | | Q(207)H |
| 169 | PVPEPLGGPSVLIFPPKPKDTLRITRTPEVTCVVLDLGR | Exemplary variant |
| | EDPEVQISWFVDGKEVHTAKTQSREQQFNGTYRVVSVLP | canine IgG-A Fc |
| | IEHQDWLTGKEFKCRVNHIDLPSPIERTISKARGRAHKP | C1q− |
| | SVYVLPPSPKELSSSDTVSITCLIKDFYPPDIDVEWQSN | Protein A+ |
| | GQQEPERKHRMTPPQLDEDGSYFLYSKLSVDKSRWQQGD | I(21)T |
| | PFTCAVMHETLHNHYTDLSLSHSPGK | Q(207)H |
| 170 | PAPEMLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc |
| | IGHQDWLKGKQFTCRVNNKALPSPIERTISKARGQAHQP | Protein A+ |
| | SVYVLPPSREELSKNTVSLTCLIKDFFPPDIDVEWQSNG | C1q− |
| | QQEPESKYRTTPPQLDEDGSYFLYSKLSVDKSRWQRGDT | K(93)R |
| | FICAVMHEALHNHYTQESLSHSPGK | |
| 171 | PAPEPLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Protein A+ |
| | SVYVLPPSREELSKNTVSLTCLIKDFFPPDIDVEWQSNG | C1q+ |
| | QQEPESKYRTTPPQLDEDGSYFLYSKLSVDKSRWQRGDT | CD16− |
| | FICAVMHEALHNHYTQESLSHSPGK | M(5)P |
| 172 | PAPEMLGGPSVFIFPPKPKDTLLIARTPEVTCVVVDLDR | Exemplary variant |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Protein A+ |
| | SVYVLPPSREELSKNTVSLTCLIKDFFPPDIDVEWQSNG | C1q+ |
| | QQEPESKYRITPPQLDEDGSYFLYSKLSVDKSRWQRGDT | CD16− |
| | FICAVMHEALHNHYTQESLSHSPGK | P(39)R |
| 173 | PAPEMLGGPSVFIFPPKPKDTLLIARTPEVTCVVVDLGP | Exemplary variant |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Protein A+ |
| | SVYVLPPSREELSKNTVSLTCLIKDFFPPDIDVEWQSNG | Clq+ |
| | QQEPESKYRTTPPQLDEDGSYFLYSKLSVDKSRWQRGDT | CD16− |
| | FICAVMHEALHNHYTQESLSHSPGK | D(38)G |
| 174 | PAPEMLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc |
| | IGHQDWLKGKQFTCKVNNIALPSPIERTISKARGQAHQP | Protein A+ |
| | SVYVLPPSREELSKNTVSLTCLIKDFFPPDIDVEWQSNG | C1q+ |
| | QQEPESKYRTTPPQLDEDGSYFLYSKLSVDKSRWQRGDT | CD16− |
| | FICAVMHEALHNHYTQESLSHSPGK | K(97)I |
| 175 | PAPEMLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc |
| | IGHQDWLKGKQFTCKVNNKGLPSPIERTISKARGQAHQP | Protein A+ |
| | SVYVLPPSREELSKNTVSLTCLIKDFFPPDIDVEWQSNG | C1q+ |
| | QQEPESKYRTTPPQLDEDGSYFLYSKLSVDKSRWQRGDT | CD16− |
| | FICAVMHEALHNHYTQESLSHSPGK | A(98)G |
| 176 | PAPEMLGGPSVFIFPPKPKDTLLIARTPEVTCVVVDLGP | Exemplary variant |
| | EDPEVQISWFVDGKQMQTAKTQPREEQFNGTYRVVSVLP | canine IgG-B Fc |
| | IGHQDWLKGKQFTCKVNNIGLPSPIERTISKARGQAHQP | Protein A+ |
| | SVYVLPPSREELSKNTVSLTCLIKDFFPPDIDVEWQSNG | C1q+ |
| | QQEPESKYRTTPPQLDEDGSYFLYSKLSVDKSRWQRGDT | CD16− |
| | FICAVMHEALHNHYTQESLSHSPGK | D(38)G |
| | | K(97)I |
| | | A(98)G |
| 177 | PAPEMLGGPSVFIFPPKPKDTLLIARTPEVTCVVVDLGP | Exemplary variant |
| | EDPEVQISWFVDGKQMQTAKTQPREEQFNGTYRVVSVLP | canine IgG-B Fc |
| | IGHQDWLKGKQFTCRVNNIGLPSPIERTISKARGQAHQP | Protein A+ |
| | SVYVLPPSREELSKNTVSLTCLIKDFFPPDIDVEWQSNG | C1q− |
| | QQEPESKYRTTPPQLDEDGSYFLYSKLSVDKSRWQRGDT | CD16− |
| | FICAVMHEALHNHYTQESLSHSPGK | D(38)G |
| | | K(93)R |
| | | K(97)I |
| | | A(98)G |
| 178 | PAPEPLGGPSVFIFPPKPKDTLLIARTPEVTCVVVDLDR | Exemplary variant |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Protein A+ |
| | SVYVLPPSREELSKNTVSLTCLIKDFFPPDIDVEWQSNG | C1q+ |
| | QQEPESKYRTTPPQLDEDGSYFLYSKLSVDKSRWQRGDT | CD16− |
| | FICAVMHEALHNHYTQESLSHSPGK | M(5)P |
| | | P(39)R |
| 179 | PAPEPLGGPSVFIFPPKPKDTLLIARTPEVTCVVVDLDR | Exemplary variant |
| | EDPEVQISWFVDGKQMQTAKTQPREEQFNGTYRVVSVLP | canine IgG-B Fc |
| | IGHQDWLKGKQFTCRVNNKALPSPIERTISKARGQAHQP | Protein A+ |
| | SVYVLPPSREELSKNTVSLTCLIKDFFPPDIDVEWQSNG | C1q− |
| | QQEPESKYRTTPPQLDEDGSYFLYSKLSVDKSRWQRGDT | CD16− |
| | FICAVMHEALHNHYTQESLSHSPGK | M(5)P |
| | | P(39)R |
| | | K(93)R |
| 180 | PGCGLLGGPSVFIFPPKPKDTLLIARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | C1q+ |
| | NVYVLPPSRDEMSKNTVTLTCLVKDFFPPEIDVEWQSNG | Protein A+ |
| | QQEPESKYRMTPPQLDEDGSYFLYSKLSVDKSRWQRGDT | I(21)T |
| | FICAVMHEALHNHYTQISLSHSPGK | V(23)L |
| | | T(24)I |
| 181 | PGCGLLGGPSVFIFPPKPKDTLVTARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | C1q+ |
| | NVYVLPPSRDEMSKNTVTLTCLVKDFFPPEIDVEWQSNG | Protein A+ |
| | QQEPESKYRMTPPQLDEDGSYFLYSKLSVDKSRWQRGDT | I(21)T |
| | FICAVMHEALHNHYTQISLSHSPGK | |
| 182 | PGCGLLGGPSVFIFPPKPKDILVTARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCRVNNKALPSPIEEIISKTPGQAHQP | Protein A- |
| | NVYVLPPSRDEMSKNTVTLTCLVKDFFPPEIDVEWQSNG | C1q− |
| | QQEPESKYRMTPPQLDEDGSYFLYSKLSVDKSRWQRGDT | K(93)R |
| | FICAVMHEALHNHYTQISLSHSPGK | |
| 183 | PGCGLLGGPSVFIFPPKPKDTLLIARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCRVNNKALPSPIEEIISKTPGQAHQP | C1q− |
| | NVYVLPPSRDEMSKNTVTLTCLVKDFFPPEIDVEWQSNG | K(93)R |
| | QQEPESKYRMTPPQLDEDGSYFLYSKLSVDKSRWQRGDT | Protein A+ |
| | FICAVMHEALHNHYTQISLSHSPGK | I(21)T |
| | | V(23)L |
| | | T(24)I |
| 184 | PGCGPLGGPSVFIFPPKPKDILVTARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | Protein A+ |
| | NVYVLPPSRDEMSKNTVTLTCLVKDFFPPEIDVEWQSNG | C1q+ |
| | QQEPESKYRMTPPQLDEDGSYFLYSKLSVDKSRWQRGDT | CD16 |
| | FICAVMHEALHNHYTQISLSHSPGK | L(5)P |
| 185 | PGCGLLGGPSVFIFPPKPKDILVTARTPTVTCVVVDLDR | Exemplary variant |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | Protein A+ |
| | NVYVLPPSRDEMSKNTVTLTCLVKDFFPPEIDVEWQSNG | C1q+ |
| | QQEPESKYRMTPPQLDEDGSYFLYSKLSVDKSRWQRGDT | CD16− |
| | FICAVMHEALHNHYTQISLSHSPGK | P(39)R |
| 186 | PGCGLLGGPSVFIFPPKPKDILVTARTPTVTCVVVDLGP | Exemplary variant |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | Protein A+ |
| | NVYVLPPSRDEMSKNTVTLTCLVKDFFPPEIDVEWQSNG | C1q+ |
| | QQEPESKYRMTPPQLDEDGSYFLYSKLSVDKSRWQRGDT | CD16− |
| | FICAVMHEALHNHYTQISLSHSPGK | D(38)G |
| 187 | PGCGLLGGPSVFIFPPKPKDILVTARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCKVNNIALPSPIEEIISKTPGQAHQP | Protein A+ |
| | NVYVLPPSRDEMSKNTVTLTCLVKDFFPPEIDVEWQSNG | C1q+ |
| | QQEPESKYRMTPPQLDEDGSYFLYSKLSVDKSRWQRGDT | CD16− |
| | FICAVMHEALHNHYTQISLSHSPGK | K(97)I |
| 188 | PGCGLLGGPSVFIFPPKPKDILVTARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCKVNNKGLPSPIEEIISKTPGQAHQP | Protein A+ |
| | NVYVLPPSRDEMSKNTVTLTCLVKDFFPPEIDVEWQSNG | C1q+ |
| | QQEPESKYRMTPPQLDEDGSYFLYSKLSVDKSRWQRGDT | CD16− |
| | FICAVMHEALHNHYTQISLSHSPGK | A(98)G |
| 189 | PGCGLLGGPSVFIFPPKPKDILVTARTPTVTCVVVDLGP | Exemplary variant |
| | ENPEVQISWFVDSKQVQTANTQPREEQSNGTYRVVSVLP | canine IgG-C Fc |
| | IGHQDWLSGKQFKCKVNNIGLPSPIEEIISKTPGQAHQP | Protein A+ |
| | NVYVLPPSRDEMSKNTVTLTCLVKDFFPPEIDVEWQSNG | C1q+ |
| | QQEPESKYRMTPPQLDEDGSYFLYSKLSVDKSRWQRGDT | CD16− |
| | FICAVMHEALHNHYTQISLSHSPGK | D(38)G |
| | | K(97)I |
| | | A(98)G |
| 190 | PGCGLLGGPSVFIFPPKPKDILVTARTPTVTCVVVDLGP | Exemplary variant |
| | ENPEVQISWFVDSKQVQTANTQPREEQSNGTYRVVSVLP | canine IgG-C Fc |
| | IGHQDWLSGKQFKCRVNNIGLPSPIEEIISKTPGQAHQP | Protein A+ |
| | NVYVLPPSRDEMSKNTVTLTCLVKDFFPPEIDVEWQSNG | Clq− |
| | QQEPESKYRMTPPQLDEDGSYFLYSKLSVDKSRWQRGDT | CD16− |
| | FICAVMHEALHNHYTQISLSHSPGK | D(38)G |
| | | K(93)R |
| | | K(97)I |
| | | A(98)G |
| 191 | PGCGPLGGPSVFIFPPKPKDILVTARTPTVTCVVVDLDR | Exemplary variant |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | Protein A+ |
| | NVYVLPPSRDEMSKNTVTLTCLVKDFFPPEIDVEWQSNG | C1q+ |
| | QQEPESKYRMTPPQLDEDGSYFLYSKLSVDKSRWQRGDT | CD16− |
| | FICAVMHEALHNHYTQISLSHSPGK | L(5)P |
| | | P(39)R |
| 192 | PGCGPLGGPSVFIFPPKPKDILVTARTPTVTCVVVDLDR | Exemplary variant |
| | ENPEVQISWFVDSKQVQTANTQPREEQSNGTYRVVSVLP | canine IgG-C Fc |
| | IGHQDWLSGKQFKCRVNNKALPSPIEEIISKTPGQAHQP | Protein A+ |
| | NVYVLPPSRDEMSKNTVTLTCLVKDFFPPEIDVEWQSNG | C1q− |
| | QQEPESKYRMTPPQLDEDGSYFLYSKLSVDKSRWQRGDT | CD16− |
| | FICAVMHEALHNHYTQISLSHSPGK | M(5)P |
| | | P(39)R |
| | | K(93)R |
| 193 | PVPESLGGPSVFIFPPKPKDTLLIARTPEITCVVLDLGR | Exemplary variant |
| | EDPEVQISWFVDGKEVHTAKTQPREQQFNSTYRVVSVLP | canine IgG-D Fc |
| | IGHQDWLTGKEFKCRVNHIGLPSPIERTISKARGQAHQP | C1q− |
| | SVYVLPPSPKELSSSDTVTLTCLIKDFFPPEIDVEWQSN | Protein A+ |
| | GQPEPESKYHTTAPQLDEDGSYFLYSKLSVDKSRWQQGD | I(21)T |
| | TFTCAVMHEALHNHYTDLSLSHSPGK | R(23)L |
| | | T(25)A |
| | | E(80)G |
| | | Q(207)H |
| 194 | PVPESLGGPSVFIFPPKPKDTLRITRTPEITCVVLDLGR | Exemplary variant |
| | EDPEVQISWFVDGKEVHTAKTQPREQQFNSTYRVVSVLP | canine IgG-D Fc |
| | IEHQDWLTGKEFKCRVNHIGLPSPIERTISKARGQAHQP | C1q− |
| | SVYVLPPSPKELSSSDTVTLTCLIKDFFPPEIDVEWQSN | Protein A+ |
| | GQPEPESKYHTTAPQLDEDGSYFLYSKLSVDKSRWQQGD | I(21)T |
| | TFTCAVMHEALHNHYTDLSLSHSPGK | Q(207)H |
| 195 | PVPEPLGGPSVLIFPPKPKDILRITRTPEVTCVVLDLGR | Exemplary variant |
| | EDPEVQISWFVDGKEVHTAKTQSREQQFNGTYRVVSVLP | canine IgG-A Fc |
| | IEHQDWLTGKEFKCRVNHIDLPSPIERTISKARGRAHKP | Bispecific knob |
| | SVYVLPPSPKELSSSDTVSIWCLIKDFYPPDIDVEWQSN | T(138)W |
| | GQQEPERKHRMTPPQLDEDGSYFLYSKLSVDKSRWQQGD | |
| | PFTCAVMHETLQNHYTDLSLSHSPGK | |
| 196 | PAPEMLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Bispecific knob |
| | SVYVLPPSREELSKNTVSLWCLIKDFFPPDIDVEWQSNG | T(137)W |
| | QQEPESKYRTTPPQLDEDGSYFLYSKLSVDKSRWQRGDT | |
| | FICAVMHEALHNHYTQESLSHSPGK | |
| 197 | PGCGLLGGPSVFIFPPKPKDILVTARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | Bispecific knob |
| | NVYVLPPSRDEMSKNTVTLWCLVKDFFPPEIDVEWQSNG | T(137)W |
| | QQEPESKYRMTPPQLDEDGSYFLYSKLSVDKSRWQRGDT | |
| | FICAVMHEALHNHYTQISLSHSPGK | |
| 198 | PVPESLGGPSVFIFPPKPKDILRITRTPEITCVVLDLGR | Exemplary variant |
| | EDPEVQISWFVDGKEVHTAKTQPREQQFNSTYRVVSVLP | canine IgG-D Fc |
| | IEHQDWLTGKEFKCRVNHIGLPSPIERTISKARGQAHQP | Bispecific knob |
| | SVYVLPPSPKELSSSDTVTLWCLIKDFFPPEIDVEWQSN | T(138)W |
| | GQPEPESKYHTTAPQLDEDGSYFLYSKLSVDKSRWQQGD | |
| | TFTCAVMHEALQNHYTDLSLSHSPGK | |
| 199 | PVPEPLGGPSVLIFPPKPKDILRITRTPEVTCVVLDLGR | Exemplary variant |
| | EDPEVQISWFVDGKEVHTAKTQSREQQFNGTYRVVSVLP | canine IgG-A Fc |
| | IEHQDWLTGKEFKCRVNHIDLPSPIERTISKARGRAHKP | Bispecific hole |
| | SVYVLPPSPKELSSSDTVSISCAIKDFYPPDIDVEWQSN | T(138)S |
| | GQQEPERKHRMTPPQLDEDGSYFLYSKLSVDKSRWQQGD | L(140)A |
| | PFTCAVMHETLQNHYTDLSLSHSPGK | |
| 200 | PAPEMLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Bispecific hole |
| | SVYVLPPSREELSKNTVSLSCAIKDFFPPDIDVEWQSNG | T(137)S |
| | QQEPESKYRTTPPQLDEDGSYFLYSKLSVDKSRWQRGDT | L(139)A |
| | FICAVMHEALHNHYTQESLSHSPGK | |
| 201 | PGCGLLGGPSVFIFPPKPKDILVTARTPTVTCVVVDLDP | Exemplary variant |
| | ENPEVQISWFVDSKQVQTANTQPREEQSNGTYRVVSVLP | canine IgG-C Fc |
| | IGHQDWLSGKQFKCKVNNKALPSPIEEIISKTPGQAHQP | Bispecific hole |
| | NVYVLPPSRDEMSKNTVTLSCAVKDFFPPEIDVEWQSNG | T(137)S |
| | QQEPESKYRMTPPQLDEDGSYFLYSKLSVDKSRWQRGDT | L(139)A |
| | FICAVMHEALHNHYTQISLSHSPGK | |
| 202 | PVPESLGGPSVFIFPPKPKDILRITRTPEITCVVLDLGR | Exemplary variant |
| | EDPEVQISWFVDGKEVHTAKTQPREQQFNSTYRVVSVLP | canine IgG-D Fc |
| | IEHQDWLTGKEFKCRVNHIGLPSPIERTISKARGQAHQP | Bispecific hole |
| | SVYVLPPSPKELSSSDTVTLSCAIKDFFPPEIDVEWQSN | T(138)S |
| | GQPEPESKYHTTAPQLDEDGSYFLYSKLSVDKSRWQQGD | L(140)A |
| | TFTCAVMHEALQNHYTDLSLSHSPGK | |
| 203 | RKTDHPPGPKPCDCPKCPPPEMLGGPSIFIFPPKPKDTL | Exemplary wild-type |
| | SISRTPEVTCLVVDLGPDDSDVQITWFVDNTQVYTAKTS | feline IgG1a Fc |
| | PREEQFNSTYRVVSVLPILHQDWLKGKEFKCKVNSKSLP | Protein A+ |
| | SPIERTISKAKGQPHEPQVYVLPPAQEELSENKVSVTCL | C1q+ |
| | IKSFHPPDIAVEWEITGQPEPENNYRTTPPQLDSDGTYF | |
| | VYSKLSVDRSHWQRGNTYTCSVSHEALHSHHTQKSLTQS | |
| | PGK | |
| 204 | RKTDHPPGPKTGEGPKCPPPEMLGGPSIFIFPPKPKDTL | Exemplary wild-type |
| | SISRTPEVTCLVVDLGPDDSDVQITWFVDNTQVYTAKTS | feline IgG1a Fc |
| | PREEQFNSTYRVVSVLPILHQDWLKGKEFKCKVNSKSLP | Protein A+ |
| | SPIERTISKAKGQPHEPQVYVLPPAQEELSENKVSVTCL | C1q+ |
| | IKSFHPPDIAVEWEITGQPEPENNYRTTPPQLDSDGTYF | |
| | VYSKLSVDRSHWQRGNTYTCSVSHEALHSHHTQKSLTQS | |
| | PGK | |
| 205 | RKTDHPPGPKPCDCPKCPPPEMLGGPSIFIFPPKPKDTL | Exemplary wild-type |
| | SISRTPEVTCLVVDLGPDDSDVQITWFVDNTQVYTAKTS | feline IgG1b Fc |
| | PREEQFNSTYRVVSVLPILHQDWLKGKEFKCKVNSKSLP | Protein A+ |
| | SPIERTISKDKGQPHEPQVYVLPPAQEELSENKVSVTCL | C1q+ |
| | IEGFYPSDIAVEWEITGQPEPENNYRTTPPQLDSDGTYF | |
| | LYSRLSVDRSRWQRGNTYTCSVSHEALHSHHTQKSLTQS | |
| | PGK | |
| 206 | RKTDHPPGPKTGEGPKCPPPEMLGGPSIFIFPPKPKDTL | Exemplary wild-type |
| | SISRTPEVTCLVVDLGPDDSDVQITWFVDNTQVYTAKTS | feline IgG1b Fc |
| | PREEQFNSTYRVVSVLPILHQDWLKGKEFKCKVNSKSLP | Protein A+ |
| | SPIERTISKDKGQPHEPQVYVLPPAQEELSENKVSVTCL | C1q+ |
| | IEGFYPSDIAVEWEITGQPEPENNYRTTPPQLDSDGTYF | |
| | LYSRLSVDRSRWQRGNTYTCSVSHEALHSHHTQKSLTQS | |
| | PGK | |
| 207 | PKTASTIESKTGEGPKCPVPEIPGAPSVFIFPPKPKDTL | Exemplary wild-type |
| | SISRTPEVTCLVVDLGPDDSNVQITWFVDNTEMHTAKTR | feline IgG2 Fc |
| | PREEQFNSTYRVVSVLPILHQDWLKGKEFKCKVNSKSLP | Protein A+ |
| | SAMERTISKAKGQPHEPQVYVLPPTQEELSENKVSVTCL | C1q− |
| | IKGFHPPDIAVEWEITGQPEPENNYQTTPPQLDSDGTYF | |
| | LYSRLSVDRSHWQRGNTYTCSVSHEALHSHHTQKSLTQS | |
| | PGK | |
| 208 | RKTDHPPGPKPCDCPPCPPPEMLGGPSIFIFPPKPKDTL | Exemplary variant feline |
| | SISRTPEVTCLVVDLGPDDSDVQITWFVDNTQVYTAKTS | IgG1a Fc with modified |
| | PREEQFNSTYRVVSVLPILHQDWLKGKEFKCKVNSKSLP | hinge |
| | SPIERTISKAKGQPHEPQVYVLPPAQEELSENKVSVTCL | K(16)P |
| | IKSFHPPDIAVEWEITGQPEPENNYRTTPPQLDSDGTYF | |
| | VYSKLSVDRSHWQRGNTYTCSVSHEALHSHHTQKSLTQS | |
| | PGK | |
| 209 | RKTDHPPGPKPCDCPKCPPPEMLGGPSIFIFPPKPKDTL | Exemplary variant feline |
| | SISRTPEVTCLVVDLGPDDSDVQITWFVDNTQVYTAKTS | IgG1a Fc |
| | PREEQFNSTYRVVSVLPILHQDWLKGKEFKCKVNSKSLP | Protein A+ |
| | SPIERTISKAKGQPHEPQVYVLPPAQEELSENKVSVTCL | C1q− |
| | IKSFHPPDIAVEWEITGQPEPENNYRTTPPQLDSDGTYF | P(198)A |
| | VYSKLSVDRSHWQRGNTYTCSVSHEALHSHHTQKSLTQS | |
| | PGK | |
| 210 | RKTDHPPGPKTGEGPKCPPPEMLGGPSIFIFPPKPKDTL | Exemplary variant feline |
| | SISRTPEVTCLVVDLGPDDSDVQITWFVDNTQVYTAKTS | IgG1a Fc |
| | PREEQFNSTYRVVSVLPILHQDWLKGKEFKCKVNSKSLP | Protein A+ |
| | SPIERTISKAKGQPHEPQVYVLPPAQEELSENKVSVTCL | C1q− |
| | IKSFHPPDIAVEWEITGQPEPENNYRTTPPQLDSDGTYF | P(198)A |
| | VYSKLSVDRSHWQRGNTYTCSVSHEALHSHHTQKSLTQS | |
| | PGK | |
| 211 | RKTDHPPGPKPCDCPPCPPPEMLGGPSIFIFPPKPKDTL | Exemplary variant feline |
| | SISRTPEVTCLVVDLGPDDSDVQITWFVDNTQVYTAKTS | IgG1b Fc with modified |
| | PREEQFNSTYRVVSVLPILHQDWLKGKEFKCKVNSKSLP | hinge |
| | SPIERTISKDKGQPHEPQVYVLPPAQEELSENKVSVTCL | K(16)P |
| | IEGFYPSDIAVEWEITGQPEPENNYRTTPPQLDSDGTYF | |
| | LYSRLSVDRSRWQRGNTYTCSVSHEALHSHHTQKSLTQS | |
| | PGK | |
| 212 | RKTDHPPGPKPCDCPKCPPPEMLGGPSIFIFPPKPKDTL | Exemplary variant feline |
| | SISRTPEVTCLVVDLGPDDSDVQITWFVDNTQVYTAKTS | IgG1b Fc |
| | PREEQFNSTYRVVSVLPILHQDWLKGKEFKCKVNSKSLP | Protein A+ |
| | SPIERTISKDKGQPHEPQVYVLPPAQEELSENKVSVTCL | Clq− |
| | IEGFYPSDIAVEWEITGQPEPENNYRTTPPQLDSDGTYF | P(198)A |
| | LYSRLSVDRSRWQRGNTYTCSVSHEALHSHHTQKSLTQS | |
| | PGK | |
| 213 | RKTDHPPGPKTGEGPKCPPPEMLGGPSIFIFPPKPKDTL | Exemplary variant feline |
| | SISRTPEVTCLVVDLGPDDSDVQITWFVDNTQVYTAKTS | IgG1b Fc |
| | PREEQFNSTYRVVSVLPILHQDWLKGKEFKCKVNSKSLP | Protein A+ |
| | SPIERTISKDKGQPHEPQVYVLPPAQEELSENKVSVTCL | C1q− |
| | IEGFYPSDIAVEWEITGQPEPENNYRTTPPQLDSDGTYF | P(198)A |
| | LYSRLSVDRSRWQRGNTYTCSVSHEALHSHHTQKSLTQS | |
| | PGK | |
| 214 | PKTASTIESKTGECPKCPVPEIPGAPSVFIFPPKPKDTL | Exemplary variant feline |
| | SISRTPEVTCLVVDLGPDDSNVQITWFVDNTEMHTAKTR | IgG2 Fc with modified |
| | PREEQFNSTYRVVSVLPILHQDWLKGKEFKCKVNSKSLP | hinge |
| | SAMERTISKAKGQPHEPQVYVLPPTQEELSENKVSVTCL | Hinge Cys |
| | IKGFHPPDIAVEWEITGQPEPENNYQTTPPQLDSDGTYF | G(14)C |
| | LYSRLSVDRSHWQRGNTYTCSVSHEALHSHHTQKSLTQS | |
| | PGK | |
| 215 | PKTASTIESKTGEGPPCPVPEIPGAPSVFIFPPKPKDTL | Exemplary variant feline |
| | SISRTPEVTCLVVDLGPDDSNVQITWFVDNTEMHTAKTR | IgG2 Fc with modified |
| | PREEQFNSTYRVVSVLPILHQDWLKGKEFKCKVNSKSLP | hinge |
| | SAMERTISKAKGQPHEPQVYVLPPTQEELSENKVSVTCL | K(16)P |
| | IKGFHPPDIAVEWEITGQPEPENNYQTTPPQLDSDGTYF | |
| | LYSRLSVDRSHWQRGNTYTCSVSHEALHSHHTQKSLTQS | |
| | PGK | |
| 216 | RKTDHPPGPKPCDCPKCPPPEMLGGPSVFIFPPKPKDTL | Exemplary variant feline |
| | SISRTPEVTCLVVDLGPDDSNVQITWFVDNTEMHTAKTR | IgG2 Fc with feline |
| | PREEQFNSTYRVVSVLPILHQDWLKGKEFKCKVNSKSLP | IgG1 hinge |
| | SAMERTISKAKGQPHEPQVYVLPPTQEELSENKVSVTCL | |
| | IKGFHPPDIAVEWEITGQPEPENNYQTTPPQLDSDGTYF | |
| | LYSRLSVDRSHWQRGNTYTCSVSHEALHSHHTQKSLTQS | |
| | PGK | |
| 217 | RKTDHPPGPKPCDCPKCPPPEMLGGPSIFIFPPKPKDTL | Exemplary variant feline |
| | SISRTPEVTCLVVDLGPDDSDVQITWFVDNTQVYTAKTS | IgG1a Fc |
| | PREEQFNSTYRVVSVLPILHQDWLKGKEFKCKVNSKSLP | Bispecific knob |
| | SPIERTISKAKGQPHEPQVYVLPPAQEELSENKVSVWCL | T(154)W |
| | IKSFHPPDIAVEWEITGQPEPENNYRTTPPQLDSDGTYF | |
| | VYSKLSVDRSHWQRGNTYTCSVSHEALHSHHTQKSLTQS | |
| | PGK | |
| 218 | RKTDHPPGPKTGEGPKCPPPEMLGGPSIFIFPPKPKDTL | Exemplary variant feline |
| | SISRTPEVTCLVVDLGPDDSDVQITWFVDNTQVYTAKTS | IgG1a Fc |
| | PREEQFNSTYRVVSVLPILHQDWLKGKEFKCKVNSKSLP | Bispecific knob |
| | SPIERTISKAKGQPHEPQVYVLPPAQEELSENKVSVWCL | T(154)W |
| | IKSFHPPDIAVEWEITGQPEPENNYRTTPPQLDSDGTYF | |
| | VYSKLSVDRSHWQRGNTYTCSVSHEALHSHHTQKSLTQS | |
| | PGK | |
| 219 | RKTDHPPGPKPCDCPKCPPPEMLGGPSIFIFPPKPKDTL | Exemplary variant feline |
| | SISRTPEVTCLVVDLGPDDSDVQITWFVDNTQVYTAKTS | IgG1b Fc |
| | PREEQFNSTYRVVSVLPILHQDWLKGKEFKCKVNSKSLP | Bispecific knob |
| | SPIERTISKDKGQPHEPQVYVLPPAQEELSENKVSVWCL | T(154)W |
| | IEGFYPSDIAVEWEITGQPEPENNYRTTPPQLDSDGTYF | |
| | LYSRLSVDRSRWQRGNTYTCSVSHEALHSHHTQKSLTQS | |
| | PGK | |
| 220 | RKTDHPPGPKTGEGPKCPPPEMLGGPSIFIFPPKPKDTL | Exemplary variant feline |
| | SISRTPEVTCLVVDLGPDDSDVQITWFVDNTQVYTAKTS | IgG1b Fc |
| | PREEQFNSTYRVVSVLPILHQDWLKGKEFKCKVNSKSLP | Bispecific knob |
| | SPIERTISKDKGQPHEPQVYVLPPAQEELSENKVSVWCL | T(154)W |
| | IEGFYPSDIAVEWEITGQPEPENNYRTTPPQLDSDGTYF | |
| | LYSRLSVDRSRWQRGNTYTCSVSHEALHSHHTQKSLTQS | |
| | PGK | |
| 221 | PKTASTIESKTGEGPKCPVPEIPGAPSVFIFPPKPKDTL | Exemplary variant feline |
| | SISRTPEVTCLVVDLGPDDSNVQITWFVDNTEMHTAKTR | IgG2 Fc |
| | PREEQFNSTYRVVSVLPILHQDWLKGKEFKCKVNSKSLP | Bispecific knob |
| | SAMERTISKAKGQPHEPQVYVLPPTQEELSENKVSVWCL | T(154)W |
| | IKGFHPPDIAVEWEITGQPEPENNYQTTPPQLDSDGTYF | |
| | LYSRLSVDRSHWQRGNTYTCSVSHEALHSHHTQKSLTQS | |
| | PGK | |
| 222 | RKTDHPPGPKPCDCPKCPPPEMLGGPSIFIFPPKPKDTL | Exemplary variant feline |
| | SISRTPEVTCLVVDLGPDDSDVQITWFVDNTQVYTAKTS | IgG1a Fc |
| | PREEQFNSTYRVVSVLPILHQDWLKGKEFKCKVNSKSLP | Bispecific hole |
| | SPIERTISKAKGQPHEPQVYVLPPAQEELSENKVSVSCA | T(154)S |
| | IKSFHPPDIAVEWEITGQPEPENNYRTTPPQLDSDGTYF | L(156)A |
| | VYSKLSVDRSHWQRGNTYTCSVSHEALHSHHTQKSLTQS | |
| | PGK | |
| 223 | RKTDHPPGPKTGEGPKCPPPEMLGGPSIFIFPPKPKDTL | Exemplary variant feline |
| | SISRTPEVTCLVVDLGPDDSDVQITWFVDNTQVYTAKTS | IgG1a Fc |
| | PREEQFNSTYRVVSVLPILHQDWLKGKEFKCKVNSKSLP | Bispecific hole |
| | SPIERTISKAKGQPHEPQVYVLPPAQEELSENKVSVSCA | T(154)S |
| | IKSFHPPDIAVEWEITGQPEPENNYRTTPPQLDSDGTYF | L(156)A |
| | VYSKLSVDRSHWQRGNTYTCSVSHEALHSHHTQKSLTQS | |
| | PGK | |
| 224 | RKTDHPPGPKPCDCPKCPPPEMLGGPSIFIFPPKPKDTL | Exemplary variant feline |
| | SISRTPEVTCLVVDLGPDDSDVQITWFVDNTQVYTAKTS | IgG1b Fc |
| | PREEQFNSTYRVVSVLPILHQDWLKGKEFKCKVNSKSLP | Bispecific hole |
| | SPIERTISKDKGQPHEPQVYVLPPAQEELSENKVSVSCA | T(154)S |
| | IEGFYPSDIAVEWEITGQPEPENNYRTTPPQLDSDGTYF | L(156)A |
| | LYSRLSVDRSRWQRGNTYTCSVSHEALHSHHTQKSLTQS | |
| | PGK | |
| 225 | RKTDHPPGPKTGEGPKCPPPEMLGGPSIFIFPPKPKDTL | Exemplary variant feline |
| | SISRTPEVTCLVVDLGPDDSDVQITWFVDNTQVYTAKTS | IgG1b Fc |
| | PREEQFNSTYRVVSVLPILHQDWLKGKEFKCKVNSKSLP | Bispecific hole |
| | SPIERTISKDKGQPHEPQVYVLPPAQEELSENKVSVSCA | T(154)S |
| | IEGFYPSDIAVEWEITGQPEPENNYRTTPPQLDSDGTYF | L(156)A |
| | LYSRLSVDRSRWQRGNTYTCSVSHEALHSHHTQKSLTQS | |
| | PGK | |
| 226 | PKTASTIESKTGEGPKCPVPEIPGAPSVFIFPPKPKDTL | Exemplary variant feline |
| | SISRTPEVTCLVVDLGPDDSNVQITWFVDNTEMHTAKTR | IgG2 Fc |
| | PREEQFNSTYRVVSVLPILHQDWLKGKEFKCKVNSKSLP | Bispecific hole |
| | SAMERTISKAKGQPHEPQVYVLPPTQEELSENKVSVSCA | T(154)S |
| | IKGFHPPDIAVEWEITGQPEPENNYQTTPPQLDSDGTYF | L(156)A |
| | LYSRLSVDRSHWQRGNTYTCSVSHEALHSHHTQKSLTQS | |
| | PGK | |
| 227 | ASTTAPSVFPLAPSCGSTSGSTVALACLVSGYFPEPVTV | Wild-type canine IgG-A |
| | SWNSGSLTSGVHTFPSVLQSSGLHSLSSMVTVPSSRWPS | CH1 |
| | ETFTCNVVHPASNTKVDKPV | |
| 228 | ASTTAPSVFPLAPSCGSTSGSTVALACLVSGYFPEPVTV | Wild-type canine IgG-B |
| | SWNSGSLTSGVHTFPSVLQSSGLYSLSSMVTVPSSRWPS | CH1 |
| | ETFTCNVAHPASKTKVDKPV | |
| 229 | ASTTAPSVFPLAPSCGSQSGSTVALACLVSGYIPEPVTV | Wild-type canine IgG-C |
| | SWNSVSLTSGVHTFPSVLQSSGLYSLSSMVTVPSSRWPS | CH1 |
| | ETFTCNVAHPATNTKVDKPV | |
| 230 | ASTTAPSVFPLAPSCGSTSGSTVALACLVSGYFPEPVTV | Wild-type canine IgG-D |
| | SWNSGSLTSGVHTFPSVLQSSGLYSLSSTVTVPSSRWPS | CH1 |
| | ETFTCNVVHPASNTKVDKPV | |
| 231 | ASTTAPSVFPLAPSCGSTSGSTVLLACLVDGYFPEPVTV | Variant canine IgG-A |
| | SWNSGSLTSGVHTFPSVLQSSGLHSLSSMVTVPSSRWPS | CH1 |
| | ETFTCNVVHPASNTKVDKPV | A(24)L |
| | | S(30)D |
| 232 | ASTTAPSVFPLAPSCGSTSGSTVLLACLVDGYFPEPVTV | Variant canine IgG-B |
| | SWNSGSLTSGVHTFPSVLQSSGLYSLSSMVTVPSSRWPS | CH1 |
| | ETFTCNVAHPASKTKVDKPV | A(24)L |
| | | S(30)D |
| 233 | ASTTAPSVFPLAPSCGSQSGSTVLLACLVDGYIPEPVTV | Variant canine IgG-C |
| | SWNSVSLTSGVHTFPSVLQSSGLYSLSSMVTVPSSRWPS | CH1 |
| | ETFTCNVAHPATNTKVDKPV | A(24)L |
| | | S(30)D |
| 234 | ASTTAPSVFPLAPSCGSTSGSTVLLACLVDGYFPEPVTV | Variant canine IgG-D |
| | SWNSGSLTSGVHTFPSVLQSSGLYSLSSTVTVPSSRWPS | CH1 |
| | ETFTCNVVHPASNTKVDKPV | A(24)L |
| | | S(30)D |
| 235 | RNDAQPAVYLFQPSPDQLHTGSASVVCLLNSFYPKDINV | Wild-type canine κ |
| | KWKVDGVIQDTGIQESVTEQDKDSTYSLSSTLTMSSTEY | constant region |
| | LSHELYSCEITHKSLPSTLIKSFQRSECQRVD | |
| 236 | RNDAQPAVYLAQPSPDQLHTGRASVVCLLNSFYPKDINV | Variant canine κ |
| | KWKVDGVIQDTGIQESVTEQDKDSTYSLSSTLTMSSTEY | constant region |
| | LSHELYSCEITHKSLPSTLIKSFQRSECQRVD | F(11)A |
| | | S(22)R |
| 237 | ASTTAPSVFPLAPSCGTTSGATVALACLVSGYFPEPVTV | Wild-type feline IgG1 |
| | SWNSGALTSGVHTFPAVLQASGLYSLSSMVTVPSSRWLS | CH1 |
| | DTFTCNVAHPPSNTKVDKTV | |
| 238 | ASTTASSVFPLAPSCGTTSGATVALACLSLGYFPEPVTV | Wild-type feline IgG2 |
| | SWNSGALTSGVHTFPSVLQASGLYSLSSMVTVPSSRWLS | CH1 |
| | DTFTCNVAHRPSSTKVDKTV | |
| 239 | ASTTAPSVFPLAPSCGTTSGATVLLACLVDGYFPEPVTV | Variant feline IgG1 CH1 |
| | SWNSGALTSGVHTFPAVLQASGLYSLSSMVTVPSSRWLS | A(24)L |
| | DTFTCNVAHPPSNTKVDKTV | S(30)D |
| 240 | ASTTASSVFPLAPSCGTTSGATVLLACLDLGYFPEPVTV | Variant feline IgG2 CH1 |
| | SWNSGALTSGVHTFPSVLQASGLYSLSSMVTVPSSRWLS | A(24)L |
| | DTFTCNVAHRPSSTKVDKTV | S(29)D |
| 241 | RSDAQPSVFLFQPSLDELHTGSASIVCILNDFYPKEVNV | Wild-type feline |
| | KWKVDGVVQNKGIQESTTEQNSKDSTYSLSSTLTMSSTE | constant region |
| | YQSHEKFSCEVTHKSLASTLVKSFNRSECQRE | |
| 242 | RSDAQPSVFLAQPSLDELHTGRASIVCILNDFYPKEVNV | Variant feline κ constant |
| | KWKVDGVVQNKGIQESTTEQNSKDSTYSLSSTLIMSSTE | region |
| | YQSHEKFSCEVTHKSLASTLVKSFNRSECQRE | F(11)A |
| | | S(22)R |
| 243 | EVQLVQSGAEVKKPGASVKVSCKASGYTFTSYVMHWVRQ | Caninized Clone I |
| | APGQGLEWMGYINPNNDGTFYNGKFQGRVTLTADTSTST | variable HC v2 and |
| | AYMELSSLRAGDIAVYYCAAFYYGFAYWGQGTLVTVSSA | variant canine IgG-B Fc |
| | STTAPSVFPLAPSCGSTSGSTVLLACLVDGYFPEPVTVS | C1q− |
| | WNSGSLTSGVHTFPSVLQSSGLYSLSSMVTVPSSRWPSE | CD16− |
| | TFTCNVAHPASKTKVDKPVPKRENGRVPRPPDCPKCPAP | Bispecific Knob |
| | EPLGGPSVFIFPPKPKDTLLIARTPEVTCVVVDLDREDP | Variant CH1 |
| | EVQISWFVDGKQMQTAKTQPREEQFNGTYRVVSVLPIGH | |
| | QDWLKGKQFTCRVNNKALPSPIERTISKARGQAHQPSVY | |
| | VLPPSREELSKNTVTLWCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 244 | DIVMTQTPLSLSVSPGETASISCRASQEISGYLSWLQQK | Caninized Clone I |
| | PGGTIKRLIYAASNRDTGVPDRFSGSGSGTDFTLRISRV | variable LC v2 and |
| | EADDTGVYYCLQYASYPWTFGGGTKVELKRNDAQPAVYL | variant canine κ constant |
| | AQPSPDQLHTGRASVVCLLNSFYPKDINVKWKVDGVIQD | region |
| | TGIQESVTEQDKDSTYSLSSTLIMSSTEYLSHELYSCEI | |
| | THKSLPSTLIKSFQRSECQRVD | |
| 245 | EVQLVESGPSLVKPGGSLRLTCSVTGDSITSGYWNWIRK | Caninized anti-canine |
| | FPGNKLEYMGYISYSGITDYNPSLKSRITISRDTSKNQY | IL31 Clone M14 |
| | YLQLNSVTTEDTATYYCARYGNYGYAMDYWGQGTLVTVS | variable HC and variant |
| | SASTTAPSVFPLAPSCGSTSGSTVALACLVSGYFPEPVT | canine IgG-B Fc |
| | VSWNSGSLTSGVHTFPSVLQSSGLYSLSSMVTVPSSRWP | C1q− |
| | SETFTCNVAHPASKTKVDKPVPKRENGRVPRPPDCPKCP | CD16− |
| | APEPLGGPSVFIFPPKPKDTLLIARTPEVTCVVVDLDRE | Bispecific Hole |
| | DPEVQISWFVDGKQMQTAKTQPREEQFNGTYRVVSVLPI | |
| | GHQDWLKGKQFTCRVNNKALPSPIERTISKARGQAHQPS | |
| | VYVLPPSREELSKNTVTLSCAIKDFFPPDIDVEWQSNGQ | |
| | QEPESKYRTTPPQLDEDGSYFLYSKLSVDKSRWQRGDTF | |
| | ICAVMHEALHNHYTQESLSHSPGK | |
| 246 | DIVMTQSPASLSVSLGQRATISCRASESVDTYGNSFMHW | Caninized anti-canine |
| | YQQKPGQSPKLLIYRASNLESGIPARFGGSGSGTDFTLT | IL31 Clone M14 |
| | IDPVQADDVATYYCQQSYEDPWTFGGGTKLEIKRNDAQP | variable LC and canine κ |
| | AVYLFQPSPDQLHTGSASVVCLLNSFYPKDINVKWKVDG | constant region |
| | VIQDTGIQESVTEQDKDSTYSLSSTLTMSSTEYLSHELY | |
| | SCEITHKSLPSTLIKSFQRSECQRVD | |
| 247 | GPSVFIFPPNPKDTLMITRTPEVTCVVVDVSQENPDVKF | Exemplary wild-type |
| | NWYMDGVEVRTATTRPKEEQFNSTYRVVSVLRIQHQDWL | equine IgG1 Fc |
| | SGKEFKCKVNNQALPQPIERTITKTKGRSQEPQVYVLAP | |
| | HPDELSKSKVSVTCLVKDFYPPEINIEWQSNGQPELETK | |
| | YSTTQAQQDSDGSYFLYSKLSVDRNRWQQGTTFTCGVMH | |
| | EALHNHYTQKNVSKNPGK | |
| 248 | GPSVFIFPPNPKDALMISRTPVVTCVVVNLSDQYPDVQF | Exemplary wild-type |
| | SWYVDNTEVHSAITKQREAQFNSTYRVVSVLPIQHQDWL | equine IgG2 Fc |
| | SGKEFKCSVTNVGVPQPISRAISRGKGPSRVPQVYVLPP | |
| | HPDELAKSKVSVTCLVKDFYPPDISVEWQSNRWPELEGK | |
| | YSTTPAQLDGDGSYFLYSKLSLETSRWQQVESFTCAVMH | |
| | EALHNHFTKTDISESLGK | |
| 249 | GPSVFIFPPKPKDVLMITRMPEVTCLVVDVSHDSSDVLF | Exemplary wild-type |
| | TWYVDGTEVKTAKTMPNEEQNNSTYRVVSVLRIQHQDWL | equine IgG3 Fc |
| | NGKKFKCKVNNQALPAPVERTISKATGQTRVPQVYVLAP | |
| | HPDELSKNKVSVTCLVKDFYPPDITVEWQSNEHPEPEGK | |
| | YRTTEAQKDSDGSYFLYSKLTVEKDRWQQGTTFTCVVMH | |
| | EALHNHVMQKNISKNPGK | |
| 250 | GPSVFIFPPKPKDVLMISRTPTVTCVVVDVGHDFPDVQF | Exemplary wild-type |
| | NWYVDGVETHTATTEPKQEQFNSTYRVVSVLPIQHKDWL | equine IgG4 Fc |
| | SGKEFKCKVNNKALPAPVERTISAPTGQPREPQVYVLAP | |
| | HRDELSKNKVSVTCLVKDFYPPDIDIEWKSNGQPEPETK | |
| | YSTTPAQLDSDGSYFLYSKLTVETNRWQQGTTFTCAVMH | |
| | EALHNHYTEKSVSKSPGK | |
| 251 | GPSVFIFPPKPKDVLMISRKPEVTCVVVDLGHDDPDVQF | Exemplary wild-type |
| | TWFVDGVETHTATTEPKEEQFNSTYRVVSVLPIQHQDWL | equine IgG5 Fc |
| | SGKEFKCSVTSKALPAPVERTISKAKGQLRVPQVYVLAP | |
| | HPDELAKNTVSVTCLVKDFYPPEIDVEWQSNEHPEPEGK | |
| | YSTTPAQLNSDGSYFLYSKLSVETSRWKQGESFTCGVMH | |
| | EAVENHYTQKNVSHSPGK | |
| 252 | GRPSVFIFPPNPKDTLMISRTPEVTCVVVDVSQENPDV | Exemplary wild-type |
| | KFNWYVDGVEAHTATTKAKEKQDNSTYRVVSVLPIQHQ | equine IgG6 Fc |
| | DWRRGKEFKCKVNNRALPAPVERTITKAKGELQDPKVY | |
| | ILAPHREEVTKNTVSVTCLVKDFYPPDINVEWQSNEEP | |
| | EPEVKYSTTPAQLDGDGSYFLYSKLTVETDRWEQGESF | |
| | TCVVMHEAIRHTYRQKSITNFPGK | |
| 253 | GPSVFIFPPKPKDVLMISRTPTVTCVVVDVGHDFPDVQF | Exemplary wild-type |
| | NWYVDGVETHTATTEPKQEQNNSTYRVVSILAIQHKDWL | equine IgG7 Fc |
| | SGKEFKCKVNNQALPAPVQKTISKPTGQPREPQVYVLAP | |
| | HPDELSKNKVSVTCLVKDFYPPDIDIEWKSNGQPEPETK | |
| | YSTTPAQLDGDGSYFLYSKLTVETNRWQQGTTFTCAVMH | |
| | EALHNHYTEKSVSKSPGK | |
| 254 | GPSVFIFPPNPKDTLMITRTPEVTCVVVDVSQENPDVKF | Exemplary variant |
| | NWYMDGVEVRTATTRPKEEQFNSTYRVVSVLRIQHQDWL | equine IgG1 Fc |
| | SGKEFKCKVNNQALPQPIERTITKTKGRSQEPQVYVLAP | Bispecific knob |
| | HPDELSKSKVSVWCLVKDFYPPEINIEWQSNGQPELETK | T(130)W |
| | YSTTQAQQDSDGSYFLYSKLSVDRNRWQQGTTFTCGVMH | |
| | EALHNHYTQKNVSKNPGK | |
| 255 | GPSVFIFPPNPKDALMISRTPVVTCVVVNLSDQYPDVQF | Exemplary variant |
| | SWYVDNTEVHSAITKQREAQFNSTYRVVSVLPIQHQDWL | equine IgG2 Fc |
| | SGKEFKCSVTNVGVPQPISRAISRGKGPSRVPQVYVLPP | Bispecific knob |
| | HPDELAKSKVSVWCLVKDFYPPDISVEWQSNRWPELEGK | T(130)W |
| | YSTTPAQLDGDGSYFLYSKLSLETSRWQQVESFTCAVMH | |
| | EALHNHFTKTDISESLGK | |
| 256 | GPSVFIFPPKPKDVLMITRMPEVTCLVVDVSHDSSDVLF | Exemplary variant |
| | TWYVDGTEVKTAKTMPNEEQNNSTYRVVSVLRIQHQDWL | equine IgG3 Fc |
| | NGKKFKCKVNNQALPAPVERTISKATGQTRVPQVYVLAP | Bispecific knob |
| | HPDELSKNKVSVWCLVKDFYPPDITVEWQSNEHPEPEGK | T(130)W |
| | YRTTEAQKDSDGSYFLYSKLTVEKDRWQQGTTFTCVVMH | |
| | EALHNHVMQKNISKNPGK | |
| 257 | GPSVFIFPPKPKDVLMISRTPTVTCVVVDVGHDFPDVQF | Exemplary variant |
| | NWYVDGVETHTATTEPKQEQFNSTYRVVSVLPIQHKDWL | equine IgG4 Fc |
| | SGKEFKCKVNNKALPAPVERTISAPTGQPREPQVYVLAP | Bispecific knob |
| | HRDELSKNKVSVWCLVKDFYPPDIDIEWKSNGQPEPETK | T(130)W |
| | YSTTPAQLDSDGSYFLYSKLTVETNRWQQGTTFTCAVMH | |
| | EALHNHYTEKSVSKSPGK | |
| 258 | GPSVFIFPPKPKDVLMISRKPEVTCVVVDLGHDDPDVQF | Exemplary variant |
| | TWFVDGVETHTATTEPKEEQFNSTYRVVSVLPIQHQDWL | equine IgG5 Fc |
| | SGKEFKCSVTSKALPAPVERTISKAKGQLRVPQVYVLAP | Bispecific knob |
| | HPDELAKNTVSVWCLVKDFYPPEIDVEWQSNEHPEPEGK | T(130)W |
| | YSTTPAQLNSDGSYFLYSKLSVETSRWKQGESFTCGVMH | |
| | EAVENHYTQKNVSHSPGK | |
| 259 | RPSVFIFPPNPKDTLMISRTPEVTCVVVDVSQENPDVK | Exemplary variant |
| | FNWYVDGVEAHTATTKAKEKQDNSTYRVVSVLPIQHQD | equine IgG6 Fc |
| | WRRGKEFKCKVNNRALPAPVERTITKAKGELQDPKVYI | Bispecific knob |
| | LAPHREEVTKNTVSVWCLVKDFYPPDINVEWQSNEEPE | T(130)W |
| | PEVKYSTTPAQLDGDGSYFLYSKLIVETDRWEQGESFT | |
| | CVVMHEAIRHTYRQKSITNFPGK | |
| 260 | GPSVFIFPPKPKDVLMISRTPTVTCVVVDVGHDFPDVQF | Exemplary variant |
| | NWYVDGVETHTATTEPKQEQNNSTYRVVSILAIQHKDWL | equine IgG7 Fc |
| | SGKEFKCKVNNQALPAPVQKTISKPTGQPREPQVYVLAP | Bispecific knob |
| | HPDELSKNKVSVWCLVKDFYPPDIDIEWKSNGQPEPETK | T(130)W |
| | YSTTPAQLDGDGSYFLYSKLTVETNRWQQGTTFTCAVMH | |
| | EALHNHYTEKSVSKSPGK | |
| 261 | GPSVFIFPPNPKDTLMITRTPEVTCVVVDVSQENPDVKF | Exemplary variant |
| | NWYMDGVEVRTATTRPKEEQFNSTYRVVSVLRIQHQDWL | equine IgG1 Fc |
| | SGKEFKCKVNNQALPQPIERTITKTKGRSQEPQVYVLAP | Bispecific hole |
| | HPDELSKSKVSVSCAVKDFYPPEINIEWQSNGQPELETK | T(130)S |
| | YSTTQAQQDSDGSYFLYSKLSVDRNRWQQGTTFTCGVMH | L(132)A |
| | EALHNHYTQKNVSKNPGK | |
| 262 | GPSVFIFPPNPKDALMISRTPVVTCVVVNLSDQYPDVQF | Exemplary variant |
| | SWYVDNTEVHSAITKQREAQFNSTYRVVSVLPIQHQDWL | equine IgG2 Fc |
| | SGKEFKCSVTNVGVPQPISRAISRGKGPSRVPQVYVLPP | Bispecific hole |
| | HPDELAKSKVSVSCAVKDFYPPDISVEWQSNRWPELEGK | T(130)S |
| | YSTTPAQLDGDGSYFLYSKLSLETSRWQQVESFTCAVMH | L(132)A |
| | EALHNHFTKTDISESLGK | |
| 263 | GPSVFIFPPKPKDVLMITRMPEVTCLVVDVSHDSSDVLF | Exemplary variant |
| | TWYVDGTEVKTAKTMPNEEQNNSTYRVVSVLRIQHQDWL | equine IgG3 Fc |
| | NGKKFKCKVNNQALPAPVERTISKATGQTRVPQVYVLAP | Bispecific hole |
| | HPDELSKNKVSVSCAVKDFYPPDITVEWQSNEHPEPEGK | T(130)S |
| | YRTTEAQKDSDGSYFLYSKLTVEKDRWQQGTTFTCVVMH | L(132)A |
| | EALHNHVMQKNISKNPGK | |
| 264 | GPSVFIFPPKPKDVLMISRTPTVTCVVVDVGHDFPDVQF | Exemplary variant |
| | NWYVDGVETHTATTEPKQEQFNSTYRVVSVLPIQHKDWL | equine IgG4 Fc |
| | SGKEFKCKVNNKALPAPVERTISAPTGQPREPQVYVLAP | Bispecific hole |
| | HRDELSKNKVSVSCAVKDFYPPDIDIEWKSNGQPEPETK | T(130)S |
| | YSTTPAQLDSDGSYFLYSKLTVETNRWQQGTTFTCAVMH | L(132)A |
| | EALHNHYTEKSVSKSPGK | |
| 265 | GPSVFIFPPKPKDVLMISRKPEVTCVVVDLGHDDPDVQF | Exemplary variant |
| | TWFVDGVETHTATTEPKEEQFNSTYRVVSVLPIQHQDWL | equine IgG5 Fc |
| | SGKEFKCSVTSKALPAPVERTISKAKGQLRVPQVYVLAP | Bispecific hole |
| | HPDELAKNTVSVSCAVKDFYPPEIDVEWQSNEHPEPEGK | T(130)S |
| | YSTTPAQLNSDGSYFLYSKLSVETSRWKQGESFTCGVMH | L(132)A |
| | EAVENHYTQKNVSHSPGK | |
| 266 | RPSVFIFPPNPKDTLMISRTPEVTCVVVDVSQENPDVK | Exemplary variant |
| | FNWYVDGVEAHTATTKAKEKQDNSTYRVVSVLPIQHQD | equine IgG6 Fc |
| | WRRGKEFKCKVNNRALPAPVERTITKAKGELQDPKVYI | Bispecific hole |
| | LAPHREEVTKNTVSVSCAVKDFYPPDINVEWQSNEEPE | T(130)S |
| | PEVKYSTTPAQLDGDGSYFLYSKLTVETDRWEQGESFT | L(132)A |
| | CVVMHEAIRHTYRQKSITNFPGK | |
| 267 | GPSVFIFPPKPKDVLMISRTPTVTCVVVDVGHDFPDVQF | Exemplary variant |
| | NWYVDGVETHTATTEPKQEQNNSTYRVVSILAIQHKDWL | equine IgG7 Fc |
| | SGKEFKCKVNNQALPAPVQKTISKPTGQPREPQVYVLAP | Bispecific hole |
| | HPDELSKNKVSVSCAVKDFYPPDIDIEWKSNGQPEPETK | T(130)S |
| | YSTTPAQLDGDGSYFLYSKLTVETNRWQQGTTFTCAVMH | L(132)A |
| | EALHNHYTEKSVSKSPGK | |
| 278 | TVSGFSLSRYSVH | Clone M3 CDR-H1 |
| 279 | GMIWGGGST | Clone M3 CDR-H2 |
| 280 | TYRGYALDY | Clone M3 CDR-H3 |
| 281 | QVQLKESGPGLVAPSQSLSITC | Clone M3 HC-FR1 |
| 282 | WVRQPPGKGLEWL | Clone M3 HC-FR2 |
| 283 | DYNSVFKSRLSISKDNSKSQVFLKMNSLQTDDTAMYYCA | Clone M3 HC-FR3 |
| | R | |
| 284 | WGQGTSVTVSS | Clone M3 HC-FR4 |
| 285 | RASQDISNYLN | Clone M3 CDR-L1 |
| 286 | YYTSRLQS | Clone M3 CDR-L2 |
| 287 | QQANTLPLT | Clone M3 CDR-L3 |
| 288 | DIQMTQTTSSLSASLGDRVTISC | Clone M3 LC-FR1 |
| 289 | WYQQKPDGTVKLLI | Clone M3 LC-FR2 |
| 290 | GVPSRFSGSGSGTDYCLTISNLEQEDIATYFC | Clone M3 LC-FR3 |
| 291 | FGSGTKLELK | Clone M3 LC-FR4 |
| 292 | QVQLKESGPGLVAPSQSLSITCTVSGFSLSRYSVHWVRQ | Clone M3 variable HC |
| | PPGKGLEWLGMIWGGGSTDYNSVFKSRLSISKDNSKSQV | |
| | FLKMNSLQTDDTAMYYCARTYRGYALDYWGQGTSVTVSS | |
| 293 | DIQMTQTTSSLSASLGDRVTISCRASQDISNYLNWYQQK | Clone M3 variable LC |
| | PDGTVKLLIYYTSRLQSGVPSRFSGSGSGTDYCLTISNL | |
| | EQEDIATYFCQQANTLPLTFGSGTKLELK | |
| 294 | TVSGFSLSRYSVH | Clone M5 CDR-H1 |
| 295 | GMIWGGGST | Clone M5 CDR-H2 |
| 296 | TYRGYALDY | Clone M5 CDR-H3 |
| 297 | QVQLKESGPGLVAPSQSLSITC | Clone M5 HC-FR1 |
| 298 | WVRQPPGKGLEWL | Clone M5 HC-FR2 |
| 299 | DYNSVFKSRLSISKDNSKSQVFLKMNSLQTDDTAMYYCA | Clone M5 HC-FR3 |
| | R | |
| 300 | WGQGTSVTVSS | Clone M5 HC-FR4 |
| 301 | RASQDISNYLN | Clone M5 CDR-L1 |
| 302 | YYTSRLQS | Clone M5 CDR-L2 |
| 303 | QQANTLPLT | Clone M5 CDR-L3 |
| 304 | DIQMTQTTSSLSASLGDRVTISC | Clone M5 LC-FR1 |
| 305 | WYQQKPDGTVKLLI | Clone M5 LC-FR2 |
| 306 | GVPSRFSGSGSGTDYCLTISNLEQEDIATYFC | Clone M5 LC-FR3 |
| 307 | FGSGTKLELK | Clone M5 LC-FR4 |
| 308 | QVQLKESGPGLVAPSQSLSITCTVSGFSLSRYSVHWVRQ | Clone M5 variable HC |
| | PPGKGLEWLGMIWGGGSTDYNSVFKSRLSISKDNSKSQV | |
| | FLKMNSLQTDDTAMYYCARTYRGYALDYWGQGTSVTVSS | |
| 309 | DIQMTQTTSSLSASLGDRVTISCRASQDISNYLNWYQQK | Clone M5 variable LC |
| | PDGTVKLLIYYTSRLQSGVPSRFSGSGSGTDYCLTISNL | |
| | EQEDIATYFCQQANTLPLTFGSGTKLELK | |
| 310 | KASGYTFTSYDIN | Clone M8 CDR-H1 |
| 311 | GWIYPGDGSTK | Clone M8 CDR-H2 |
| 312 | SSFVV | Clone M8 CDR-H3 |
| 313 | QVQLQQSGPELVKPGALVKISC | Clone M8 HC-FR1 |
| 314 | WVKQRPGQGLEWI | Clone M8 HC-FR2 |
| 315 | YNEKFKGKATLTADKSSSTAYMQLSSLTSEDSAVYFCAR | Clone M8 HC-FR3 |
| 316 | WGAGTTVTVSS | Clone M8 HC-FR4 |
| 317 | KASDPINNWLA | Clone M8 CDR-L1 |
| 318 | SGATSLET | Clone M8 CDR-L2 |
| 319 | HQYWSIPYT | Clone M8 CDR-L3 |
| 320 | DIQMTQSSSYLSVSLGGRVTITC | Clone M8 LC-FR1 |
| 321 | WYQQKPGNAPRLLI | Clone M8 LC-FR2 |
| 322 | GVPSRISGSGSGKDYSLSITSLQTEDIATYYC | Clone M8 LC-FR3 |
| 323 | FGGGTKLEIK | Clone M8 LC-FR4 |
| 324 | QVQLQQSGPELVKPGALVKISCKASGYTFTSYDINWVKQ | Clone M8 variable HC |
| | RPGQGLEWIGWIYPGDGSTKYNEKFKGKATLTADKSSST | |
| | AYMQLSSLTSEDSAVYFCARSSFVVWGAGTTVTVSS | |
| 325 | DIQMTQSSSYLSVSLGGRVTITCKASDPINNWLAWYQQK | Clone M8 variable LC |
| | PGNAPRLLISGATSLETGVPSRISGSGSGKDYSLSITSL | |
| | QTEDIATYYCHQYWSIPYTFGGGTKLEIK | |
| 326 | AASGFTFSSFGMH | Clone M9 CDR-H1 |
| 407 | ASGFTFSSFGMH | Alternative M9 CDR-H1 |
| 327 | AYIRSDSSTIY | Clone M9 CDR-H2 |
| 328 | SGYYGSFSLTY | Clone M9 CDR-H3 |
| 329 | DVQLVESGGGLVQPGGSRKLSC | Clone M9 HC-FR1 |
| 330 | WVRQAPEKGLEWV | Clone M9 HC-FR2 |
| 331 | YADTVKGRFTISRDNPKNTLFLQMTSLRSEDTAMYYCAR | Clone M9 HC-FR3 |
| 332 | WGQGTLVTVSA | Clone M9 HC-FR4 |
| 333 | SASSSVSSNYLH | Clone M9 CDR-L1 |
| 334 | YRTSNLPS | Clone M9 CDR-L2 |
| 335 | QQGSGMLT | Clone M9 CDR-L3 |
| 336 | EIVLTQSPTTMAASPGEKITITC | Clone M9 LC-FR1 |
| 337 | WYQQKPGFSPKLLI | Clone M9 LC-FR2 |
| 338 | GVPARFFGSGSGTSYSLTIGTMEAEDVATYYC | Clone M9 LC-FR3 |
| 339 | FGAGTKLELK | Clone M9 LC-FR4 |
| 340 | DVQLVESGGGLVQPGGSRKLSCAASGFTFSSFGMHWVRQ | Clone M9 variable HC |
| | APEKGLEWVAYIRSDSSTIYYADTVKGRFTISRDNPKNT | |
| | LFLQMTSLRSEDTAMYYCARSGYYGSFSLTYWGQGTLVT | |
| | VSA | |
| 341 | EIVLTQSPTTMAASPGEKITITCSASSSVSSNYLHWYQQ | Clone M9 variable LC |
| | KPGFSPKLLIYRTSNLPSGVPARFFGSGSGTSYSLTIGT | |
| | MEAEDVATYYCQQGSGMLTFGAGTKLELK | |
| 342 | EVQLVESGGDLVKPAGSLRLSCTVSGFSLSRYSVHWVRQ | Caninized Clone M3 |
| | APGKGLEWLGMIWGGGSTDYNSVVKGRFTISRDNAKNTV | variable HC v1 |
| | YLQMNSLRAEDTAMYYCARTYRGYALDYWGQGTLVTVSS | |
| 343 | ELTLQESGPGLVKPSQTLSLTCTVSGFSLSRYSVHWIRQ | Caninized Clone M3 |
| | PRGRGLELLGMIWGGGSTDYNPAFQGRISITADTAKNQF | variable HC v2 |
| | SLQLSSMTTEDTAVYYCARTYRGYALDYWGQGTLVTVSS | |
| 344 | DIVMTQTPLSLSVSPGEPASISCRASQDISNYLNWYLQK | Caninized Clone M3 |
| | AGQSPRLLIYYTSRLQSGVPDRFSGSGSGTDFTLRIGRV | variable LC v1 |
| | EAEDAGIYFCQQANTLPLTFGQGTRLEVR | |
| 345 | EVQLVESGGDLVKPGGSLRLSCAASGFTFSSFGMHWVRQ | Caninized Clone M9 |
| | APGKGLEWVAYIRSDSSTIYYADAVKGRFTISRDNAKNT | variable HC v1 |
| | LYLQMNSLRAEDTAMYYCARSGYYGSFSLTYWGQGTLVT | |
| | VSS | |
| 346 | EVQLVESGGDLVKPGGSLRLSCVASGFTFSSFGMHWVRQ | Caninized Clone M9 |
| | APGKGLQWVAYIRSDSSTIYYADAVKGRFTISRDNAKNT | variable HC v2 |
| | LYLQMNSLRAEDTAMYYCARSGYYGSFSLTYWGQGTLVT | |
| | VSS | |
| 347 | DIMLTQTPLSLSVSPGEPASISCSASSSVSSNYLHWYLQ | Caninized Clone M9 |
| | KAGQSPRLLIYRTSNLPSGVPDRFSGSGSGTDFTLRIGR | variable LC v1 |
| | VEAEDAGIYYCQQGSGMLTFGQGTRLEVR | |
| 408 | DIVMTQTPLSLSVSPGEPASISCSASSSVSSNYLHWYLQ | Caninized Clone M9 |
| | KAGQSPRLLIYRTSNLPSGVPDRFSGSGSGTDFTLRISR | variable LC v2 |
| | VEAEDAGIYYCQQGSGMLTFGQGTKLEIK | |
| 348 | EVQLVESGGDLVKPAGSLRLSCTVSGFSLSRYSVHWVRQ | Caninized Clone M3 |
| | APGKGLEWLGMIWGGGSTDYNSVVKGRFTISRDNAKNTV | variable HC v1 and |
| | YLQMNSLRAEDTAMYYCARTYRGYALDYWGQGTLVTVSS | variant canine IgG-B |
| | ASTTAPSVFPLAPSCGSTSGSTVALACLVSGYFPEPVTV | Clq−, CD16− |
| | SWNSGSLTSGVHTFPSVLQSSGLYSLSSMVTVPSSRWPS | |
| | ETFTCNVAHPASKTKVDKPVPKRENGRVPRPPDCPKCPA | |
| | PEPLGGPSVFIFPPKPKDTLLIARTPEVTCVVVDLDRED | |
| | PEVQISWFVDGKQMQTAKTQPREEQFNGTYRVVSVLPIG | |
| | HQDWLKGKQFTCRVNNKALPSPIERTISKARGQAHQPSV | |
| | YVLPPSREELSKNTVSLTCLIKDFFPPDIDVEWQSNGQQ | |
| | EPESKYRTTPPQLDEDGSYFLYSKLSVDKSRWQRGDTFI | |
| | CAVMHEALHNHYTQESLSHSPGK | |
| 349 | ELTLQESGPGLVKPSQTLSLTCTVSGFSLSRYSVHWIRQ | Caninized Clone M3 |
| | PRGRGLELLGMIWGGGSTDYNPAFQGRISITADTAKNQF | variable HC v2 and |
| | SLQLSSMTTEDTAVYYCARTYRGYALDYWGQGTLVTVSS | variant canine IgG-B |
| | ASTTAPSVFPLAPSCGSTSGSTVALACLVSGYFPEPVTV | C1q−, CD16− |
| | SWNSGSLTSGVHTFPSVLQSSGLYSLSSMVTVPSSRWPS | |
| | ETFTCNVAHPASKTKVDKPVPKRENGRVPRPPDCPKCPA | |
| | PEPLGGPSVFIFPPKPKDTLLIARTPEVTCVVVDLDRED | |
| | PEVQISWFVDGKQMQTAKTQPREEQFNGTYRVVSVLPIG | |
| | HQDWLKGKQFTCRVNNKALPSPIERTISKARGQAHQPSV | |
| | YVLPPSREELSKNTVSLTCLIKDFFPPDIDVEWQSNGQQ | |
| | EPESKYRTTPPQLDEDGSYFLYSKLSVDKSRWQRGDTFI | |
| | CAVMHEALHNHYTQESLSHSPGK | |
| 350 | DIVMTQTPLSLSVSPGEPASISCRASQDISNYLNWYLQK | Caninized Clone M3 |
| | AGQSPRLLIYYTSRLQSGVPDRFSGSGSGTDFTLRIGRV | variable LC v1 and |
| | EAEDAGIYFCQQANTLPLTFGQGTRLEVRRNDAQPAVYL | canine κ light constant |
| | FQPSPDQLHTGSASVVCLLNSFYPKDINVKWKVDGVIQD | region |
| | TGIQESVTEQDKDSTYSLSSTLTMSSTEYLSHELYSCEI | |
| | THKSLPSTLIKSFQRSECQRVD | |
| 351 | EVQLVESGGDLVKPGGSLRLSCAASGFTFSSFGMHWVRQ | Caninized Clone M9 |
| | APGKGLEWVAYIRSDSSTIYYADAVKGRFTISRDNAKNT | variable HC v1 and |
| | LYLQMNSLRAEDTAMYYCARSGYYGSFSLTYWGQGTLVT | variant canine IgG-B |
| | VSSASTTAPSVFPLAPSCGSTSGSTVALACLVSGYFPEP | C1q−, CD16− |
| | VTVSWNSGSLTSGVHTFPSVLQSSGLYSLSSMVTVPSSR | |
| | WPSETFTCNVAHPASKTKVDKPVPKRENGRVPRPPDCPK | |
| | CPAPEPLGGPSVFIFPPKPKDTLLIARTPEVTCVVVDLD | |
| | REDPEVQISWFVDGKQMQTAKTQPREEQFNGTYRVVSVL | |
| | PIGHQDWLKGKQFTCRVNNKALPSPIERTISKARGQAHQ | |
| | PSVYVLPPSREELSKNTVSLTCLIKDFFPPDIDVEWQSN | |
| | GQQEPESKYRTTPPQLDEDGSYFLYSKLSVDKSRWQRGD | |
| | TFICAVMHEALHNHYTQESLSHSPGK | |
| 352 | EVQLVESGGDLVKPGGSLRLSCVASGFTFSSFGMHWVRQ | Caninized Clone M9 |
| | APGKGLQWVAYIRSDSSTIYYADAVKGRFTISRDNAKNT | variable HC v2 and |
| | LYLQMNSLRAEDTAMYYCARSGYYGSFSLTYWGQGTLVT | variant canine IgG-B |
| | VSSASTTAPSVFPLAPSCGSTSGSTVALACLVSGYFPEP | Clq−, CD16− |
| | VTVSWNSGSLTSGVHTFPSVLQSSGLYSLSSMVTVPSSR | |
| | WPSETFTCNVAHPASKTKVDKPVPKRENGRVPRPPDCPK | |
| | CPAPEPLGGPSVFIFPPKPKDTLLIARTPEVTCVVVDLD | |
| | REDPEVQISWFVDGKQMQTAKTQPREEQFNGTYRVVSVL | |
| | PIGHQDWLKGKQFTCRVNNKALPSPIERTISKARGQAHQ | |
| | PSVYVLPPSREELSKNTVSLTCLIKDFFPPDIDVEWQSN | |
| | GQQEPESKYRTTPPQLDEDGSYFLYSKLSVDKSRWQRGD | |
| | TFICAVMHEALHNHYTQESLSHSPGK | |
| 353 | DIMLTQTPLSLSVSPGEPASISCSASSSVSSNYLHWYLQ | Caninized Clone M9 |
| | KAGQSPRLLIYRTSNLPSGVPDRFSGSGSGTDFTLRIGR | variable LC v1 and |
| | VEAEDAGIYYCQQGSGMLTFGQGTRLEVRRNDAQPAVYL | canine κ light constant |
| | FQPSPDQLHTGSASVVCLLNSFYPKDINVKWKVDGVIQD | region |
| | TGIQESVTEQDKDSTYSLSSTLTMSSTEYLSHELYSCEI | |
| | THKSLPSTLIKSFQRSECQRVD | |
| 409 | DIVMTQTPLSLSVSPGEPASISCSASSSVSSNYLHWYLQ | Caninized Clone M9 |
| | KAGQSPRLLIYRTSNLPSGVPDRFSGSGSGTDFTLRISR | variable LC v2 and |
| | VEAEDAGIYYCQQGSGMLTFGQGTKLEIKRNDAQPAVYL | canine κ light constant |
| | FQPSPDQLHTGSASVVCLLNSFYPKDINVKWKVDGVIQD | region |
| | TGIQESVTEQDKDSTYSLSSTLTMSSTEYLSHELYSCEI | |
| | THKSLPSTLIKSFQRSECQRVD | |
| 354 | DFMGSENHTCVPEN | M3 canine IL4R epitope |
| 355 | GSVKVLHEPSCFSDYISTSVCQWKMDHPTNCSA | M8 canine IL4R |
| | | epitope 1 |
| 356 | REDSVCVCSMPI | M8 canine ILAR |
| | | epitope 2 |
| 357 | REDSVCVCSMPIDDAVEADV | M9 canine IL4R epitope |
| 379 | MGVPRPRSWGLGFLLFLLPTLRAADSHLSLLYHLTAVSAPPPG | Exemplary canine FcRn |
| | TPAFWASGWLGPQQYLSYNNLRAQAEPYGAWVWENQVSWYWEK | with poly-His |
| | ETTDLRTKEGLFLEALKALGDGGPYTLQGLLGCELGPDNTSVP | |
| | VAKFALNGEDFMTFDPKLGTWNGDWPETETVSKRWMQQAGAVS | |
| | KERTFLLYSCPQRLLGHLERGRGNLEWKEPPSMRLKARPGSPG | |
| | FSVLTCSAFSFYPPELQLRFLRNGLAAGSGEGDFGPNGDGSFH | |
| | AWSSLTVKSGDEHHYRCLVQHAGLPQPLTVELESPAKSSGSHH | |
| | HHHH | |
| 380 | MAPRPALATAGFLALLLILLAACRLDAVQHPPKIQVYSRHPAE | Exemplary canine B2M |
| | NGKPNFLNCYVSGFHPPEIEIDLLKNGKEMKAEQTDLSFSKDW | |
| | TFYLLVHTEFTPNEQDEFSCRVKHVTLSEPQIVKWDRDN | |
| 381 | PAPEMLGGPSVFIFPPKPKDTLFIARTPEVTCVVVDLDP | Exemplary variant |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Protein A+ |
| | SVYVLPPSREELSKNTVSLTCLIKDFFPPDIDVEWQSNG | C1q+ |
| | QQEPESKYRTTPPQLDEDGSYFLYSKLSVDKSRWQRGDT | CD16+ |
| | FICAVMHEALHNHYTQESLSHSPGK | L(23)F(F00) |
| 382 | PAPEMLGGPSVFIFPPKPKDTLYIARTPEVTCVVVDLDP | Exemplary variant |
| | EDPEVQISWFVDGKQMQTAKTQPREEQFNGTYRVVSVLP | canine IgG-B Fc |
| | IGHQDWLKGKQFTCKVNNKALPSPIERTISKARGQAHQP | Protein A+ |
| | SVYVLPPSREELSKNTVSLTCLIKDFFPPDIDVEWQSNG | C1q+ |
| | QQEPESKYRTTPPQLDEDGSYFLYSKLSVDKSRWQRGDT | CD16+ |
| | FICAVMHEALHNHYTQESLSHSPGK | L(23)Y (Y00) |
| 383 | PVPEPLGGPSVLIFPPKPKDTLFIARTPEVTCVVLDLGR | Exemplary variant |
| | EDPEVQISWFVDGKEVHTAKTQSREQQFNGTYRVVSVLP | canine IgG-A Fc (F00; |
| | IGHQDWLTGKEFKCRVNHIDLPSPIERTISKARGRAHKP | Protein A+; Clq−; |
| | SVYVLPPSPKELSSSDTVSITCLIKDFYPPDIDVEWQSN | CD16−) |
| | GQQEPERKHRMTPPQLDEDGSYFLYSKLSVDKSRWQQGD | I(21)T; R(23)F; T(25)A; |
| | PFTCAVMHEALHNHYTDLSLSHSPGK | E(80)G; T(205)A; |
| | | Q(207)H |
| 384 | PAPEMLGGPSVLIFPPKPKDTLLIARTPEVTCVVVDLDP | Exemplary variant |
| | EDPEVQISWFVDGKEVHTAKTQSREEQFNGTYRVVSVLP | canine IgG-A Fc |
| | IGHQDWLTGKEFKCKVNNKALPSPIERTISKARGRAHKP | (Protein A+; C1q+; |
| | SVYVLPPSPKELSSSDTVSITCLIKDFYPPDIDVEWQSN | CD16+) |
| | GQQEPERKHRMTPPQLDEDGSYFLYSKLSVDKSRWQQGD | V2A; P5M; I21T; R23L; |
| | PFTCAVMHEALHNHYTDLSLSHSPGK | T25A; L35V; G38D; |
| | | R39P; Q65E; E80G; |
| | | R93K; H96N; 197K; |
| | | D98A; T205A; Q207H |
| 385 | PVPESLGGPSVFIFPPKPKDTLFIARTPEITCVVLDLGR | Exemplary variant |
| | EDPEVQISWFVDGKEVHTAKTQPREQQFNSTYRVVSVLP | canine IgG-D Fc (F00; |
| | IGHQDWLTGKEFKCRVNHIGLPSPIERTISKARGQAHQP | Protein A+; Clq−; |
| | SVYVLPPSPKELSSSDTVTLTCLIKDFFPPEIDVEWQSN | CD16−) |
| | GQPEPESKYHTTAPQLDEDGSYFLYSKLSVDKSRWQQGD | I(21)T; R(23)F; T(25)A; |
| | TFTCAVMHEALHNHYTDLSLSHSPGK | E(80)G; Q(205)A; |
| | | Q(207)H |
| 386 | PAPEMLGGPSVFIFPPKPKDTLLIARTPEITCVVVDLDP | Exemplary variant |
| | EDPEVQISWFVDGKEVHTAKTQPREEQFNSTYRVVSVLP | canine IgG-D Fc |
| | IGHQDWLTGKEFKCKVNNKALPSPIERTISKARGQAHQP | (Protein A+; C1q+; |
| | SVYVLPPSPKELSSSDTVTLTCLIKDFFPPEIDVEWQSN | CD16+) |
| | GQPEPESKYHTTAPQLDEDGSYFLYSKLSVDKSRWQQGD | V2A; S5M; I21T; R23L; |
| | TFTCAVMHEALHNHYTDLSLSHSPGK | T25A; L35V; G38D; |
| | | R39P; Q65E; E80G; |
| | | R93K; H96N; 197K; |
| | | G98A; Q207H |
| 387 | PAPEMLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc (OY0) |
| | IGHYDWLKGKQFTCKVNNKALPSPIERTISKARGQAHQP | Protein A+ |
| | SVYVLPPSREELSKNTVSLTCLIKDFFPPDIDVEWQSNG | C1q+ |
| | QQEPESKYRTTPPQLDEDGSYFLYSKLSVDKSRWQRGDT | CD16+ |
| | FICAVMHEALHNHYTQESLSHSPGK | Q(82)Y (OYO) |
| 388 | PAPEMLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc (0YH) |
| | IGHYDWLKGKQFTCKVNNKALPSPIERTISKARGQAHQP | Gln82Tyr |
| | SVYVLPPSREELSKNTVSLTCLIKDFFPPDIDVEWQSNG | Asn207His |
| | QQEPESKYRTTPPQLDEDGSYFLYSKLSVDKSRWQRGDT | |
| | FICAVMHEALHHHYTQESLSHSPGK | |
| 389 | PAPEMLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc (OYY) |
| | IGHYDWLKGKQFTCKVNNKALPSPIERTISKARGQAHQP | Gln82Tyr |
| | SVYVLPPSREELSKNTVSLTCLIKDFFPPDIDVEWQSNG | Asn207Tyr |
| | QQEPESKYRTTPPQLDEDGSYFLYSKLSVDKSRWQRGDT | |
| | FICAVMHEALHYHYTQESLSHSPGK | |
| 390 | PAPEMLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc (00Y) |
| | IGHQDWLKGKQFTCKVNNKALPSPIERTISKARGQAHQP | Asn207Tyr |
| | SVYVLPPSREELSKNTVSLTCLIKDFFPPDIDVEWQSNG | |
| | QQEPESKYRITPPQLDEDGSYFLYSKLSVDKSRWQRGDT | |
| | FICAVMHEALHYHYTQESLSHSPGK | |
| 391 | PAPEMLGGPSVFIFPPKPKDTLYITREPEVTCVVVDLDP | Exemplary variant |
| | EDPEVQISWFVDGKQMQTAKTQPREEQFNGTYRVVSVLP | canine IgG-B Fc (YTE) |
| | IGHQDWLKGKQFTCKVNNKALPSPIERTISKARGQAHQP | Leu23Tyr |
| | SVYVLPPSREELSKNTVSLTCLIKDFFPPDIDVEWQSNG | Ala25Thr |
| | QQEPESKYRTTPPQLDEDGSYFLYSKLSVDKSRWQRGDT | Thr27Glu |
| | FICAVMHEALHNHYTQESLSHSPGK | |
| 392 | PAPEMLGGPSVFIFPPKPKDTLFIARTPEVTCVVVDLDP | Exemplary variant |
| | EDPEVQISWFVDGKQMQTAKTQPREEQFNGTYRVVSVLP | canine IgG-B Fc |
| | IGHQDWLKGKQFTCRVNNIGLPSPIERTISKARGQAHQP | Protein A+ |
| | SVYVLPPSREELSKNTVSLTCLIKDFFPPDIDVEWQSNG | C1q− |
| | QQEPESKYRTTPPQLDEDGSYFLYSKLSVDKSRWQRGDT | CD16− |
| | FICAVMHEALHNHYTQESLSHSPGK | K(93)R |
| | | K(97)I |
| | | A(98)G |
| | | L(23)F (F00) |
| 393 | PAPEMLGGPSVFIFPPKPKDTLYIARTPEVTCVVVDLDP | Exemplary variant |
| | EDPEVQISWFVDGKQMQTAKTQPREEQFNGTYRVVSVLP | canine IgG-B Fc |
| | IGHQDWLKGKQFTCRVNNIGLPSPIERTISKARGQAHQP | Protein A+ |
| | SVYVLPPSREELSKNTVSLTCLIKDFFPPDIDVEWQSNG | C1q− |
| | QQEPESKYRTTPPQLDEDGSYFLYSKLSVDKSRWQRGDT | CD16 |
| | FICAVMHEALHNHYTQESLSHSPGK | K(93)R |
| | | K(97)I |
| | | A(98)G |
| | | L(23) Y (Y00) |
| 394 | PAPEMLGGPSVFIFPPKPKDTLLIARTPEVTCVVVDLDP | Exemplary variant |
| | EDPEVQISWFVDGKQMQTAKTQPREEQFNGTYRVVSVLP | canine IgG-B Fc |
| | IGHYDWLKGKQFTCRVNNIGLPSPIERTISKARGQAHQP | Protein A+ |
| | SVYVLPPSREELSKNTVSLTCLIKDFFPPDIDVEWQSNG | Clq− |
| | QQEPESKYRTTPPQLDEDGSYFLYSKLSVDKSRWQRGDT | CD16− |
| | FICAVMHEALHNHYTQESLSHSPGK | K(93)R |
| | | K(97)I |
| | | A(98)G |
| | | Q(82)Y (OY0) |
| 395 | EVQLVESGGDLVKPGGSLRLSCKASGYTFTSYVMHWVRQ | Caninized Clone I |
| | APGQGLEWVAYINPNNDGTFYNGAVKGRFTISRDNARNT | variable HC v1 and |
| | LYLQMNSLRSEDTAVYYCAAFYYGFAYWGQGTLVTVSSA | variant canine IgG-B |
| | STTAPSVFPLAPSCGSTSGSTVALACLVSGYFPEPVTVS | C1q−, CD16−, F00 |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EMLGGPSVFIFPPKPKDTLFIARTPEVTCVVVDLDPEDP | |
| | EVQISWFVDGKQMQTAKTQPREEQFNGTYRVVSVLPIGH | |
| | QDWLKGKQFTCRVNNIGLPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 396 | EVQLVQSGAEVKKPGASVKVSCKASGYTFTSYVMHWVRQ | Caninized Clone I |
| | APGQGLEWMGYINPNNDGTFYNGKFQGRVTLTADTSTST | variable HC v2 and |
| | AYMELSSLRAGDIAVYYCAAFYYGFAYWGQGTLVTVSSA | variant canine IgG-B |
| | STTAPSVFPLAPSCGSTSGSTVALACLVSGYFPEPVTVS | C1q−, CD16−, F00 |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EMLGGPSVFIFPPKPKDTLFIARTPEVTCVVVDLDPEDP | |
| | EVQISWFVDGKQMQTAKTQPREEQFNGTYRVVSVLPIGH | |
| | QDWLKGKQFTCRVNNIGLPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 397 | EVQLVQSAAEVKKPGASVKVSCKASGYTFTSYVMHWVRQ | Caninized Clone I |
| | APGQGLEWIGYINPNNDGTFYNGKFQGRVTLTADTSTGT | variable HC v3 and |
| | TYTELSSLRAEDTAVYYCAAFYYGFAYWGQGTLVTVSSA | variant canine IgG-B |
| | STTAPSVEPLAPSCGSTSGSTVALACLVSGYFPEPVTVS | Clq−, CD16−, F00 |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EMLGGPSVFIFPPKPKDTLFIARTPEVTCVVVDLDPEDP | |
| | EVQISWFVDGKQMQTAKTQPREEQFNGTYRVVSVLPIGH | |
| | QDWLKGKQFTCRVNNIGLPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 398 | EVQLVQSGAEVKKPGASVKVSCKASGYTFTSYVMHWVRQ | Caninized Clone I |
| | APGQGLEWMGYINPNNDGTFYNGKFQGRVTLTADTSTST | variable HC v4 and |
| | AYMELSSLRAGDIAVYYCAAFYYGFAYWGQGTLVTVSSA | variant canine IgG-B |
| | STTAPSVFPLAPSCGSTSGSTVALACLVSGYFPEPVTVS | Clq−, CD16−, F00 |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EMLGGPSVFIFPPKPKDTLFIARTPEVTCVVVDLDPEDP | |
| | EVQISWFVDGKQMQTAKTQPREEQFNGTYRVVSVLPIGH | |
| | QDWLKGKQFTCRVNNIGLPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 410 | EVQLVESGGDLVKPAGSLRLSCTVSGFSLSRYSVHWVRQ | Caninized Clone M3 |
| | APGKGLEWLGMIWGGGSTDYNSVVKGRFTISRDNAKNTV | variable HC v1 and |
| | YLQMNSLRAEDTAMYYCARTYRGYALDYWGQGTLVTVSS | variant canine IgG-B |
| | ASTTAPSVFPLAPSCGSTSGSTVALACLVSGYFPEPVTV | Clq−, CD16−, F00 |
| | SWNSGSLTSGVHTFPSVLQSSGLYSLSSMVTVPSSRWPS | |
| | ETFTCNVAHPASKTKVDKPVPKRENGRVPRPPDCPKCPA | |
| | PEMLGGPSVFIFPPKPKDTLFIARTPEVTCVVVDLDPED | |
| | PEVQISWFVDGKQMQTAKTQPREEQFNGTYRVVSVLPIG | |
| | HQDWLKGKQFTCRVNNIGLPSPIERTISKARGQAHQPSV | |
| | YVLPPSREELSKNTVSLTCLIKDFFPPDIDVEWQSNGQQ | |
| | EPESKYRTTPPQLDEDGSYFLYSKLSVDKSRWQRGDTFI | |
| | CAVMHEALHNHYTQESLSHSPGK | |
| 411 | ELTLQESGPGLVKPSQTLSLTCTVSGFSLSRYSVHWIRQ | Caninized Clone M3 |
| | PRGRGLELLGMIWGGGSTDYNPAFQGRISITADTAKNQF | variable HC v2 and |
| | SLQLSSMTTEDTAVYYCARTYRGYALDYWGQGTLVTVSS | variant canine IgG-B |
| | ASTTAPSVFPLAPSCGSTSGSTVALACLVSGYFPEPVTV | C1q−, CD16−, F00 |
| | SWNSGSLTSGVHTFPSVLQSSGLYSLSSMVTVPSSRWPS | |
| | ETFTCNVAHPASKTKVDKPVPKRENGRVPRPPDCPKCPA | |
| | PEMLGGPSVFIFPPKPKDTLFIARTPEVTCVVVDLDPED | |
| | PEVQISWFVDGKQMQTAKTQPREEQFNGTYRVVSVLPIG | |
| | HQDWLKGKQFTCRVNNIGLPSPIERTISKARGQAHQPSV | |
| | YVLPPSREELSKNTVSLTCLIKDFFPPDIDVEWQSNGQQ | |
| | EPESKYRTTPPQLDEDGSYFLYSKLSVDKSRWQRGDTFI | |
| | CAVMHEALHNHYTQESLSHSPGK | |
| 412 | EVQLVESGGDLVKPGGSLRLSCAASGFTFSSFGMHWVRQ | Caninized Clone M9 |
| | APGKGLEWVAYIRSDSSTIYYADAVKGRFTISRDNAKNT | variable HC v1 and |
| | LYLQMNSLRAEDTAMYYCARSGYYGSFSLTYWGQGTLVT | variant canine IgG-B |
| | VSSASTTAPSVFPLAPSCGSTSGSTVALACLVSGYFPEP | Clq−, CD16−, F00 |
| | VTVSWNSGSLTSGVHTFPSVLQSSGLYSLSSMVTVPSSR | |
| | WPSETFTCNVAHPASKTKVDKPVPKRENGRVPRPPDCPK | |
| | CPAPEMLGGPSVFIFPPKPKDTLFIARTPEVTCVVVDLD | |
| | PEDPEVQISWFVDGKQMQTAKTQPREEQFNGTYRVVSVL | |
| | PIGHQDWLKGKQFTCRVNNIGLPSPIERTISKARGQAHQ | |
| | PSVYVLPPSREELSKNTVSLTCLIKDFFPPDIDVEWQSN | |
| | GQQEPESKYRTTPPQLDEDGSYFLYSKLSVDKSRWQRGD | |
| | TFICAVMHEALHNHYTQESLSHSPGK | |
| 413 | EVQLVESGGDLVKPGGSLRLSCVASGFTFSSFGMHWVRQ | Caninized Clone M9 |
| | APGKGLQWVAYIRSDSSTIYYADAVKGRFTISRDNAKNT | variable HC v2 and |
| | LYLQMNSLRAEDTAMYYCARSGYYGSFSLTYWGQGTLVT | variant canine IgG-B |
| | VSSASTTAPSVFPLAPSCGSTSGSTVALACLVSGYFPEP | Clq−, CD16−, F00 |
| | VTVSWNSGSLTSGVHTFPSVLQSSGLYSLSSMVTVPSSR | |
| | WPSETFTCNVAHPASKTKVDKPVPKRENGRVPRPPDCPK | |
| | CPAPEMLGGPSVFIFPPKPKDTLFIARTPEVTCVVVDLD | |
| | PEDPEVQISWFVDGKQMQTAKTQPREEQFNGTYRVVSVL | |
| | PIGHQDWLKGKQFTCRVNNIGLPSPIERTISKARGQAHQ | |
| | PSVYVLPPSREELSKNTVSLTCLIKDFFPPDIDVEWQSN | |
| | GQQEPESKYRTTPPQLDEDGSYFLYSKLSVDKSRWQRGD | |
| | TFICAVMHEALHNHYTQESLSHSPGK | |
| 399 | EVQLVESGGDLVKPGGSLRLSCKASGYTFTSYVMHWVRQ | Caninized Clone I |
| | APGQGLEWVAYINPNNDGTFYNGAVKGRFTISRDNARNT | variable HC v1 and |
| | LYLQMNSLRSEDTAVYYCAAFYYGFAYWGQGTLVTVSSA | variant canine IgG-B |
| | STTAPSVFPLAPSCGSTSGSTVALACLVSGYFPEPVTVS | Clq−, CD16−, Y00 |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EMLGGPSVFIFPPKPKDTLYIARTPEVTCVVVDLDPEDP | |
| | EVQISWFVDGKQMQTAKTQPREEQFNGTYRVVSVLPIGH | |
| | QDWLKGKQFTCRVNNIGLPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 400 | EVQLVQSGAEVKKPGASVKVSCKASGYTFTSYVMHWVRQ | Caninized Clone I |
| | APGQGLEWMGYINPNNDGTFYNGKFQGRVTLTADTSTST | variable HC v2 and |
| | AYMELSSLRAGDIAVYYCAAFYYGFAYWGQGTLVTVSSA | variant canine IgG-B |
| | STTAPSVFPLAPSCGSTSGSTVALACLVSGYFPEPVTVS | Clq−, CD16−, Y00 |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EMLGGPSVFIFPPKPKDTLYIARTPEVTCVVVDLDPEDP | |
| | EVQISWFVDGKQMQTAKTQPREEQFNGTYRVVSVLPIGH | |
| | QDWLKGKQFTCRVNNIGLPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 401 | EVQLVQSAAEVKKPGASVKVSCKASGYTFTSYVMHWVRQ | Caninized Clone I |
| | APGQGLEWIGYINPNNDGTFYNGKFQGRVTLTADTSTGT | variable HC v3 and |
| | TYTELSSLRAEDTAVYYCAAFYYGFAYWGQGTLVTVSSA | variant canine IgG-B |
| | STTAPSVEPLAPSCGSTSGSTVALACLVSGYFPEPVTVS | C1q−, CD16−, Y00 |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EMLGGPSVFIFPPKPKDTLYIARTPEVTCVVVDLDPEDP | |
| | EVQISWFVDGKQMQTAKTQPREEQFNGTYRVVSVLPIGH | |
| | QDWLKGKQFTCRVNNIGLPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 402 | EVQLVQSGAEVKKPGASVKVSCKASGYTFTSYVMHWVRQ | Caninized Clone I |
| | APGQGLEWMGYINPNNDGTFYNGKFQGRVTLTADTSTST | variable HC v4 and |
| | AYMELSSLRAGDIAVYYCAAFYYGFAYWGQGTLVTVSSA | variant canine IgG-B |
| | STTAPSVFPLAPSCGSTSGSTVALACLVSGYFPEPVTVS | Clq−, CD16−, Y00 |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EMLGGPSVFIFPPKPKDTLYIARTPEVTCVVVDLDPEDP | |
| | EVQISWFVDGKQMQTAKTQPREEQFNGTYRVVSVLPIGH | |
| | QDWLKGKQFTCRVNNIGLPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 414 | EVQLVESGGDLVKPAGSLRLSCTVSGFSLSRYSVHWVRQ | Caninized Clone M3 |
| | APGKGLEWLGMIWGGGSTDYNSVVKGRFTISRDNAKNTV | variable HC v1 and |
| | YLQMNSLRAEDTAMYYCARTYRGYALDYWGQGTLVTVSS | variant canine IgG-B |
| | ASTTAPSVFPLAPSCGSTSGSTVALACLVSGYFPEPVTV | Clq−, CD16−, Y00 |
| | SWNSGSLTSGVHTFPSVLQSSGLYSLSSMVTVPSSRWPS | |
| | ETFTCNVAHPASKTKVDKPVPKRENGRVPRPPDCPKCPA | |
| | PEMLGGPSVFIFPPKPKDTLYIARTPEVTCVVVDLDPED | |
| | PEVQISWFVDGKQMQTAKTQPREEQFNGTYRVVSVLPIG | |
| | HQDWLKGKQFTCRVNNIGLPSPIERTISKARGQAHQPSV | |
| | YVLPPSREELSKNTVSLTCLIKDFFPPDIDVEWQSNGQQ | |
| | EPESKYRTTPPQLDEDGSYFLYSKLSVDKSRWQRGDTFI | |
| | CAVMHEALHNHYTQESLSHSPGK | |
| 415 | ELTLQESGPGLVKPSQTLSLTCTVSGFSLSRYSVHWIRQ | Caninized Clone M3 |
| | PRGRGLELLGMIWGGGSTDYNPAFQGRISITADTAKNQF | variable HC v2 and |
| | SLQLSSMTTEDTAVYYCARTYRGYALDYWGQGTLVTVSS | variant canine IgG-B |
| | ASTTAPSVFPLAPSCGSTSGSTVALACLVSGYFPEPVTV | C1q−, CD16−, Y00 |
| | SWNSGSLTSGVHTFPSVLQSSGLYSLSSMVTVPSSRWPS | |
| | ETFTCNVAHPASKTKVDKPVPKRENGRVPRPPDCPKCPA | |
| | PEMLGGPSVFIFPPKPKDTLYIARTPEVTCVVVDLDPED | |
| | PEVQISWFVDGKQMQTAKTQPREEQFNGTYRVVSVLPIG | |
| | HQDWLKGKQFTCRVNNIGLPSPIERTISKARGQAHQPSV | |
| | YVLPPSREELSKNTVSLTCLIKDFFPPDIDVEWQSNGQQ | |
| | EPESKYRTTPPQLDEDGSYFLYSKLSVDKSRWQRGDTFI | |
| | CAVMHEALHNHYTQESLSHSPGK | |
| 416 | EVQLVESGGDLVKPGGSLRLSCAASGFTFSSFGMHWVRQ | Caninized Clone M9 |
| | APGKGLEWVAYIRSDSSTIYYADAVKGRFTISRDNAKNT | variable HC v1 and |
| | LYLQMNSLRAEDTAMYYCARSGYYGSFSLTYWGQGTLVT | variant canine IgG-B |
| | VSSASTTAPSVFPLAPSCGSTSGSTVALACLVSGYFPEP | C1q−, CD16−, Y00 |
| | VTVSWNSGSLTSGVHTFPSVLQSSGLYSLSSMVTVPSSR | |
| | WPSETFTCNVAHPASKTKVDKPVPKRENGRVPRPPDCPK | |
| | CPAPEMLGGPSVFIFPPKPKDTLYIARTPEVTCVVVDLD | |
| | PEDPEVQISWFVDGKQMQTAKTQPREEQFNGTYRVVSVL | |
| | PIGHQDWLKGKQFTCRVNNIGLPSPIERTISKARGQAHQ | |
| | PSVYVLPPSREELSKNTVSLTCLIKDFFPPDIDVEWQSN | |
| | GQQEPESKYRITPPQLDEDGSYFLYSKLSVDKSRWQRGD | |
| | TFICAVMHEALHNHYTQESLSHSPGK | |
| 417 | EVQLVESGGDLVKPGGSLRLSCVASGFTFSSFGMHWVRQ | Caninized Clone M9 |
| | APGKGLQWVAYIRSDSSTIYYADAVKGRFTISRDNAKNT | variable HC v2 and |
| | LYLQMNSLRAEDTAMYYCARSGYYGSFSLTYWGQGTLVT | variant canine IgG-B |
| | VSSASTTAPSVFPLAPSCGSTSGSTVALACLVSGYFPEP | Clq−, CD16−, Y00 |
| | VTVSWNSGSLTSGVHTFPSVLQSSGLYSLSSMVTVPSSR | |
| | WPSETFTCNVAHPASKTKVDKPVPKRENGRVPRPPDCPK | |
| | CPAPEMLGGPSVFIFPPKPKDTLYIARTPEVTCVVVDLD | |
| | PEDPEVQISWFVDGKQMQTAKTQPREEQFNGTYRVVSVL | |
| | PIGHQDWLKGKQFTCRVNNIGLPSPIERTISKARGQAHQ | |
| | PSVYVLPPSREELSKNTVSLTCLIKDFFPPDIDVEWQSN | |
| | GQQEPESKYRTTPPQLDEDGSYFLYSKLSVDKSRWQRGD | |
| | TFICAVMHEALHNHYTQESLSHSPGK | |
| 403 | EVQLVESGGDLVKPGGSLRLSCKASGYTFTSYVMHWVRQ | Caninized Clone I |
| | APGQGLEWVAYINPNNDGTFYNGAVKGRFTISRDNARNT | variable HC v1 and |
| | LYLQMNSLRSEDTAVYYCAAFYYGFAYWGQGTLVTVSSA | variant canine IgG-B |
| | STTAPSVFPLAPSCGSTSGSTVALACLVSGYFPEPVTVS | Clq−, CD16−, 0Y0 |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EMLGGPSVFIFPPKPKDTLLIARTPEVTCVVVDLDPEDP | |
| | EVQISWFVDGKQMQTAKTQPREEQFNGTYRVVSVLPIGH | |
| | YDWLKGKQFTCRVNNIGLPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 404 | EVQLVQSGAEVKKPGASVKVSCKASGYTFTSYVMHWVRQ | Caninized Clone I |
| | APGQGLEWMGYINPNNDGTFYNGKFQGRVTLTADTSTST | variable HC v2 and |
| | AYMELSSLRAGDIAVYYCAAFYYGFAYWGQGTLVTVSSA | variant canine IgG-B |
| | STTAPSVFPLAPSCGSTSGSTVALACLVSGYFPEPVTVS | C1q−, CD16−, 0Y0 |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EMLGGPSVFIFPPKPKDTLLIARTPEVTCVVVDLDPEDP | |
| | EVQISWFVDGKQMQTAKTQPREEQFNGTYRVVSVLPIGH | |
| | YDWLKGKQFTCRVNNIGLPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 405 | EVQLVQSAAEVKKPGASVKVSCKASGYTFTSYVMHWVRQ | Caninized Clone I |
| | APGQGLEWIGYINPNNDGTFYNGKFQGRVTLTADTSTGT | variable HC v3 and |
| | TYTELSSLRAEDTAVYYCAAFYYGFAYWGQGTLVTVSSA | variant canine IgG-B |
| | STTAPSVFPLAPSCGSTSGSTVALACLVSGYFPEPVTVS | C1q−, CD16−,0Y0 |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EMLGGPSVFIFPPKPKDTLLIARTPEVTCVVVDLDPEDP | |
| | EVQISWFVDGKQMQTAKTQPREEQFNGTYRVVSVLPIGH | |
| | YDWLKGKQFTCRVNNIGLPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 406 | EVQLVQSGAEVKKPGASVKVSCKASGYTFTSYVMHWVRQ | Caninized Clone I |
| | APGQGLEWMGYINPNNDGTFYNGKFQGRVTLTADTSTST | variable HC v4 and |
| | AYMELSSLRAGDIAVYYCAAFYYGFAYWGQGTLVTVSSA | variant canine IgG-B |
| | STTAPSVFPLAPSCGSTSGSTVALACLVSGYFPEPVTVS | Clq−, CD16−,0Y0 |
| | WNSGSLTSGVHTFPSVLQSSGLYSLSSMVTVPSSRWPSE | |
| | TFTCNVAHPASKTKVDKPVPKRENGRVPRPPDCPKCPAP | |
| | EMLGGPSVFIFPPKPKDTLLIARTPEVTCVVVDLDPEDP | |
| | EVQISWFVDGKQMQTAKTQPREEQFNGTYRVVSVLPIGH | |
| | YDWLKGKQFTCRVNNIGLPSPIERTISKARGQAHQPSVY | |
| | VLPPSREELSKNTVSLTCLIKDFFPPDIDVEWQSNGQQE | |
| | PESKYRTTPPQLDEDGSYFLYSKLSVDKSRWQRGDTFIC | |
| | AVMHEALHNHYTQESLSHSPGK | |
| 418 | EVQLVESGGDLVKPAGSLRLSCTVSGFSLSRYSVHWVRQ | Caninized Clone M3 |
| | APGKGLEWLGMIWGGGSTDYNSVVKGRFTISRDNAKNTV | variable HC v1 and |
| | YLQMNSLRAEDTAMYYCARTYRGYALDYWGQGTLVTVSS | variant canine IgG-B |
| | ASTTAPSVFPLAPSCGSTSGSTVALACLVSGYFPEPVTV | C1q−, CD16−, 0Y0 |
| | SWNSGSLISGVHTFPSVLQSSGLYSLSSMVTVPSSRWPS | |
| | ETFTCNVAHPASKTKVDKPVPKRENGRVPRPPDCPKCPA | |
| | PEMLGGPSVFIFPPKPKDTLLIARTPEVTCVVVDLDPED | |
| | PEVQISWFVDGKQMQTAKTQPREEQFNGTYRVVSVLPIG | |
| | HYDWLKGKQFTCRVNNIGLPSPIERTISKARGQAHQPSV | |
| | YVLPPSREELSKNTVSLTCLIKDFFPPDIDVEWQSNGQQ | |
| | EPESKYRTTPPQLDEDGSYFLYSKLSVDKSRWQRGDTFI | |
| | CAVMHEALHNHYTQESLSHSPGK | |
| 419 | ELTLQESGPGLVKPSQTLSLTCTVSGFSLSRYSVHWIRQ | Caninized Clone M3 |
| | PRGRGLELLGMIWGGGSTDYNPAFQGRISITADTAKNQF | variable HC v2 and |
| | SLQLSSMTTEDTAVYYCARTYRGYALDYWGQGTLVTVSS | variant canine IgG-B |
| | ASTTAPSVFPLAPSCGSTSGSTVALACLVSGYFPEPVTV | Clq−, CD16−, 0Y0 |
| | SWNSGSLTSGVHTFPSVLQSSGLYSLSSMVTVPSSRWPS | |
| | ETFTCNVAHPASKTKVDKPVPKRENGRVPRPPDCPKCPA | |
| | PEMLGGPSVFIFPPKPKDTLLIARTPEVTCVVVDLDPED | |
| | PEVQISWFVDGKQMQTAKTQPREEQFNGTYRVVSVLPIG | |
| | HYDWLKGKQFTCRVNNIGLPSPIERTISKARGQAHQPSV | |
| | YVLPPSREELSKNTVSLTCLIKDFFPPDIDVEWQSNGQQ | |
| | EPESKYRTTPPQLDEDGSYFLYSKLSVDKSRWQRGDIFI | |
| | CAVMHEALHNHYTQESLSHSPGK | |
| 420 | EVQLVESGGDLVKPGGSLRLSCAASGFTFSSFGMHWVRQ | Caninized Clone M9 |
| | APGKGLEWVAYIRSDSSTIYYADAVKGRFTISRDNAKNT | variable HC v1 and |
| | LYLQMNSLRAEDTAMYYCARSGYYGSFSLTYWGQGTLVT | variant canine IgG-B |
| | VSSASTTAPSVFPLAPSCGSTSGSTVALACLVSGYFPEP | Clq−, CD16−, 0Y0 |
| | VTVSWNSGSLTSGVHTFPSVLQSSGLYSLSSMVTVPSSR | |
| | WPSETFTCNVAHPASKTKVDKPVPKRENGRVPRPPDCPK | |
| | CPAPEMLGGPSVFIFPPKPKDTLLIARTPEVTCVVVDLD | |
| | PEDPEVQISWFVDGKQMQTAKTQPREEQFNGTYRVVSVL | |
| | PIGHYDWLKGKQFTCRVNNIGLPSPIERTISKARGQAHQ | |
| | PSVYVLPPSREELSKNTVSLTCLIKDFFPPDIDVEWQSN | |
| | GQQEPESKYRTTPPQLDEDGSYFLYSKLSVDKSRWQRGD | |
| | TFICAVMHEALHNHYTQESLSHSPGK | |
| 421 | EVQLVESGGDLVKPGGSLRLSCVASGFTFSSFGMHWVRQ | Caninized Clone M9 |
| | APGKGLQWVAYIRSDSSTIYYADAVKGRFTISRDNAKNT | variable HC v2 and |
| | LYLQMNSLRAEDTAMYYCARSGYYGSFSLTYWGQGTLVT | variant canine IgG-B |
| | VSSASTTAPSVFPLAPSCGSTSGSTVALACLVSGYFPEP | C1q−, CD16−,0Y0 |
| | VTVSWNSGSLTSGVHTFPSVLQSSGLYSLSSMVTVPSSR | |
| | WPSETFTCNVAHPASKTKVDKPVPKRENGRVPRPPDCPK | |
| | CPAPEMLGGPSVFIFPPKPKDTLLIARTPEVTCVVVDLD | |
| | PEDPEVQISWFVDGKQMQTAKTQPREEQFNGTYRVVSVL | |
| | PIGHYDWLKGKQFTCRVNNIGLPSPIERTISKARGQAHQ | |
| | PSVYVLPPSREELSKNTVSLTCLIKDFFPPDIDVEWQSN | |
| | GQQEPESKYRTTPPQLDEDGSYFLYSKLSVDKSRWQRGD | |
| | TFICAVMHEALHNHYTQESLSHSPGK | |

### DESCRIPTION OF CERTAIN EMBODIMENTS

Antibodies that bind canine IL4R and/or feline IL4R are provided. Antibody heavy chains and light chains that are capable of forming antibodies that bind IL4R are also provided. In addition, antibodies, heavy chains, and light chains comprising one or more particular complementary determining regions (CDRs) are provided. Polynucleotides encoding antibodies to canine or feline IL4R are provided. Methods of producing or purifying antibodies to canine or feline IL4R are also provided. Methods of treatment using antibodies to canine and/or feline IL4/IL13 are provided. Such methods include, but are not limited to, methods of treating IL4-induced conditions and/or IL13-induced conditions in companion animal species. Methods of detecting soluble IL4R in a sample from a companion animal species are provided. Methods of screening for molecules that inhibit IL4 and/or IL13 signaling function (e.g., anti-IL4R, anti-IL13R, anti-IL4, and anti-IL13 antibodies and small molecule antagonists of IL4R, IL13R, IL4, and IL13) are also provided.

Also provided are variant IgG Fc polypeptides from companion animals, such as canine and feline, having increased binding to Protein A, decreased binding to C1q, decreased binding to CD16, increased binding to FcRn, increased stability, increased recombinant production, and/or increased hinge disulfide formation that may be used in the context of the canine or feline IL4R antibodies provided herein. In addition, provided herein are variant IgG Fc polypeptides and variant light chain constant regions from companion animals, such as canine, feline, and equine, for preparation of bispecific antibodies, including anti-IL4R antibodies. In some embodiments, anti-IL4R antibodies or antibody fragments comprise a variant IgG Fc polypeptide or a variant light chain constant region. Methods for preparing anti-IL4R antibodies and bispecific antibodies incorporating variant IgG Fc polypeptides are provided.

Novel antibodies directed against IL4R are provided, for example antibodies that bind to canine IL4R and/or feline IL4R. Anti-IL4R antibodies provided herein include, but are not limited to, monoclonal antibodies, mouse antibodies, chimeric antibodies, caninized antibodies, felinized antibodies, and bispecific antibodies. In some embodiments, an anti-IL4R antibody is an isolated mouse monoclonal antibody, such as Clone B, Clone I, M3, M5, M8, or M9.

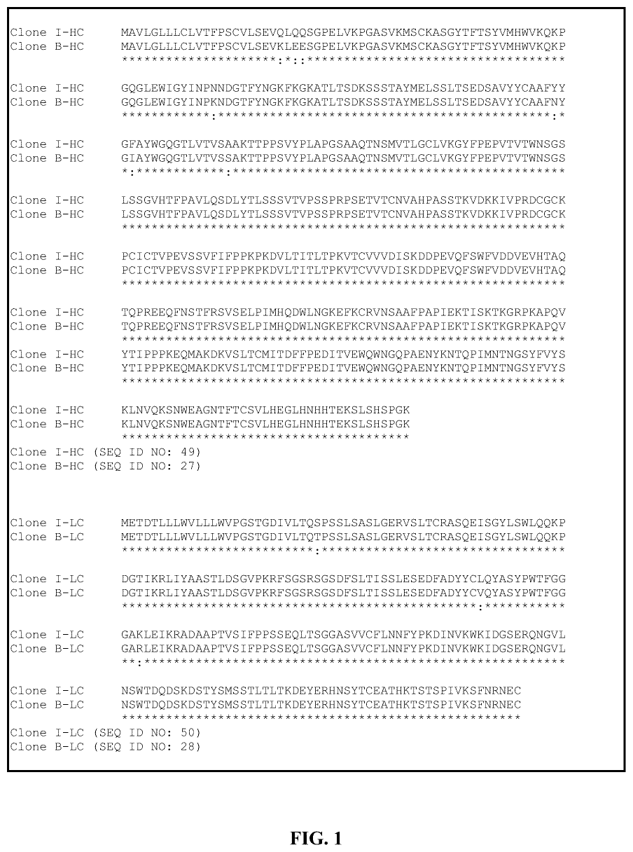

Hybridoma clones were obtained after immunization of mice with canine IL4R using standard hybridoma technology. Monoclonal antibody Clone B, Clone I, M3, M5, M8, and M9 were selected for further investigation following enzyme linked immunosorbent assay (ELISA) screening, binding affinity assays, and in vitro neutralization assay. The heavy and light chains of Clone B and Clone I were sequenced and analyzed by sequence alignment (FIG. 1; SEQ ID NO: 27 (Clone B HC), SEQ ID NO: 28 (Clone B LC), SEQ ID NO: 49 (Clone I HC), and SEQ ID NO: 50 (Clone I LC)). The variable heavy (VH) and variable light (VL) chains of M3, M5, M8, and M9 were also sequenced (SEQ ID NO: 292 (M3 VH), SEQ ID NO: 293 (M3 VL), SEQ ID NO: 308 (M5 VH), SEQ ID NO: 309 (M5 VL), SEQ ID NO: 324 (M8 VH), SEQ ID NO: 325 (M8 VL), SEQ ID NO: 340 (M9 VH), and SEQ ID NO: 341 (M9 VL).

Also provided herein are amino acid sequences of monoclonal antibody Clone B and Clone I. Exemplary consensus CDR sequences were identified as CDR-H1: GYTFTSYVMX₁ (SEQ ID NO: 1), wherein X1 is H or N, CDR-H2: YINPX₂NDGTFYX₃GX₄X₅X₆G (SEQ ID NO: 2), wherein X₂ is K, A, or N; X₃ is N or A; X₄ K or A; X₅ is F or V; and X₆ is K or Q, or YINPX₂NDGT (SEQ ID NO: 268), wherein X₂ is K, A, or N; CDR-H3: FX₇YGX₈AY (SEQ ID NO: 3), wherein X₇ is N or Y; and X₈ is I or F, CDR-L1: RASQEISGYLX₉ (SEQ ID NO: 4), wherein X₉ is S or A; CDR-L2: AASX₁₀X₁₁DX₁₂ (SEQ ID NO: 5), wherein X₁₀ is T or N; X₁₁ is R or L; and X₁₂ is S or T; and CDR-L3: X₁₃QYASYPWT (SEQ ID NO: 6), wherein X₁₃ is V or L.

In addition, for example, variable heavy chain CDRs (SEQ ID NO: 7, SEQ ID NO: 8, SEQ ID NO: 269, and SEQ ID NO: 9), variable light chain CDRs (SEQ ID NO: 14, SEQ ID NO: 15, and SEQ ID NO: 16), variable region heavy chain framework sequences (SEQ ID NO: 10, SEQ ID NO: 11, SEQ ID NO: 12, SEQ ID NO: 270, and SEQ ID NO: 13), and variable region light chain framework sequences (SEQ ID NO: 17, SEQ ID NO: 18, SEQ ID NO: 19, and SEQ ID NO: 20) for monoclonal antibody Clone B are provided Amino acid sequences of the variable heavy chain and variable light chain of monoclonal antibody Clone B are provided with and without leader sequence (SEQ ID NO: 21, SEQ ID NO: 22, SEQ ID NO: 23, and SEQ ID NO: 24). Amino acid sequences of the heavy chain and light chain of Clone B are provided with and without leader sequence (SEQ ID NO: 25, SEQ ID NO: 26, SEQ ID NO: 27, and SEQ ID NO: 28).

As another example, variable heavy chain CDRs (SEQ ID NO: 29, SEQ ID NO: 358, SEQ ID NO: 30, SEQ ID NO: 271, SEQ ID NO: 359, SEQ ID NO: 272, and SEQ ID NO: 31), variable light chain CDRs (SEQ ID NO: 36, SEQ ID NO: 360, SEQ ID NO: 37, SEQ ID NO: 361, SEQ ID NO: 362, and SEQ ID NO: 38), variable region heavy chain framework sequences (SEQ ID NO: 32, SEQ ID NO: 33, SEQ ID NO: 34, SEQ ID NO: 273, and SEQ ID NO: 35), and variable region light chain framework sequences (SEQ ID NO: 39, SEQ ID NO: 40, SEQ ID NO: 41, and SEQ ID NO: 42) for monoclonal antibody Clone I are provided. Amino acid sequences of the variable heavy chain and variable light chain of monoclonal antibody Clone I are provided with and without leader sequence (SEQ ID NO: 43, SEQ ID NO: 44, SEQ ID NO: 45, and SEQ ID NO: 46). Amino acid sequences of the heavy chain and light chain of Clone I are provided with and without leader sequence (SEQ ID NO: 47, SEQ ID NO: 48, SEQ ID NO: 49, and SEQ ID NO: 50).

As a further example, variable heavy chain CDRs (SEQ ID NO: 278, SEQ ID NO: 279, SEQ ID NO: 280), variable light chain CDRs (SEQ ID NO: 285, SEQ ID NO: 286, and SEQ ID NO: 287), variable region heavy chain framework sequences (SEQ ID NOs: 281-284), and variable region light chain framework sequences (SEQ ID NOs: 288-291) for M3 are provided.

As another example, variable heavy chain CDRs (SEQ ID NO: 294, SEQ ID NO: 295, SEQ ID NO: 296), variable light chain CDRs (SEQ ID NO: 301, SEQ ID NO: 302, and SEQ ID NO: 303), variable region heavy chain framework sequences (SEQ ID NOs: 297-300), and variable region light chain framework sequences (SEQ ID NOs: 304-307) for M5 are provided.

Further exemplified herein are variable heavy chain CDRs (SEQ ID NO: 310, SEQ ID NO: 311, SEQ ID NO: 312), variable light chain CDRs (SEQ ID NO: 317, SEQ ID NO: 318, and SEQ ID NO: 319), variable region heavy chain framework sequences (SEQ ID NOs: 313-316), and variable region light chain framework sequences (SEQ ID NOs: 320-323) for M8.

Also exemplified herein are variable heavy chain CDRs (SEQ ID NO: 326, SEQ ID NO: 407, SEQ ID NO: 327, and SEQ ID NO: 328), variable light chain CDRs (SEQ ID NO: 333, SEQ ID NO: 334, and SEQ ID NO: 335), variable region heavy chain framework sequences (SEQ ID NOs: 329-332), and variable region light chain framework sequences (SEQ ID NOs: 336-339) for M9.

Also provided herein are chimeric, caninized, and felinized antibodies derived from monoclonal antibody Clone B, Clone I, M3, M5, M8, and M9. In some embodiments, amino acid sequences of chimeric antibodies derived from Clone B are provided, such as SEQ ID NO: 51, SEQ ID NO: 52, SEQ ID NO: 53, and SEQ ID NO: 54, In some embodiments, amino acid sequences of chimeric antibodies derived from Clone I are provided, such as SEQ ID NO: 55, SEQ ID NO: 56, SEQ ID NO: 57, and SEQ ID NO: 58. In some embodiments, amino acid sequences of caninized Clone B are provided, such as SEQ ID NO: 59, SEQ ID NO: 60, SEQ ID NO: 61, SEQ ID NO: 62, SEQ ID NO: 71, SEQ ID NO: 72, SEQ ID NO: 73, and SEQ ID NO: 74. In some embodiments, amino acid sequences of caninized Clone I are provided, such as SEQ ID NO: 63, SEQ ID NO: 64, SEQ ID NO: 274, SEQ ID NO: 363, SEQ ID NO: 65, SEQ ID NO: 66, SEQ ID NO: 275, SEQ ID NO: 364, SEQ ID NO: 75, SEQ ID NO: 76, SEQ ID NO: 276, SEQ ID NO: 370, SEQ ID NO: 77, SEQ ID NO: 78, SEQ ID NO: 277, SEQ ID NO: 371, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, and SEQ ID NO: 421. In some embodiments, amino acid sequences of felinized antibodies derived from Clone B are provided, such as SEQ ID NO: 67, SEQ ID NO: 68, SEQ ID NO: 79, SEQ ID NO: 80, and SEQ ID NO: 81. In some embodiments, amino acid sequences of felinized antibodies derived from Clone I are provided, such as SEQ ID NO: 69, SEQ ID NO: 70, SEQ ID NO: 82, SEQ ID NO: 372, SEQ ID NO: 373, SEQ ID NO: 83, SEQ ID NO: 374, SEQ ID NO: 375, SEQ ID NO: 84, SEQ ID NO: 376, SEQ ID NO: 377, and SEQ ID NO: 378. In some embodiments, amino acid sequences of caninized antibodies derived from M3 are provided, such as SEQ ID NO: 342, SEQ ID NO: 343, SEQ ID NO: 344, SEQ ID NO: 348, SEQ ID NO: 349, and SEQ ID NO: 350. In some embodiments, amino acid sequences of caninized antibodies derived from M9 are provided, such as SEQ ID NO: 345, SEQ ID NO: 346, SEQ ID NO: 347, SEQ ID NO: 408, SEQ ID NO: 351, SEQ ID NO: 352, SEQ ID NO: 353, and SEQ ID NO: 409.

As used herein, numerical terms such as Kd are calculated based upon scientific measurements and, thus, are subject to appropriate measurement error. In some instances, a numerical term may include numerical values that are rounded to the nearest significant figure.

As used herein, “a” or “an” means “at least one” or “one or more” unless otherwise specified. As used herein, the term “or” means “and/or” unless specified otherwise. In the context of a multiple dependent claim, the use of “or” when referring back to other claims refers to those claims in the alternative only.

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments (such as Fab, F(ab′) 2, ScFv, minibody, diabody, triabody, and tetrabody) so long as they exhibit the desired antigen-binding activity. Canine, feline, and equine species have different varieties (classes) of antibodies that are shared by many mammalians.

The term antibody includes, but is not limited to, fragments that are capable of binding to an antigen, such as Fv, single-chain Fv (scFv), Fab, Fab′, di-scFv, sdAb (single domain antibody) and (Fab′) 2 (including a chemically linked F(ab′) 2). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen combining sites and is still capable of cross-linking antigen. The term antibody also includes, but is not limited to, chimeric antibodies, humanized antibodies, and antibodies of various species such as mouse, human, cynomolgus monkey, canine, feline, equine, etc. Furthermore, for all antibody constructs provided herein, variants having the sequences from other organisms are also contemplated. Thus, if a murine version of an antibody is disclosed, one of skill in the art will appreciate how to transform the murine sequence-based antibody into a cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, mouse scFv or a canine scFv). This denotes the sequences of at least part of the non-CDR regions, rather than the source of the construct. In some embodiments, the antibodies comprise a label or are conjugated to a second moiety.

The terms “label” and “detectable label” mean a moiety attached to an antibody or its analyte to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

The term “monoclonal antibody” refers to an antibody of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

In some embodiments, the monoclonal antibody is an isolated mouse antibody selected from Clone B, Clone I, M3, M5, M8, and M9.

“Amino acid sequence,” means a sequence of amino acids residues in a peptide or protein. The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “homology” with respect to a peptide, polypeptide, or antibody sequence are defined as the percentage of amino acid residues in a candidate sequence that are identical with the amino acid residues in the specific peptide or polypeptide sequence, after aligning the sequences and introducing gaps, if necessary to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALINE™ (DNASTAR) software. Those skilled in the art can determine appropriate parameters for measuring alignment, including any algorithms needed to achieve maximal alignment over the full length of sequences being compared.

In some embodiments, a variant has at least about 50% sequence identity with the reference nucleic acid molecule or polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, deleted, at the N- or C-terminus of the polypeptide. In some embodiments, a variant has at least about 50% sequence identity, at least about 60% sequence identity, at least about 65% sequence identity, at least about 70% sequence identity, at least about 75% sequence identity, at least about 80% sequence identity, at least about 85% sequence identity, at least about 90% sequence identity, at least about 95% sequence identity, at least about 97% sequence identity, at least about 98% sequence identity, or at least about 99% sequence identity with the sequence of the reference nucleic acid or polypeptide.

A “point mutation” is a mutation that involves a single amino acid residue. The mutation may be the loss of an amino acid, substitution of one amino acid residue for another, or the insertion of an additional amino acid residue.

An “amino acid substitution” refers to the replacement of one amino acid in a polypeptide with another amino acid. In some embodiments, an amino acid substitution is a conservative substitution. Nonlimiting exemplary conservative amino acid substitutions are shown in Table 2 Amino acid substitutions may be introduced into a molecule of interest and the products screened for a desired activity, for example, retained/improved antigen binding, decreased immunogenicity, improved ADCC or CDC, improved recombinant production, and/or enhanced pharmacokinetics.

TABLE 2

| Original | |
|---|---|
| Residue | Exemplary Substitutions |
| Ala (A) | Val; Leu; Ile |
| Arg (R) | Lys; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; |
| | Norleucine |
| Leu (L) | Norleucine; Ile; Val; Met; Ala; |
| | Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; |
| | Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes with another class.

An “amino acid derivative,” as used herein, refers to any amino acid, modified amino acid, and/or amino acid analogue, that is not one of the 20 common natural amino acids found in humans. Exemplary amino acid derivatives include natural amino acids not found in humans (e.g., seleno cysteine and pyrrolysine, which may be found in some microorganisms) and unnatural amino acids. Exemplary amino acid derivatives, include, but are not limited to, amino acid derivatives commercially available through chemical product manufacturers (e.g., sigmaaldrich.com/chemistry/chemistry-products.html?TablePage=16274965, accessed on May 6, 2017, which is incorporated herein by reference). One or more amino acid derivatives may be incorporated into a polypeptide at a specific location using a translation system that utilizes host cells, orthogonal aminoacyl-tRNA synthetases derived from eubacterial synthetases, orthogonal tRNAs, and an amino acid derivative. For further descriptions, see, e.g., U.S. Pat. No. 9,624,485.

In some embodiments, a polypeptide comprises an amino acid substitution with an amino acid derivative. In some embodiments, the amino acid derivative is an alanine derivative, a cysteine derivative, an aspartic acid derivative, a glutamic acid derivative, a phenylalanine derivative, a glycine derivative, a histidine derivative, an isoleucine derivative, a lysine derivative, a leucine derivative, a methionine derivative, an asparagine derivative, a proline derivative, a glutamine derivative, an arginine derivative, a serine derivative, a threonine derivative, a valine derivative, a tryptophan derivative, or a tyrosine derivative.

“IL4R,” as used herein, is a polypeptide comprising the entirety or a fragment of IL4 receptor subunit alpha that binds to IL4.

For example, “IL4R” refers to an IL4R polypeptide from any vertebrate source, including mammals such as primates (e.g., humans and cynomolgus monkeys), rodents (e.g., mice and rats), and companion animals (e.g., dogs, cats, and equine), unless otherwise indicated. The term also includes naturally occurring variants of IL4R, e.g., splice variants or allelic variants, or man-made variants of IL4R, e.g., labeled IL4R polypeptides. In some embodiments, IL4R is an extracellular domain fragment that binds IL4. In some such embodiments, the IL4R may be referred to as an IL4R extracellular domain (ECD). In some embodiments, IL4R comprises the amino acid sequence of SEQ ID NOs: 94, 95, 96, 97, 98, 99, 100, 101, 102, 103, 104, 105, 106, 107, 108, 109, 110, 111, 112, 113, 114, 115, 116, or 117.

“IL4,” as used herein, is a polypeptide comprising the entirety or a fragment of IL4 that binds to IL4R.

For example, IL4 refers to a IL4 polypeptide from any vertebrate source, including mammals such as primates (e.g., humans and cynomolgus monkeys), rodents (e.g., mice and rats), and companion animals (e.g., dogs, cats, and equine), unless otherwise indicated. The term also includes naturally occurring variants of IL4, e.g., splice variants or allelic variants, or man-made variants of IL4, e.g., labeled IL4 polypeptides. In some embodiments, IL4 comprises the amino acid sequence of SEQ ID NO: 118, 119, 120, 121, 122, or 123, or a processed version thereof. In some embodiments, IL4 comprises the amino acid sequence of SEQ ID NO: 124, 125, 126, 127, 128, 129, 130, or 131.

“IL13,” as used herein, is a polypeptide comprising the entirety or a fragment of IL13 that binds to IL4R.

For example, IL13 refers to a IL13 polypeptide from any vertebrate source, including mammals such as primates (e.g., humans and cynomolgus monkeys), rodents (e.g., mice and rats), and companion animals (e.g., dogs, cats, and equine), unless otherwise indicated. The term also includes naturally occurring variants of IL13, e.g., splice variants or allelic variants, or man-made variants of IL13, e.g., labeled IL13 polypeptides. In some embodiments, IL13 comprises the amino acid sequence of SEQ ID NO: 154 or 155, or a processed version thereof. In some embodiments, IL31 comprises the amino acid sequence of SEQ ID NO: 156, 157, 158, or 159.

“IL13R” or “IL13Ra1,” as used herein, is a polypeptide comprising the entirety or a fragment of IL13R that pairs with IL4R to bind to IL4 or IL13.

“Gamma C receptor,” as used herein, is a polypeptide comprising the entirety or a fragment of common gamma chain receptor that pairs with IL4R to bind to IL4.

The term “IL4R binding domain” of an antibody means the binding domain formed by a light chain and heavy chain of an anti-IL4R antibody, which binds IL4R.

In some embodiments, the IL4R binding domain binds canine IL4R with greater affinity than it binds human IL4R. In some embodiments, the IL4R binding domain binds feline IL4R.

“IL4/IL13 signaling function,” as used herein refers to any cellular effect that results when IL4 binds to IL4R paired with IL13R or Gamma C receptor, or when IL13 binds to IL4R paired with IL13R. Cellular effects may include STAT6 phosphorylation, differentiation of T helper cells into Th2 cells, activation of B cell and/or T cell proliferation, and/or induction of B cell class switching to IgE.

As used herein, the term “epitope” refers to a site on a target molecule (for example, an antigen, such as a protein, nucleic acid, carbohydrate or lipid) to which an antigen-binding molecule (for example, an antibody, antibody fragment, or scaffold protein containing antibody binding regions) binds. Epitopes often include a chemically active surface grouping of molecules such as amino acids, polypeptides or sugar side chains and have specific three-dimensional structural characteristics as well as specific charge characteristics. Epitopes can be formed both from contiguous or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some examples an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between an antibody residue and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antibody. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

In some embodiments, the epitope is within L41 and T50 of canine IL4R ECD (SEQ ID NO: 99) or feline IL4R ECD (SEQ ID NO: 100), such as within R36 and N55. For example, the epitope may comprise the amino acid sequence of SEQ ID NO: 88, SEQ ID NO: 89, SEQ ID NO: 91, or SEQ ID NO: 92. In some embodiments, the epitope comprises the amino acid sequence LX₁₀FMGSENX₁₁T, wherein X₁₀ is D or N and X₁₁ is H or R (SEQ ID NO: 85). In some embodiments, the epitope comprises the amino acid sequence RLSYQLX₁₀FMGSENX₁₁TCVPEN, wherein X₁₀ is D or N and X₁₁ is H or R (SEQ ID NO: 86).

In some embodiments, the epitope is within amino acids S64 and Q85 of canine IL4R ECD (SEQ ID NO: 99) or feline IL4R ECD (SEQ ID NO: 100). For example, the epitope may comprise the amino acid sequence of SEQ ID NO: 90 or SEQ ID NO: 93. In some embodiments, the epitope comprises the amino acid sequence SMX₁₂X₁₃DDX₁₄VEADVYQLX₁₅LWAGXQ, wherein X₁₂ is P or L, X₁₃ is I or M, X₁₄ is A or F, X₁₅ is D or H, and X₁₆ is Q or T (SEQ ID NO: 87).

In some embodiments, the epitope is within amino acids D65 and N78 of canine IL4R ECD (SEQ ID NO: 99). For example, the epitope may comprise the amino acid sequence of SEQ ID NO: 354.

In some embodiments, a first epitope is within amino acids G24 and A56 of canine IL4R ECD (SEQ ID NO: 99) and a second epitope is within amino acids R79 and 190 of canine IL4R ECD. For example, the first epitope may comprise the amino acid sequence of SEQ ID NO: 355 and the second epitope may comprise the amino acid sequence of SEQ ID NO: 356.

In some embodiments, the epitope is within amino acids R79 and V98 of canine IL4R ECD (SEQ ID NO: 99). For example, the epitope may comprise the amino acid sequence of SEQ ID NO: 357.

The term “CDR” means a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the contact definition, or a combination of the Kabat, Chothia, AbM, or contact definitions. The various CDRs within an antibody can be designated by their appropriate number and chain type, including, without limitation as CDR-H1, CDR-H2, CDR-HC3, CDR-L1, CDR-L2, and CDR-L3. The term “CDR” is used herein to also encompass a “hypervariable region” or HVR, including hypervariable loops.

In some embodiments, an anti-IL4R antibody comprises a heavy chain comprising (a) a CDR-H1 comprising the amino acid sequence of SEQ ID NO: 1, SEQ ID NO: 7, or SEQ ID NO: 29; (b) a CDR-H2 comprising the amino acid sequence of SEQ ID NO: 2, SEQ ID NO: 268, SEQ ID NO: 8, SEQ ID NO: 269, SEQ ID NO: 30, SEQ ID NO: 271, or SEQ ID NO: 272; or (c) a CDR-H3 comprising the amino acid sequence of SEQ ID NO: 3, SEQ ID NO: 9, or SEQ ID NO: 31. In some embodiments, an anti-IL4R antibody comprises a light chain comprising (a) a CDR-L1 comprising the amino acid sequence of SEQ ID NO: 4, SEQ ID NO: 14, or SEQ ID NO: 36; (b) a CDR-L2 comprising the amino acid sequence of SEQ ID NO: 5, SEQ ID NO: 15, or SEQ ID NO: 37; or (c) a CDR-L3 comprising the amino acid sequence of SEQ ID NO: 6, SEQ ID NO: 16, or SEQ ID NO: 38.

In some embodiments, an anti-IL4R antibody comprises a heavy chain comprising (a) a CDR-H1 sequence having at least 85%, at least 90%, at least 95%, or at least 98% sequence identity to the amino acid sequence of SEQ ID NO: 1, SEQ ID NO: 7, or SEQ ID NO: 29; (b) a CDR-H2 sequence having at least 85%, at least 90%, at least 95%, or at least 98% sequence identity to the amino acid sequence of SEQ ID NO: 2, SEQ ID NO: 268, SEQ ID NO: 8, SEQ ID NO: 269, SEQ ID NO: 30, SEQ ID NO: 271, or SEQ ID NO: 272; or (c) a CDR-H3 sequence having at least 85%, at least 90%, at least 95%, or at least 98% sequence identity to the amino acid sequence of SEQ ID NO: 3, SEQ ID NO: 9, or SEQ ID NO: 31. In some embodiments, an anti-IL4R antibody comprises a light chain comprising (a) a CDR-L1 sequence having at least 85%, at least 90%, at least 95%, or at least 98% sequence identity to the amino acid sequence of SEQ ID NO: 4, SEQ ID NO: 14, or SEQ ID NO: 36; (b) a CDR-L2 sequence having at least 85%, at least 90%, at least 95%, or at least 98% sequence identity to the amino acid sequence of SEQ ID NO: 5, SEQ ID NO: 15, or SEQ ID NO: 37; or (c) a CDR-L3 sequence having at least 85%, at least 90%, at least 95%, or at least 98% sequence identity to the amino acid sequence of SEQ ID NO: 6, SEQ ID NO: 16, or SEQ ID NO: 38.

In some embodiments, an anti-IL4R antibody comprises a heavy chain comprising: a) a CDR-H1 sequence having at least 85%, at least 90%, at least 95%, at least 98%, or 100% sequence identity to the amino acid sequence of SEQ ID NO: 278; b) a CDR-H2 sequence having at least 85%, at least 90%, at least 95%, at least 98%, or 100% sequence identity to the amino acid sequence of SEQ ID NO: 279; and c) a CDR-H3 sequence having at least 85%, at least 90%, at least 95%, at least 98%, or 100% sequence identity to the amino acid sequence of SEQ ID NO: 280. In some embodiments, an anti-IL4R antibody comprises a light chain comprising: a) a CDR-L1 sequence having at least 85%, at least 90%, at least 95%, at least 98%, or 100% sequence identity to the amino acid sequence of SEQ ID NO: 285; b) a CDR-L2 sequence having at least 85%, at least 90%, at least 95%, at least 98%, or 100% sequence identity to the amino acid sequence of SEQ ID NO: 286; and c) a CDR-L3 sequence having at least 85%, at least 90%, at least 95%, at least 98%, or 100% sequence identity to the amino acid sequence of SEQ ID NO: 287.

In some embodiments, an anti-IL4R antibody comprises a heavy chain comprising: a) a CDR-H1 sequence having at least 85%, at least 90%, at least 95%, at least 98%, or 100% sequence identity to the amino acid sequence of SEQ ID NO: 310; b) a CDR-H2 sequence having at least 85%, at least 90%, at least 95%, at least 98%, or 100% sequence identity to the amino acid sequence of SEQ ID NO: 311; and c) a CDR-H3 sequence having at least 85%, at least 90%, at least 95%, at least 98%, or 100% sequence identity to the amino acid sequence of SEQ ID NO: 312. In some embodiments, an anti-IL4R antibody comprises a light chain comprising: a) a CDR-L1 sequence having at least 85%, at least 90%, at least 95%, at least 98%, or 100% sequence identity to the amino acid sequence of SEQ ID NO: 317; b) a CDR-L2 sequence having at least 85%, at least 90%, at least 95%, at least 98%, or 100% sequence identity to the amino acid sequence of SEQ ID NO: 318; and c) a CDR-L3 sequence having at least 85%, at least 90%, at least 95%, at least 98%, or 100% sequence identity to the amino acid sequence of SEQ ID NO: 319.

In some embodiments, an anti-IL4R antibody comprises a heavy chain comprising: a) a CDR-H1 sequence having at least 85%, at least 90%, at least 95%, at least 98%, or 100% sequence identity to the amino acid sequence of SEQ ID NO: 326 or SEQ ID NO: 407; b) a CDR-H2 sequence having at least 85%, at least 90%, at least 95%, at least 98%, or 100% sequence identity to the amino acid sequence of SEQ ID NO: 327; and c) a CDR-H3 sequence having at least 85%, at least 90%, at least 95%, at least 98%, or 100% sequence identity to the amino acid sequence of SEQ ID NO: 328. In some embodiments, an anti-IL4R antibody comprises a light chain comprising a) a CDR-L1 sequence having at least 85%, at least 90%, at least 95%, at least 98%, or 100% sequence identity to the amino acid sequence of SEQ ID NO: 333; b) a CDR-L2 sequence having at least 85%, at least 90%, at least 95%, at least 98%, or 100% sequence identity to the amino acid sequence of SEQ ID NO: 334; and c) a CDR-L3 sequence having at least 85%, at least 90%, at least 95%, at least 98%, or 100% sequence identity to the amino acid sequence of SEQ ID NO: 335.

In some embodiments, an anti-IL4R antibody comprises a heavy chain comprising: a) a CDR-H1 sequence having at least 85%, at least 90%, at least 95%, at least 98%, or 100% sequence identity to the amino acid sequence of SEQ ID NO: 294; b) a CDR-H2 sequence having at least 85%, at least 90%, at least 95%, at least 98%, or 100% sequence identity to the amino acid sequence of SEQ ID NO: 295; and c) a CDR-H3 sequence having at least 85%, at least 90%, at least 95%, at least 98%, or 100% sequence identity to the amino acid sequence of SEQ ID NO: 296. In some embodiments, an anti-IL4R antibody comprises a light chain comprising: a) a CDR-L1 sequence having at least 85%, at least 90%, at least 95%, at least 98%, or 100% sequence identity to the amino acid sequence of SEQ ID NO: 301; b) a CDR-L2 sequence having at least 85%, at least 90%, at least 95%, at least 98%, or 100% sequence identity to the amino acid sequence of SEQ ID NO: 302; and c) a CDR-L3 sequence having at least 85%, at least 90%, at least 95%, at least 98%, or 100% sequence identity to the amino acid sequence of SEQ ID NO: 303.

The term “variable region” as used herein refers to a region comprising at least three CDRs. In some embodiments, the variable region includes the three CDRs and at least one framework region (“FR”). The terms “heavy chain variable region” or “variable heavy chain” are used interchangeably to refer to a region comprising at least three heavy chain CDRs. The terms “light chain variable region” or “variable light chain” are used interchangeably to refer to a region comprising at least three light chain CDRs. In some embodiments, the variable heavy chain or variable light chain comprises at least one framework region. In some embodiments, an antibody comprises at least one heavy chain framework region selected from HC-FR1, HC-FR2, HC-FR3, and HC-FR4. In some embodiments, an antibody comprises at least one light chain framework region selected from LC-FR1, LC-FR2, LC-FR3, and LC-FR4. The framework regions may be juxtaposed between light chain CDRs or between heavy chain CDRs. For example, an antibody may comprise a variable heavy chain having the following structure: (HC-FR1)-(HC-CDR1)-(HC-FR2)-(HC-CDR2)-(HC-FR3)-(HC-CDR3)-(HC-1-R4). An antibody may comprise a variable heavy chain having the following structure: (HC-CDR1)-(HC-FR2)-(HC-CDR2)-(HC-FR3)-(HC-CDR3). An antibody may also comprise a variable light chain having the following structure: (LC-FR1)-(LC-CDR1)-(LC-FR2)-(LC-CDR2)-(LC-FR3)-(LC-CDR3)-(LC-FR4). An antibody may also comprise a variable light chain having the following structure: (LC-CDR1)-(LC-FR2)-(LC-CDR2)-(LC-FR3)-(LC-CDR3).

In some embodiments, an anti-IL4R antibody comprises one or more of (a) a variable region heavy chain framework 1 (HC-FR1) sequence of SEQ ID NO: 10 or SEQ ID NO: 32, (b) a HC-FR2 sequence of SEQ ID NO: 11 or SEQ ID NO: 33, (c) a HC-FR3 sequence of SEQ ID NO: 12, SEQ ID NO: 270, SEQ ID NO: 34, SEQ ID NO: 273, (d) a HC-FR4 sequence of SEQ ID NO: 13 or SEQ ID NO: 35, (e) a variable region light chain framework 1 (LC-FR1) sequence of SEQ ID NO: 17 or SEQ ID NO: 39, (f) an LC-FR2 sequence of SEQ ID NO: SEQ ID NO: 18 or SEQ ID NO: 40, (g) an LC-FR3 sequence of SEQ ID NO: 19 or SEQ ID NO: 41, or (h) an LC-FR4 sequence of SEQ ID NO: 20 or SEQ ID NO: 42.

In some embodiments, an anti-IL4R antibody comprises:
a. (i) a variable heavy chain sequence having at least 85%, at least 90%, at least 95%, at least 98%, or 100% sequence identity to the amino acid sequence of SEQ ID NO: 21 or SEQ ID NO: 43; (ii) a variable light chain sequence having at least 85%, at least 90%, at least 95%, at least 98%, or 100% sequence identity to the amino acid sequence of SEQ ID NO: 22 or SEQ ID NO: 44; or (iii) a variable heavy chain sequence as in (i) and a variable light chain sequence as in (ii); or
b. (i) a variable heavy chain sequence having at least 85%, at least 90%, at least 95%, at least 98%, or 100% sequence identity to the amino acid sequence of SEQ ID NO: 59, SEQ ID NO: 60, SEQ ID NO: 63, or SEQ ID NO: 64, SEQ ID NO: 274, or SEQ ID NO: 363; (ii) a variable light chain sequence having at least 85%, at least 90%, at least 95%, at least 98%, or 100% sequence identity to the amino acid sequence of SEQ ID NO: 61, SEQ ID NO: 62, SEQ ID NO: 65, or SEQ ID NO: 66, SEQ ID NO: 275, SEQ ID NO: 364; or (iii) a variable heavy chain sequence as in (i) and a variable light chain sequence as in (ii); or
c. (i) a variable heavy chain sequence having at least 85%, at least 90%, at least 95%, at least 98%, or 100% sequence identity to the amino acid sequence of SEQ ID NO: 67 or SEQ ID NO: 69; (ii) a variable light chain sequence having at least 85%, at least 90%, at least 95%, at least 98%, or 100% sequence identity to the amino acid sequence of SEQ ID NO: 68 or SEQ ID NO: 70; or (iii) a variable heavy chain sequence as in (i) and a variable light chain sequence as in (ii).

In some embodiments, an anti-IL4R antibody comprises a variable heavy chain sequence of SEQ ID NO: 21, SEQ ID NO: 43, SEQ ID NO: 59, SEQ ID NO: 60, SEQ ID NO: 63, SEQ ID NO: 64, SEQ ID NO: 274, SEQ ID NO: 363, SEQ ID NO: 67, SEQ ID NO: 69, SEQ ID NO: 365, or SEQ ID NO: 366, and/or a variable light chain sequence of (a) SEQ ID NO: 22, SEQ ID NO: 44, SEQ ID NO: 61, SEQ ID NO: 62, SEQ ID NO: 65, SEQ ID NO: 66, SEQ ID NO: 275, SEQ ID NO: 364, SEQ ID NO: 68, SEQ ID NO: 70, SEQ ID NO: 367, SEQ ID NO: 368, or SEQ ID NO: 369.

In some embodiments, an anti-IL4R antibody comprises a heavy chain sequence of SEQ ID NO: 25, SEQ ID NO: 47, SEQ ID NO: 51, SEQ ID NO: 53, SEQ ID NO: 55, SEQ ID NO: 71, SEQ ID NO: 72, SEQ ID NO: 73, SEQ ID NO: 74, SEQ ID NO: 79, SEQ ID NO: 80, SEQ ID NO: 82, SEQ ID NO: 372, SEQ ID NO: 373, SEQ ID NO: 83, SEQ ID NO: 374, or SEQ ID NO: 375 and/or a light chain sequence of SEQ ID NO: 26, SEQ ID NO: 48, SEQ ID NO: 52, SEQ ID NO: 54, SEQ ID NO: 56, SEQ ID NO: 73, SEQ ID NO: 74, SEQ ID NO: 77, SEQ ID NO: 78, SEQ ID NO: 277, SEQ ID NO: 371, SEQ ID NO: 81, SEQ ID NO: 84, SEQ ID NO: 376, SEQ ID NO: 377, SEQ ID NO: 378.

In some embodiments, an anti-IL4R antibody comprises:
a. (i) a variable heavy chain sequence having at least 85%, at least 90%, at least 95%, at least 98%, or 100% sequence identity to the amino acid sequence of SEQ ID NO: 292; (ii) a variable light chain sequence having at least 85%, at least 90%, at least 95%, at least 98%, or 100% sequence identity to the amino acid sequence of SEQ ID NO: 293; or (iii) a variable heavy chain sequence as in (i) and a variable light chain sequence as in (ii); or
b. (i) a variable heavy chain sequence having at least 85%, at least 90%, at least 95%, at least 98%, or 100% sequence identity to the amino acid sequence of SEQ ID NO: 342 or SEQ ID NO: 343; (ii) a variable light chain sequence having at least 85%, at least 90%, at least 95%, at least 98%, or 100% sequence identity to the amino acid sequence of SEQ ID NO: 344; or (iii) a variable heavy chain sequence as in (i) and a variable light chain sequence as in (ii).

In some embodiments, an anti-IL4R antibody comprises:
a) a variable heavy chain sequence comprising the amino acid sequence of SEQ ID NO: 292, and a variable light chain sequence comprising the amino acid sequence of SEQ ID NO: 293; or
b) a variable heavy chain sequence comprising the amino acid sequence of SEQ ID NO: 342 or SEQ ID NO: 343, and a variable light chain sequence comprising the amino acid sequence of SEQ ID NO: 344.

In some embodiments, an anti-IL4R antibody comprises a variable heavy chain sequence of SEQ ID NO: 292, SEQ ID NO: 342, or SEQ ID NO: 343 and/or a variable light chain sequence of (a) SEQ ID NO: 293 or SEQ ID NO: 344.

In some embodiments, an anti-IL4R antibody comprises a heavy chain sequence of SEQ ID NO: SEQ ID NO: 348 or SEQ ID NO: 349 and/or a light chain sequence of SEQ ID NO: 350.

In some embodiments, an anti-IL4R antibody comprises: (a) a variable heavy chain sequence having at least 85%, at least 90%, at least 95%, at least 98%, or 100% sequence identity to the amino acid sequence of SEQ ID NO: 324; (b) a variable light chain sequence having at least 85%, at least 90%, at least 95%, at least 98%, or 100% sequence identity to the amino acid sequence of SEQ ID NO: 325; or (c) a variable heavy chain sequence as in (a) and a variable light chain sequence as in (b).

In some embodiments, an anti-IL4R antibody comprises a variable heavy chain sequence of SEQ ID NO: 324 and/or a variable light chain sequence of (a) SEQ ID NO: 325.

In some embodiments, an anti-IL4R antibody comprises a heavy chain sequence of SEQ ID NO: SEQ ID NO: 348 or SEQ ID NO: 349 and/or a light chain sequence of SEQ ID NO: 350.

In some embodiments, an anti-IL4R antibody comprises:
a. (i) a variable heavy chain sequence having at least 85%, at least 90%, at least 95%, at least 98%, or 100% sequence identity to the amino acid sequence of SEQ ID NO: 340; (ii) a variable light chain sequence having at least 85%, at least 90%, at least 95%, at least 98%, or 100% sequence identity to the amino acid sequence of SEQ ID NO: 341; or (iii) a variable heavy chain sequence as in (i) and a variable light chain sequence as in (ii); or
b. (i) a variable heavy chain sequence having at least 85%, at least 90%, at least 95%, at least 98%, or 100% sequence identity to the amino acid sequence of SEQ ID NO: 345 or SEQ ID NO: 346; (ii) a variable light chain sequence having at least 85%, at least 90%, at least 95%, at least 98%, or 100% sequence identity to the amino acid sequence of SEQ ID NO: 347 or SEQ ID NO: 408; or (iii) a variable heavy chain sequence as in (i) and a variable light chain sequence as in (ii).

In some embodiments, an anti-IL4R antibody comprises a variable heavy chain sequence of SEQ ID NO: 340, SEQ ID NO: 345, or SEQ ID NO: 346 and/or a variable light chain sequence of (a) SEQ ID NO: 341, SEQ ID NO: 347, or SEQ ID NO: 408.

In some embodiments, an anti-IL4R antibody comprises a heavy chain sequence of SEQ ID NO: SEQ ID NO: 348 or SEQ ID NO: 349 and/or a light chain sequence of SEQ ID NO: 350.

In some embodiments, an anti-IL4R antibody comprises: The isolated antibody of any one of claims 75 to 85, wherein the antibody comprises: (a) a variable heavy chain sequence having at least 85%, at least 90%, at least 95%, at least 98%, or 100% sequence identity to the amino acid sequence of SEQ ID NO: 308; (b) a variable light chain sequence having at least 85%, at least 90%, at least 95%, at least 98%, or 100% sequence identity to the amino acid sequence of SEQ ID NO: 309; or (c) a variable heavy chain sequence as in (a) and a variable light chain sequence as in (b).

In some embodiments, an anti-IL4R antibody comprises a variable heavy chain sequence of SEQ ID NO: 308 and/or a variable light chain sequence of (a) SEQ ID NO: 341 or SEQ ID NO: 309.

The term “constant region” as used herein refers to a region comprising at least three constant domains. The terms “heavy chain constant region” or “constant heavy chain” are used interchangeably to refer to a region comprising at least three heavy chain constant domains, CH1, CH2, and CH3. Nonlimiting exemplary heavy chain constant regions include γ, δ, α, ε, and μ. Each heavy chain constant region corresponds to an antibody isotype. For example, an antibody comprising a γ constant region is an IgG antibody, an antibody comprising a δ constant region is an IgD antibody, an antibody comprising an α constant region is an IgA antibody, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to IgM1 and IgM2. The terms “light chain constant region” or “constant light chain” are used interchangeably to refer to a region comprising a light chain constant domain, CL. Nonlimiting exemplary light chain constant regions include λ and κ (e.g., SEQ ID NO: 235 or 241). Non-function-altering deletions and alterations within the domains are encompassed within the scope of the term “constant region” unless designated otherwise. Canine, feline, and equine have antibody classes such as IgG, IgA, IgD, IgE, and IgM. Within the canine IgG antibody class are IgG-A, IgG-B, IgG-C, and IgG-D. Within the feline IgG antibody class are IgG1a, IgG1b, and IgG2. Within the equine IgG antibody class are IgG1, IgG2, IgG3, IgG4, IgG5, IgG6, and IgG7.

A “fragment crystallizable polypeptide” or “Fc polypeptide” is the portion of an antibody molecule that interacts with effector molecules and cells. It comprises the C-terminal portions of the immunoglobulin heavy chains. As used herein, an Fc polypeptide includes fragments of the Fc domain having one or more biological activities of an entire Fc polypeptide. In some embodiments, a biological activity of an Fc polypeptide is the ability to bind FcRn. In some embodiments, a biological activity of an Fc polypeptide is the ability to bind C1q. In some embodiments, a biological activity of an Fc polypeptide is the ability to bind CD16. In some embodiments, a biological activity of an Fc polypeptide is the ability to bind protein A. An “effector function” of the Fc polypeptide is an action or activity performed in whole or in part by any antibody in response to a stimulus and may include complement fixation and/or ADCC (antibody-dependent cellular cytotoxicity) induction.

The term “IgX Fc” means the Fc region is derived from a particular antibody isotype (e.g., IgG, IgA, IgD, IgE, IgM, etc.), where “X” denotes the antibody isotype. Thus, “IgG Fc” denotes the Fc region of a γ chain, “IgA Fc” denotes the Fc region of an α chain, “IgD Fc” denotes the Fc region of a δ chain, “IgE Fc” denotes the Fc region of an ε chain, “IgM Fc” denotes the Fc region of a μ chain, etc. In some embodiments, the IgG Fc region comprises CH1, hinge, CH2, CH3, and CL1. “IgX-N-Fc” denotes that the Fc region is derived from a particular subclass of antibody isotype (such as canine IgG subclass A, B, C, or D; feline IgG subclass 1, 2a, or 2b; or equine IgG subclass IgG1, IgG2, IgG3, IgG4, IgG5, IgG6, or IgG7, etc.), where “N” denotes the subclass.

In some embodiments, an IgX Fc polypeptide or IgX-N-Fc polypeptide is derived from a companion animal, such as a dog, a cat, or a horse. In some embodiments, IgG Fc polypeptides are isolated from canine γ heavy chains, such as IgG-A, IgG-B, IgG-C, or IgG-D. In some instances, IgG Fc polypeptides are isolated from feline γ heavy chains, such as IgG1, IgG2a, or IgG2b. In other instances, IgG Fc polypeptides are isolated from equine γ heavy chains, such as IgG1, IgG2, IgG3, IgG4, IgG5, IgG6, or IgG7.

The terms “IgX Fc” and “IgX Fc polypeptide” include wild-type IgX Fc polypeptides and variant IgX Fc polypeptides, unless indicated otherwise.

“Wild-type” refers to a non-mutated version of a polypeptide that occurs in nature, or a fragment thereof. A wild-type polypeptide may be produced recombinantly.

In some embodiments, a wild-type IgG Fc polypeptide comprises the amino acid sequence of SEQ ID NO: 162, SEQ ID NO: 163, SEQ ID NO: 164, SEQ ID NO: 165, SEQ ID NO: 166, SEQ ID NO: 167, SEQ ID NO: 203, SEQ ID NO: 204, SEQ ID NO: 205, SEQ ID NO: 206, SEQ ID NO: 207, SEQ ID NO: 247, SEQ ID NO: 248, SEQ ID NO: 249, SEQ ID NO: 250, SEQ ID NO: 251, SEQ ID NO: 252, or SEQ ID NO: 253.

A “variant” is a polypeptide that differs from a reference polypeptide by single or multiple non-native amino acid substitutions, deletions, and/or additions. In some embodiments, a variant retains at least one biological activity of the reference polypeptide (e.g., wild-type polypeptide).

A “variant IgG Fc polypeptide” as used herein is an IgG Fc polypeptide that differs from a reference IgG Fc polypeptide by single or multiple amino acid substitutions, deletions, and/or additions and substantially retains at least one biological activity of the reference IgG Fc polypeptide.

In some embodiments, a variant IgG Fc polypeptide comprises a variant IgG Fc polypeptide of a companion animal species. In some embodiments, a variant IgG Fc polypeptide comprises a variant canine IgG Fc polypeptide, a variant equine IgG Fc polypeptide, or a feline IgG Fc polypeptide. In some embodiments, a variant IgG Fc polypeptide (e.g., a variant canine IgG-A Fc polypeptide, a variant canine IgG-C Fc polypeptide, a variant canine IgG-D Fc polypeptide, variant feline IgG1a Fc polypeptide, variant feline IgG1b Fc polypeptide, or variant feline IgG2 Fc polypeptide) has an activity that the reference (e.g., wild-type) polypeptide substantially lacks. For example, in some embodiments, a variant canine IgG-A Fc polypeptide, a variant canine IgG-C Fc polypeptide, or a variant canine IgG-D Fc polypeptide binds Protein A.

In some embodiments, a variant IgG Fc polypeptide has modified Protein A binding affinity. In some embodiments, a variant IgG Fc polypeptide has increased binding affinity to Protein A. In some embodiments, a variant IgG Fc polypeptide may be purified using Protein A column chromatography. In some embodiments, a variant IgG Fc polypeptide has modified CD16 binding affinity. In some embodiments, a variant IgG Fc polypeptide has decreased binding affinity to CD16. In some embodiments, a variant IgG Fc may have a reduced ADCC immune response. In some embodiments, a variant IgG Fc polypeptide has modified C1q binding affinity. In some embodiments, a variant IgG Fc polypeptide has reduced binding affinity to C1q. In some embodiments, a variant IgG Fc polypeptide may have reduced complement fixation. In some embodiments, a variant IgG Fc may have a reduced complement-mediated immune response. In some embodiments, a variant IgG Fc polypeptide has modified FcRn binding affinity. In some embodiments, a variant IgG Fc polypeptide has increased binding affinity to FcRn.

“Hinge” refers to any portion of an Fc polypeptide or variant Fc polypeptide that is proline-rich, comprises at least one cysteine residue, and is located between CH1 and CH2 of a heavy chain constant region.

In some embodiments, a hinge is capable of forming a disulfide linkage within the same hinge region, within the same Fc polypeptide, with a hinge region of a separate Fc polypeptide, or with a separate Fc polypeptide. In some embodiments, a hinge comprises at least one, at least two, at least three, at least four, at least five, at least six, at least seven, at least eight, at least nine, or at least ten proline residues.

In some embodiments, a variant feline IgG Fc polypeptide has at least one additional inter-chain disulfide linkage relative to the wild-type feline IgG Fc polypeptide, such as in the hinge region. In some embodiments, a variant feline IgG2 Fc polypeptide with at least one additional inter-chain disulfide linkage has increased inter-chain stability relative to the wild-type feline IgG Fc polypeptide. In some embodiments, a variant IgG polypeptide has at least one amino acid modification to a hinge region relative to a wild-type IgG Fc polypeptide, such as a wild-type feline IgG Fc polypeptide.

In some embodiments, a variant IgG Fc polypeptide comprises a hinge region or a portion of a hinge region from an IgG Fc polypeptide of a different isotype. In some embodiments, the variant IgG Fc polypeptide, such as a canine IgG2 Fc polypeptide, comprises a hinge region from a wild-type feline IgG1a or IgG1b Fc polypeptide. In some embodiments, a variant IgG Fc polypeptide has increased recombinant production and/or increased hinge disulfide formation relative to the wild-type IgG Fc polypeptide. In some embodiments, the increased recombinant production and/or increased hinge disulfide formation can be determined by SDS-PAGE analysis under reducing and/or non-reducing conditions.

In some embodiments, a variant IgG Fc polypeptide comprises: a) at least one amino acid substitution at a position corresponding to position 21, 23, 25, 80, 205, and/or 207 of SEQ ID NO: 162; b) at least one amino acid substitution at a position corresponding to position 5, 38, 39, 94, 97, and/or 98 of SEQ ID NO: 163; c) at least one amino acid substitution at a position corresponding to position 5, 21, 23, 24, 38, 39, 93, 97, and/or 98 of SEQ ID NO: 165; d) at least one amino acid substitution at a position corresponding to position 21, 23, 25, 80, and/or 207 of SEQ ID NO: 167; e) at least one amino acid substitution at a position corresponding to position 16 and/or 198 of SEQ ID NO: 203, SEQ ID NO: 204, SEQ ID NO: 205, or SEQ ID NO: 206; and/or f) at least one amino acid substitution at a position corresponding to position 14 and/or 16 of SEQ ID NO: 207.

In some embodiments, a variant IgG Fc polypeptide comprises: a) at least one amino acid substitution at position 21, 23, 25, 80, 205, and/or 207 of SEQ ID NO: 162; b) at least one amino acid substitution at position 5, 38, 39, 94, 97, and/or 98 of SEQ ID NO: 163; c) at least one amino acid substitution at position 5, 21, 23, 24, 38, 39, 93, 97, and/or 98 of SEQ ID NO: 164; d) at least one amino acid substitution at position 21, 23, 25, 80, and/or 207 of SEQ ID NO: 165; e) at least one amino acid substitution at position 16 and/or 198 of SEQ ID NO: 203, SEQ ID NO: 204, SEQ ID NO: 205, or SEQ ID NO: 206; and/or f) at least one amino acid substitution at position 14 and/or 16 of SEQ ID NO: 207.

In some embodiments, a variant IgG Fc polypeptide comprises:
a) a threonine at a position corresponding to position 21, a leucine at a position corresponding to position 23, an alanine at a position corresponding to position 25, a glycine at a position corresponding to position 80, an alanine at a position corresponding to position 205, and/or a histidine at a position corresponding to position 207 of SEQ ID NO: 162;
b) a proline at a position corresponding to position 5, a glycine at a position corresponding to position 38, an arginine at a position corresponding to position 39, an arginine at a position corresponding to position 93, an isoleucine at a position corresponding to position 97, and/or a glycine at a position corresponding to position 98 of SEQ ID NO: 163;
c) a proline at a position corresponding to position 5, a threonine at a position corresponding to position 21, a leucine at a position corresponding to position 23, an isoleucine at a position corresponding to position 24, a glycine at a position corresponding to position 38, an arginine at a position corresponding to position 39, an arginine at a position corresponding to position 93, an isoleucine at a position corresponding to position 97, and/or a glycine at a position corresponding to position 98 of SEQ ID NO: 164;
d) a threonine at a position corresponding to position 21, a leucine at a position corresponding to position 23, an alanine at a position corresponding to position 25, a glycine at a position corresponding to position 80, and/or a histidine at a position corresponding to position 207 of SEQ ID NO: 165;
e) a proline at a position corresponding to position 16 and/or an alanine at a position corresponding to position 198 of SEQ ID NO: 203, SEQ ID NO: 204, SEQ ID NO: 205, or SEQ ID NO: 206; and/or
f) a cysteine at a position corresponding to position 14 and/or a proline at a position corresponding to position 16 of SEQ ID NO: 207.

In some embodiments, a variant IgG Fc polypeptide comprises:
a) a threonine at position 21, a leucine at position 23, an alanine at position 25, a glycine at position 80, an alanine at position 205, and/or a histidine at position 207 of SEQ ID NO: 162;
b) a proline at position 5, a glycine at position 38, an arginine at position 39, an arginine at position 93, an isoleucine at position 97, and/or a glycine at position 98 of SEQ ID NO: 163;
c) a proline at position 5, a threonine at position 21, a leucine at position 23, an isoleucine at position 24, a glycine at position 38, an arginine at position 39, an arginine at position 93, an isoleucine at position 97, and/or a glycine at position 98 of SEQ ID NO: 164;
d) a threonine at position 21, a leucine at position 23, an alanine at position 25, a glycine at position 80, and/or a histidine at position 207 of SEQ ID NO: 165;
e) a proline at position 16 and/or an alanine at position 198 of SEQ ID NO: 203, SEQ ID NO: 204, SEQ ID NO: 205, or SEQ ID NO: 206; and/or
f) a cysteine at position 14 and/or a proline at position 16 of SEQ ID NO: 207.

In some embodiments, a variant IgG Fc polypeptide comprises the amino acid sequence of SEQ ID NO: 168, 169, 170, 171, 172, 173, 174, 175, 176, 177, 178, 179, 180, 181, 182, 183, 184, 185, 186, 187, 188, 189, 190, 191, 192, 193, 194, 208, 209, 210, 211, 212, 213, 214, 215, 216, 381, 382, 383, 384, 385, 386, 387, 388, 389, 390, 391, 392, 393, or 394.

A bispecific antibody has a binding specificity for two different epitopes or target molecules. In some embodiments, a bispecific antibody binds two different epitopes of the same target molecule. Bispecific antibodies may be full length antibodies or antibody fragments.

In some embodiments, an antibody comprises a first variant IgG Fc polypeptide comprising a “knob” mutation and a second variant IgG Fc polypeptide comprising a “hole” mutation. Nonlimiting exemplary knob and hole mutations are described, for example, in Merchant, A. M. et al. An efficient route to human bispecific IgG. Nat Biotechnol, 16(7):677-81 (1998).

In some embodiments, a variant IgG Fc polypeptide comprises a knob mutation. In some embodiments, a variant IgG Fc polypeptide comprises an amino acid substitution at a position corresponding to position 138 of SEQ ID NO: 162; position 137 of SEQ ID NO: 163, position 137 of SEQ ID NO: 165; position 138 of SEQ ID NO: 167; position 154 of SEQ ID NO: 203, SEQ ID NO: 204, SEQ ID NO: 205, SEQ ID NO: 206, or SEQ ID NO: 207; or position 130 of SEQ ID NO: 247, SEQ ID NO: 248, SEQ ID NO: 249, SEQ ID NO: 250, SEQ ID NO: 251, SEQ ID NO: 252, or SEQ ID NO: 253. In some embodiments, a variant IgG Fc polypeptide comprises an amino acid substitution at position 138 of SEQ ID NO: 162; position 137 of SEQ ID NO: 163; position 137 of SEQ ID NO: 165; position 138 of SEQ ID NO: 167; position 154 of SEQ ID NO: 203, SEQ ID NO: 204, SEQ ID NO: 205, SEQ ID NO: 206, or SEQ ID NO: 207; or position 130 of SEQ ID NO: 247, SEQ ID NO: 248, SEQ ID NO: 249, SEQ ID NO: 250, SEQ ID NO: 251, SEQ ID NO: 252, or SEQ ID NO: 253. In some embodiments, a variant IgG Fc polypeptide comprises a tryptophan at a position corresponding to position 138 of SEQ ID NO: 162; position 137 of SEQ ID NO: 163; position 137 of SEQ ID NO: 165; position 138 of SEQ ID NO: 167, or position 154 of SEQ ID NO: 203, SEQ ID NO: 204, SEQ ID NO: 205, SEQ ID NO: 206, or SEQ ID NO: 207; or position 130 of SEQ ID NO: 247, SEQ ID NO: 248, SEQ ID NO: 249, SEQ ID NO: 250, SEQ ID NO: 251, SEQ ID NO: 252, or SEQ ID NO: 253. In some embodiments, a variant IgG Fc polypeptide comprises a tryptophan at position 138 of SEQ ID NO: 162; position 137 of SEQ ID NO: 163; position 137 of SEQ ID NO: 165; position 138 of SEQ ID NO: 167; position 154 of SEQ ID NO: 203, SEQ ID NO: 204, SEQ ID NO: 205, SEQ ID NO: 206, or SEQ ID NO: 207; or position 130 of SEQ ID NO: 247, SEQ ID NO: 248, SEQ ID NO: 249, SEQ ID NO: 250, SEQ ID NO: 251, SEQ ID NO: 252, or SEQ ID NO: 253. In some embodiments, a variant IgG Fc polypeptide comprises the amino acid sequence of SEQ ID NO: 195, 196, 197, 198, 217, 218, 219, 220, 221, 254, 255, 256, 257, 258, 259, or 260.

In some embodiments, a variant IgG Fc polypeptide comprises a hole mutation. In some embodiments, a variant IgG Fc polypeptide comprises an amino acid substitution at a position corresponding to position 138 and/or position 140 of SEQ ID NO: 162; position 137 and/or position 139 of SEQ ID NO: 163; position 137 and/or position 139 of SEQ ID NO: 165; position 138 and/or position 140 of SEQ ID NO: 167; position 154 and/or position 156 of SEQ ID NO: 203, SEQ ID NO: 204, SEQ ID NO: 205, SEQ ID NO: 206, or SEQ ID NO: 207; and/or position 130 and/or position 132 of SEQ ID NO: 247, SEQ ID NO: 248, SEQ ID NO: 249, SEQ ID NO: 250, SEQ ID NO: 251, SEQ ID NO: 252, or SEQ ID NO: 253. In some embodiments, a variant IgG Fc polypeptide comprises an amino acid substitution at position 138 and/or position 140 of SEQ ID NO: 162; position 137 and/or position 139 of SEQ ID NO: 163; position 137 and/or position 139 of SEQ ID NO: 165; position 138 and/or position 140 of SEQ ID NO: 167; position 154 and/or position 156 of SEQ ID NO: 203, SEQ ID NO: 204, SEQ ID NO: 205, SEQ ID NO: 206, or SEQ ID NO: 207; or position 130 and/or position 132 of SEQ ID NO: 247, SEQ ID NO: 248, SEQ ID NO: 249, SEQ ID NO: 250, SEQ ID NO: 251, SEQ ID NO: 252, or SEQ ID NO: 253. In some embodiments, a variant IgG Fc polypeptide comprises a serine at a position corresponding to position 138 and/or an alanine at a position corresponding to position 140 of SEQ ID NO: 162; a serine at a position corresponding to position 137 and/or an alanine at a position corresponding to position 139 of SEQ ID NO: 163; a serine at a position corresponding to position 137 and/or an alanine at a position corresponding to position 139 of SEQ ID NO: 165; a serine at a position corresponding to position 138 and/or an alanine at a position corresponding to position 140 of SEQ ID NO: 167; a serine at a position corresponding to position 154 and/or an alanine at a position corresponding to position 156 of SEQ ID NO: 203, SEQ ID NO: 204, SEQ ID NO: 205, SEQ ID NO: 206, or SEQ ID NO: 207; or a serine at a position corresponding to position 130 and/or an alanine at a position corresponding to position 132 of SEQ ID NO: 247, SEQ ID NO: 248, SEQ ID NO: 249, SEQ ID NO: 250, SEQ ID NO: 251, SEQ ID NO: 252, or SEQ ID NO: 253. In some embodiments, a variant IgG Fc polypeptide comprises a serine at position 138 and/or an alanine at position 140 of SEQ ID NO: 162; a serine at position 137 and/or an alanine at position 139 of SEQ ID NO: 163; a serine at position 137 and/or an alanine at position 139 of SEQ ID NO: 165; a serine at position 138 and/or an alanine at position 140 of SEQ ID NO: 167; a serine at position 154 and/or an alanine at position 156 of SEQ ID NO: 203, SEQ ID NO: 204, SEQ ID NO: 205, SEQ ID NO: 206, or SEQ ID NO: 207; or a serine at position 130 and/or an alanine at position 132 of SEQ ID NO: 247, SEQ ID NO: 248, SEQ ID NO: 249, SEQ ID NO: 250, SEQ ID NO: 251, SEQ ID NO: 252, or SEQ ID NO: 253. In some embodiments, a variant IgG Fc polypeptide comprises the amino acid sequence of SEQ ID NO: 199, 200, 201, 202, 222, 223, 224, 225, 226, 261, 262, 263, 264, 265, 266, or 267.

Furthermore, to facilitate a heavy chain to specifically pair with its intended light chain, interface amino acids between CH1 and the light chain may be mutated to be complementary in shape and charge-charge interaction.

In some embodiments, a variant IgG Fc polypeptide comprises a CH1 region comprising at least one amino acid substitution at a position corresponding to position 24 and/or position 30 of SEQ ID NO: 227, SEQ ID NO: 228, SEQ ID NO: 229, SEQ ID NO: 230, or SEQ ID NO: 237; or at least one amino acid substitution at a position corresponding to position 24 and/or position 29 of SEQ ID NO: 238. In some embodiments, a variant IgG Fc polypeptide comprises a CH1 region comprising at least one amino acid substitution at position 24 and/or position 30 of SEQ ID NO: 227, SEQ ID NO: 228, SEQ ID NO: 229, SEQ ID NO: 230, or SEQ ID NO: 237; or at least one amino acid substitution at position 24 and/or position 29 of SEQ ID NO: 238. In some embodiments, a variant IgG Fc polypeptide comprises a CH1 region comprising a leucine at a position corresponding to position 24 and/or an asparagine at a position corresponding to position 30 of SEQ ID NO: 227, SEQ ID NO: 228, SEQ ID NO: 229, SEQ ID NO: 230, or SEQ ID NO: 237; or a leucine at a position corresponding to position 24 and/or an asparagine at a position corresponding to position 29 of SEQ ID NO: 238. In some embodiments, a variant IgG Fc polypeptide comprises a CH1 region comprising a leucine at position 24 and/or an asparagine at position 30 of SEQ ID NO: 227, SEQ ID NO: 228, SEQ ID NO: 229, SEQ ID NO: 230, or SEQ ID NO: 237; or a leucine at position 24 and/or an asparagine at position 29 of SEQ ID NO: 238. In some embodiments, a variant IgG Fc polypeptide comprises a CH1 region comprising the amino acid sequence of SEQ ID NO: 231, 232, 233, 234, 239, or 240.

In some embodiments, a complementary variant light chain constant region comprises at least one amino acid substitution at a position corresponding to position 11 and/or position 22 of SEQ ID NO: 235 or SEQ ID NO: 241. In some embodiments, a variant light chain constant region comprises at least one amino acid substitution at position 11 and/or position 22 of SEQ ID NO: 235 or SEQ ID NO: 241. In some embodiments, a variant light chain constant region comprises an alanine at a position corresponding to position 11 and/or an arginine at a position corresponding to position 22 of SEQ ID NO: 235 or SEQ ID NO: 241. In some embodiments, a variant light chain constant region comprises an alanine at position 11 and/or an arginine at position 22 of SEQ ID NO: 235 or SEQ ID NO: 241. In some embodiments, a variant light chain constant region comprises the amino acid sequence of SEQ ID NO: 236 or 242.

The term “chimeric antibody” or “chimeric” refers to an antibody in which a portion of the heavy chain or light chain is derived from a particular source or species, while at least a part of the remainder of the heavy chain or light chain is derived from a different source or species. In some embodiments, a chimeric antibody refers to an antibody comprising at least one variable region from a first species (such as mouse, rat, cynomolgus monkey, etc.) and at least one constant region from a second species (such as human, dog, cat, equine, etc.). In some embodiments, a chimeric antibody comprises at least one mouse variable region and at least one canine constant region. In some embodiments, a chimeric antibody comprises at least one mouse variable region and at least one feline constant region. In some embodiments, all of the variable regions of a chimeric antibody are from a first species and all of the constant regions of the chimeric antibody are from a second species. In some embodiments, a chimeric antibody comprises a constant heavy chain region or constant light chain region from a companion animal. In some embodiments, a chimeric antibody comprises a mouse variable heavy and light chains and a companion animal constant heavy and light chains. For example, a chimeric antibody may comprise a mouse variable heavy and light chains and a canine constant heavy and light chains; a chimeric antibody may comprise a mouse variable heavy and light chains and a feline constant heavy and light chains; or a chimeric antibody may comprise a mouse variable heavy and light chains and an equine constant heavy and light chains.

A “canine chimeric” or “canine chimeric antibody” refers to a chimeric antibody having at least a portion of a heavy chain or a portion of a light chain derived from a dog. A “feline chimeric” or “feline chimeric antibody” refers to a chimeric antibody having at least a portion of a heavy chain or a portion of a light chain derived from a cat. In some embodiments, a canine chimeric antibody comprises a mouse variable heavy and light chains and a canine constant heavy and light chains. In some embodiments, a feline chimeric antibody comprises a mouse variable heavy and light chains and a feline constant heavy and light chains. In some embodiments, the antibody is a chimeric antibody comprising murine variable heavy chain framework regions or murine variable light chain framework regions.

In some embodiments, an anti-IL4R antibody comprises a chimeric antibody comprising: (i) a heavy chain amino acid sequence of SEQ ID NO: 51 or SEQ ID NO: 55; (ii) a light chain amino acid sequence of SEQ ID NO: 52 or SEQ ID NO: 56; or (iii) a heavy chain amino acid sequence as in (i) and a light chain sequence as in (ii).

A “canine antibody,” as used herein, encompasses antibodies produced in a canine; antibodies produced in non-canine animals that comprise canine immunoglobulin genes or comprise canine immunoglobulin peptides; or antibodies selected using in vitro methods, such as phage display, wherein the antibody repertoire is based on a canine immunoglobulin sequence. The term “canine antibody” denotes the genus of sequences that are canine sequences. Thus, the term is not designating the process by which the antibody was created, but the genus of sequences that are relevant.

A “caninized antibody” means an antibody in which at least one amino acid in a portion of a non-canine variable region has been replaced with the corresponding amino acid from a canine variable region. In some embodiments, a caninized antibody comprises at least one canine constant region (e.g., a γ constant region, an a constant region, a δ constant region, an ε constant region, a μ constant region, or etc.) or fragment thereof. In some embodiments, a caninized antibody is an antibody fragment, such as Fab, scFv, (Fab′)₂, etc. The term “caninized” also denotes forms of non-canine (for example, murine) antibodies that are chimeric immunoglobulins, immunoglobulin chains, or fragments thereof (such as Fv, Fab, Fab′, F(ab′)₂ or other antigen-binding sequences of antibodies) that contain minimal sequence of non-canine immunoglobulin. Caninized antibodies can include canine immunoglobulins (recipient antibody) in which residues from a CDR of the recipient are substituted by residues from a CDR of a non-canine species (donor antibody) such as mouse, rat, or rabbit having the desired specificity, affinity, and capacity. In some instances, Fv framework region (FR) residues of the canine immunoglobulin are replaced by corresponding non-canine residues. Furthermore, the caninized antibody can comprise residues that are found neither in the recipient antibody nor in the imported CDR or framework sequences, but are included to further refine and optimize antibody performance.

In some embodiments, at least one amino acid residue in a portion of a mouse variable heavy chain or a mouse variable light chain has been replaced with the corresponding amino acid from a canine variable region. In some embodiments, the modified chain is fused to a canine constant heavy chain or a canine constant light chain. In some embodiments, an anti-IL4R antibody is a caninized antibody comprising a variable heavy chain amino acid sequence of SEQ ID NO: 59, 60, 63, or 64 and/or a variable light chain amino acid sequence of SEQ ID NO: 61, 62, 65, or 66.

In some embodiments, an anti-IL4R antibody comprises a canine heavy chain constant region selected from an IgG-A, IgG-B, IgG-C, and IgG-D constant region. In some embodiments, an anti-IL4R antibody comprises is a wild-type or variant canine IgG-A, IgG-B, IgG-C, or IgG-D Fc polypeptide, as described herein. In some embodiments, an anti-IL4R antibody comprises a canine IgG-A Fc polypeptide comprising the amino acid sequence of SEQ ID NO: 162; a canine IgG-B Fc polypeptide comprising the amino acid sequence of SEQ ID NO: 163 or 164; (c) a canine IgG-C Fc polypeptide comprising the amino acid sequence of SEQ ID NO: 165 or 166; or (d) a canine IgG-D Fc polypeptide comprising the amino acid sequence of SEQ ID NO: 167. In some embodiments, an anti-IL4R antibody comprises a variant canine IgG-A Fc polypeptide comprising the amino acid sequence of SEQ ID NO: 168, 169, 195, 199, 383, 384; a variant canine IgG-B Fc polypeptide comprising the amino acid sequence of SEQ ID NO: 170, 171, 172, 173, 174, 175, 176, 177, 178, 179, 196, 200, 381, 382, 387, 388, 389, 390, 391, 392, 393, or 394; (c) a variant canine IgG-C Fc polypeptide comprising the amino acid sequence of SEQ ID NO: 180, 181, 182, 183, 184, 185, 186, 187, 188, 189, 190, 191, 192, 197, or 201; or (d) a variant canine IgG-D Fc polypeptide comprising the amino acid sequence of SEQ ID NO: 194, 198, 202, 385, or 386.

In some embodiments, an anti-IL4R antibody comprises a canine light chain constant region, such as a canine κ light constant region. In some embodiments, an anti-IL4R antibody comprises is a wild-type canine κ light constant region (e.g., SEQ ID NO: 235) or variant canine κ light constant region (e.g., SEQ ID NO: 236).

In some embodiments, an anti-IL4R antibody comprises a caninized variable heavy chain of Clone B, Clone I, M3, M5, M8, or M9 and a variant canine IgG Fc polypeptide, such as SEQ ID NO: 71, 72, 75, 76, 276, 370, 348, 349, 351, 352, 395, 396, 397, 398, 399, 400, 401, 402, 403, 404, 405, 406, 410, 411, 412, 413, 414, 415, 416, 417, 418, 419, 420, or 421. In some embodiments, an anti-IL4R antibody comprises a caninized variable light chain of Clone B, Clone I, M3, M5, M8, or M9 and a wild-type canine κ light chain constant region, such as SEQ ID NO: 73, 74, 77, 78, 277, 371, 350, or 352.

A “feline antibody,” as used herein, encompasses antibodies produced in a feline; antibodies produced in non-feline animals that comprise feline immunoglobulin genes or comprise feline immunoglobulin peptides; or antibodies selected using in vitro methods, such as phage display, wherein the antibody repertoire is based on a feline immunoglobulin sequence. The term “feline antibody” denotes the genus of sequences that are feline sequences. Thus, the term is not designating the process by which the antibody was created, but the genus of sequences that are relevant.

A “felinized antibody” means an antibody in which at least one amino acid in a portion of a non-feline variable region has been replaced with the corresponding amino acid from a feline variable region. In some embodiments, a felinized antibody comprises at least one feline constant region (e.g., a γ constant region, an a constant region, a δ constant region, an ε constant region, a μ constant region, or etc.) or fragment thereof. In some embodiments, a felinized antibody is an antibody fragment, such as Fab, scFv, (Fab′)₂, etc. The term “felinized” also denotes forms of non-feline (for example, murine) antibodies that are chimeric immunoglobulins, immunoglobulin chains, or fragments thereof (such as Fv, Fab, Fab′, F(ab′)₂ or other antigen-binding sequences of antibodies) that contain minimal sequence of non-feline immunoglobulin. Felinized antibodies can include feline immunoglobulins (recipient antibody) in which residues from a CDR of the recipient are substituted by residues from a CDR of a non-feline species (donor antibody) such as mouse, rat, or rabbit having the desired specificity, affinity, and capacity. In some instances, Fv framework region (FR) residues of the feline immunoglobulin are replaced by corresponding non-feline residues. Furthermore, the felinized antibody can comprise residues that are found neither in the recipient antibody nor in the imported CDR or framework sequences, but are included to further refine and optimize antibody performance.

In some embodiments, at least one amino acid residue in a portion of a mouse variable heavy chain or a mouse variable light chain has been replaced with the corresponding amino acid from a feline variable region. In some embodiments, the modified chain is fused to a feline constant heavy chain or a feline constant light chain. In some embodiments, an anti-IL4R antibody is a felinized antibody comprising a variable heavy chain amino acid sequence of SEQ ID NO: 67, or SEQ ID NO: 69, SEQ ID NO: 365, or SEQ ID NO: 366, and/or a variable light chain amino acid sequence of SEQ ID NO: 68, SEQ ID NO: 70, SEQ ID NO: 367, SEQ ID NO: 368, or SEQ ID NO: 369.

In some embodiments, an anti-IL4R antibody comprises a feline heavy chain constant region selected from an IgG1a, IgG1b, and IgG2 constant region. In some embodiments, an anti-IL4R antibody comprises is a wild-type or variant feline IgG1a, IgG1b, or IgG2 Fc polypeptide, as described herein. In some embodiments, an anti-IL4R antibody comprises a feline IgG1a Fc polypeptide comprising the amino acid sequence of SEQ ID NO: 203 or 204; a feline IgG1b Fc polypeptide comprising the amino acid sequence of SEQ ID NO: 205 or 206; I(c) a feline IgG2 Fc polypeptide comprising the amino acid sequence of SEQ ID NO: 207. In some embodiments, an anti-IL4R antibody comprises a variant feline IgG1a Fc polypeptide comprising the amino acid sequence of SEQ ID NO: 208, 209, 210, 217, 218, 222, or 223; a variant feline IgG1b Fc polypeptide comprising the amino acid sequence of SEQ ID NO: 211, 212, 213, 219, 220, 224, or 225; or (c) a variant feline IgG2 Fc polypeptide comprising the amino acid sequence of SEQ ID NO: 214, 215, 216, 221, or 226.

In some embodiments, an anti-IL4R antibody comprises a feline light chain constant region, such as a feline κ light constant region. In some embodiments, an anti-IL4R antibody comprises is a wild-type feline κ light constant region (e.g., SEQ ID NO: 241) or variant feline κ light constant region (e.g., SEQ ID NO: 242).

In some embodiments, an anti-IL4R antibody comprises a felinized variable heavy chain of Clone B or Clone I and a variant feline IgG Fc polypeptide, such as SEQ ID NO: 79, 80, 82, 372, 373, 83, 374, or 375. In some embodiments, an anti-IL4R antibody comprises a felinized variable light chain of Clone B or Clone I and a feline κ light chain constant region, such as SEQ ID NO: 81, 84, 376, 377, or 378.

In some embodiments, an anti-IL4R antibody is a bispecific antibody having a binding specificity for IL4R and a different target molecule, such as IL17, IL31, TNFα, CD20, CD19, CD25, IL4, IL13, IL23, IgE, CD11α, IL6R, α4-Intergrin, IL12, IL1β, or BlyS. In some embodiments, a bispecific antibody comprises a caninized or felinized Clone B or Clone I variable heavy chain and a “knob” variant canine or feline IgG Fc polypeptide that can pair with a variant κ constant region (e.g., SEQ ID NO: 243). In some embodiments, a bispecific antibody comprises a variable heavy chain directed to a different target molecule (e.g., canine or feline IL31) and a “hole” variant canine or feline IgG Fc polypeptide (e.g., SEQ ID NO: 245). In some embodiments, a bispecific antibody comprises a caninized or felinized Clone B or Clone I variable light chain and a variable κ constant region that can pair with the knob Fc polypeptide (e.g., SEQ ID NO: 244). In some embodiments, a bispecific antibody comprises a variable light chain directed to a different target molecule (e.g., canine or feline IL31) and a wild-type κ constant region that can pair with the hole Fc polypeptide (e.g., SEQ ID NO: 246).

Other bispecific antibody arrangements may be prepared. For example, in some embodiments, a bispecific antibody comprises a caninized or felinized Clone B or Clone I variable heavy chain and a “hole” variant canine or feline IgG Fc polypeptide that can pair with a variant κ constant region. In some embodiments, a bispecific antibody comprises a variable heavy chain directed to a different target molecule (e.g., canine or feline IL31) and a “knob” variant canine or feline IgG Fc polypeptide. In some embodiments, a bispecific antibody comprises a caninized or felinized Clone B or Clone I variable light chain and a variable κ constant region that can pair with the hole Fc polypeptide. In some embodiments, a bispecific antibody comprises a variable light chain directed to a different target molecule (e.g., canine or feline IL31) and a wild-type κ constant region that can pair with the knob Fc polypeptide.

The term “affinity” means the strength of the sum total of noncovalent interactions between a single binding site of a molecule (for example, an antibody) and its binding partner (for example, an antigen). The affinity of a molecule X for its partner Y can generally be represented by the dissociation constant (K_{D}). Affinity can be measured by common methods known in the art, such as, for example, immunoblot, ELISA KD, KinEx A, biolayer interferometry (BLI), or surface plasmon resonance devices.

The terms “K_{D},” “K_{d},” “Kd” or “Kd value” as used interchangeably to refer to the equilibrium dissociation constant of an antibody-antigen interaction. In some embodiments, the K_{d} of the antibody is measured by using biolayer interferometry assays using a biosensor, such as an Octet® System (Pall ForteBio LLC, Fremont, CA) according to the supplier's instructions. Briefly, biotinylated antigen is bound to the sensor tip and the association of antibody is monitored for ninety seconds and the dissociation is monitored for 600 seconds. The buffer for dilutions and binding steps is 20 mM phosphate, 150 mM NaCl, pH 7.2. A buffer only blank curve is subtracted to correct for any drift. The data are fit to a 2:1 binding model using ForteBio data analysis software to determine association rate constant (kₒₙ), dissociation rate constant (k_{off}), and the K_{d}. The equilibrium dissociation constant (K_{d}) is calculated as the ratio of k_{off}/kₒₙ. The term “kon” refers to the rate constant for association of an antibody to an antigen and the term “koff” refers to the rate constant for dissociation of an antibody from the antibody/antigen complex.

The term “binds” to an antigen or epitope is a term that is well understood in the art, and methods to determine such binding are also well known in the art. A molecule is said to exhibit “binding” if it reacts, associates with, or has affinity for a particular cell or substance and the reaction, association, or affinity is detectable by one or more methods known in the art, such as, for example, immunoblot, ELISA KD, KinEx A, biolayer interferometry (BLI), surface plasmon resonance devices, or etc.

“Surface plasmon resonance” denotes an optical phenomenon that allows for the analysis of real-time biospecific interactions by detection of alterations in protein concentrations within a biosensor matrix, for example using the BIAcore™ system (BIAcore International AB, a GE Healthcare company, Uppsala, Sweden and Piscataway, N.J.). For further descriptions, see Jonsson et al. (1993) Ann. Biol. Clin. 51: 19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is an Octet® system (Pall ForteBio LLC). See, e.g., Abdiche et al., 2008, Anal. Biochem. 377: 209-277.

In some embodiments, an anti-IL4R antibody binds to canine IL4R or feline IL4R with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁹ M, less than 1×10⁻¹⁹ M, less than 5×10⁻¹¹ M, less than 1×10⁻¹¹ M, less than 5×10⁻¹² M, or less than 1×10⁻¹² M, as measured by biolayer interferometry. In some embodiments, an anti-IL4R antibody binds to canine IL4R or feline IL4R with a Kd of between 5×10⁻⁶ M and 1×10⁻⁶ M, between 5×10⁻⁶ M and 5×10⁻⁷ M, between 5×10⁻⁶ M and 1×10⁻⁷ M, between 5×10⁻⁶ M and 5×10⁻⁸ M, 5×10⁻⁶ M and 1×10⁻⁸ M, between 5×10⁻⁶ M and 5×10⁻⁹ M, between 5×10⁻⁶ M and 1×10⁻⁹ M, between 5×10⁻⁶ M and 5×10⁻¹⁰ M, between 5×10⁻⁶ M and 1×10⁻¹⁰ M, between 5×10⁻⁶ M and 5×10⁻¹¹ M, between 5×10⁻⁶ M and 1×10⁻¹¹ M, between 5×10⁻⁶ M and 5×10⁻¹² M, between 5×10⁻⁶ M and 1×10⁻¹² M, between 1×10⁻⁶ M and 5×10⁻⁷ M, between 1×10⁻⁶ M and 1×10⁻⁷ M, between 1×10⁻⁶ M and 5×10⁻⁸ M, 1×10⁻⁶ M and 1×10⁻⁸ M, between 1×10⁻⁶ M and 5×10⁻⁹ M, between 1×10⁻⁶ M and 1×10⁻⁹ M, between 1×10⁻⁶ M and 5×10⁻¹⁰ M, between 1×10⁻⁶ M and 1×10⁻¹⁰ M, between 1×10⁻⁶ M and 5×10⁻¹¹ M, between 1×10⁻⁶ M and 1×10⁻¹¹ M, between 1×10⁻⁶ M and 5×10⁻¹² M, between 1×10⁻⁶ M and 1×10⁻¹² M, between 5×10⁻⁷ M and 1×10⁻⁷ M, between 5×10⁻⁷ M and 5×10⁻⁸ M, 5×10⁻⁷ M and 1×10⁻⁸ M, between 5×10⁻⁷ M and 5×10⁻⁹ M, between 5×10⁻⁷ M and 1×10⁻⁹ M, between 5×10⁻⁷ M and 5×10⁻¹⁰ M, between 5×10⁻⁷ M and 1×10⁻¹⁰ M, between 5×10⁻⁷ M and 5×10⁻¹¹ M, between 5×10⁻⁷ M and 1×10⁻¹¹ M, between 5×10⁻⁷ M and 5×10⁻¹² M, between 5×10⁻⁷ M and 1×10⁻¹² M, between 1×10⁻⁷ M and 5×10⁻⁸ M, 1×10⁻⁷ M and 1×10⁻⁸ M, between 1×10⁻⁷ M and 5×10⁻⁹ M, between 1×10⁻⁷ M and 1×10⁻⁹ M, between 1×10⁻⁷ M and 5×10⁻¹⁰ M, between 1×10⁻⁷ M and 1×10⁻¹⁰ M, between 1×10⁻⁷ M and 5×10⁻¹¹ M, between 1×10⁻⁷ M and 1×10⁻¹¹ M, between 1×10⁻⁷ M and 5×10⁻¹² M, between 1×10⁻⁷ M and 1×10⁻¹² M, between 5×10⁻⁸ M and 1×10⁻⁸ M, between 5×10⁻⁸ M and 5×10⁻⁹ M, between 5×10⁻⁸ M and 1×10⁻⁹ M, between 5×10⁻⁸ M and 5×10⁻¹⁰ M, between 5×10⁻⁸ M and 1×10⁻¹⁰ M, between 5×10⁻⁸ M and 5×10⁻¹¹ M, between 5×10⁻⁸ M and 1×10⁻¹¹ M, between 5×10⁻⁸ M and 5×10⁻¹² M, between 5×10⁻⁸ M and 1×10⁻¹² M, 1×10⁻⁸ M and 5×10⁻⁹ M, between 1×10⁻⁸ M and 1×10⁻⁹ M, between 1×10⁻⁸ M and 5×10⁻¹⁰ M, between 1×10⁻⁸ M and 1×10⁻¹⁰ M, between 1×10⁻⁸ M and 5×10⁻¹¹ M, between 1×10⁻⁸ M and 1×10⁻¹¹ M, between 1×10⁻⁸ M and 5×10⁻¹² M, between 1×10⁻⁸ M and 1×10⁻¹² M, between 5×10⁻⁹ M and 1×10⁻⁹ M, between 5×10⁻⁹ M and 5×10⁻¹⁰ M, between 5×10⁻⁹ M and 1×10⁻¹⁰ M, between 5×10⁻⁹ M and 5×10⁻¹¹ M, between 5×10⁻⁹ M and 1×10⁻¹¹ M, between 5×10⁻⁹ M and 5×10⁻¹² M, between 5×10⁻⁹ M and 1×10⁻¹² M, between 1×10⁻⁹ M and 5×10⁻¹⁰ M, between 1×10⁻⁹ M and 1×10⁻¹⁰ M, between 1×10⁻⁹ M and 5×10⁻¹¹ M, between 1×10⁻⁹ M and 1×10⁻¹¹ M, between 1×10⁻⁹ M and 5×10⁻¹² M, between 1×10⁻⁹ M and 1×10⁻¹² M, between 5×10⁻¹⁰ M and 1×10⁻¹⁰ M, between 5×10⁻¹⁰ M and 5×10⁻¹¹ M, between, 1×10⁻¹⁰ M and 5×10⁻¹¹ M, 1×10⁻¹⁰ M and 1×10⁻¹¹ M, between 1×10⁻¹⁰ M and 5×10⁻¹² M, between 1×10⁻¹⁰ M and 1×10⁻¹² M, between 5×10⁻¹¹ M and 1×10⁻¹² M, between 5×10⁻¹¹ M and 5×10⁻¹² M, between 5×10⁻¹¹ M and 1×10⁻¹² M, between 1×10⁻¹¹ M and 5×10⁻¹² M, or between 1×10⁻¹¹ M and 1×10⁻¹² M, as measured by biolayer interferometry. In some embodiments, an anti-IL4R antibody binds to canine IL4R or feline IL4R, as determined by immunoblot analysis.

In some embodiments, an anti-IL4R antibody is provided that competes with an anti-IL4R antibody described herein (such as Clone B or Clone I) for binding to IL4R. In some embodiments, an antibody that competes with binding with any of the antibodies provided herein can be made or used. In some embodiments, an anti-IL4R antibody is provided that competes with monoclonal Clone B or Clone I antibody in binding to canine IL4R or feline IL4R.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters or enhancers) that regulate the expression of the polypeptide of interest, or one or more selectable marker genes (such as, for example, antibiotic resistance genes and genes that can be used in colorimetric assays, for example, (3-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NS0 cells, PER.C6® cells (Crucell), 293 cells, and CHO cells, and their derivatives, such as 293-6E, DG44, CHO-S, and CHO-K cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) encoding an amino acid sequence(s) provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated.” In some embodiments, the anti-IL4R antibody is purified using chromatography, such as size exclusion chromatography, ion exchange chromatography, protein A column chromatography, hydrophobic interaction chromatography, and CHT chromatography.

The term “companion animal species” refers to an animal suitable to be a companion to humans. In some embodiments, a companion animal species is a small mammal, such as a canine, feline, dog, cat, horse, rabbit, ferret, guinea pig, rodent, etc. In some embodiments, a companion animal species is a farm animal, such as a horse, cow, pig, etc.

To “reduce” or “inhibit” means to decrease, reduce, or arrest an activity, function, or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time. A “reference” as used herein, refers to any sample, standard, or level that is used for comparison purposes. A reference may be obtained from a healthy or non-diseased sample. In some examples, a reference is obtained from a non-diseased or non-treated sample of a companion animal. In some examples, a reference is obtained from one or more healthy animals of a particular species, which are not the animal being tested or treated.

The term “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 15% 20%, 25%, 30%, 35%, 40%, 45%, 50%, 60%, 70%, 80%, 90%, or 100% compared to the reference value.

The terms “pharmaceutical formulation” and “pharmaceutical composition” refer to a preparation which is in such form as to permit the biological activity of the active ingredient(s) to be effective, and which contains no additional components that are unacceptably toxic to a subject to which the formulation would be administered.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine.

The pharmaceutical composition can be stored in lyophilized form. Thus, in some embodiments, the preparation process includes a lyophilization step. The lyophilized composition may then be reformulated, typically as an aqueous composition suitable for parenteral administration, prior to administration to the dog, cat, or horse. In other embodiments, particularly where the antibody is highly stable to thermal and oxidative denaturation, the pharmaceutical composition can be stored as a liquid, i.e., as an aqueous composition, which may be administered directly, or with appropriate dilution, to the dog, cat, or horse. A lyophilized composition can be reconstituted with sterile Water for Injection (WFI). Bacteriostatic reagents, such benzyl alcohol, may be included. Thus, the invention provides pharmaceutical compositions in solid or liquid form.

The pH of the pharmaceutical compositions may be in the range of from about pH 5 to about pH 8, when administered. The compositions of the invention are sterile if they are to be used for therapeutic purposes. Sterility can be achieved by any of several means known in the art, including by filtration through sterile filtration membranes (e.g., 0.2 micron membranes). Sterility may be maintained with or without anti-bacterial agents.

The antibodies or pharmaceutical compositions comprising the antibodies of the invention may be useful for treating an IL4/IL13-induced condition. As used herein, an “IL4/IL13-induced condition” means a disease associated with, caused by, or characterized by, elevated levels or altered gradients of IL4/1L13 concentration. Such IL4/IL13-induced conditions include, but are not limited to, a pruritic or an allergic disease. In some embodiments, the IL4/IL13-induced condition is atopic dermatitis, allergic dermatitis, pruritus, asthma, psoriasis, scleroderma, or eczema. An IL4/IL13-induced condition may be exhibited in a companion animal, including, but not limited to, canine or feline.

As used herein, “treatment” is an approach for obtaining beneficial or desired clinical results. “Treatment” as used herein, covers any administration or application of a therapeutic for disease in a mammal, including a companion animal. For purposes of this disclosure, beneficial or desired clinical results include, but are not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In some embodiments, an anti-IL4R antibody or a pharmaceutical composition comprising an anti-IL4R antibody can be utilized in accordance with the methods herein to treat IL4/IL13-induced conditions. In some embodiments, an anti-IL4R antibody or a pharmaceutical composition comprising an anti-IL4R antibody is administered to a companion animal, such as a canine or feline, to treat an IL4/IL13-induced condition.

A “therapeutically effective amount” of a substance/molecule, agonist or antagonist may vary according to factors such as the type of disease to be treated, the disease state, the severity and course of the disease, the type of therapeutic purpose, any previous therapy, the clinical history, the response to prior treatment, the discretion of the attending veterinarian, age, sex, and weight of the animal, and the ability of the substance/molecule, agonist or antagonist to elicit a desired response in the animal. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic or prophylactic result.

In some embodiments, an anti-IL4R antibody or pharmaceutical composition comprising an anti-IL4R antibody is administered parenterally, by subcutaneous administration, intravenous infusion, or intramuscular injection. In some embodiments, an anti-IL4R antibody or pharmaceutical composition comprising an anti-IL4R antibody is administered as a bolus injection or by continuous infusion over a period of time. In some embodiments, an anti-IL4R antibody or pharmaceutical composition comprising an anti-IL4R antibody is administered by an intramuscular, an intraperitoneal, an intracerebrospinal, a subcutaneous, an intra-arterial, an intrasynovial, an intrathecal, or an inhalation route.

Anti-IL4R antibodies described herein may be administered in an amount in the range of 0.01 mg/kg body weight to 100 mg/kg body weight per dose. In some embodiments, anti-IL4R antibodies may be administered in an amount in the range of 0.5 mg/kg body weight to 50 mg/kg body weight per dose. In some embodiments, anti-IL4R antibodies may be administered in an amount in the range of 0.1 mg/kg body weight to 10 mg/kg body weight per dose. In some embodiments, anti-IL4R antibodies may be administered in an amount in the range of 0.1 mg/kg body weight to 100 mg/kg body weight per dose. In some embodiments, anti-IL4R antibodies may be administered in an amount in the range of 1 mg/kg body weight to 10 mg/kg body weight per dose. In some embodiments, anti-IL4R antibodies may be administered in an amount in the range of 0.5 mg/kg body weight to 100 mg/kg body, in the range of 1 mg/kg body weight to 100 mg/kg body weight, in the range of 5 mg/kg body weight to 100 mg/kg body weight, in the range of 10 mg/kg body weight to 100 mg/kg body weight, in the range of 20 mg/kg body weight to 100 mg/kg body weight, in the range of 50 mg/kg body weight to 100 mg/kg body weight, in the range of 1 mg/kg body weight to 10 mg/kg body weight, in the range of 5 mg/kg body weight to 10 mg/kg body weight, in the range of 0.5 mg/kg body weight to 10 mg/kg body weight, in the range of 0.01 mg/kg body weight to 0.5 mg/kg body weight, in the range of 0.01 mg/kg body weight to 0.1 mg/kg body weight, or in the range of 5 mg/kg body weight to 50 mg/kg body weight.

An anti-IL4R antibody or a pharmaceutical composition comprising an anti-IL4R antibody can be administered to a companion animal at one time or over a series of treatments. For example, an anti-IL4R antibody or a pharmaceutical composition comprising an anti-IL4R antibody may be administered at least once, more than once, at least twice, at least three times, at least four times, or at least five times.

In some embodiments, the dose is administered once per week for at least two or three consecutive weeks, and in some embodiments, this cycle of treatment is repeated two or more times, optionally interspersed with one or more weeks of no treatment. In other embodiments, the therapeutically effective dose is administered once per day for two to five consecutive days, and in some embodiments, this cycle of treatment is repeated two or more times, optionally interspersed with one or more days or weeks of no treatment.

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

In some embodiments, the method comprises administering in combination with an anti-IL4R antibody or a pharmaceutical composition comprising an anti-IL4R antibody, a Jak inhibitor, a Tyk2 inhibitor, a PI3K inhibitor, ERK inhibitor. In some embodiments, the method comprises administering in combination with an anti-IL4R antibody or a pharmaceutical composition comprising an anti-IL4R antibody, an anti-IL31 antibody, an anti-IL17 antibody, an anti-TNFα antibody, an anti-CD20 antibody, an anti-CD19 antibody, an anti-CD25 antibody, an anti-IL4 antibody, an anti-IL13 antibody, an anti-IL23 antibody, an anti-IgE antibody, an anti-CD11α antibody, anti-IL6R antibody, anti-α4-Intergrin antibody, an anti-IL12 antibody, an anti-IL1β antibody, or an anti-BlyS antibody.

Provided herein are methods of exposing to a cell an anti-IL4R antibody or a pharmaceutical composition comprising an anti-IL4R antibody under conditions permissive for binding of the antibody to IL4R. In some embodiments, the cell is a canine cell, a feline cell, or an equine cell. In some embodiments, the cell is a canine DH82 cell. In some embodiments, the cell is exposed to the antibody or pharmaceutical composition ex vivo. In some embodiments, the cell is exposed to the antibody or pharmaceutical composition in vivo. In some embodiments, a cell is exposed to the anti-IL4R antibody. In some embodiments, a cell is exposed to the anti-IL4R antibody or the pharmaceutical composition under conditions permissive for binding of the antibody to extracellular IL4R. In some embodiments, a cell may be exposed in vivo to the anti-IL4R antibody or the pharmaceutical composition by any one or more of the administration methods described herein, including but not limited to, intraperitoneal, intramuscular, intravenous injection into the subject. In some embodiments, a cell may be exposed ex vivo to the anti-IL4R antibody or the pharmaceutical composition by exposing the cell to a culture medium comprising the antibody or the pharmaceutical composition. In some embodiments, the permeability of the cell membrane may be affected by the use of any number of methods understood by those of skill in the art (such as electroporating the cells or exposing the cells to a solution containing calcium chloride) before exposing the cell to a culture medium comprising the antibody or the pharmaceutical composition.

Provided herein are methods of using the anti-IL4R antibodies, polypeptides and polynucleotides for detection, diagnosis and monitoring of an IL4R-induced condition. Provided herein are methods of determining whether a companion animal will respond to anti-IL4R antibody therapy. In some embodiments, the method comprises detecting whether the animal has cells that express IL4R using an anti-IL4R antibody. In some embodiments, the method of detection comprises contacting the sample with an antibody, polypeptide, or polynucleotide and determining whether the level of binding differs from that of a reference or comparison sample (such as a control). In some embodiments, the method may be useful to determine whether the antibodies or polypeptides described herein are an appropriate treatment for the subject animal.

In some embodiments, the sample is a biological sample. The term “biological sample” means a quantity of a substance from a living thing or formerly living thing. In some embodiments, the biological sample is a cell or cell/tissue lysate. In some embodiments, the biological sample includes, but is not limited to, blood, (for example, whole blood), plasma, serum, urine, synovial fluid, and epithelial cells.

In some embodiments, the cells or cell/tissue lysate are contacted with an anti-IL4R antibody and the binding between the antibody and the cell is determined. When the test cells show binding activity as compared to a reference cell of the same tissue type, it may indicate that the subject would benefit from treatment with an anti-IL4R antibody. In some embodiments, the test cells are from tissue of a companion animal.

Various methods known in the art for detecting specific antibody-antigen binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Appropriate labels include, without limitation, radionuclides (for example ¹²⁵I, ¹³¹I, ³⁵S, ³H, or ³²P), enzymes (for example, alkaline phosphatase, horseradish peroxidase, luciferase, or β-galactosidase), fluorescent moieties or proteins (for example, fluorescein, rhodamine, phycoerythrin, GFP, or BFP), or luminescent moieties (for example, Qdot™ nanoparticles supplied by the Quantum Dot Corporation, Palo Alto, Calif.). General techniques to be used in performing the various immunoassays noted above are known to those of ordinary skill in the art.

For purposes of diagnosis, the polypeptide including antibodies can be labeled with a detectable moiety including but not limited to radioisotopes, fluorescent labels, and various enzyme-substrate labels know in the art. Methods of conjugating labels to an antibody are known in the art. In some embodiments, the anti-IL4R antibodies need not be labeled, and the presence thereof can be detected using a second labeled antibody which binds to the first anti-IL4R antibody. In some embodiments, the anti-IL4R antibody can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987). The anti-IL4R antibodies and polypeptides can also be used for in vivo diagnostic assays, such as in vivo imaging. Generally, the antibody or the polypeptide is labeled with a radionuclide (such as ¹¹¹In, ⁹⁹Tc, ¹⁴C, ¹³¹I, ¹²⁵I, ³H, or any other radionuclide label, including those outlined herein) so that the cells or tissue of interest can be localized using immunoscintiography. The antibody may also be used as staining reagent in pathology using techniques well known in the art.

In some embodiments, a first antibody is used for a diagnostic and a second antibody is used as a therapeutic. In some embodiments, the first and second antibodies are different. In some embodiments, the first and second antibodies can both bind to the antigen at the same time, by binding to separate epitopes.

Provided herein are methods for screening for a molecule that inhibits IL4 and/or IL13 signaling function comprising exposing to a canine DH82 cell the molecule and detecting whether there is a reduction in STAT6 phosphorylation. In some embodiments, the molecule comprises an anti-IL4R antibody or small molecule antagonist of IL4R. In some embodiments, the molecule comprises an anti-IL4R antibody or small molecule antagonist of IL13R. In some embodiments, the molecule comprises an anti-IL4R antibody or small molecule antagonist of IL4. In some embodiments, the molecule comprises an anti-IL4R antibody or small molecule antagonist of IL13.

The following examples illustrate particular aspects of the disclosure and are not intended in any way to limit the disclosure.

### EXAMPLES

### Example 1

### Preparation of IL4 and IL4R ECD Reagents

Nucleotide sequences encoding fusion proteins comprising (1) either full length canine IL4R (SEQ ID NO: 94), a canine, feline, equine, murine, or human IL4R ECDs (SEQ ID NO: 99, 100, 101, 102, or 103), or a canine, feline, or equine IL4 (SEQ ID NOs: 121, 122, or 123), (2) one or more His6, human Fc, and/or FLAG tag, (3) one or more linker sequences, and (4) a leader sequence were synthesized and cloned into separate mammalian expression plasmids. The plasmids were separately transfected into 293 cells, cultured, and supernatants containing secreted IL4R ECD or IL4 fusion polypeptides were separately collected and filtered. The poly-His fusion proteins were affinity purified using Ni-NTA column (GE Healthcare Life Sciences) and human Fc fusion proteins were affinity purified using CaptivA® Protein A Affinity Resin (Repligen). The purified fusion proteins were confirmed by SDS-PAGE analysis (data not shown). The fusion proteins (before and after processing) are summarized in Table 3, below.

TABLE 3

| SEQ ID NO: | Description |
|---|---|
| 104 | Canine IL4R_C-FLAG (with leader) |
| 105 | Canine IL4R_C-FLAG (processed) |
| 106 | Canine IL4R-ECD_C-His6 (with leader) |
| 107 | Canine IL4R-ECD_C-His6 (processed) |
| 108 | Canine IL4R-ECD_C-HuFc_His6 (with leader) |
| 109 | Canine IL4R-ECD_C-HuFc_His6 (processed) |
| 110 | Feline IL4R-ECD_C-HuFc_His6 (with leader) |
| 111 | Feline IL4R-ECD_C-HuFc_His6 (processed) |
| 112 | Equine IL4R-ECD_C-HuFc_His6 (with leader) |
| 113 | Equine IL4R-ECD_C-HuFc_His6 (processed) |
| 114 | Murine IL4R-ECD_C-HuFc_His6 (with leader) |
| 115 | Murine IL4R-ECD_C-HuFc_His6 (processed) |
| 116 | Human IL4R-ECD_C-HuFc_His6 (with leader) |
| 117 | Human IL4R-ECD_C-HuFc_His6 (processed) |
| 124 | Canine IL4_N-His6 (with leader) |
| 125 | Canine IL4_N-His6 (processed) |
| 126 | Canine IL4_C-His6 (with leader) |
| 127 | Canine IL4_C-His6 (processed) |
| 128 | Feline IL4_C-His6 (with leader) |
| 129 | Feline IL4_C-His6 (processed) |
| 130 | Equine IL4_C-His6 (with leader) |
| 131 | Equine IL4_C-His6 (processed) |
| 156 | Canine IL13_C-His6 (with leader) |
| 157 | Canine IL13_C-His6 (processed) |
| 158 | Feline IL13_C-His6 (with leader) |
| 159 | Feline IL13_C-His6 (processed) |

### Example 2

### Identification of Mouse Monoclonal Antibodies that Bind to Canine IL4R

Mouse monoclonal antibodies were identified following standard immunization with purified canine IL4R-ECD_C-His6 (SEQ ID NO: 107) as immunogen. Different adjuvants were used during immunizations (Akesobio, Inc, China) and monoclonal antibodies were obtained through standard hybridoma technology.

Enzyme linked immunosorbent assay (ELISA) was developed to screen for clones that produce IL4R binding antibodies. First, biotinylated IL4R-ECD_C-His6 (SEQ ID NO: 107) was introduced into streptavidin-coated wells Immunized serum was then added to the wells followed by washing and detection with HRP-conjugated anti-mouse antibodies. The presence of canine IL4R binding antibodies developed a positive signal. Over 121 ELISA-positive top clones were identified.

The 121 antibody clones were screened for the ability to block interaction between canine IL4 and canine IL4R ECD by ELISA. Canine IL4R-ECD_C-HuFc_His6 (SEQ ID NO: 109) was immobilized to wells coated with anti-human Fc. Hybridoma supernatant was added, followed by biotinylated canine IL4_C-His6 (SEQ ID NO: 127), and then Streptavidin-HRP. Diminished signal suggested reduced interaction between canine IL4R ECD and canine IL4. Eleven clones were identified and designated as Clones A, B, C, D, E, F, G, H, I, J, and K. Each of the clones was further cultured and the IgG antibodies produced were purified using standard Protein A affinity chromatography.

The binding of each clone to canine IL4R ECD was confirmed by biosensor assay (Forte Bio Octet). First, biotinylated canine IL4R-ECD_C-His6 (SEQ ID NO: 107) was bound to streptavidin sensor tips. Then, binding of each of the eleven antibody clones to the canine IL4R-ECD-bound sensor tip was assessed.

### Example 3

### Clone B and Clone I Antibodies Block Binding of IL4 to Canine IL4R

The eleven antibodies were evaluated by biosensor assay (Forte Bio Octet) for the ability of an antibody-IL4R ECD complex to reduce binding of ligand canine IL4. Biotinylated canine IL4R-ECD_C-His6 (SEQ ID NO: 107) was captured on streptavidin sensor tips. IL4R ECD-bound tips were separately exposed to each of the eleven murine antibodies (Clones A, B, C, D, E, F, G, H, I, J, and K) at 20 μg/mL to form IL4R ECD-antibody binary complexes. The complex-bound tips were then exposed to canine IL4_C-His6 (SEQ ID NO: 127) at a high concentration (240 μg/mL). Canine IL4R ECD-antibody complexes of Clones B and I failed to bind to canine IL4, suggesting that both Clones B and I are neutralizing antibodies.

### Example 4

### Identification of DNA Sequences Encoding VH and VL of Monoclonal Antibodies

Hybridoma Clones B and I were pelleted, and total RNA was extracted. Oligonucleotide primers for amplifying mouse immunoglobulin (Ig) variable domains were used to obtain cDNA using standard techniques. The heavy and light chains of each clone were sequenced and analyzed by sequence alignment (FIG. 1A and FIG. 1B, respectively). Exemplary CDR sequences of Clone B were identified as SEQ ID NOs 7-9 and 14-16 and of Clone I were identified as SEQ ID NOs 29-31 and 36-38. Exemplary consensus CDR sequences were identified as CDR-H1: GYTFTSYVMH (SEQ ID NO: 1), CDR-H2: YINPX₁NDGTFYNGX₂X₃X₄G (SEQ ID NO: 2), wherein X₁ is K or A, X₂ K or A, X₃ is F or V, and X₄ is K or Q, or YINPX₁NDGT, wherein X₁ is K or A (SEQ ID NO: 268); CDR-H3: FX₅YGX₆AY (SEQ ID NO: 3), wherein X₅ is N or Y, and X₆ s I or F, CDR-L1: RASQEISGYLS (SEQ ID NO: 4); CDR-L2: AASX₇X₈DX₉ (SEQ ID NO: 5), wherein X₇ is T or N, X₈ is R or L, and X₉ is S or T; and CDR-L3: VQYASYPWT (SEQ ID NO: 6).

### Example 5

### Expression and Purification of Anti-IL4R-mAb Clone B and I

Nucleotide sequences encoding full length Clone B and I heavy and light chain polypeptides with leader sequences (SEQ ID NOs 27, 28, 49, and 50) were chemically synthesized and cloned into separate expression vectors suitable for transfection into a CHO host cell. Clone B and Clone I vectors were transfected into separate CHO host cells and cultured. Clone B and Clone I antibodies were purified from the culture medium by single step Protein A column chromatography.

Thermostability of Clone B and I antibodies as a function of pH was measured by differential scanning fluorimetry (DSF). The melting temperature (Tm) of each antibody at the different pHs is listed in Table 4, below. Buffer and 12 μg of antibody were mixed together with 1× Protein thermal shift dye (Applied Biosystem, Catalog No. 4461146). A melting curve was performed with StepOne Real Time PCR System (Applied Biosystem, Catalog No. 4376357). The temperature was increased from 25° C. to 99° C. with a ramp rate of 1% according to the manufacturer's instructions. The data was analyzed by Protein Thermal Shift™ Software v1.0 (Applied Biosystem, Catalog No. 4466038) to determine the Tm, which was calculated as the highest value derived from taking the first derivative of the protein melting curve.

TABLE 4

| pH Tested | Assay buffer | Clone B Tm (° C.) | Clone I Tm (° C.) |
|---|---|---|---|
| 4.5 | 0.1M NaAc | 58.5 | 59.2 |
| 6 | 0.1M NaPO₄ | 65.1 | 65.6 |
| 7.5 | 0.1M NaPO₄ | 66.5 | 67.8 |
| 9 | 0.1M TrisHCl | 66.7 | 67.1 |

### Example 6

### Demonstration of Canine IL4R Binding Activity

Clone B and I antibodies each exhibited affinity to canine IL4R with kinetics potentially sufficient for therapeutic activity. The binding analysis was performed using an Octet Biosensor as follows. Briefly, canine IL4R-ECD_C-His6 (SEQ ID NO: 107) was biotinylated through amine chemistry. The free unreacted biotin was removed by extensive dialysis. Biotinylated canine IL4R-ECD_C-His6 was captured on streptavidin sensor tips. The association of either Clone B or I antibody and canine IL4R-ECD_C-His6 (25 μg/mL) was monitored for 600 seconds. Dissociation was monitored for 600 seconds. A buffer only blank curve was subtracted to correct for any drift. The data were fit to a 1:1 binding model using ForteBio™ data analysis software to determine the kₒₙ, k_{off}, and the Kd. The buffer for dilutions and all binding steps was 20 mM phosphate, 150 mM NaCl, pH 7.2. The Kd of Clone B antibody and canine IL4R-ECD_C-His6 was 2.03×10⁻⁹ M and of Clone I antibody and canine IL4R-ECD_C-His6 was 1.79×10⁻⁹ M.

An alternative binding assay was performed also using an Octet Biosensor. Canine IL4R-ECD_C-HuFc_His6 (SEQ ID NO: 109) was captured on anti-human Fc-bound sensor tips. The association of either Clone B or I antibody and canine IL4R-ECD_C-HuFc_His6 was monitored for 600 seconds. Dissociation was monitored for 600 seconds. The buffer for dilutions and all binding steps was 20 mM phosphate, 150 mM NaCl, pH 7.2. The Kd of Clone B antibody and canine IL4R-ECD_C-HuFc_His6 was about 10⁻¹⁰ M and of Clone I antibody and canine IL4R-ECD_C-HuFc_His6 was 2.75×10⁻¹⁰ M. The increased affinity observed with the second assay may be due to increased avidity of Clone B and I antibodies for canine IL4R-ECD_C-HuFc_His6 over canine IL4R-ECD_C-His6. In addition, amine conjugation may affect the affinity of canine IL4R-ECD_C-His6 to interact with Clone B and I antibodies.

### Example 7

### Clone B and I Antibodies Compete for the Same IL4R Epitope Group

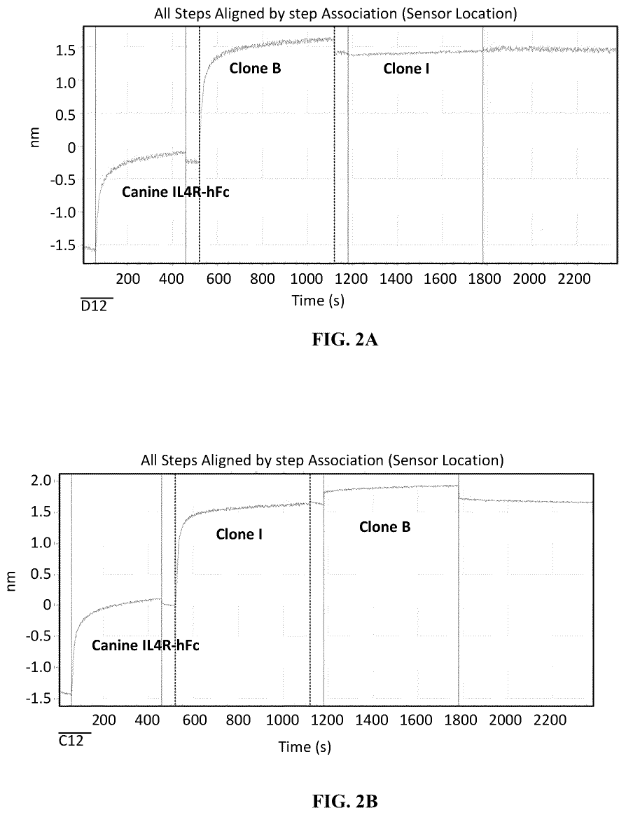

Epitope binding analysis was performed using an Octet Biosensor. Canine IL4R-ECD_C-HuFc_His6 (SEQ ID NO: 109) was captured on anti-human Fc-bound sensor tips. The association of Clone B antibody (50 μg/mL) and canine IL4R-ECD_C-HuFc_His6 was monitored for 600 seconds. The complex-bound tips were washed briefly and then exposed to Clone I antibody (50 μg/mL). After the wash step and exposure to Clone I antibody, no further association for canine IL4R-ECD_C-HuFc_His6 was observed (FIG. 2A), suggesting that Clone B and I antibodies bind to same epitope group. The opposite binding assay was also performed. The association between Clone I antibody (50 μg/mL) and canine IL4R-ECD_C-HuFc_His6 captured on anti-human Fc-bound sensor tips was monitored for 600 seconds. The complex-bound tips were washed briefly and then exposed to Clone B antibody (50 μg/mL). After the wash step and exposure to Clone B antibody, no further association for canine IL4R-ECD_C-HuFc_His6 was observed (FIG. 2B), again suggesting that Clone B and Clone I antibodies bind to same epitope group.

### Example 8

### Clone B and Clone I Antibodies Block IL4 and IL13 Binding to IL4R

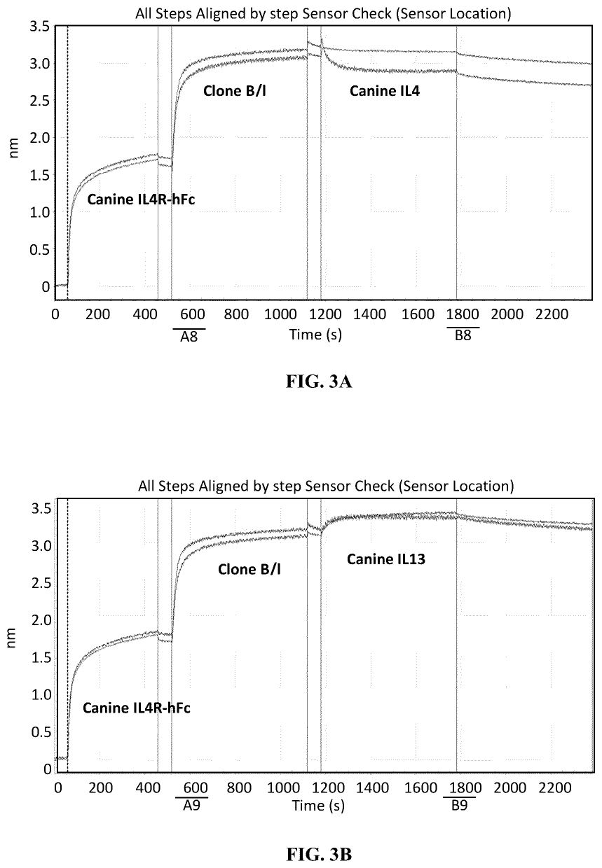

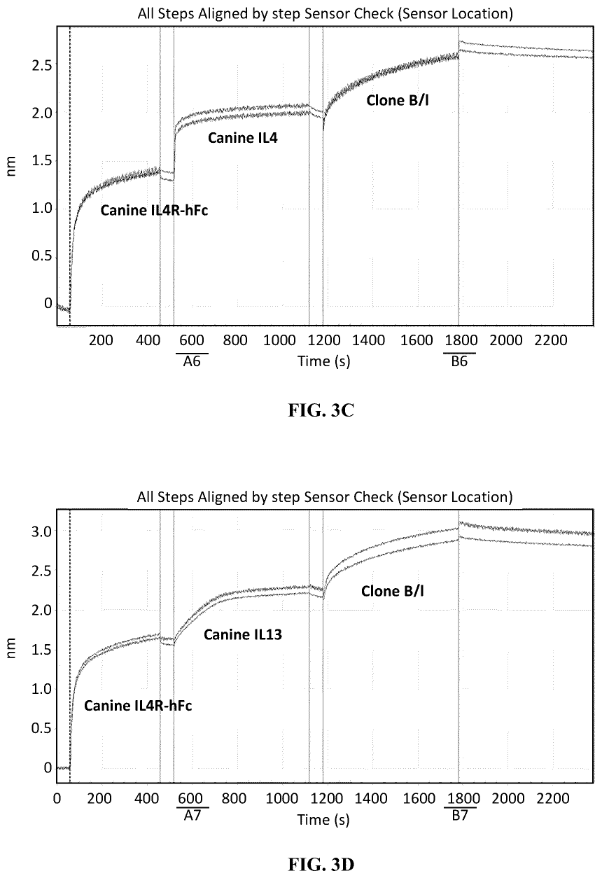

Various binding analyses of Clone B, Clone I, canine IL4, and canine IL13 ligands to canine IL4R were performed using an Octet Biosensor. Canine IL4R-ECD_C-HuFc_His6 (SEQ ID NO: 109) was captured on anti-human Fc-bound sensor tips. The association of Clone B or Clone I antibody (25 μg/mL) and canine IL4R-ECD_C-HuFc_His6 was monitored for 600 seconds. The complex-bound tips were washed briefly and then exposed to canine IL4_C-His6 (SEQ ID NO: 127; 50 μg/mL) or canine IL13_C-His6 (SEQ ID NO: 157; 50 μg/mL) and monitored for 600 seconds. Little to no binding of canine IL4 (FIG. 3A) or canine IL13 (FIG. 3B) was observed, suggesting that Clone B and I antibodies block the binding of canine IL4 and canine IL13 to canine IL4R.

The opposite binding assay was also performed. The association between canine IL4_C-His6 (SEQ ID NO: 127; 50 μg/mL) or canine IL13_C-His6 (SEQ ID NO: 157; 50 μg/mL) and canine IL4R-ECD_C-HuFc_His6 captured on anti-human Fc-bound sensor tips was monitored for 600 seconds. The complex-bound tips were washed briefly and then exposed to Clone B antibody (50 μg/mL) or Clone I antibody (50 μg/mL) and monitored for 600 seconds. Following IL4 and IL13 binding to IL4R (FIG. 3C and FIG. 3D, respectively), further association with Clone B or Clone I was observed. These results suggest that Clone B and Clone I each have a higher affinity for IL4R than either canine IL4 or canine IL13.

### Example 9

### Immunoreactivity of Clone B and I Antibodies to IL4R by Western Analysis

The ability of Clone B and I antibodies to recognize canine IL4R-ECD_C-HuFc_His6 (SEQ ID NO: 109) by Western blot was investigated. Purified canine IL4R-ECD_C-HuFc_His6 was separated by SDS-PAGE under reducing conditions (in the presence of DTT) or non-reducing conditions (absence of DTT). The proteins were transferred to a PVDF membrane and probed using either Clone B or I antibody followed by goat anti-mouse IgG-HRP. Immunoreactive positive signals with Clone B and I antibodies were only observed with samples under non-reducing conditions, suggesting that disulfide binding may be important for maintaining epitope conformation and that the epitope for Clone B and I antibodies may be discontinuous or conformational.

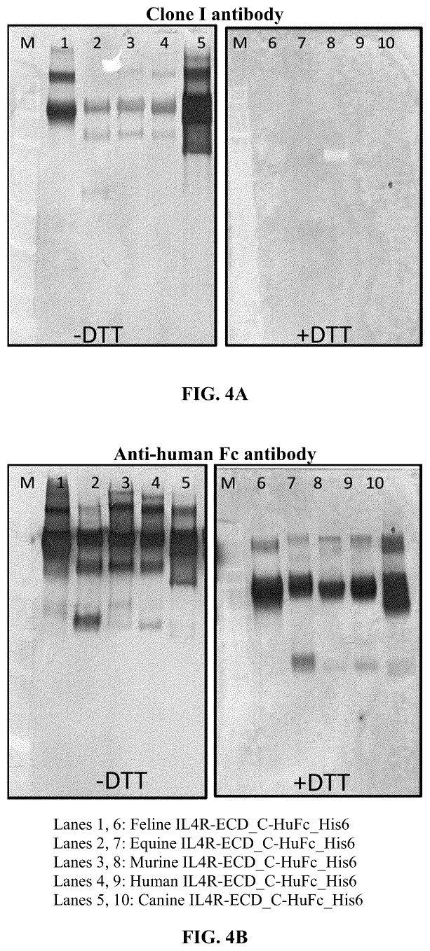

Cross-reactivity of Clone I antibody to feline, equine, murine, and human IL4R was also investigated. Canine IL4R-ECD_C-HuFc_His6 (SEQ ID NO: 109), feline IL4R-ECD_C-HuFc_His6 (SEQ ID NO: 111), equine IL4R-ECD_C-HuFc_His6 (SEQ ID NO: 113), murine IL4R-ECD_C-HuFc_His6 (SEQ ID NO: 115), and human IL4R-ECD_C-HuFc_His6 (SEQ ID NO: 117) (0.1 μg/lane) were each separated by SDS-PAGE under reducing (+DTT) or non-reducing (−DTT) conditions. The proteins were transferred to PVDF membranes and the blots were probed with Clone I antibody (0.3 μg/mL) and visualized by goat anti-mouse IgG-HRP (FIG. 4A). As a control, the blot was stripped and probed with goat anti-human IgG Fc-HRP to visualize the presence of the IL4R-ECD proteins (FIG. 4B). Clone I antibody immunoreacted with canine IL4R-ECD_C-HuFc_His6 and to a lesser extent with feline IL4R-ECD_C-HuFc_His6 under non-reducing conditions (FIG. 4A, lanes 5 and 1, respectively). Low background reactivity was observed with equine, murine, and human IL4R-ECD_C-HuFc_His6 fusion polypeptides (FIG. 4A, lanes 2, 3, and 4, respectively).

### Example 10

### Feline IL4R Binding Affinity

The immunoblot assay detected slightly reduced binding between Clone I and feline IL4R-ECD_C-HuFc_His6 compared to binding between Clone I and canine IL4R-ECD_C-HuFc_His6. This finding was consistent with in vitro binding affinity measured by Octet Biosensor. Biotinylated feline IL4R-ECD_C-HuFc_His6 (SEQ ID NO: 111) was captured on streptavidin sensor tips. The association of Clone I antibody (25 μg/mL) and feline IL4R-ECD_C-HuFc_His6 was monitored for 600 seconds. Dissociation was monitored for 600 seconds. A buffer only blank curve was subtracted to correct for any drift. The data were fit to a 1:1 binding model using ForteBio™ data analysis software to determine the kₒₙ, k_{off}, and the Kd. The buffer for dilutions and all binding steps was 20 mM phosphate, 150 mM NaCl, pH 7.2. The Kd of Clone I antibody and feline IL4R-ECD_C-HuFc_His6 was 1.1×10⁻⁹ M.

### Example 11

### Identification of Canine IL4R Binding Epitope for Clone I Antibody

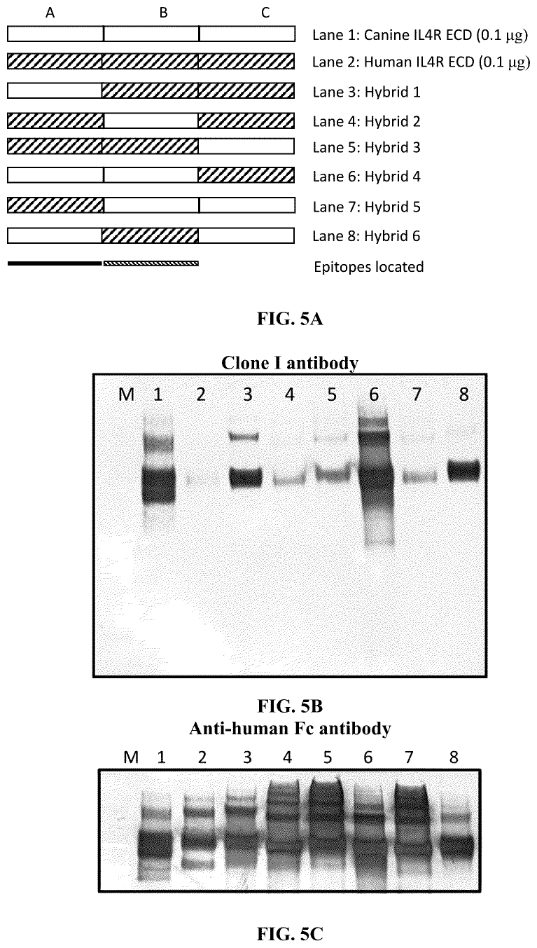

The canine IL4R epitope that is recognized by Clone I antibody (and presumably also by Clone B antibody) was investigated. Since Clone I antibody exhibited a low background of cross reactivity with human IL4R-ECD, numerous hybrid proteins of canine IL4R ECD (SEQ ID NO: 99) and human IL4R ECD (SEQ ID NO: 103) sequences were designed with a leader sequence (SEQ ID NO: 132) and a C-terminal human Fc-His6 tag to facilitate canine IL4R epitope mapping. Canine and human ECD sequences were divided into three segments (A, B, and C) and six different hybrid polypeptide constructs were prepared based on those segments in the order of A to C (see FIG. 5A and Table 5, below).

TABLE 5

| | SEQ | | | |
|---|---|---|---|---|
| Hybrid | ID | Canine IL4R ECD | Human IL4R ECD | FIG. 4 |
| IL4R | NO: | segment(s) | segment(s) | Lane |
| Hybrid 1 | 133 | A (G1-N55) | B and C (N56-H209) | 3 |
| Hybrid 2 | 134 | B (R56-H109) | A and C | 4 |
| | | | (G1-N55, T110-H209) | |
| Hybrid 3 | 135 | C (P110-P204) | A and B (G1-H109) | 5 |
| Hybrid 4 | 136 | A and B (G1-H109) | C (T110-H209) | 6 |
| Hybrid 5 | 137 | B and C (R56-P204) | A (G1-N55) | 7 |
| Hybrid 6 | 138 | A and C (G1-N55, | B (N56-H109) | 8 |
| | | P110-P204) | | |

Plasmid constructs containing nucleotide sequences encoding each of Hybrid 1-6 polypeptides were transiently transfected into 293 cells and the supernatants concentrated 3-fold. Each fusion polypeptide was separated by SDS-PAGE under non-reducing (−DTT) conditions and the proteins transferred to a PVDF membrane. The blot was probed using Clone I antibody (FIG. 5B) or anti-human Fc antibody as a control (FIG. 5C). The presence of both canine IL4R ECD segments A and B gave the strongest signal (Hybrid 4, FIG. 5B, lane 6). Canine IL4R ECD segment A alone (Hybrid 1, FIG. 5B, lane 3) or with segment C (Hybrid 6, FIG. 5B, lane 8) gave appreciable signal, suggesting that segment A may contain the major epitope. Whereas segment B alone (Hybrid 2, FIG. 5B, lane 4) or with segment C (Hybrid 5, FIG. 5B, lane 7) gave a weaker signal, suggesting that segment B may contain an accessary (or minor) epitope.

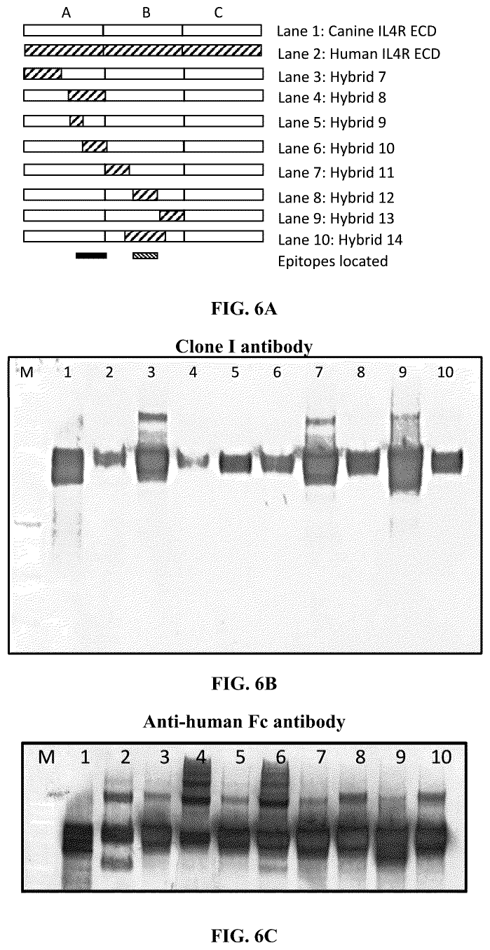

Based on this information, additional hybrid proteins of canine IL4R ECD (SEQ ID NO: 99) and human IL4R ECD (SEQ ID NO: 103) sequences were designed with a leader sequence (SEQ ID NO: 132) and C-terminal human Fc-His6 tag to further localize the canine IL4R epitope(s). Segments A and B of canine and human ECD sequences were further divided and an additional eight hybrid polypeptide constructs (Hybrids 7-14) were prepared based on increasing amino acid residue number of either canine or human sequences (see FIG. 6A and Table 6, below).

TABLE 6

| | SEQ | | | |
|---|---|---|---|---|
| Hybrid | ID | Canine IL4R ECD | Human IL4R | FIG. 5 |
| IL4R | NO: | segment(s) | ECD segment(s) | Lane |
| Hybrid 7 | 139 | C31-P204 | G1-N30 | 3 |
| Hybrid 8 | 140 | G1-N30 and R56-P204 | C31-N55 | 4 |
| Hybrid 9 | 141 | G1-N30 and D42-P204 | C31-L41 | 5 |
| Hybrid 10 | 142 | G1-L41 and R56-P204 | V42-N55 | 6 |
| Hybrid 11 | 143 | G1-N55 and E72-P204 | N56-V71 | 7 |
| Hybrid 12 | 144 | G1-V71 and G90-P204 | S72-K89 | 8 |
| Hybrid 13 | 145 | G1-S89 and P110-P204 | G90-H109 | 9 |
| Hybrid 14 | 146 | G1-I67 and S95-P204 | D68-P94 | 10 |

Plasmid constructs containing nucleotide sequences encoding each of Hybrid 7-14 polypeptides were transiently transfected into 293 cells and the supernatants concentrated 3-fold. Each hybrid polypeptide was separated by SDS-PAGE under non-reducing (— DTT) conditions and the proteins transferred to a PVDF membrane. The blot was probed using Clone I antibody (FIG. 6B) or anti-human Fc antibody as a control (FIG. 6C). The presence of both the C-terminal half of segment A and the central part of segment B gave the strongest signals (FIG. 6B; Hybrids 7, 11, and 13; lanes 3, 7, and 9, respectively).

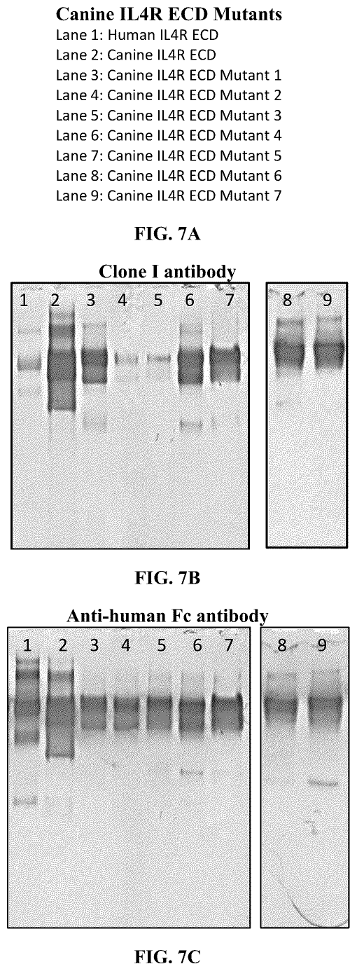

To further identify amino acid residues of the canine IL4R epitopes recognized by Clone I, multiple mutant canine IL4 ECD sequences carrying alanine mutations based on SEQ ID NO: 99 were designed with a leader sequence (SEQ ID NO: 132) and a C-terminal human Fc-His6 tag and expressed in 293 cells. The cell supernatants were concentrated 3-fold, separated by SDS-PAGE under non-reducing (— DTT) conditions, and transferred to a PVDF membrane. The blot was probed using Clone I antibody (FIG. 7B) or anti-human Fc antibody as a control (FIG. 7C). The results of the fine epitope mapping are summarized in Table 7, below. The results suggest that amino acids M44 and G45 of canine IL4R ECD (SEQ ID NO: 95) are involved in epitope binding.

TABLE 7

| Mutant | FIG. 7 | SEQ | Canine IL4R ECD | Was a decreased |
|---|---|---|---|---|
| IL4R | Lane | ID NO: | Substitution | signal observed? |
| Mutant 1 | 3 | 147 | A33T | No |
| Mutant 2 | 4 | 148 | M44A | Yes |
| Mutant 3 | 5 | 149 | G45A | Yes |
| Mutant 4 | 6 | 150 | N48A | No |
| Mutant 5 | 7 | 151 | S38A | No |
| Mutant 6 | 8 | 152 | D42A | No |
| Mutant 7 | 9 | 153 | H49A | No |

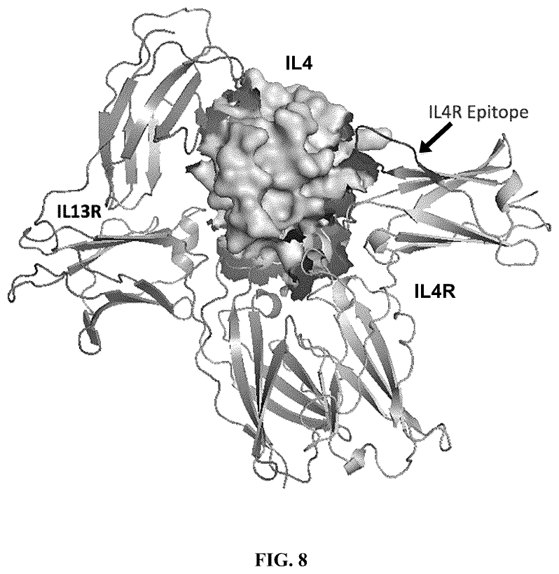

A three-dimensional model of a complex of canine IL4 (SEQ ID NO: 121), canine IL4R ECD (SEQ ID NO: 99) and canine IL13R ECD (SEQ ID NO: 161) was constructed (FIG. 8). Canine IL4R epitope 1 is identified in FIG. 8. Analysis of the study results described above, and three-dimensional protein modeling analysis suggests that Clones B and I bind to a first epitope residing within L41 and T50 of canine IL4R ECD (SEQ ID NO: 99) or feline IL4R ECD (SEQ ID NO: 100), such as within R36 and N55. For example, the first epitope may comprise the amino acid sequence of SEQ ID NO: 88, SEQ ID NO: 89, SEQ ID NO: 91, or SEQ ID NO: 92. In some embodiments, the first epitope comprises the amino acid sequence LX₁₀FMGSENX₁₁T, wherein X₁₀ is D or N and x₁₁ is H or R (SEQ ID NO: 85). In some embodiments, the first epitope comprises the amino acid sequence RLSYQLX₁₀FMGSENX₁₁TCVPEN, wherein X₁₀ is D or N and x₁₁ is H or R (SEQ ID NO: 86).

Analysis of the study results and three-dimensional protein modeling also suggests that Clones B and I bind to a second epitope within amino acids S64 and Q85 of canine IL4R ECD (SEQ ID NO: 99) or feline IL4R ECD (SEQ ID NO: 100). For example, the second epitope may comprise the amino acid sequence of SEQ ID NO: 90 or SEQ ID NO: 93. In some embodiments, the second epitope comprises the amino acid sequence SMX₁₂X₁₃DDX₁₄VEADVYQLX₁₅LWAGXQ, wherein X₁₂ is P or L, X₁₃ is I or M, X₁₄ is A or F, X₁₅ is D or H, and X₁₆ is Q or T (SEQ ID NO: 87).

### Example 12

### Expression and Purification of Chimeric, Caninized, and Felinized Antibodies

Clone B or I variable heavy chain polypeptides may be fused to the heavy chain constant region of a different animal species, such as a wild-type canine IgG-A, IgG-B, IgG-C, or IgG-D or a wild-type feline IgG-1a, IgG-1b, or IgG-2 Fc polypeptide (e.g., IgG Fc polypeptides comprising SEQ ID NO: 162, 163, 164, 165, 166, 167, 203, 204, 205, 206, 207, 227, 228, 229, 230, 237, 238, 239, or 240), or variants of such IgG Fc polypeptides (e.g., variant IgG Fc polypeptides comprising SEQ ID NO: 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 208, 209, 210, 211, 212, 213, 214, 215, 216, 217, 218, 219, 220, 221, 222, 223, 224, 225, 226, 231, 232, 233, 234, 239, 240, 381, 382, 383, 384, 385, 386, 387, 388, 389, 390, 391, 392, 393, or 394). Exemplary amino acid sequences of chimeric heavy chains include SEQ ID NO: 51 (Clone B variable HC and canine IgG-B) and SEQ ID NO: 55 (Clone I variable HC and canine IgG-B). In addition, Clone B or I variable light chain polypeptides may be fused to the light chain constant region of a companion animal species, such as a wild-type canine or feline light chain constant region (e.g., SEQ ID NO: 235 or 241), or variants of such polypeptides (e.g., SEQ ID NO: 236 or 242). Exemplary amino acid sequences of chimeric light chains include SEQ ID NO: 52 (Clone B variable LC and canine κ light chain constant region) and SEQ ID NO: 56 (Clone I variable LC and canine κ light chain constant region).

Clone B and I variable heavy and variable light chains were caninized and felinized by searching and selecting proper canine and feline germline antibody amino acid sequences as a template for CDR grafting. The sequences were further optimized using 3-dimensional structural modeling. Examples of caninized and felinized variants of Clone B and I variable heavy and variable light chain polypeptides that were designed include SEQ ID NO 59, SEQ ID NO: 60, SEQ ID NO: 61, SEQ ID NO: 62, SEQ ID NO: 63, SEQ ID NO: 64, SEQ ID NO: 274, SEQ ID NO: 363, SEQ ID NO: 65, SEQ ID NO: 66, SEQ ID NO: 275, SEQ ID NO: 364, SEQ ID NO: 67, SEQ ID NO: 68, SEQ ID NO: 69, and SEQ ID NO: 70.

Caninized or felinized heavy chain polypeptides may be fused to the heavy chain constant region of a wild-type canine IgG-A, IgG-B, IgG-C, or IgG-D or a wild-type feline IgG-1a, IgG-1b, or IgG-2 Fc polypeptide (e.g., IgG Fc polypeptides comprising SEQ ID NO: 162, 163, 164, 165, 166, 167, 203, 204, 205, 206, 207, 227, 228, 229, 230, 237, 238, 239, or 240), or variants of such IgG Fc polypeptides (e.g., variant IgG Fc polypeptides comprising SEQ ID NO: 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 208, 209, 210, 211, 212, 213, 214, 215, 216, 217, 218, 219, 220, 221, 222, 223, 224, 225, 226, 231, 232, 233, 234, 239, 240, 381, 382, 383, 384, 385, 386, 387, 388, 389, 390, 391, 392, 393, or 394). Exemplary amino acid sequences of a caninized or felinized variable heavy chain and a variant IgG Fc polypeptide include SEQ ID NOs: 71, 72, 75, 76, 79, 80, 82, 372, 373, 83, 374, and 375.

Caninized or felinized light chain polypeptides may be fused to the light chain constant region of a companion animal species, such as a wild-type canine or feline light chain constant region (e.g., SEQ ID NO: 235 or 241), or variants of such polypeptides (e.g., SEQ ID NO: 236 or 242). Exemplary amino acid sequences of a caninized or felinized variable light chain and a κ light chain constant region include SEQ ID NOs: 73, 74, 77, 78, 81, 84, 376, 377, and 378.

Nucleotide sequences encoding chimeric polypeptides of Clone B and I variable heavy chain fused to canine or feline IgG and Clone B and I variable light chain fused to canine or feline constant light chain were synthesized and cloned into expression vectors suitable for transfection into a CHO host cell. Nucleotide sequences encoding caninized and felinized Clone I polypeptides were also synthesized and cloned into expression vectors. Each pair of HC and LC nucleotide sequences was transfected into a CHO host cell. The cells were cultured and Clone B, Clone I, Chimeric B, and Chimeric I antibodies were purified from the culture medium by Protein A column chromatography.

Vectors may also be used to perform pilot-scale transfection in CHO-S cells using the FreestyleMax™ transfection reagent (Life Technologies). The supernatant is harvested by clarifying the conditioned media. Antibodies may be purified with a single pass Protein A chromatography step and used for further investigation.

Purified antibody preparations may be admixed with one or more pharmaceutically acceptable excipients and sterilized by filtration to prepare a pharmaceutical composition of the invention. Exemplary antibody preparations or pharmaceutical compositions may be administered to a dog or cat with an IL4R-induced condition, such as atopic dermatitis, allergic dermatitis, pruritus, asthma, psoriasis, scleroderma, or eczema in a therapeutically effective amount.

### Example 13

### Variant Canine IgG Fc Polypeptides for Increased Protein a Binding and/or Decreased Complement Binding and/or Decreased CD16 Binding

Purification of antibodies using Protein A affinity is a well-developed process. However, among four subtypes of canine IgG, only IgG-B Fc (e.g., SEQ ID NO: 163 or SEQ ID NO: 164) has Protein A binding affinity. Canine IgG-A Fc (e.g., SEQ ID NO: 162), IgG-C Fc (e.g., SEQ ID NO: 165 or SEQ ID NO: 166), and IgG-D Fc (e.g., SEQ ID NO: 167) have weak or no measurable Protein A binding affinity. Variant canine IgG-A Fc, IgG-C Fc, and IgG-D Fc polypeptides were designed for altered Protein A binding.

In addition, canine IgG-B Fc and IgG-C Fc have complement activity and bind to C1q, while canine IgG-A Fc and IgG-D Fc have weak or no measurable binding affinity to C1q. To potentially reduce the C1q binding and/or potentially reduce complement-mediated immune responses, variant canine IgG-B Fc and IgG-C Fc polypeptides were designed.

Furthermore, canine IgG-B Fc and IgG-C Fc have CD16 binding activity. To potentially reduce the binding of CD16 to IgG-B Fc and IgG-C Fc, and/or potentially reduce ADCC, variant canine IgG-B Fc and IgG-C Fc polypeptides were designed.

Table 8, below summarizes the Protein A and C1q binding characteristics of canine IgG Fc subtypes. Notably, none of the wild-type canine IgG Fc subtypes lacks C1q binding and binds Protein A.

TABLE 8

| Wild-type | Protein A | C1q | CD16 |
|---|---|---|---|
| Canine IgG Fc | Binding | Binding | Binding |
| IgG-A Fc | − | − | − |
| IgG-B Fc | + | + | + |
| IgG-C Fc | − | + | + |
| IgG-D Fc | − | − | − |

(−) denotes low or no measurable binding activity.

Two approaches were used to design variant canine IgG-A, IgG-C, and IgG-D Fc polypeptides for increased Protein A binding. For the first approach, variant canine IgG-A, IgG-C, and IgG-D Fc polypeptides were designed to have the same Protein A binding motif sequences as canine IgG-B Fc (e.g., SEQ ID NO: 163, SEQ ID NO: 165, and SEQ ID NO: 167, respectively). For the second approach, variant canine IgG-A Fc I(21)T/Q(207)H (SEQ ID NO: 169), variant canine IgG-C Fc I(21)T (SEQ ID NO: 181), and variant canine IgG-D Fc I(21)T/Q(207)H (SEQ ID NO: 194) were designed with one or two amino acid substitutions in the Protein A binding region to correspond with the canine IgG-B Fc sequence.

In addition, variant canine IgG-A Fc, IgG-C Fc, and IgG-D Fc polypeptides with increased Protein A binding may be prepared having one or more of the amino acid substitutions listed in Table 9.

TABLE 9
Variant Canine IgG Fc Amino Acid Substitutions* (Protein A+)

| Canine IgG-A Fc | Canine IgG-C Fc | Canine IgG-D Fc |
|---|---|---|
| (SEQ ID NO: 162) | (SEQ ID NO: 165) | (SEQ ID NO: 167) |
| Ile (21) Thr | Ile (21) Thr | Ile (23) Thr |
| Arg (23) Leu | Val (23) Leu | Arg (23) Leu |
| Thr (25) Ala | Thr (24) Ile | Thr (25) Ala |
| Glu (80) Gly | | Glu (80) Gly |
| Thr (205) Ala | | Gln (207) His |
| Gln (207) His | | |

*The amino acid positions listed are relative to the SEQ ID NO. indicated.

To potentially reduce the binding of C1q to canine IgG-B Fc and IgG-C Fc, and/or potentially reduce complement-mediated immune responses, variant canine IgG-B Fc and IgG-C Fc polypeptides may be prepared having an amino acid substitution of Lys with any amino acid except Lys at an amino acid position corresponding to position 93 of SEQ ID NO: 163 or of SEQ ID NO: 165, respectively. These amino acid substitutions were identified after analysis of the protein sequence and 3-D structure modeling of canine IgG-B Fc and IgG-C Fc compared to canine IgG-A Fc and IgG-D Fc, which are understood to not exhibit complement activity. For example, variant canine IgG-B Fc K(93)R (SEQ ID NO: 170) and variant canine IgG-C Fc K(93)R (SEQ ID NO: 182) may be prepared. Reduced binding between human C1q and a fusion protein comprising variant canine IgG-B Fc K(93)R was observed when compared to a fusion protein comprising wild-type canine IgG-B Fc.

To potentially reduce the binding of CD16 to IgG-B Fc and IgG-C Fc, and/or potentially reduce ADCC, variant canine IgG-B Fc and IgG-C Fc polypeptides may be prepared having one or more of the amino acid substitutions listed in Table 10. The amino acid substitution(s) were identified after analysis of the protein sequence and 3-D structure modeling of canine IgG-B and IgG-C compared to IgG-A and IgG-D, which are understood to not exhibit ADCC activity.

TABLE 10

| Original residue position* | | |
|---|---|---|
| Canine IgG-B Fc | Canine IgG-C Fc | |
| (SEQ ID NO: 163) | (SEQ ID NO: 165) | Substitution(s) |
| Met (5) | Leu (5) | Any amino acid |
| | | except original |
| | | residue, such as Pro |
| Asp (38) | Asp (38) | Any amino acid |
| | | except original |
| | | residue, such as |
| | | Gly |
| Pro (39) | Pro (39) | Any amino acid |
| | | except original |
| | | residue, such as |
| | | Arg |
| Lys (97) | Lys (97) | Any amino acid |
| | | except original |
| | | residue, such as Ile |
| Ala (98) | Ala (98) | Any amino acid |
| | | except original |
| | | residue, such as |
| | | Gly |

*The amino acid positions listed are relative to the SEQ ID NO. indicated.

Since wild-type canine IgG-C Fc lacks Protein A binding and has C1q binding, a double variant canine IgG-C Fc that binds Protein A and has reduced binding to C1q may be prepared by combining one or more of the amino acid substitutions listed in Table 9 with a K(93)R substitution or K(93)X substitution, wherein X is any amino acid except Lys. A double variant canine IgG-B Fc or double variant canine IgG-C Fc with reduced binding to C1q and reduced binding to CD16 may be prepared by combining one or more of the amino acid substitutions listed in Table 10 with a K(93)R substitution or K(93)X substitution, wherein X is any amino acid except Lys. A triple variant canine-IgG-C Fc that binds Protein A and has reduced binding to C1q and CD16 may be prepared by combining one or more of the amino acid substitutions listed in Table 9 and one or more of the amino acid substitutions listed in Table 10 with a K(93)R substitution or K(93)X substitution, wherein X is any amino acid except Lys.

The binding of any variant canine IgG Fc to Protein A, CD16, and/or C1q may be determined and compared to the binding of another IgG Fc to Protein A, CD16, and/or C1q (e.g., the corresponding wild-type canine IgG Fc, another wild-type or variant canine IgG Fc, or a wild-type or variant IgG Fc of another companion animal, etc.).

Binding analysis may be performed using an Octet biosensor. Briefly, the target molecule (e.g., Protein A, C1q, CD16, etc.) may be biotinylated and free unreacted biotin removed (e.g., by dialysis). The biotinylated target molecule is captured on streptavidin sensor tips. Association of the target molecule with various concentrations (e.g., 10 μg/mL) of IgG Fc polypeptide is monitored for a specified time or until steady state is reached. Dissociation is monitored for a specified time or until steady state is reached. A buffer only blank curve may be subtracted to correct for any drift. The data are fit to a 1:1 binding model using ForteBio™ data analysis software to determine the kₒₙ, k_{off}, and the K_{d}.

Exemplary variant canine IgG polypeptides for increased Protein A binding (e.g., for ease of purification), decreased C1q binding (e.g., for reduced complement-mediated immune responses), reduced CD16 binding (e.g., reduced antibody-dependent cell-mediated cytotoxicity), and/or increased stability include SEQ ID NOs: 169, 170, 171, 172, 173, 174, 175, 176, 177, 178, 179, 180, 181, 182, 183, 184, 185, 186, 187, 188, 189, 190, 191, 192, 193, and 194. Such variant canine IgG polypeptides may be incorporated with Clone B or I variable heavy chain sequences, caninized Clone B or I variable heavy chain sequences, or CDR sequences described in the above examples.

### Example 14

### Variant Feline IgG Fc Polypeptides for Decreased Complement Binding and/or Enhanced Hinge Disulfide Formation and/or Enhanced Recombinant Production

Each of the three subtypes of feline IgG, IgG1a Fc (SEQ ID NO: 203 or SEQ ID NO: 204), IgG1b Fc (SEQ ID NO: 205 or SEQ ID NO: 206), and IgG2 Fc (SEQ ID NO: 207) have Protein A binding affinity. However, only feline IgG2 Fc has weak or no measurable binding affinity to C1q, while feline IgG1a Fc, IgG1b Fc bind to C1q. To potentially reduce the C1q binding and/or potentially reduce complement-mediated immune responses, variant feline IgG1a Fc and IgG1b Fc polypeptides were designed.

Table 11, below summarizes the Protein A and C1q binding characteristics of feline IgG Fc subtypes. Notably, none of the wild-type equine IgG Fc subtypes lacks C1q binding and binds Protein A.

TABLE 11

| Wild-type | Protein A | C1q |
|---|---|---|
| Feline IgG Fc | Binding | Binding |
| IgG1a Fc | + | + |
| IgG1b Fc | + | + |
| IgG2 Fc | + | − |

(−) denotes low or no measurable binding activity.

To potentially reduce the binding of C1q to feline IgG1a Fc and IgG1b Fc, and/or potentially reduce complement-mediated immune responses, variant feline IgG1a Fc and IgG1b Fc polypeptides may be prepared having an amino acid substitution of Pro with any amino acid except Pro at an amino acid position corresponding to position 198 of SEQ ID NO: 203, SEQ ID NO: 204, SEQ ID NO: 205, or SEQ ID NO: 206. These amino acid substitutions were identified after analysis of the protein sequence and 3-D structure modeling of feline IgG1a Fc and IgG1b Fc compared to feline IgG2 Fc, which is understood to not exhibit complement activity. For example, variant feline IgG1 Fc polypeptides P(198)A (SEQ ID NOs: 209, 210, 212, and 213) may be prepared.

The binding of any variant feline IgG Fc to C1q may be determined and compared to the binding of another IgG Fc to C1q (e.g., the corresponding wild-type feline IgG Fc, another wild-type or variant feline IgG Fc, or a wild-type or variant IgG Fc of another companion animal, etc.). The binding assay described in Example 13 may be used.

Three-dimensional protein modeling analysis of several ortholog hinge structures was used to modify feline IgG hinges to enhance disulfide formation. To enhance disulfide formation at the feline IgG hinge, the hinge sequence may be modified by substituting lysine with proline at a position corresponding to position 16 of feline IgG1a (SEQ ID NO: 203 or SEQ ID NO: 204) (e.g., K16P), feline IgG1b (SEQ ID NO: 205 or SEQ ID NO: 206), or feline IgG2 (SEQ ID NO: 207) (e.g., K(16)P). Examples of amino acid sequences of variant feline IgG polypeptides having a modified hinge include SEQ ID NO: 208, SEQ ID NO: 211, and SEQ ID NO: 215.

To increase disulfide formation at the feline IgG2 hinge, the hinge sequence may be modified by substituting an amino acid with cysteine. For example, a variant feline IgG2 Fc (SEQ ID NO: 214) having a modified hinge was prepared by substituting Gly with Cys at an amino acid position corresponding to position 14 of SEQ ID NO: 207.

Three-dimensional protein modeling was used to design feline variant IgG Fc polypeptides comprising sequences from the hinge region from a different IgG isotype for enhanced recombinant production and improved hinge disulfide formation. Variant feline IgG2 Fc polypeptides may be prepared that comprise sequences from the hinge region of feline IgG1a or IgG1b (e.g., SEQ ID NO: 216). Levels of recombinant production of variant IgG Fc polypeptides and/or levels of hinge disulfide formation may be determined and compared to that of another IgG Fc by SDS-PAGE analysis under reducing and non-reducing conditions (e.g., the corresponding wild-type IgG Fc of the same or different isotype, or a wild-type or variant IgG Fc of another companion animal, etc.).

Exemplary variant feline IgG polypeptides for decreased C1q binding (e.g., for reduced complement-mediated immune responses) and/or enhanced hinge disulfide formation and/or enhanced recombinant production include SEQ ID NOs: 208, 209, 210, 211, 212, 213, 214, 215, and 216. Such variant feline IgG polypeptides may be incorporated with Clone B or I variable heavy chain sequences, felinized Clone B or I variable heavy chain sequences, or CDR sequences described in the above examples.

### Example 15

### Variant Canine, Feline, and Equine IgG Fc Polypeptides for Bispecific Antibodies

To enable the preparation of a bispecific canine, feline, or equine antibody using a knob-in-hole heterodimerization approach, pairing of variant canine IgG Fc polypeptides, variant feline IgG Fc polypeptides, and variant equine IgG Fc polypeptides was investigated. Bispecific antibodies combine specificities of two antibodies against two different targets. First, heavy chain pairing was designed by introducing CH3 interfacing mutations so that one chain comprises a bulky amino acid (knob) and the other chain comprises smaller amino acids in the same general location (hole). Furthermore, to facilitate a heavy chain to specifically pair with its intended light chain, interface amino acids between CH1 and the light chain may be mutated to be complementary in shape and charge-charge interaction.

An amino acid substitution of tryptophan to tyrosine at a position corresponding to position 138 of canine IgG-A (SEQ ID NO: 162), at a position corresponding to position 137 of canine IgG-B Fc (SEQ ID NO: 163), at a position corresponding to position 137 of canine IgG-C Fc (SEQ ID NO: 165), or at a position corresponding to position 138 of canine IgG-D Fc (SEQ ID NO: 167) (T138W or T137W) can be introduced as a knob. Examples of amino acid sequences of a first variant canine IgG-A Fc, IgG-B Fc, IgG-C Fc, and IgG-D Fc are SEQ ID NO: 195, SEQ ID NO: 196, SEQ ID NO: 197, and SEQ ID NO: 198, respectively.

An amino acid substitution of threonine to serine at a position corresponding to position 138 and of leucine to alanine at a position corresponding to position 140 of canine IgG-A (SEQ ID NO: 162) or of IgG-D (SEQ ID NO: 167) (T138S, L140A), or of threonine to serine at a position corresponding to position 137 and of leucine to alanine at a position corresponding to position 139 of canine IgG-B Fc (SEQ ID NO: 163) or of IgG-C(SEQ ID NO: 165) (T137S, L139A) can be introduced as a hole. Examples of amino acid sequences of a second variant canine IgG-A Fc, IgG-B Fc, IgG-C Fc, and IgG-D Fc are SEQ ID NO: 199, SEQ ID NO: 200, SEQ ID NO: 201, and SEQ ID NO: 202.

An amino acid substitution of alanine to leucine at a position corresponding to position 24 and of serine to asparagine at a position corresponding to position 30 of a canine IgG-A CH1 (SEQ ID NO: 227), canine IgG-B CH1 (SEQ ID NO: 228), canine IgG-C CH1 (SEQ ID NO: 229), or canine IgG-D CH1 (SEQ ID NO: 230) (A24L, 530D) may be introduced. Examples of amino acid sequences of a variant canine IgG-A CH1, IgG-B CH1, IgG-C CH1, and IgG-D CH1 are SEQ ID NO: 231, SEQ ID NO: 232, SEQ ID NO: 233, and SEQ ID NO: 234.

An amino acid substitution of a phenylalanine to alanine at a position corresponding to position 11 and of serine to arginine at a position corresponding to position 22 of a canine κ constant region (SEQ ID NO: 235) (F11A, S22R) may be introduced. An example of an amino acid sequence of a variant canine κ constant region is SEQ ID NO: 236.

An amino acid substitution of threonine to tryptophan at a position corresponding to position 154 of feline IgG1a Fc (SEQ ID NO: 203 or SEQ ID NO: 204), feline IgG1b Fc (SEQ ID NO: 205 or SEQ ID NO: 206), or of feline IgG2 (SEQ ID NO: 207) (T154W) can be introduced as a knob. Examples of amino acid sequences of a first variant feline IgG2 Fc, IgG1a Fc, and IgG1b Fc are SEQ ID NO: 217, SEQ ID NO: 218, SEQ ID NO: 219, SEQ ID NO: 220, and SEQ ID NO: 221.

An amino acid substitution of threonine to serine at a position corresponding to position 154 and of leucine to alanine at a position corresponding to position 156 of feline IgG1a (SEQ ID NO: 203 or SEQ ID NO: 204), feline IgG-b Fc (SEQ ID NO: 205 or SEQ ID NO: 206), or feline IgG2 Fc (SEQ ID NO: 207) (T154S, L156A) can be introduced as a hole. Examples of amino acid sequences of a second variant feline IgG2 Fc, IgG1a Fc, and IgG1b Fc are SEQ ID NO: 222, SEQ ID NO: 223, SEQ ID NO: 224, SEQ ID NO: 225, and SEQ ID NO: 226.

An amino acid substitution of alanine to leucine at a position corresponding to position 24 and of serine to asparagine at a position corresponding to position 30 of feline IgG1 CH1 (SEQ ID NO: 237), or an amino acid substitution of alanine to leucine at a position corresponding to position 24 and of serine to asparagine at a position corresponding to position 29 of feline IgG2 CH1 (SEQ ID NO: 238) may be introduced. Examples of amino acid sequences of a variant feline IgG1 CH1 and IgG2 CH1 are SEQ ID NO: 239 and SEQ ID NO: 240.

An amino acid substitution of a phenylalanine to alanine at a position corresponding to position 11 and of serine to arginine at a position corresponding to position 22 of a feline κ constant region (SEQ ID NO: 241) (F11A, S22R) may be introduced. An example of an amino acid sequence of a variant feline κ constant region is SEQ ID NO: 242.

An amino acid substitution of threonine to tryptophan at a position corresponding to position 130 of equine IgG1 Fc (SEQ ID NO: 247), of equine IgG2 Fc (SEQ ID NO: 248), of equine IgG3 Fc (SEQ ID NO: 249), of equine IgG4 Fc (SEQ ID NO: 250), of equine IgG5 Fc (SEQ ID NO: 251), of equine IgG6 Fc (SEQ ID NO: 252), or of equine IgG7 Fc (SEQ ID NO: 253) (T130W) can be introduced as a knob. Examples of amino acid sequences of a first variant equine IgG1 Fc, IgG2 Fc, IgG3 Fc, IgG4 Fc, IgG5 Fc, IgG6 Fc, and IgG7 Fc are SEQ ID NO: 254, SEQ ID NO: 255, SEQ ID NO: 256, SEQ ID NO: 257, SEQ ID NO: 258, SEQ ID NO: 259, and SEQ ID NO: 260, respectively.

An amino acid substitution of threonine to serine at a position corresponding to position 130 and of leucine to alanine at a position corresponding to position 132 of equine IgG1 Fc (SEQ ID NO: 247), of equine IgG2 Fc (SEQ ID NO: 248), of equine IgG3 Fc (SEQ ID NO: 249), of equine IgG4 Fc (SEQ ID NO: 250), of equine IgG5 Fc (SEQ ID NO: 251), of equine IgG6 Fc (SEQ ID NO: 252), or of equine IgG7 Fc (SEQ ID NO: 253) (T130W) can be introduced as a hole. Examples of amino acid sequences of a second variant equine IgG1 Fc, IgG2 Fc, IgG3 Fc, IgG4 Fc, IgG5 Fc, IgG6 Fc, and IgG7 Fc are SEQ ID NO: 261, SEQ ID NO: 262, SEQ ID NO: 263, SEQ ID NO: 264, SEQ ID NO: 265, SEQ ID NO: 266, and SEQ ID NO: 267, respectively.

The above described approach may be used to prepare bispecific antibodies against IL4R and other targets, such as IL17, IL31, TNFα, CD20, CD19, CD25, IL4, IL13, IL23, IgE, CD11α, IL6R, α4-Intergrin, IL12, IL1β, or BlyS. For example, a bispecific antibody against canine IL4R and canine IL31 may be prepared with the amino acid sequences of SEQ ID NO: 243 (caninized Clone I variable HC v2 and variant IgG-B Fc C1q-, CD16-with bispecific knob), SEQ ID NO: 244 (caninized Clone I variable LC v2 and variable canine κ constant region), SEQ ID NO: 245 (caninized anti-canine IL31 Clone M14 variable HC and variant canine IgG-B Fc C1q-, CD16-with bispecific hole), and SEQ ID NO: 246 (caninized anti-canine IL31 Clone M14 variable LC and canine κ constant region).

### Example 16

### Identification of Additional Mouse Monoclonal Antibodies that Bind to Canine IL4R

Mouse monoclonal antibodies were identified using standard immunization using canine IL4R extracellular domains produced by 293 cells as the immunogen. Different adjuvants were used during immunizations (Antibody Solutions, Sunnyvale, CA) and monoclonal antibodies were obtained through standard hybridoma technology. Enzyme linked immunosorbent assay (ELISA) was developed to screen the clones that produce canine IL4R binding antibodies. First canine IL4R was biotinylated and then it was introduced to streptavidin-coated wells. Immunized serum was then added to the wells followed by washing and detection with HRP-conjugated anti-mouse antibodies. The presence of canine IL4R binding antibodies developed a positive signal. Over 100 ELISA-positive clones with high binding signals were identified.

Furthermore, a neutralization (canine IL4-blocking) ELISA was performed. Four clones were identified that their binding to canine IL4R can be inhibited by canine IL4. Binding of canine IL4 to canine IL4R was inhibited by four clones: M3, M5, M8 and M9.

The four mouse antibodies were purified from the hybridoma cell cultures and their affinities to canine IL4R were measured using biosensor (ForteBio OctetRed). Biotinylated canine IL4R was bound to the streptavidin sensor tip. The Kds of the 4 candidates were all less than 10 nM.

### Example 17

### Identification of VH and VL Sequences of M3, M5, M8, and M9

Hybridoma cells producing M3, M5, M8 and M9 were pelleted. RNA was extracted and oligonucleotide primers for amplifying mouse immunoglobulin (Ig) variable domains were used to obtain cDNA using standard techniques. The variable heavy chain (VH) and variable light chain (VL) of each of the four clones were determined (SEQ ID NO: 292 (M3 VH), SEQ ID NO: 293 (M3 VL), SEQ ID NO: 308 (M5 VH), SEQ ID NO: 309 (M5 VL), SEQ ID NO: 324 (M8 VH), SEQ ID NO: 325 (M8 VL), SEQ ID NO: 340 (M9 VH), SEQ ID NO: 341 (M9 VL).

### Example 18

### Expression and Purification of Murine-Canine Chimeric Antibodies from CHO Cells

DNA sequences encoding a chimeric antibody were designed for a fusion of murine VH and murine VL to canine constant heavy chain IgG-B and canine constant light chain (kappa). The nucleotide sequences were synthesized chemically and inserted into an expression vector suitable for transfection into a CHO host cell. After transfection into CHO cells, the light chain or heavy chain protein or both were secreted from the cell. All four chimeric canine IgG-B were purified by single step Protein A column chromatography. Their affinities to canine IL4R were confirmed to be <10 nM using biosensor (ForteBio OctetRed).

### Example 19

### Caninization and Expression from CHO Cells

Murine (M3, M9) VH and VL were caninized by searching and selecting proper canine germline antibody sequences as a template for CDR grafting, followed by protein modeling. Caninized variable light chain M3 (SEQ ID NO: 344) and caninized variable light chain M9 (SEQ ID NO: 347) were fused to canine CL kappa (resulting in SEQ ID NOs: 350 and 353, respectively) Caninized variable heavy chains of M3 (SEQ ID NO: 342 and SEQ ID NO: 343) and caninized variable heavy chains of M9 (SEQ ID NO: 344 and SEQ ID NO: 345) were fused to variant canine IgG-B resulting in SEQ ID NOs 348, SEQ ID NO: 349, SEQ ID NO: 351, and SEQ ID NO: 352, respectively). The heavy and light chains were readily expressed and purified in a single step with a protein A column.

The caninized antibodies expressed well and maintained binding affinity to canine IL4R of ≤1 nM.

### Example 20

### Development of Canine IL4R Cell-Based Signaling Assay

Canine DH82 cells, a canine macrophage-like cell line derived from Malignant histiocytosis was purchased from ATCC (CRL10389), were seeded at 10e5 cells/well in 96-well plates and incubated at 37° C., 5% CO₂ overnight (in 15% FBS D-MEM as recommended by ATCC). Serum starvation and antibody pre-incubation of cells were done by replacing medium in each well with serial diluted anti-IL4R or hybridoma supernatant preparations in D-MEM without FBS for 1 hour at 5% CO₂, 37° C. Then, canine IL4 cytokine (RnD AF754) was added at 1 ng/mL (final concentration) to each well for 15 min at 5% CO₂, 37° C. Twenty microliters of stop solution (M-per Thermo Fisher #78501) was added each well. The IL4-inducible STAT6 phosphorylation in each well was assayed by anti-phospho-STAT6 (RnD MAB3717) western blotting.

M3, M8 and M9 mouse antibodies inhibited STAT6 phosphorylation as assessed by anti-phospho STAT6 Western blot.

### Example 21

### Identification of M3, M8 and M9 Canine IL4R Binding Epitopes

Since M3, M8 and M9 did not recognize human IL4R based on Western blot, yet strongly react to canine IL4R, combinations chimeric canine-human IL4R fusion molecules were made and expressed to identify the minimal canine segment(s) to which each antibody binds.

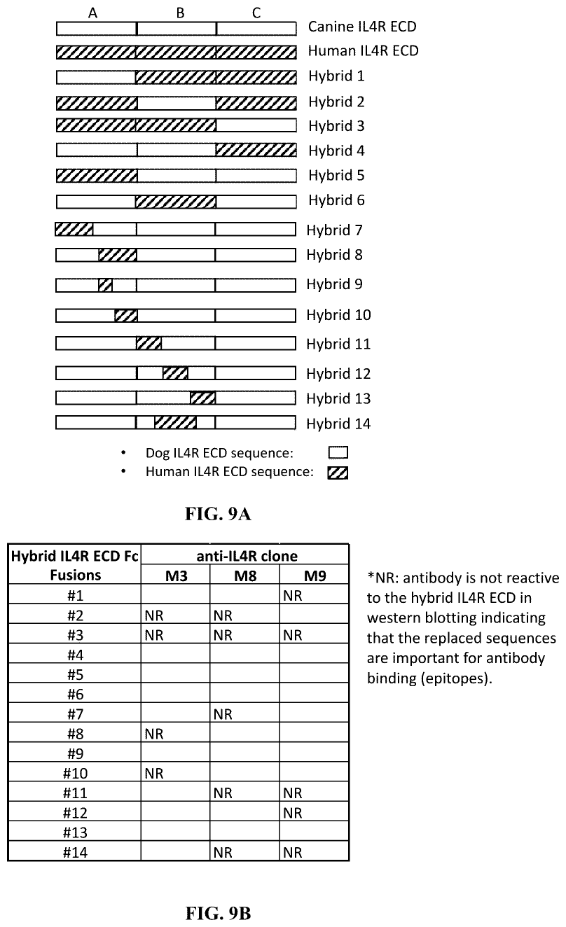

FIG. 9A illustrates the canine/human IL4R ECD hybrid polypeptides used for canine IL4R epitope mapping analyses (the same hybrid constructs used in the epitope mapping of Example 11, above). FIG. 9B summarizes the western blotting analysis in which canine IL4R ECD, human IL4R ECD, and the different canine/human IL4R ECD hybrid polypeptides were probed with M3, M8, and M9 antibodies. NR in the table represents that the antibody was not reactive to the hybrid IL4R ECD in the respective western blot indicating that the canine IL4R sequences replaced by human IL4R sequences are important for antibody binding (epitopes).

M3 was determined to bind an epitope having the sequence DFMGSENHTCVPEN (SEQ ID NO: 354). M8 was determined to bind to a first epitope having the sequence GSVKVLHEPSCFSDYISTSVCQWKMDHPTNCSA (SEQ ID NO: 355) and a second epitope having the sequence REDSVCVCSMPI (SEQ ID NO: 356). M9 was determined to bind to an epitope having the sequence REDSVCVCSMPIDDAVEADV (SEQ ID NO: 357).

### Example 22

### Screening Variant Canine IgG-B Polypeptides with Enhanced Canine FcRn/B2M Binding

Canine FcRn with a poly-His tag (SEQ ID NO: 379) and canine B2M (SEQ ID NO: 380) heterodimer complex was transiently expressed in HEK cells and purified using Ni-NTA chromatography.

Fast Screening for Expression, Biophysical Properties and Affinity (FASEBA) of canine IgG-B Fc phage libraries was performed. Briefly, the open reading frame of canine IgG-B Fc polypeptide was subcloned into plasmid pFASEBA. Based on three-dimensional protein modeling of the canine IgG-B/canine FcRn/canine B2M complex, twelve amino acid positions of canine IgG-B were identified as being potentially involved in the binding between IgG-B and FcRn/B2M. The twelve positions of canine IgG-B identified were Thr(21), Leu(22), Leu(23), Ile(24), Ala(25), Thr (27), Gly (80), His (81), Gln (82), Leu (85), Met (201), and Asn (207) of SEQ ID NO: 163 or SEQ ID NO: 164.

Twelve single site NNK mutation libraries of canine IgG-B Fc were prepared such that each library should have included variant IgG-B Fc polypeptides having each of the 20 possible amino acids substituted at each of the twelve sites. Each phage library was panned against canine FcRn/B2M complex at pH 6.0. After three rounds of panning, a total of 53 Fc phage clones were identified as potentially having enhanced FcRn/B2M binding and the mutations were identified by sequencing.

Single E. coli colonies expressing each of the 53 variant canine IgG-B Fc polypeptides with an SASA tag were cultured and induced to express the Fc polypeptides. Cell culture media containing the variant canine IgG-B Fc polypeptides was exposed to immobilized BSA either on a plate or a Biacore chip. The plates or chips with bound variant canine IgG-B Fc polypeptides were exposed to soluble canine FcRn/B2M complex to screen for slow off rate (koff) at pH 6. Each variant IgG-B Fc polypeptide exhibiting a slower koff with canine FeRn/B2M complex compared to wildtype IgG-B Fc polypeptide was identified. Four lead variant canine IgG-B polypeptides were identified: L(23)Y (SEQ ID NO: 382; “Y00”); L(23)F (SEQ ID NO: 381; “F00”); L(23)M; and L(23)S.

The koff of each of the lead variant canine IgG-B polypeptides was further investigated. Biotinylated canine FcRn/B2M complex was immobilized on a Biacore chip and exposed to each variant canine IgG-B polypeptide as an analyte using a Biacore T200 at pH 6.0. The koff (1/s) for wild-type canine IgG-B Fc polypeptide was 1.22×10⁻¹; the koff (1/s) for variant canine IgG-B Fc polypeptide L(23)Y (“Y00”) was 1.38×10⁻²; the koff (1/s) for variant IgG-B Fc polypeptide L(23)F (“F00”) was 6.31×10⁻² and 8.47×10⁻²; the koff (1/s) for variant canine IgG-B polypeptide L(23)M was 1.26×10⁻¹; and the koff (1/s) for variant canine IgG-B polypeptide L(23)S was 2.41×10⁻¹.

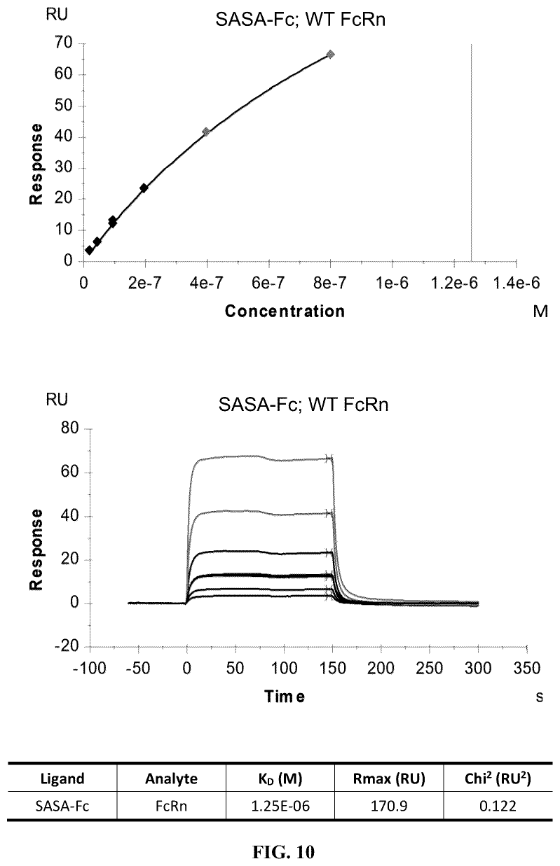

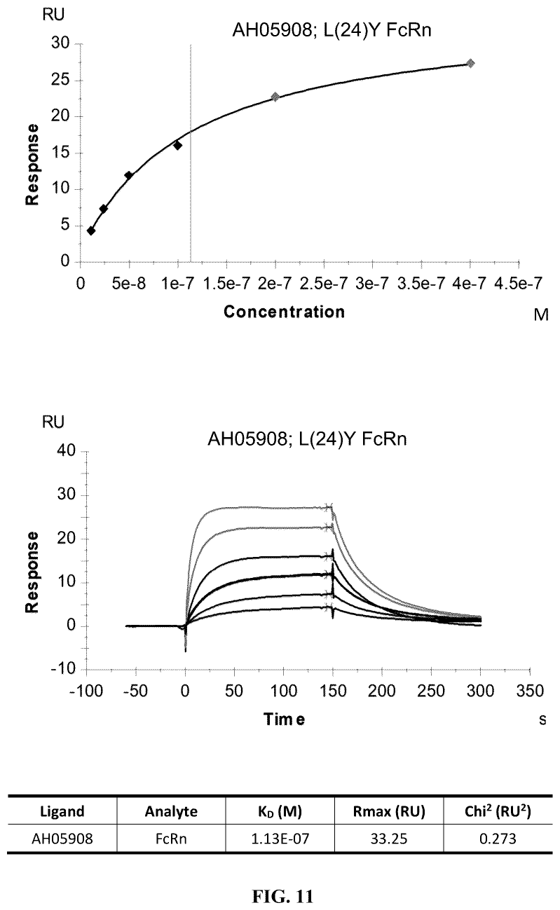

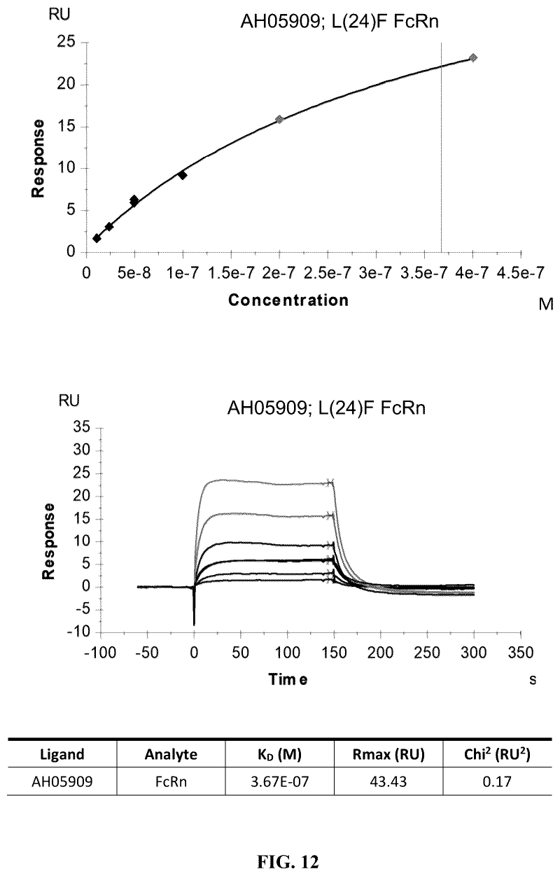

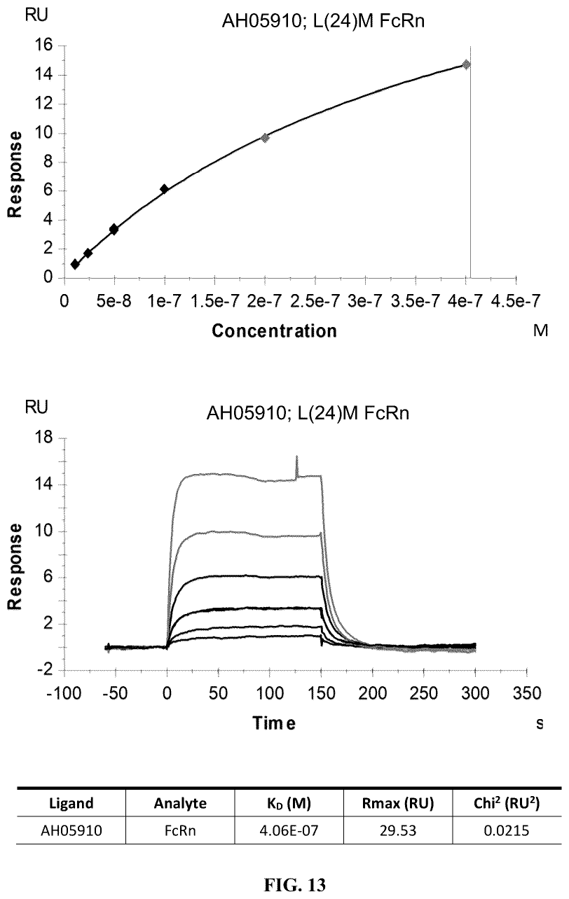

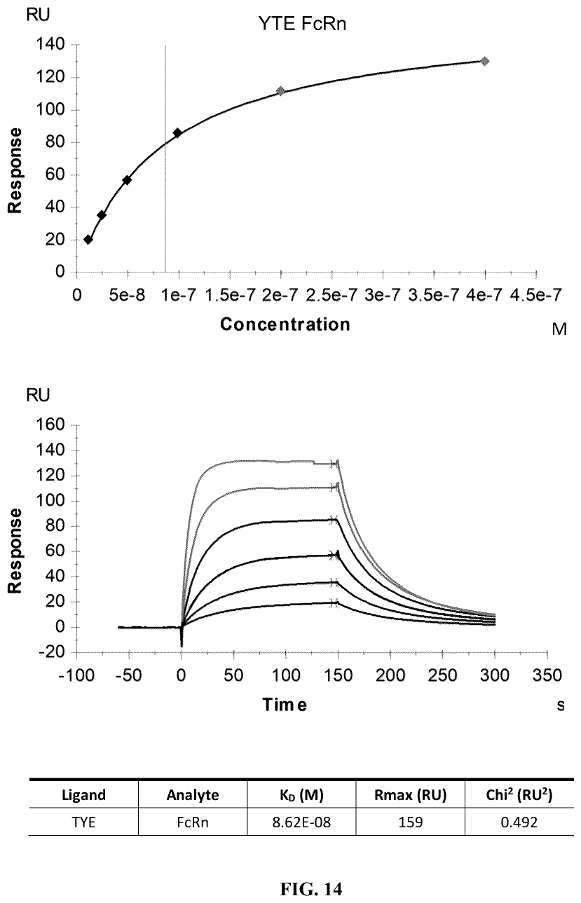

Binding analysis was performed using a Biacore T200. Briefly, the lead variant canine IgG-B Fc polypeptides with an SASA tag were each immobilized to a Series S Sensor Chip CM5. Association of each variant IgG-B Fc polypeptide with various concentrations of canine FcRn/B2M complex (12.5, 25, 50, 100, and 200 nM) was monitored at 25° C. until steady state was reached. A running buffer of 10 mM HEPES, 500 mM NaCl, 3 mM EDTA, 0.005% Tween-20, pH 6.0 was used. A buffer only blank curve was used as a control. The results are presented in FIGS. 10-14. The steady state Kd for wild-type canine IgG-B Fc polypeptide was 1.25×10⁻⁶ (FIG. 10); the steady state Kd for variant canine IgG-B Fc polypeptide L(23)Y (“Y00”) was 1.13×10⁻⁷ (FIG. 11); the steady state Kd for variant canine IgG-B Fc polypeptide L(23)F (“F00”) was 3.67×10⁻⁷ (FIG. 12); and the steady state Kd for variant canine IgG-B Fc polypeptide L(23)M was 4.06×10⁻⁷ (FIG. 13); and the steady state Kd for variant canine IgG-B Fc polypeptide YTE was 8.62×10⁻⁸ (FIG. 14).

### Example 23

### Phe Mutation in Canine IgG Enhances Canine FcRn Interaction

The affinity of variant canine Fc polypeptides for FcRn was evaluated in the context of a chimeric antibody. Antibody variable light chains fused to canine kappa light chain and variable heavy chains fused to variant canine IgG-A Fc polypeptides comprising SEQ ID NO: 383 (F00; Protein A+; C1q−; CD16−) or SEQ ID NO: 384 (Protein A+; C1q+; CD16+) and to variant canine IgG-D Fc polypeptides comprising SEQ ID NO: 385 (F00; Protein A+; C1q−; CD16−), or SEQ ID NO: 386 (Protein A+; C1q+; CD16+) were expressed.

The binding analysis was performed using a biosensor OctetRed as follows. Briefly, biotinylated TNFα was captured on streptavidin sensor tips. The association of antibody at 20 μg/mL was bound to TNFα. The complex was then used to bind to canine FcRn (50 μg/mL) at pH 6.0. Dissociation was performed at pH 7.2.

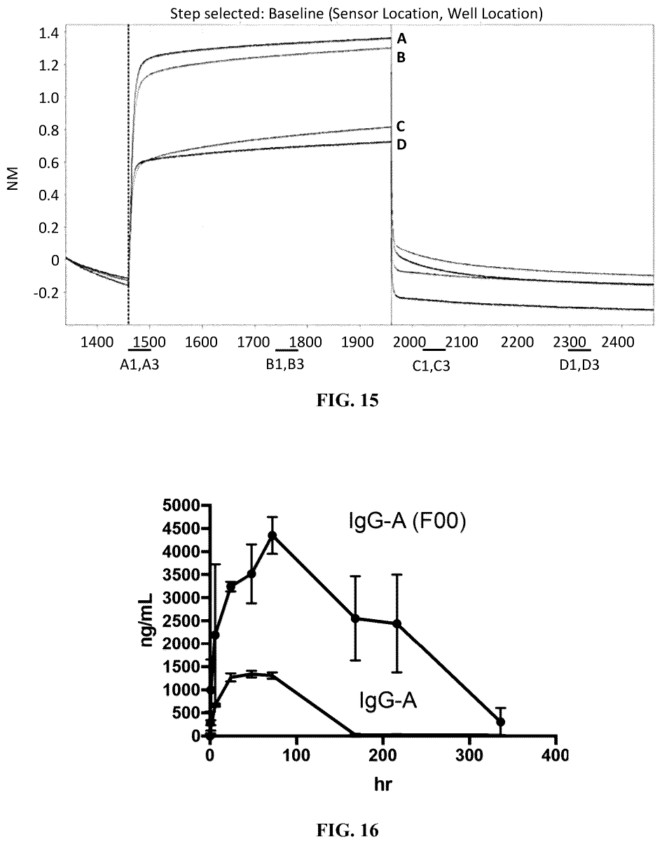

The Phe mutation enhanced canine FcRn binding at low pH (pH6.0, 20 mM NaCitrate, 140 mM NaCl), as illustrated by the binding profiles of chimeric variant canine IgG-A “F00” antibody (FIG. 15, A) and IgG-D “F00” antibody (FIG. 15, B) compared to chimeric variant canine IgG-A without the Phe mutation (FIG. 15, C) and IgG-D without the Phe mutation (FIG. 15, D). The chimeric variant canine IgG-A and IgG-D antibodies with the Phe mutation (FIG. 15, A and B) exhibited enhanced association with canine FcRn at low pH (pH 6.0) and fast dissociation at neutral pH (PBS pH7.2). A similar enhanced binding profile was also observed with chimeric variant canine IgG-B “F00” antibody.

### Example 24

### Pharmacokinetics of Phe Mutation in Canine IgG

Pharmacokinetics analysis was performed using Sprague Dawley rats. The rats were subcutaneously administered with 2 mg/kg of chimeric variant canine IgG-A “F00” antibody and chimeric variant canine IgG-A without the Phe mutation (two rats per group). Serum samples were collected from the rats at pre-injection and at 0.5, 1, 6, 24, 48, 72, 168, 216, and 336 hours post injection. The canine chimeric antibody concentrations in the serum samples were determined by ELISA, as follows.

Capture antibody (1 μg/mL in PBS) was coated on a 96-well Maxisorp plate with 100 μl in each well. The plate was incubated overnight at 4° C. and washed five times with PBST (PBS containing 0.05% Tween-20). Each well was blocked with 200 μl 5% BSA in PBST and the plate incubated for 1 hour at room temperature. The plate was washed five times with PBST. Dilutions of control antibody (1,000 ng/mL to 0.1 ng/mL) were added to the plate in duplicate and along with a blank well containing no control antibody were used to generate a standard curve. The serum samples were prepared by 10-fold, 20-fold, and 40-fold dilutions in 5% BSA-PBST and added to the plate. The plate was incubated at room temperature for 1 hour and washed 5 times with PBST. 100 μl HRP-conjugated antibody (Bio-Rad, catalog no. HCA204P) was added to each well at 0.25 μg/mL in 5% BSA-PBST. The plate was incubated for 1 hour at room temperature and washed 5 times with PBST. 100 μl QuantaBlu (Thermo Scientific, catalog no. 15169) was added to each well. The fluorescence was measured after 10⁻¹⁵ minutes incubation at 325 nm/420 nm (emission/excitation). The titer of anti-TNFα in the serum samples was calculated against the standard curve.

The AUC_{0-336 h} for IgG-A was 150970, while IgG-A “F00” was 848924 ng/mL*hr (FIG. 16). The terminal half-life was estimated to be 33 hours and 152 hours, respectively. Thus, the single Phe mutation significantly improved the pharmacokinetic profile of the antibody in rat.

### Example 25

### Phe Mutation in Canine, Feline, and Equine IgG Fcs

The interaction between the Phe mutation in canine IgG-A, IgG-B, IgG-C, and IgG-D Fc and FcRn was modeled using three-dimensional protein structure analysis. The aromatic side chain of Phe appears to have a hydrophobic interaction with canine FcRn at the Pro hydrophobic ring (π-CH) of the “WPE” motif. In addition, the Phe hydrophobic side chain may be in direct contact with the Glu side chain next to the Pro of the same “WPE” motif. This interaction may have energy penalty if the Glu side chain is deprotonated to be negative charged, such as at a neutral pH. Thus, some level of protonation of the Glu residue may be required to minimize the aromatics to Glu-H interaction. That may explain why the interaction between variant IgGs having the Phe mutation and FcRn is reduced at neutral pH. Based on protein structure analysis, the interaction appears to be conserved among canine IgG-A, IgG-B, IgG-C, and IgG-D Fc.

Furthermore, the interactions between a Phe mutation in feline IgG1a and IgG2 Fc were modeled when complexed with feline FcRn. The same interactions observed with the canine IgG Fcs appeared to be conserved with the feline IgG Fcs.

The interactions between a Phe mutation in equine IgG1, IgG2, IgG3, IgG4, IgG5, IgG6, and IgG7 Fc in complex with equine FcRn were also modeled. The same interactions appeared to be maintained with the equine IgG Fcs.

### Example 26

### Other Exemplary Variant Canine IgG Fcs Enhance Canine FcRn Interaction

The affinity of additional variant canine Fc polypeptides for FcRn was evaluated in the context of a chimeric antibody. Antibody variable light chain fused to canine kappa light chain and variable heavy chain sequences fused to wild-type IgG-B Fc polypeptide (comprising SEQ ID NO: 163), variant canine IgG-B Fc polypeptide 0Y0 (comprising SEQ ID NO: 387), variant canine IgG-B Fc polypeptide 0YH (comprising SEQ ID NO: 388), variant canine IgG-B Fc polypeptide 0YY (comprising SEQ ID NO: 389), and variant canine IgG-B Fc polypeptide 00Y (comprising SEQ ID NO: 390) were expressed.

The binding analysis was performed using a biosensor OctetRed as follows. Briefly, biotinylated target was captured on streptavidin sensor tips. The association of antibody at 20 μg/mL was bound to the biotinylated target. The complex was then used to bind to canine FcRn (50 μg/mL) at pH 6.0. Dissociation was performed at pH 7.2.

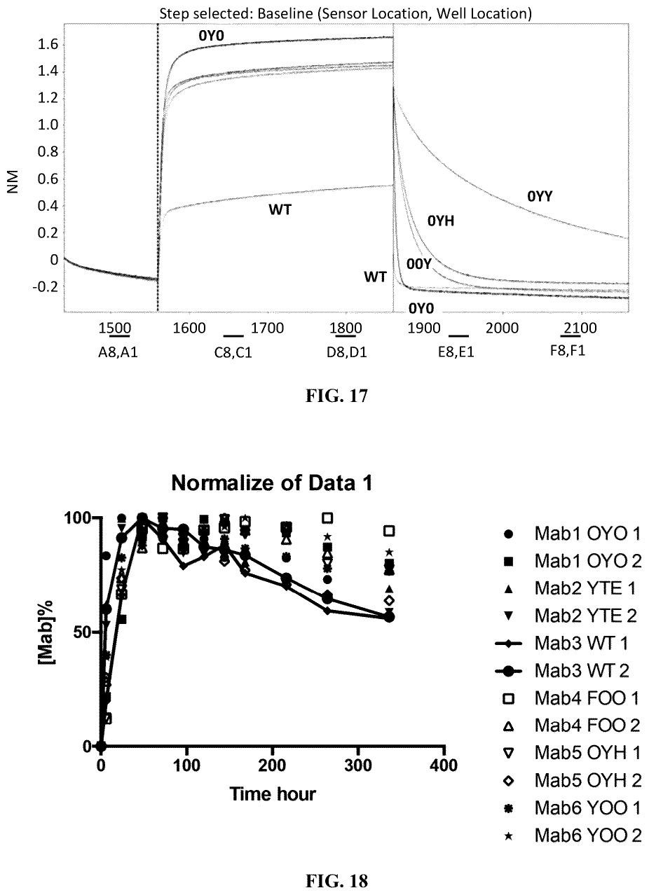

Each of the chimeric variant canine IgG-B antibodies exhibited enhanced binding to canine FcRn at pH 6.0 compared to the chimeric wild-type canine IgG-B antibody and each had an appreciable rate of dissociation at neutral pH (FIG. 17).

### Example 27

### Variant Canine IgG Fcs Extend Half-Life of Antibodies In Vivo in Canine

In vivo half-life of variant canine Fc polypeptides for FcRn was evaluated in the context of a chimeric antibody. Antibody variable light chain fused to canine kappa light chain and variable heavy chains fused to wild-type IgG-B Fc polypeptide (comprising SEQ ID NO: 163), variant canine IgG-B Fc polypeptide YTE (comprising SEQ ID NO: 391), variant canine IgG-B Fc polypeptide 0Y0 (comprising SEQ ID NO: 387), variant canine IgG-B Fc polypeptide F00 (comprising SEQ ID NO: 381), variant canine IgG-B Fc polypeptide 0YH (comprising SEQ ID NO: 388), and variant canine IgG-B Fc polypeptide Y00 (comprising SEQ ID NO: 382) were expressed and purified to 40 mg/mL in PBS, pH7.2.

Canine pharmacokinetics were performed at Absorption Systems California, LLC. Male beagles (˜8-14 kg) were obtained from Marshall Bioresources, North Rose, New York. A total of 12 dogs were used for study with n=2 dogs per group. The six antibodies were subcutaneously administered to the dogs at 4 mg/Kg. Serum samples were collected at pre-injection and at 6, 24, 48, 72, 96, 120, 144, 168, 216, 264, 336, 504 and 672 hours post-injection. The canine chimeric antibody concentrations were determined by ELISA as described. The Cp between time at 144 hour and 336 hour was transformed to Ln [Cp], then fit to linear equation in the form of Ln[Cp]ₜ=−k*t+Ln[Cp]_{144 h}. The terminal half-life was then calculated from slope k, as listed in Table 12, below. The 0Y0, F00, 0YH, and Y00 mutations in canine IgG-B Fc greatly improved the half-life of the antibody in vivo in dogs.

TABLE 12
Effect of variant canine IgG Fcs on antibody half-life in dog

| Dog | Half-life (days) |
|---|---|
| WT 1 | 13 |
| WT 2 | 13 |
| YTE 1 | *NR |
| YTE 2 | 15 |
| 0Y0 1 | *NR |
| 0Y0 2 | 28 |
| F00 1 | *NR |
| F00 2 | 23 |
| 0YH 1 | 22 |
| 0YH 2 | 23 |
| Y00 1 | 33 |
| Y00 2 | 39 |

*NR: result was not reliable

1.-15. (canceled)

16. An isolated antibody that binds to canine IL4R or feline IL4R, wherein the antibody comprises a heavy chain comprising:
a) a CDR-H1 sequence having at least 85%, at least 90%, at least 95%, at least 98%, or 100% sequence identity to the amino acid sequence of GYTFTSYVMX₁ (SEQ ID NO: 1), wherein X₁ is H or N;
b) a CDR-H2 sequence having at least 85%, at least 90%, at least 95%, at least 98%, or 100% sequence identity to the amino acid sequence of YINPX₂NDGTFYX₃GX₄X₅X₆G (SEQ ID NO: 2), wherein X₂ is K, A, or N, X₃ is N or A, X₄ is K or A, X₅ is F or V, and X₆ is K or Q, or YINPX₂NDGT (SEQ ID NO: 268), wherein X₂ is K, A, or N; and
c) a CDR-H3 sequence having at least 85%, at least 90%, at least 95%, at least 98%, or 100% sequence identity to the amino acid sequence of FX₇YGX₈AY (SEQ ID NO: 3), wherein X₇ is N or Y, and X₈ s I or F; and
wherein the antibody comprises a variant canine or feline IgG Fc polypeptide capable of binding to neonatal Fc receptor (FcRn) with an increased affinity relative to the wild-type Fc polypeptide.

17. The isolated antibody of claim 16, comprising a heavy chain comprising:
a) a CDR-H1 sequence having at least 85%, at least 90%, at least 95%, at least 98%, or 100% sequence identity to the amino acid sequence of GYTFTSYVMX₁ (SEQ ID NO: 1), wherein X₁ is H or N;
b) a CDR-H2 sequence having at least 85%, at least 90%, at least 95%, at least 98%, or 100% sequence identity to the amino acid sequence of YINPX₂NDGTFYX₃GX₄X₅X₆G (SEQ ID NO: 2), wherein X₂ is K, A, or N, X₃ is N or A, X₄ is K or A, X₅ is F or V, and X₆ is K or Q, or YINPX₂NDGT (SEQ ID NO: 268), wherein X₂ is K, A, or N; and
c) a CDR-H3 sequence having at least 85%, at least 90%, at least 95%, at least 98%, or 100% sequence identity to the amino acid sequence of FX₇YGX₈AY (SEQ ID NO: 3), wherein X₇ is N or Y, and X₈ s I or F.

18. The isolated antibody of claim 16, comprising a heavy chain comprising:
a) a CDR-H1 sequence having at least 85%, at least 90%, at least 95%, at least 98%, or 100% sequence identity to the amino acid sequence of SEQ ID NO: 7, SEQ ID NO: 29, or SEQ ID NO: 358;
b) a CDR-H2 sequence having at least 85%, at least 90%, at least 95%, at least 98%, or 100% sequence identity to the amino acid sequence of SEQ ID NO: 8, SEQ ID NO: 269, SEQ ID NO: 30, SEQ ID NO: 271, SEQ ID NO: 359, or SEQ ID NO: 272; and
c) a CDR-H3 sequence having at least 85%, at least 90%, at least 95%, at least 98%, or 100% sequence identity to the amino acid sequence of SEQ ID NO: 9 or SEQ ID NO: 31.

19. The isolated antibody of claim 16, comprising a heavy chain comprising:
a) a CDR-H1 comprising the amino acid sequence of SEQ ID NO:7, SEQ ID NO: 29, or SEQ ID NO: 358;
b) a CDR-H2 comprising the amino acid sequence of SEQ ID NO: 8, SEQ ID NO: 269, SEQ ID NO: 30, SEQ ID NO: 271, SEQ ID NO: 359, or SEQ ID NO: 272; and
c) a CDR-H3 comprising the amino acid sequence of SEQ ID NO: 9 or SEQ ID NO: 31.

20. (canceled)

21. The isolated antibody of claim 16, comprising a light chain comprising:
a) a CDR-L1 sequence having at least 85%, at least 90%, at least 95%, at least 98%, or 100% sequence identity to the amino acid sequence of RASQEISGYLX₉ (SEQ ID NO: 4) wherein X₉ is S or A;
b) a CDR-L2 sequence having at least 85%, at least 90%, at least 95%, at least 98%, or 100% sequence identity to the amino acid sequence of AASX₁₀X₁₁DX₁₂ (SEQ ID NO: 5), wherein X₁₀ is T or N, X₁₁ is R or L, and X₁₂ is S or T; and
c) a CDR-L3 sequence having at least 85%, at least 90%, at least 95%, at least 98%, or 100% sequence identity to the amino acid sequence of X₁₃QYASYPWT (SEQ ID NO: 6), wherein X₁₃ is V or L.

22. An isolated antibody that binds to canine IL4R or feline IL4R, wherein the antibody comprises:
a) a CDR-H1 sequence having at least 85%, at least 90%, at least 95%, at least 98%, or 100% sequence identity to the amino acid sequence of GYTFTSYVMX₁ (SEQ ID NO: 1), wherein X₁ is H or N;
b) a CDR-H2 sequence having at least 85%, at least 90%, at least 95%, at least 98%, or 100% sequence identity to the amino acid sequence of YINPX₂NDGTFYX₃GX₄X₅X₆G (SEQ ID NO: 2), wherein X₂ is K, A, or N, X₃ is N or A, X₄ is K or A, X₅ is F or V, and X₆ is K or Q, or YINPX₂NDGT (SEQ ID NO: 268), wherein X₂ is K, A, or N; and
c) a CDR-H3 sequence having at least 85%, at least 90%, at least 95%, at least 98%, or 100% sequence identity to the amino acid sequence of FX₇YGX₉AY (SEQ ID NO: 3), wherein X₇ is N or Y, and X₈ s I or F;
d) a CDR-L1 sequence having at least 85%, at least 90%, at least 95%, at least 98%, or 100% sequence identity to the amino acid sequence of RASQEISGYLX₉ (SEQ ID NO: 4) wherein X₉ is S or A;
e) a CDR-L2 sequence having at least 85%, at least 90%, at least 95%, at least 98%, or 100% sequence identity to the amino acid sequence of AASX₁₀X₁₁DX₁₂ (SEQ ID NO: 5), wherein X₁₀ is T or N, X₁₁ is R or L, and X₁₂ is S or T; and
f) a CDR-L3 sequence having at least 85%, at least 90%, at least 95%, at least 98%, or 100% sequence identity to the amino acid sequence of X₁₃QYASYPWT (SEQ ID NO: 6), wherein X₁₃ is V or L; and
wherein the antibody comprises a variant canine or feline IgG Fc polypeptide capable of binding to neonatal Fc receptor (FcRn) with an increased affinity relative to the wild-type Fc polypeptide.

23. The isolated antibody of claim 16, comprising a light chain comprising:
a) a CDR-L1 sequence having at least 85%, at least 90%, at least 95%, at least 98%, or 100% sequence identity to the amino acid sequence of SEQ ID NO: 14, SEQ ID NO: 36, or SEQ ID NO: 360;
b) a CDR-L2 sequence having at least 85%, at least 90%, at least 95%, at least 98%, or 100% sequence identity to the amino acid sequence of SEQ ID NO: 15, SEQ ID NO: 37, SEQ ID NO: 361, or SEQ ID NO: 362; and
c) a CDR-L3 sequence having at least 85%, at least 90%, at least 95%, at least 98%, or 100% sequence identity to the amino acid sequence of SEQ ID NO: 16 or SEQ ID NO: 38.

24. The isolated antibody of claim 16, comprising a light chain comprising:
a) a CDR-L1 comprising the amino acid sequence of SEQ ID NO: 14, SEQ ID NO: 36, or SEQ ID NO: 360;
b) a CDR-L2 comprising the amino acid sequence of SEQ ID NO: 15, SEQ ID NO: 37, SEQ ID NO: 361, or SEQ ID NO: 362; and
c) a CDR-L3 comprising the amino acid sequence of SEQ ID NO:16 or SEQ ID NO: 38.

25. The antibody of claim 16, further comprising one or more of (a) a variable region heavy chain framework 1 (HC-FR1) sequence of SEQ ID NO: 10 or SEQ ID NO: 32; (b) a HC-FR2 sequence of SEQ ID NO: 11 or SEQ ID NO: 33; (c) a HC-FR3 sequence of SEQ ID NO: 12, SEQ ID NO: 270, SEQ ID NO: 34, SEQ ID NO: 273; (d) a HC-FR4 sequence of SEQ ID NO: 13 or SEQ ID NO: 35; (e) a variable region light chain framework 1 (LC-FR1) sequence of SEQ ID NO: 17 or SEQ ID NO: 39; (f) an LC-FR2 sequence of SEQ ID NO: 18 or SEQ ID NO: 40; (g) an LC-FR3 sequence of SEQ ID NO: 19 or SEQ ID NO: 41; or (h) an LC-FR4 sequence of SEQ ID NO: 20 or SEQ ID NO: 42.

26. The antibody of claim 16, wherein the antibody comprises:
a. (i) a variable heavy chain sequence having at least 85%, at least 90%, at least 95%, at least 98%, or 100% sequence identity to the amino acid sequence of SEQ ID NO: 21 or SEQ ID NO: 43; (ii) a variable light chain sequence having at least 85%, at least 90%, at least 95%, at least 98%, or 100% sequence identity to the amino acid sequence of SEQ ID NO: 22 or SEQ ID NO: 44; or (iii) a variable heavy chain sequence as in (i) and a variable light chain sequence as in (ii); or
b. (i) a variable heavy chain sequence having at least 85%, at least 90%, at least 95%, at least 98%, or 100% sequence identity to the amino acid sequence of SEQ ID NO: 59, SEQ ID NO: 60, SEQ ID NO: 63, SEQ ID NO: 64, SEQ ID NO: 274, or SEQ ID NO: 363; (ii) a variable light chain sequence having at least 85%, at least 90%, at least 95%, at least 98%, or 100% sequence identity to the amino acid sequence of SEQ ID NO: 61, SEQ ID NO: 62, SEQ ID NO: 65, SEQ ID NO: 66, SEQ ID NO: 275, or SEQ ID NO: 364; or (iii) a variable heavy chain sequence as in (i) and a variable light chain sequence as in (ii); or
c. (i) a variable heavy chain sequence having at least 85%, at least 90%, at least 95%, at least 98%, or 100% sequence identity to the amino acid sequence of SEQ ID NO: 67 or SEQ ID NO: 69; (ii) a variable light chain sequence having at least 85%, at least 90%, at least 95%, at least 98%, or 100% sequence identity to the amino acid sequence of SEQ ID NO: 68 or SEQ ID NO: 70; or (iii) a variable heavy chain sequence as in (i) and a variable light chain sequence as in (ii).

27.-29. (canceled)

30. An isolated antibody, wherein the antibody comprises:
a) a variable heavy chain sequence comprising the amino acid sequence of SEQ ID NO: 21 or SEQ ID NO: 43, and a variable light chain sequence comprising the amino acid sequence of SEQ ID NO: 22 or SEQ ID NO: 44;
b) a variable heavy chain sequence comprising the amino acid sequence of SEQ ID NO: 59, SEQ ID NO: 60, SEQ ID NO: 63, SEQ ID NO: 64, SEQ ID NO: 274, or SEQ ID NO: 363, and a variable light chain sequence comprising the amino acid sequence of SEQ ID NO: 61, SEQ ID NO: 62, SEQ ID NO: 65, SEQ ID NO: 66, SEQ ID NO: 275, or SEQ ID NO: 364; or
c) a variable heavy chain sequence comprising the amino acid sequence of SEQ ID NO: 67, SEQ ID NO: 69, SEQ ID NO: 365, or SEQ ID NO: 366, and a variable light chain sequence comprising the amino acid sequence of SEQ ID NO: 68, SEQ ID NO: 70, SEQ ID NO: 367, SEQ ID NO: 368, or SEQ ID NO: 369, and
wherein the antibody comprises a variant canine or feline IgG Fc polypeptide capable of binding to neonatal Fc receptor (FcRn) with an increased affinity relative to the wild-type Fc polypeptide.

31.-129. (canceled)

130. The antibody of claim 16, wherein the antibody is a bispecific antibody that binds to IL4R and one or more antigens selected from IL17, IL31, TNFα, CD20, CD19, CD25, IL4, IL13, IL23, IgE, CD11α, IL6R, α4-Intergrin, IL12, IL1β, or BlyS.

131. (canceled)

132. The antibody of claim 16, wherein the antibody is an antibody fragment, optionally such as an Fv, scFv, Fab, Fab′, F(ab′)₂, or Fab′-SH fragment.

133. An isolated nucleic acid encoding the antibody of claim 16.

134. A host cell comprising the nucleic acid of claim 133.

135. (canceled)

136. A method of producing an antibody comprising culturing the host cell of claim 134 and isolating the antibody.

137. A pharmaceutical composition comprising the antibody of claim 16 and a pharmaceutically acceptable carrier.

138. A method of treating a companion animal species having an IL4/1L13-induced condition, the method comprising administering to the companion animal species a therapeutically effective amount of the antibody of claim 16 or a composition thereof.

139.-145. (canceled)

146. A method of reducing IL4 and/or IL13 signaling function in a cell, the method comprising exposing to the cell the antibody of claim 16 or a composition thereof under conditions permissive for binding of the antibody to extracellular IL4 and/or IL13, thereby reducing binding of IL4 and/or IL13 to IL4R and/or reducing IL4 and/or IL13 signaling function by the cell.

147.-151. (canceled)

152. A method for detecting IL4R in a sample from a companion animal species comprising contacting the sample with the antibody of claim 16 or a composition thereof under conditions permissive for binding of the antibody to IL4R.

153. (canceled)
